(19) United States
(12) Patent Application Publication
(10) Pub. No.: US 2026/0070935 A1
(43) Pub. Date: Mar. 12, 2026

(54) BISPHOSPHONATE LIPIDS, LIPID NANOPARTICLE COMPOSITIONS COMPRISING THE SAME, MINERAL TISSUE-ADSORBED COMPOSITIONS THEREOF, AND METHODS OF USE THEREOF

(71) Applicant: The Trustees of the University of Pennsylvania, Philadelphia, PA (US)

(72) Inventors: Michael J. Mitchell, Philadelphia, PA (US); Lulu Xue, Philadelphia, PA (US); Drew Weissman, Philadelphia, PA (US); Mohamad-Gabriel Alameh, Philadelphia, PA (US); Kyle Vining, Philadelphia, PA (US); ll-Chul Yoon, Philadelphia, PA (US); Qinyuan Chen, Philadelphia, PA (US); Jingyi Liu, Philadelphia, PA (US)

(21) Appl. No.: 19/389,975

(22) Filed: Nov. 14, 2025

Related U.S. Application Data
(63) Continuation-in-part of application No. 18/865,098.
(60) Provisional application No. 63/341,753, filed on May 13, 2022.
(60) Provisional application No. 63/721,206, filed on Nov. 15, 2024.

(51) Int. Cl. C07F 9/6509; A61K 9/51; A61K 31/7105; A61K 47/69; A61K 48/00; A61L 27/36; A61L 27/38; A61L 27/54

(52) U.S. Cl. CPC C07F 9/650952; A61K 9/5123; A61K 31/7105; A61K 47/6929; A61K 48/0008; A61L 27/3608; A61L 27/3612; A61L 27/365; A61L 27/3683; A61L 27/3865; A61L 27/54; A61L 2400/12; A61L 2430/02; A61L 2430/06

(57) ABSTRACT

Described herein, in part, are bisphosphonate lipid compounds, lipid nanoparticles (LNPs) thereof, and methods of use thereof. In various embodiments, the LNP selectively targets a cell of interest (e.g., a bone cell and/or bone marrow cell, such as a stem cell, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, and multiple myeloma cell, inter alia). In other aspects, the present disclosure relates to methods for in vivo delivery of therapeutic agents to prevent or treat diseases, disorders, or conditions using the LNP compositions of the disclosure.

### CROSS-REFERENCE TO RELATED APPLICATIONS

This application claims priority under 35 U.S. § 119(e) to U.S. Provisional Patent Application No. 63/721,206, filed Nov. 15, 2024, and is a continuation-in-part of and claims priority to U.S. patent application Ser. No. 18/865,098, filed Nov. 12, 2024, which is a 35 U.S.C. § 371 national phase application from and claims priority to PCT International Patent Application No. PCT/US2023/066956, filed May 12, 2023, which claims priority under 35 U.S.C. § 119(e) to U.S. Provisional Patent Application No. 63/341,753, filed May 13, 2022, the disclosures of all of which are hereby incorporated by reference in their entireties.

### STATEMENT REGARDING FEDERALLY SPONSORED RESEARCH OR DEVELOPMENT

This invention was made with government support under TR002776 awarded by the National Institutes of Health. The government has certain rights in the invention.

### BACKGROUND

The bone microenvironment is a distinct, highly dynamic region that comprises bone cells, cells of the hematopoietic and immune system, fibroblasts, stromal cells, and endothelial cells, as well as extracellular matrix (ECM) with abundant growth and/or signaling factors. These various cell types and ECM proteins enable orchestrated bone remodeling, hematopoiesis, immune function regulation, and tissue regeneration. Recently, increased incidences of various skeletal disease and age-related bone abnormalities, including osteoporosis, osteoarthritis, osteomyelitis and bone cancer, has inspired the investigation of novel biomaterials for bone microenvironment targeting.

However, various characteristics of the bone microenvironment, including reduced blood flow and vascularization compared to other organs, the blood-bone marrow barrier, poorly perfused bone sections, highly dense hierarchic structures, and low affinity between drugs and bone minerals, has generated several challenges for successful delivery of therapeutics into bone. Currently, organic/inorganic nanocomposites, and hydrogels have been developed for targeted and local delivery of pharmacological-based therapeutics, such as antibiotics, growth factors, anti-inflammatory molecules, anticancer agents, and hormones.

However, low delivery efficiency of these materials necessitated high dosing to achieve ideal therapeutic dosage at diseased sites and long-term bioavailability, increasing the incidence of severe side effects. These limitations indicated the need for efficient and safe therapeutic approaches for targeted drug delivery to the bone microenvironment.

Thus, there is a need in the art for LNP delivery systems with high delivery efficacy and low toxicity to deliver RNA therapeutics, such as mRNA, gene therapy, gene editing technologies, etc., in a precise and efficient way to a bone cell and/or bone marrow cell. The present disclosure satisfies this unmet need.

### BRIEF SUMMARY

In one aspect, the disclosure provides a compound of Formula (I), or a racemate, enantiomer, diastereomer, pharmaceutically acceptable salt, solvate, or a derivative thereof.

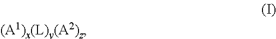

wherein:
each occurrence of A¹ is independently

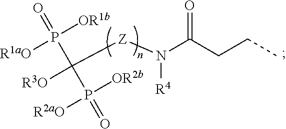

each occurrence of A² is independently

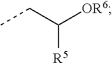

each occurrence of L is an amine linker independently selected from the group consisting of aminoalkyl linker, substituted aminoalkyl linker, diaminoalkyl linker, substituted diaminoalkyl linker, triaminoalkyl linker, substituted triaminoalkyl linker, tetraaminoalkyl linker, substituted tetraaminoalkyl linker, pentaaminoalkyl linker, substituted pentaaminoalkyl linker, polyaminoalkyl linker, substituted polyaminoalkyl linker, aminocycloalkyl linker, substituted aminocycloalkyl linker, diaminocycloalkyl linker, substituted diaminocycloalkyl linker, triaminocycloalkyl linker, substituted triaminocycloalkyl linker, tetraaminocycloalkyl linker, substituted tetraaminocycloalkyl linker, pentaaminocycloalkyl linker, substituted pentaaminocycloalkyl linker, polyaminocycloalkyl linker, substituted polyaminocycloalkyl linker, and any combination thereof;
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof,
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si;
each occurrence of z′ and z″ is independently an integer represented by 0, 1, or 2;
wherein x, y, and z are independently an integer from 0 to 20;
each occurrence of n is independently an integer from 0 to 10.

In another aspect, the disclosure provides a lipid nanoparticle (LNP) comprising:
(a) at least one compound having the structure of Formula (I), wherein the compound is present in a concentration range of about 1 mol % to about 99 mol %;
(b) at least one neutral phospholipid, wherein the neutral phospholipid is present in a concentration range of about 5 mol % to about 45 mol %;
(c) at least one cholesterol lipid, wherein the total cholesterol lipid is in a concentration range of about 5 mol % to about 55 mol %; and
(d) at least one polymer conjugated lipid (e.g., polyethylene glycol (PEG)-conjugated lipid), wherein the total polymer conjugated lipid is present in a concentration range of about 0.5 mol % to about 12.5 mol %.

In another aspect, the disclosure provides a composition comprising at least one LNP of the disclosure, optionally further comprising at least one pharmaceutically acceptable excipient.

In another aspect, the disclosure provides a method of delivering an agent to a subject in need thereof. In certain embodiments, the method comprises administering a therapeutically effectively amount of at least one LNP of the disclosure or a pharmaceutical composition thereof to the subject.

In another aspect, the disclosure provides a method of delivering an agent to a bone in a subject, bone marrow, or a combination thereof. In certain embodiments, the method comprises administering a therapeutically effectively amount of at least one LNP of the disclosure or a pharmaceutical composition thereof to the subject.

In another aspect, the disclosure provides a method of treating, ameliorating, and/or preventing at least one disease, disorder, or condition in a subject in need thereof. In certain embodiments, the method comprises administering a therapeutically effectively amount of at least one LNP of the disclosure or a pharmaceutical composition thereof to the subject.

In another aspect, the disclosure provides a method of inducing a bone regeneration in a subject in need thereof. In certain embodiments, the method comprises administering a therapeutically effectively amount of at least one LNP of the disclosure or a pharmaceutical composition thereof to the subject.

In another aspect, the disclosure provides a method of replacing at least one protein in a subject in need thereof. In certain embodiments, the method comprises administering a therapeutically effectively amount of at least one LNP of the disclosure or a pharmaceutical composition thereof to the subject.

In another aspect, the disclosure provides a method of gene editing in a subject in need thereof. In certain embodiments, the method comprises administering a therapeutically effectively amount of at least one LNP of the disclosure or a pharmaceutical composition thereof to the subject.

In another aspect, the disclosure provides a method of inducing an immune response in a subject in need thereof. In certain embodiments, the method comprises administering a therapeutically effectively amount of at least one LNP of the disclosure or a pharmaceutical composition thereof to the subject.

In another aspect, the disclosure provides a compound of Formula (V), or a salt, stereoisomer, or isotopologue thereof:

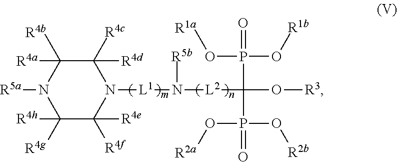

wherein:
R^{1a}, R^{1b}, R^{2a}, R^{2b}, and R³ are each independently selected from the group consisting of H, C(═O)R^{A}, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
R^{4a}, R^{4b}, R^{4c}, R^{4d}, R^{4e}, R^{4f}, R^{4g}, and R^{4h} are each independently selected from the group consisting of H, halogen, and optionally substituted C₁-C₆ alkyl;
R^{5a} and R^{5b} are each independently selected from the group consisting of H, optionally substituted C₁-C₂₄ alkyl, —C(═O)(optionally substituted C₁-C₂₄ alkyl), —C(═O)O(optionally substituted C₁-C₂₄ alkyl), and R⁶,
   wherein at least one of R^{5a} and R^{5b} is R⁶;
each occurrence of R⁶ is independently

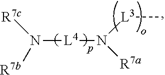

or
   two occurrences of R⁶ can combine with the atoms to which they are
   bound to form

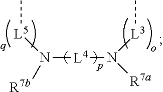

each occurrence of L¹, L², L³, L⁵, and L⁶, if present, is independently selected from the group consisting of -(optionally substituted C₁-C₃ alkylenyl)-, —C(═O)—, —O—, and —N(R^{A})—;
each occurrence of L⁴ is independently selected from the group consisting of —X—, -(optionally substituted C₁-C₁₂ alkylenyl)-, -(optionally substituted C₂-C₁₂ alkenylenyl)-, -(optionally substituted C₁-C₁₂ alkynylenyl)-, -(optionally substituted C₁-C₁₂ heteroalkylenyl)-, -(optionally substituted C₃-C₈ cycloalkylenyl)-, -(optionally substituted C₂-C₈ heterocyloalkylenyl)-, -(optionally substituted C₆-C₁₀ arylenyl)-, and -(optionally substituted C₂-C₈ heteroarylenyl)-;
each occurrence of X, if present, is independently selected from the group consisting of —N(R^{7d})—, —N(R⁸)—, —C(═O)—, and —O—;
each occurrence of R^{7a}, R^{7b}, R^{7c}, R^{7d}, R^{7e}, and R^{7f}, if present, are each independently selected from the group consisting of H, optionally substituted C₁-C₂₄ alkyl and optionally substituted C₁-C₂₄ heteroalkyl;
each occurrence of R⁸ is independently

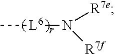

each occurrence of m and n, o, p, q, and r, if present, are each independently an integer selected from the group consisting of 1, 2, 3, 4, 5, 6, 7, 8, 9, and 10; and
each occurrence of R^{A} is independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

In another aspect, the disclosure provides a lipid nanoparticle (LNP) composition comprising:
(a) at least one ionizable lipid comprising at least one compound of Formula (V), or a salt, stereoisomer, or isotopologue thereof,
(b) at least one neutral lipid;
(c) at least one cholesterol lipid and/or a modified derivative thereof, and
(d) at least one polymer-conjugated lipid and/or a modified derivative thereof.

In another aspect, the disclosure provides a pharmaceutical composition comprising the lipid nanoparticle (LNP) of the disclosure and at least one pharmaceutically acceptable carrier.

In another aspect, the disclosure provides a method of delivering an agent to a bone of a subject. In certain embodiments, the method comprises administering at least one LNP of the disclosure or a pharmaceutical composition thereof to the subject.

In another aspect, the disclosure provides a method of treating, ameliorating, and/or preventing at least one disease, disorder, or condition in a subject in need thereof. In certain embodiments, the method comprises administering at least one LNP of the disclosure or a pharmaceutical composition thereof to the subject.

In another aspect, the disclosure provides a method of inducing bone regeneration in a subject in need thereof. In certain embodiments, the method comprises administering at least one LNP of the disclosure or a pharmaceutical composition thereof to the subject.

In another aspect, the disclosure provides a composition comprising a mineralized tissue and the lipid nanoparticle (LNP) of the disclosure, wherein the LNP is adsorbed to a surface of the mineralized tissue.

In another aspect, the disclosure provides a method for treating, preventing, and/or ameliorating an orthopedic or dental disease in a subject in need thereof. In certain embodiments, the method comprises contacting a mineralized tissue of the subject with a LNP-adsorbed mineral tissue composition under conditions effective to graft the mineralized tissue of the composition to the mineralized tissue of the subject.

In another aspect, the disclosure provides a method for delivering a nucleic acid molecule and/or therapeutic agent to a mineralized tissue of a subject in need thereof. In certain embodiments, the method comprises contacting a mineralized tissue of the subject with a LNP-adsorbed mineral tissue composition under conditions effective to graft the mineralized tissue of the composition to the mineralized tissue of the subject.

In another aspect method for repairing, restoring, or reducing degradation of a mineralized tissue in a subject in need thereof. In certain embodiments, the method comprises contacting a mineralized tissue of the subject with a LNP-adsorbed mineral tissue composition under conditions effective to graft the mineralized tissue of the composition to the mineralized tissue of the subject.

### BRIEF DESCRIPTION OF THE DRAWINGS

The following detailed description of illustrative embodiments of the disclosure will be better understood when read in conjunction with the appended drawings. For the purpose of illustrating the disclosure, illustrative embodiments are shown in the drawings. It should be understood, however, that the disclosure is not limited to the precise arrangements and instrumentalities of the embodiments shown in the drawings.

FIGS. 1A-1D depict a schematic representation of the rational design of BP lipid-like materials for mRNA delivery to the bone microenvironment in vivo. FIG. 1A depicts a schematic representation of lipid nanoparticles (LNPs) containing BP lipid-like materials (BP-LNPs) to enable systemic delivery of mRNA into the bone microenvironment. LNPs were prepared by combining four components (BP-lipid, DOPE, cholesterol and C14PEG2000) into each formulation using a microfluidic mixing device. After systemic delivery via intravenous injection, BP-LNPs coordinated with calcium ions (Ca2+) in the bone microenvironment to enable specific bone-targeting. FIG. 1B depicts representative results of ex vivo luminescence and fluorescence imaging of bones (left leg, spine, and right leg) after LNP delivery of mRNA encoding for luciferase and labelled with 1,1′-dioctadecyl-3,3,3′,3′-tetramethylindotricarbocyanine iodide (DiR) at a concentration of 10 g/mL. Luminescence signal was generated where luciferase mRNA was translated, and fluorescence signal detected DiR signal from LNP biodistribution. FIG. 1C depicts representative cryogenic transmission electron microscopy (cryo-TEM) image of the morphology of BP-LNPs. Scale bar: 100 nm. FIG. 1D depicts representative results demonstrating hydrodynamic size distribution of representative BP-LNP. I.V., intravenous.

FIGS. 2A-2C depict a representative combinatorial library of BP lipid-like materials for mRNA delivery. Error bars represent SD. FIG. 2A depicts schematic representations of seven exemplary alendronate-conjugated polyamine cores (top) and three exemplary epoxide terminated alkyl tails (bottom) for generating twenty-one exemplary BP-lipids used in this study. BP-LNPs were named based on their BP-conjugated ionizable lipid component consisting of a BP-functionalized polyamine core (110BP, 197BP, T3ABP, 200BP, 488BP, 490BP and 494BP) and alkyl tail length (C12, C14 and C16). FIG. 2B depicts representative results demonstrating hydrodynamic size and PDI of the twenty-one exemplary formulated BP-LNPs. FIG. 2C depicts representative characterization of zeta potential and pKa for each BP-LNP formulation.

FIG. 3 depicts a schematic representation of the synthetic route of BP-lipids used in this study.

FIG. 4 depicts a representative proton nuclear magnetic resonance (¹H NMR) spectrum of N-acryloxysuccinimide (NAS).

FIG. 5 depicts a representative ¹H NMR spectrum of alendronate acrylamide.

FIG. 6 depicts a representative ¹H NMR spectrum of alendronate amide-bearing lipid core as exemplified by 110BP.

FIG. 7 depicts a representative ¹H NMR spectrum of alendronate amide-bearing lipid core T3ABP.

FIG. 8 depicts a representative ¹H NMR spectrum of alendronate amide-bearing lipid core 197BP.

FIG. 9 depicts a representative ¹H NMR spectrum of alendronate amide-bearing lipid core 200BP.

FIG. 10 depicts a representative ¹H NMR spectrum of alendronate amide-bearing lipid core 488BP.

FIG. 11 depicts a representative ¹H NMR spectrum of alendronate amide-bearing lipid core 490BP.

FIG. 12 depicts a representative ¹H NMR spectrum of alendronate amide-bearing lipid core 494BP.

FIGS. 13A-13B depict a schematic representation of 490BP-C14 and its corresponding ¹H NMR spectrum. FIG. 13A depicts a schematic representation of 490BP-C14. FIG. 13B depicts a representative ¹H NMR spectrum of 490BP-C14.

FIG. 14 depicts schematic representations of the lipid cores and tails used as a control in the library described herein. The figure depicts schematic representations of seven exemplary polyamine cores used in preparation of the library of twenty-one exemplary non-BP-lipids. The box on the right depicts schematic representations of three exemplary epoxide terminal alkyl tails used in preparation of the library of twenty-one exemplary non-BP-lipids.

FIG. 15 depicts representative results demonstrating mRNA encapsulation efficiency of twenty-one exemplary BP-LNPs. n=3 biological replicates. Error bars represent SEM.

FIGS. 16A-16G depict representative results demonstrating TNS fluorescence curves for BP-LNPs. The apparent pKa of an LNP was computed as the pH at which 50% of the TNS fluorescence was measured. FIG. 16A depicts representative results demonstrating TNS fluorescence curves for BNP-lipids derived from the 110BP-polyamine core. FIG. 16B depicts representative results demonstrating TNS fluorescence curves for BNP-lipids derived from the 197BP-polyamine core. FIG. 16C depicts representative results demonstrating TNS fluorescence curves for BNP-lipids derived from the T3ABP-polyamine core. FIG. 16D depicts representative results demonstrating TNS fluorescence curves for BNP-lipids derived from the 200BP-polyamine core. FIG. 16E depicts representative results demonstrating TNS fluorescence curves for BNP-lipids derived from the 484BP-polyamine core. FIG. 16F depicts representative results demonstrating TNS fluorescence curves for BNP-lipids derived from the 490BP-polyamine core. FIG. 16G depicts representative results demonstrating TNS fluorescence curves for BNP-lipids derived from the 494BP-polyamine core.

FIGS. 17A-17C depict representative results demonstrating in vitro screening of BP-LNPs and their binding capability to HA nanoparticles and bone fragments. Error bars in FIG. 17A and FIG. 17B represent SEM. FIG. 17A depicts representative results demonstrating luciferase expression in HeLa cells after treatment with the BP-LNP library. 5000 cells were incubated with LNPs at a dose of 10 ng/well. Results were normalized to untreated cells. n=3 biological replicates. FIG. 17B depicts representative results demonstrating quantitative evaluation of HA binding kinetics of 490BP-C14 LNPs and 490-C14 LNPs in the absence of BPs (single emulsion). FIG. 17C depicts representative fluorescence images of bone fragments before and after incubation with DiO solution (left), DiO labeled 490-C14 LNPs (center), and DiO labeled targeted 490BP-C14 LNPs (right). All samples were washed by PBS before imaging. Scale bar: 200 μm.

FIGS. 18A-18B depict representative results demonstrating in vitro screening and cell viability after 24 h transfection of LNPs in the absence of BPs of HeLa cells. 5000 cells were plated per well and treated with 10 ng mRNA. Error bars represent SEM. FIG. 18A depicts representative results demonstrating in vitro screening after 24 h transfection of LNPs in the absence of BPs of HeLa cells. FIG. 18B depicts representative results demonstrating in vitro cell viability after 24 h transfection of LNPs in the absence of BPs of HeLa cells.

FIG. 19 depicts representative results demonstrating cell viability after 24 h transfection of LNPs in the presence of BPs in HeLa cells. 5000 cells were plated per well and treated by 10 ng mRNA. Error bars represent SEM.

FIGS. 20A-20B depict representative results demonstrating in vitro and mRNA dependent transfection studies comparing 490BP-C14 LNP to 490-C14 LNP. mRNA dosages were 10 ng, 20 ng, 40 ng, 80 ng and 160 ng per well to treat cells. 5000 HeLa cells per well were used. Error bars represent SEM. FIG. 20A depicts representative results demonstrating in vitro transfection of HeLa cells by 490BP-C14 LNP and 490-C14 LNP at an mRNA dosage of 10 ng mRNA. FIG. 20B depicts representative results demonstrating mRNA dependent transfection of HeLa cells by 490BP-C14 LNP.

FIGS. 21A-21G depict representative results of in vivo delivery of mRNA and biodistribution of BP-LNPs to the bone microenvironment. Statistical significance in FIG. 21D and FIG. 21G was calculated using Student's t test with unpaired design. ****P<30 0.0001; ***P<0.001; **P<0.01. Error bars represent SEM. FIG. 21A depicts a schematic representation of DiR labeled LNPs delivering mRNA to the bone of mice. Bone fragments were further dissected for quantifying the transfection and homing efficiency of BP-LNPs. FIG. 21B depicts representative IVIS imaging of FLuc mRNA delivery to mice by 490-C14 and 490BP-C14 LNPs. Bone fragments show high luminescence intensity after 490BP-C14 LNP treatment. n=3 mice. FIG. 21C depicts representative left leg, spine and right leg dissected for luminescence imaging. FIG. 21D depicts representative results demonstrating the total luminescence quantification of dissected left leg, spine, and right leg for 490-C14 LNP and 490BP-C14 LNP treated mice. FIG. 21E depicts representative results demonstrating in vivo biodistribution of DiR labeled BP-LNPs. After introducing BP lipid-like materials into LNPs, the whole skeleton showed increased fluorescent signal. n=3 mice. FIG. 21F depicts representative results depicting the left leg, spine, and right leg that were dissected for fluorescence imaging. FIG. 21G depicts representative results demonstrating the total fluorescence radiance of dissected left leg, spine, and right leg for 490-C14 LNP and 490BP-C14 LNP treated mice.

FIGS. 22A-22D depict representative results demonstrating ex vivo luminescence and fluorescence intensity of organs in mice after delivering FLuc-mRNA LNPs labelled by DiR. n=3 mice H: heart; Li: liver; S: spleen; Lu: lung; K: Kidney. Error bars represent SEM. FIG. 22A depicts representative luminescence imaging of the main organs from mice with 490-C14 and 490BP-C14 LNPs treatment. FIG. 22B depicts representative results demonstrating luminescence quantification of organs from FIG. 22A. FIG. 22C depicts representative fluorescence imaging of the main organs from mice with 490-C14 and 490BP-C14 LNP treatment. FIG. 22D depicts representative results demonstrating fluorescence quantification of the organs from FIG. 22C.

FIG. 23 depicts representative results demonstrating time-dependence in vivo biodistribution of 490-C14 LNP and 490BP-C14 LNP.

FIGS. 24A-24D depict representative results demonstrating bone and bone marrow cell transfection and distribution following mRNA delivery using BP-LNPs. Statistical significance in FIG. 24B and FIG. 24D was calculated using Student's t test with unpaired design. ****P<0.0001; ***P<0.001; **P<0.01. Error bars represent SEM. FIG. 24A depicts representative results demonstrating gating for EGFP mRNA transfection in different cell types by 490-C14 and 490BP-C14 LNPs. T cells, monocytic lineage cells, B lineage cells, and monocytes were labelled by their corresponding markers. FIG. 24B depicts representative results demonstrating different EGFP⁺ cell populations following treatment of LNPs that incorporated (490BP-C14) or did not incorporate (490-C14) bone-targeting BPs. FIG. 24C depicts representative results demonstrating gating for DiR fluorescence in different cell types by 490-C14 and 490BP-C14 LNPs. T cells, monocytic lineage cells, B lineage cells, and monocytes were labelled by their corresponding markers. FIG. 24D depicts representative results demonstrating different DiR-loaded cell populations among DiR⁺ cells. n=3 mice.

FIGS. 25A-25E depict representative results demonstrating EGFP mRNA transfects diverse types of cells in the bone marrow. Experiments were performed for three or more replicates. Error bars represent SEM. FIG. 25A depicts a representative results demonstrating gating strategy to identify cell types in bone marrow. FIG. 25B depicts representative results demonstrating EGFP mRNA transfection in B cells, endothelial cells, and granulocytes by 490-C14 LNPs and 490BP-C14 LNPs. FIG. 25C depicts representative results demonstrating EGFP⁺ populations of LNPs treated B cells, endothelial cells, granulocytes, and HSC cells. FIG. 25D depicts representative results demonstrating DiR signal in B cells, endothelial cells, and granulocytes after administration of 490-C14 LNPs and 490BP-C14 LNPs. FIG. 25E depicts representative results demonstrating DiR⁺ populations of LNP treated B cells, endothelial cells, granulocytes, and HSC cells.

FIGS. 26A-26B depict representative results demonstrating EGFP transfection and fluorescent biodistribution of 490BP-C14 LNPs relative to 490-C14 LNPs in various cell types. FIG. 26A depicts representative results demonstrating EGFP transfection in various cell types in bone marrow treated with 490BP-C14 LNPs, represented as fold of increase in the EGFP⁺ cell population treated with 490BP-C14 LNPs compared to those treated with 490-C14 LNPs. FIG. 26B depicts representative results demonstrating fluorescent biodistribution in various cell types in bone marrow treated with 490BP-C14 LNPs, represented as fold of increase in the DiR⁺ cell population treated with 490BP-C14 LNPs compared to 490-C14 LNPs.

FIGS. 27A-27B depict representative result demonstrating BMP-2 protein secretion in the bone microenvironment following delivery of BP-LNPs encapsulating mRNA encoding for BMP-2 via intravenous injection. Statistical significance in FIG. 27A and FIG. 27B was calculated using Student's t test with unpaired design. **P<0.01; *P<0.05. Error bars represent SEM. FIG. 27A depicts representative results demonstrating BMP-2 secretion from the bone surface following BP-LNP mRNA delivery. BMP-2 was extracted from the bone surface by incubating bone samples with 4M guanidine hydrochloride overnight. FIG. 27B depicts representative results demonstrating BMP-2 secretion in the bone marrow with increased BMP-2 mRNA dosing. BMP-2 was extruded out from the bone marrow. n=3 mice.

FIGS. 28A-28B depict representative results demonstrating liver toxicity assay after injection of LNPs encapsulating luciferase-encoding mRNA. C57BL/6J mice were dosed with 1.5 mg/kg luciferase mRNA LNPs, and liver enzymes were quantified 12 h after injection. Error bars represent SEM. FIG. 28A depicts representative results demonstrating alanine transaminase (ALT) quantification (±standard deviation) for controls, 490-C14 LNPs and 490BP-C14 LNPs. n=3 mice. FIG. 28B depicts representative results demonstrating aspartate transaminase (AST) quantification (±standard deviation) for controls, 490-C14 LNPs and 490BP-C14 LNPs. n=3 mice.

FIG. 29 provides representative images of EGFP expression in the femur after administration of 490BP-C14 LNPs encapsulating EGFP mRNA to the bone microenvironment by intravenous administration. The bone femur was dissected 12 h post injection. DAPI was used for nuclear staining. The mRNA dosage was 0.5 mg/kg. EGFP expression was detected in the bone marrow, especially in the endosteum, rather than on the bone surface.

FIGS. 30A-30C depict representative images of BMP-2 expression in the tibia bones of mice. FIG. 30A: tibia bone from PBS treated mice. FIG. 30B: tibia bone was dissected from mice treated with 490-C14 LNPs delivering BMP-2 mRNA. FIG. 30C: tibia bond was dissected from mice treated with 490BP-C14 LNPs delivering BMP-2 mRNA. The magnified regions represent BMP-2 expression. Scale bar: 500 μm. DAPI (blue) was used for nuclear staining, and BMP2 expression is shown in green.

FIGS. 31A-31C depict stability of 490BP-C14 LNPs after storage at 4° C. for 21 days. FIG. 31A: particle size quantification at different time points. FIG. 31B: mRNA encapsulation efficiency at different time points. FIG. 31C: luciferase expression in HeLa cells after treatment with 490BP-C14 LNPs at different time points. 5000 cells were incubated with LNPs at a dose of 10 ng/well. Results were normalized to untreated cells; n=3 replicates.

FIGS. 32A-32B depict ex vivo imaging of bones after Fluc mRNA delivery to mice by 490-C14 LNPs and 490BP-C14 LNPs (Fluc mRNA dose: 0.25 mg/kg). Fore limbs, hind limbs, and spine were dissected for imaging. Both the luminescence signal and fluorescent signal increased in 490BP-LNP treated group compared to the 490-C14 LNP control. FIG. 32A: luminescent intensity of the bone sites. FIG. 32B: fluorescence intensity of the bone sites. DiR fluorescent dye was used to label the LNPs.

FIGS. 33A-33C depict representative H&E staining of spine from PBS (FIG. 33A), 490-C14 LNP (FIG. 33B), and 490BP-C14 LNP (FIG. 33C).

FIG. 34A depicts EGFR transfection in various cell types in bone marrow treated with 490BP-C14 LNPs, represented as fold of increase in the EGFP⁺ cell population treated with 490BP-C14 LNPs compared to those treated with 490-C14 LNPs. FIG. 34B depicts fluorescent biodistribution in various cell types in bone marrow treated with 490BP-C14 LNPs, represented as fold of increase in the DiR⁺ cell population treated with 490BP-C14 LNPs compared to 490-C14 LNPs.

FIGS. 35A-35B depict representative immunofluorescence of Smad1/5 activation in the bone microenvironemtn of mouse tibia before and after BMP-2 mRNA delivery by LNPs. FIG. 35A: Smad1/5 activation in metaphyseal region of mice tibia. FIG. 35B: Smad1/5 activation in endosteal region of mice tibia. Scale bar: 200 μm. DAPI (blue) was used for nuclear staining and phosphorylated Smad1/5 was shown in green.

FIGS. 36A-36B depict H&H staining of representative bone sites of PBS, 490-C14 LNP, and 490BP-C14 LNP treated mice. FIG. 36A depicts representative images of cortical bone and endosteal bone marrow of femur bones. FIG. 36B depicts representative images of trabecular bone and metaphysis bone marrow of femur bones. Scale bar: 100 μm.

FIGS. 37A-37F illustrate systemic delivery of piperazine-based bisphosphonate-linked LNPs (PIP-BP-LNPs) to bone (FIG. 37A), and the hydrodynamic diameter and TEM image of top-performing PIP-BP-LNPs (‘Type3-P1-C12 (10%)’+‘C12-200 (90%)’) (FIG. 37B). FIG. 37C: Components of the LNPs with 5 different types of piperazine-bisphosphonate ionizable lipids. FIG. 37D: Luciferase expression in the BJ cell line, 5,000 cells of the BJ cell line, and 10 ng of FLuc mRNA were treated in each well. FIGS. 37E-37F: Bioluminescence of in vivo transfection results (FIG. 37E) and quantification of in vivo transfection results (FIG. 37F). ****p<0.0001.

FIGS. 38A-38B depict schematic illustrations of bone-targeting ionizable lipid synthesis (FIG. 38A) and ‘PIP-BP capping ligands (5)’×‘Amine cores (7)’×‘Epoxide-based alkyl chains (4)’=PIP-BP ionizable lipids (140) (FIG. 38B).

FIGS. 39A-39B depict in vitro screening results of piperazine-based bisphosphonate-linked (PIP-BP LNPs) in the Hep-G2 cell line utilizing 100% of PIP-BP ionizable lipids alongside C14-PEG2000 and DOPE lipids. FIG. 39A: The hydrodynamic diameters and TEM images of top-performing PIP-BP LNPs in the Hep-G2 cell line and top-performing PIP-BP LNPs in the BJ cell line. FIG. 39B: Analysis of the relationship between the chemical structure of PIP-BP ionizable lipids and the protein expression efficiency of BP-LNPs in vitro. 5,000 cells of the Hep-G2 cell line and 10 ng of FLuc mRNA were treated in each well.

FIGS. 40A-40D depict simulation results on the correlation among bone-targeting ionizable, C14-PEG, and DOPE lipids on the surface of LNPs, based on structural conformation, energy, and electrostatic potential. FIG. 40A: Pictorial representation of the correlation. FIGS. 40B-40D: Chemical structures (FIG. 40B) corresponding to the computed chemical structures on the LNP surface (FIG. 40C) and single-point electronic energy values (FIG. 40D).

FIGS. 41A-41B depict changes in physicochemical properties of piperazine-based bisphosphonate-linked (PIP-BP) LNPs according to variations in the composition ratio of PIP-BP ionizable lipids. FIG. 41A: 100% PIP-BP ionizable lipids with C14-PEG2000 and DOPE lipids. FIG. 41B: PIP-BP ionizable lipids mixed with C12-200, C14-PEG2000, and DOPE lipids. (a1 and b1) ‘Type1-P1-C12’+‘C12-200’, and (a2 and b2) ‘Type3-P1-C12’+‘C12-200’ were utilized. The PIP-BP LNPs were formulated with the following ratios: Ionizable lipid(s)/DOPE/Cholesterol/C14-PEG2000=35/16/46.5/2.5%.

FIGS. 42A-42B depict binding study on hydroxyapatite (HA) using the PIP-BP LNPs. FIG. 42A: Analytical results of AFM measurements with PIP-BP LNPs composed of 100% bone-targeting lipids, C14-PEG2000, and DOPE lipids on the HA surface. FIG. 42B: Binding study of PIP-BP LNPs to the HA surface in the BJ cell line. Luciferase expression in the BJ cell line, 5,000 cells of the BJ cell line, and 10 ng of FLuc mRNA were treated in each well. The BJ cells were seeded on the bottom surface of the well plate and the HA surface for 24 hours. The PIP-BP LNPs were applied to the HA surface in the chamber, and luciferase expression was measured in BJ cells on the bottom surface of the well plate, excluding the chamber. The luciferase expression in BJ cells on the bottom surface of the well plate was measured, after incubating PIP-BP LNPs on HA discs for 24 hours. The number of replicates for this binding study was more than five, and each experiment was repeated at least three times. The discrepancy values represent the differences in luciferase expression between ‘LNPs’ and ‘LNPs+HA’. The PIP-BP LNPs were formulated with the following ratios: Ionizable lipid(s)/DOPE/Cholesterol/C14-PEG2000=35/16/46.5/2.5%. ****p<0.0001, and ns=not significant. Error bars represent SEM.

FIGS. 43A-43C depict bioluminescence results in vivo and ex vivo. PIP-BP LNPs are composed of 10% PIP-BP and 90% C12-200 ionizable lipids (FIG. 43A) or 40% PIP-BP and 60% C12-200 ionizable lipids (FIG. 43B). FIG. 43C: LNPs are composed of 100% C12-200 ionizable lipids. Each mouse received an intravenous injection of 0.5 mg mRNA/kg, and bioluminescence intensity was measured after 6 hours. The number of replicates was three, with the experiment repeated three times. The PIP-BP LNPs were formulated with the following ratios: Ionizable lipids/DOPE/Cholesterol/C14-PEG2000=35/16/46.5/2.5%. ****p<0.0001, ***p<0.001, **p<0.01, *p<0.05, and ns=not significant. Error bars represent SEM.

FIG. 44 depicts time-dependent bioluminescence changes. The bone-targeted LNPs were formulated with the following ratios: Ionizable lipids/DOPE/Cholesterol/C14-PEG2000=35/16/46.5/2.5%. Error bars represent SEM.

FIG. 45 depicts targeting molecules for the ‘Type 1’ bone-targeting ionizable lipids.

FIG. 46 depicts targeting molecules for the ‘Type 2’ bone-targeting ionizable lipids.

FIG. 47 depicts targeting molecules for the ‘Type 3’ bone-targeting ionizable lipids.

FIG. 48 depicts targeting molecules for the ‘Type 4’ bone-targeting ionizable lipids.

FIG. 49 depicts targeting molecules for the ‘Type 5’ bone-targeting ionizable lipids.

FIG. 50 depicts TEM image of bone-targeted LNPs composed of the ‘Type3-P1-C12 (10%)’ and ‘C12-200 (90%)’ ionizable lipids.

FIG. 51 depicts solubility of the ‘Product 4’, ‘Product 7’, and ‘Product 3’ (Left to right) in ethanol.

FIG. 52 depicts UV absorption peaks of Alendronate™

FIG. 53 depicts changes in the physicochemical properties of bone-targeted LNPs, consisting of 100% bone-targeting ionizable lipid (‘Type1-P1-C12’ or ‘Type3-P1-C12’), C14-PEG2000, and DOPE lipids, and their influence on encapsulation efficiency, luciferase expression, and cell viability in the Hep-G2 cell line. Cholesterol:C14-PEG2000=46.5:2.5%, and 5,000 cells of the BJ cell line and 10 ng of FLuc mRNA were treated in each well. Error bars represent SEM.

FIG. 54 depicts changes in the physicochemical properties of bone-targeted LNPs, consisting of bone-targeting ionizable lipid (‘Type1-P1-C12’ or ‘Type3-P1-C12’) mixed with ‘C12-200’ lipid, C14-PEG2000, and DOPE lipids, and their influence on encapsulation efficiency, luciferase expression, and cell viability in the Hep-G2 cell line. Cholesterol:C14-PEG2000=46.5:2.5%, and 5,000 cells of the BJ cell line and 10 ng of FLuc mRNA were treated in each well. Error bars represent SEM.

FIG. 55 depicts change in hydrodynamic diameters based on the composition of ionizable lipids, and the bone-targeted LNPs were formulated with the following ratios: Ionizable lipid(s)/DOPE/Cholesterol/C14-PEG2000=35/16/46.5/2.5%.

FIGS. 56A-56D depict binding study on hydroxyapatite using the bone-targeted LNPs. FIG. 56A and FIGS. 56C-56D: Confocal microscopy images. FIG. 56B: Pictorial representation for the Interaction between the fluorescence dyes (DiO or DiD) and the surface of bone-targeted LNPs. Images from confocal microscopy of bone-targeted LNPs stained with DiO and DiD dyes. The bone-targeted LNPs were formulated with the following ratios: Ionizable lipid(s)/DOPE/Cholesterol/C14-PEG2000=35/16/46.5/2.5%.

FIGS. 57A-57F depict in vitro screening results in Hep-G2 cell line and the physicochemical properties of bone-targeted LNPs with ‘Type 1’ ionizable lipids. 5,000 cells of the Hep-G2 cell line and 10 ng of FLuc mRNA were treated in each well. Error bars represent SEM. FIG. 57A: graph showing luciferase expression. FIG. 57B: graph showing relative cell viability. FIG. 57C: heat map. FIG. 57D: graph showing zeta potential. FIG. 57E: graph showing hydrodynamic diameter. FIG. 57F: graph showing PDI.

FIGS. 58A-58F depict in vitro screening results in Hep-G2 cell line and the physicochemical properties of bone-targeted LNPs with ‘Type 2’ ionizable lipids. 5,000 cells of the Hep-G2 cell line and 10 ng of FLuc mRNA were treated in each well. Error bars represent SEM. FIG. 58A: graph showing luciferase expression. FIG. 58B: graph showing relative cell viability. FIG. 58C: heat map. FIG. 58D: graph showing zeta potential. FIG. 58E: graph showing hydrodynamic diameter. FIG. 58F: graph showing PDI.

FIGS. 59A-59F depict in vitro screening results in Hep-G2 cell line and the physicochemical properties of bone-targeted LNPs with ‘Type 3’ ionizable lipids. 5,000 cells of the Hep-G2 cell line and 10 ng of FLuc mRNA were treated in each well. Error bars represent SEM. FIG. 59A: graph showing luciferase expression. FIG. 59B: graph showing relative cell viability. FIG. 59C: heat map. FIG. 59D: graph showing zeta potential. FIG. 59E: graph showing hydrodynamic diameter. FIG. 59F: graph showing PDI.

FIGS. 60A-60F depict in vitro screening results in Hep-G2 cell line and the physicochemical properties of bone-targeted LNPs with ‘Type 4’ ionizable lipids. 5,000 cells of the Hep-G2 cell line and 10 ng of FLuc mRNA were treated in each well. Error bars represent SEM. FIG. 60A: graph showing luciferase expression. FIG. 60B: graph showing relative cell viability. FIG. 60C: heat map. FIG. 60D: graph showing zeta potential. FIG. 60E: graph showing hydrodynamic diameter. FIG. 60F: graph showing PDI.

FIGS. 61A-61F depict in vitro screening results in Hep-G2 cell line and the physicochemical properties of bone-targeted LNPs with ‘Type 5’ ionizable lipids. 5,000 cells of the Hep-G2 cell line and 10 ng of FLuc mRNA were treated in each well. Error bars represent SEM. FIG. 61A: graph showing luciferase expression. FIG. 61B: graph showing relative cell viability. FIG. 61C: heat map. FIG. 61D: graph showing zeta potential. FIG. 61E: graph showing hydrodynamic diameter. FIG. 61F: graph showing PDI.

FIG. 62 depicts in vitro screening results and cell viability of the top-performing 16 bone-targeting ionizable lipids in the Hep-G2 cell line. 5,000 cells of the BJ cell line and 10 ng of FLuc mRNA were treated in each well. Error bars represent SEM.

FIG. 63 depicts in vitro experiments for the bone-targeted LNPs based on the ‘Type1-P1-C12 (100%)’ and ‘Type3-P1-C12 (100%)’ bone-targeting ionizable lipids in the Hep-G2 cell line. 5,000 cells of the Hep-G2 cell line were treated in each well. Error bars represent SEM.

FIG. 64 depicts in vitro experiments for the bone-targeted LNPs based on the ‘Type1-P1-C12 (100%)’ and ‘Type3-P1-C12 (100%)’ bone-targeting ionizable lipids in the BJ cell line. 5,000 cells of the BJ cell line were treated in each well. Error bars represent SEM.

FIGS. 65A-65B depict luciferase expression changes with formulation variations of bone-targeted LNPs composed of bone-targeting ionizable lipid (‘Type1-P1-C12’ (FIG. 65A) and ‘Type3-P1-C12’ (FIG. 65B)) mixed with ‘C12-200’ lipid. Cholesterol:C14-PEG2000=46.5:2.5%.

FIG. 66 depicts luciferase expression of bone-targeted LNPs excluding those composed of 10% bone-targeting ionizable lipid (‘Type3-P1-C12’) mixed with ‘C12-200’ lipid, with up to 50% bone-targeting ionizable lipid.

FIGS. 67A-67C depict luciferase expression in 5 different mouse organs (heart, liver, spleen, lung, and kidney) treated with bone-targeted LNPs based on ‘C12-200 (100%)’ (FIG. 67A), ‘Type3-P1-C12 (10%)’+‘C12-200 (90%)’ (FIG. 67B), and ‘Type3-P1-C12 (40%)’+‘C12-200 (60%)’ (FIG. 67C) ionizable lipids. Error bars represent SEM.

FIGS. 68A-68D depict representative H&E staining of leg tissue from mice treated with PBS (FIG. 68A), LNPs composed of ‘C12-200 (100%)’ (FIG. 68B), ‘Type3-P1-C12 (10%)’+‘C12-200 (90%)’ (FIG. 68C), and ‘Type3-P1-C12 (40%)’+‘C12-200 (60%)’ (FIG. 68D).

FIGS. 69A-69D depict representative H&E staining of 5 different organ tissues (heart, liver, spleen, lung, and kidney) from mice treated with PBS (FIG. 69A), LNPs composed of ‘C12-200 (100%)’ (FIG. 69B), ‘Type3-P1-C12 (10%)’+‘C12-200 (90%)’ (FIG. 69C), and ‘Type3-P1-C12 (40%)’+‘C12-200 (60%)’ (FIG. 69D).

FIGS. 70A-70D depict overall synthetic route for the synthesis of bone-targeting ionizable lipids. FIG. FIG. 70A: exemplary starting materials. FIG. 70B: synthetic scheme depicting synthesis of Type 1 and Type 2 ionizable lipids. FIG. 70C: synthetic scheme depicting synthesis of Type 3 and Type 4 ionizable lipids. FIG. 70D: synthetic scheme depicting synthesis of Type 5 ionizable lipid.

FIGS. 71A-71G depict formulation and characterization of MiTEX component, bisphosphonate lipid nanoparticles (BP-LNPs), for targeted mRNA delivery to mineralized tissues. FIG. 71A: Schematic overview depicting the process of formulating and validating MiTEX for local delivery to mineralized tissues. FIG. 71B: Chemical structures of bisphosphonate capping ligand, 7 polyamine cores, and 3 epoxide-terminated alkyl tails for generating 21 BP ionizable lipids used in this study. BP-LNPs are named based on their BP-linked polyamine cores (110, T3A, 197, 200, 488, 490, 494) and alkyl tails (C10, C12, C14) of varying lengths. FIG. 71C: The heat map of luciferase expression upon transfecting BJ cells in vitro with FLuc mRNA (5000 cells, 20 ng mRNA per well) in BP-LNPs. In vitro screening was recorded by relative light units (RLU). FIG. 71D: Hydrodynamic size and polydispersity index (PDI) (left) and zeta potential (right) of top-3 performing MiTEXs. For hydrodynamic size and PDI measurement, n=4 technical replicates, error bar represents SEM. FIG. 71E: Characterization of encapsulation efficiency of top-3 performing MiTEXs. n 30=3 technical replicates, error bar represents SEM. FIG. 71F: Dose-dependent luciferase expression and relative cell viability of MiTEX in tdROSA mouse bone marrow mesenchymal stem cells (mMSCs). n=3 biological replicates, error bar represents SEM. Statistical significance was calculated using a Two-way ANOVA test. FIG. 71G: tdTomato expression (red) after 24 h Cre mRNA-LNP transfection in mMSCs (20,000 cells, 200 ng mRNA per well). Stained for nuclei (blue). Scale bar: 100 μm.

FIGS. 72A-72D depict characterization of surface interactions between MiTEX and hydroxyapatite (HA). FIG. 72A: Schematic of the adsorption of BP-LNPs on hydroxyapatite surface. FIG. 72B: FT-IR of HA surface after loading MiTEX and control for 24 h. FIG. 72C: Quantitative evaluation of HA binding kinetics and sigmoidal fit of MiTEX and control in 24 h (left). A zoom-in binding kinetics of MiTEX and control in 4h (right). n=3 biological replicates, error bar represents SEM. FIG. 72D: Half-maximal time of hydroxyapatite binding kinetics of MiTEX and control in 24 h. n=3 biological replicates, error bar represents SEM. Statistical significance was calculated using a Multiple unpaired t-test.

FIGS. 73A-73F depict in vitro illustration of the adsorption and binding affinity of MiTEX to HA substrates and bone graft materials. FIG. 73A: Schematic showing in vitro LNP adsorption and transfection timeline on HA substrate (top). Schematic depicting the hypothesized mechanism of RNA transfection mediated by the adsorption of MiTEX and adjacent cellular uptake interaction on HA substrate surface (bottom). FIG. 73B: RNA accumulation mass on hydroxyapatite substrates by the adsorption of MiTEXs, compared with control. Statistical significance was calculated using a Two-way ANOVA test. FIG. 73C: Luciferase expression in the BJ cells seeded on HA substrate after 48 h transfection by HA adsorbed MiTEXs and control (50,000 cells, 2000 ng mRNA per substrate). FIG. 73D: Luciferase expression in the BJ cells 48 h after being seeded on different concentrations of alendronate sodium pre-treated HA substrate and MiTEXs adsorption. FIG. 73E: Quantification of cre-mRNA genetic labeling rate of tdROSA mMSCs transfected by HA adsorbed MiTEXs and control on HA substrate surface. For FIGS. 73C-73D, Statistical significance was calculated using One-way ANOVA tests. FIG. 73F: tdTomato expression after 48 h Cre mRNA-LNP transfection in mMSCs by HA adsorbed MiTEXs and control on HA substrate surface (100,000 cells, 2000 ng mRNA per substrate). Stained for nuclei (blue) and F-actin (green). Scale bar: 300 μm. For FIGS. 73B-73C and FIGS. 73E-73F, n=3 biological replicates; for FIG. 73D, n=6 biological replicates; error bar represents SEM.

FIGS. 74A-74E depict in vivo local gene expression by the adsorption of MiTEX in bone graft materials. FIG. 74A: In vitro dose-dependent luciferase expression in gingival fibroblasts transfected by allogenic bone graft particulates loaded with MiTEX and control (20,000 cells per well, 5 to 100 ng mRNA per bone graft cluster). n=3 biological replicates, error bar represents SEM. Statistical significance was calculated using a Two-way ANOVA test. FIG. 74B: De novo bone formation in immunocompromised mice with HA bone graft scaffold and mMSCs (4,000,000 cells) after 60 days. FIG. 74C: In vivo tdTomato expression within newly forming bones after 60 days. Stained for nuclei (blue) and alkaline phosphatase (green). Scale bar: 200 μm. FIG. 74D: Quantification of tdTomato intensity by cre-mRNA genetic labeling in newly forming bones. n=3 biological replicates; error bar represents SEM. Statistical significance was calculated using One-way ANOVA tests. FIG. 74E: H&E staining of bone tissues forming surrounding HA bone graft. B=bone, HA=HA bone graft. Scale bar: 50 μm.

FIGS. 75A-75D depict STAT3 pathway modulation following the delivery of siMiTEX and control encapsulating STAT3 siRNA. FIG. 75A: Schematic illustrating the STAT3 pathway and the role of siRNA-LNP. FIG. 75B: Gene expression from gingival fibroblasts 24 h after treatment with 5-100 nM control STAT3 siLNP (20,000 cells per well). FIG. 75C: Gene silencing and cytotoxicity of STAT3 siMiTEX and control from gingival fibroblasts in vitro (20,000 cells, 50 nM siRNA per well). Statistical significance was calculated using Multiple unpaired t-tests. FIG. 75D: IL-8 release of STAT3 siMiTEX or control treated gingival fibroblast 24 h and 48 h post-LPS stimulation (20,000 cells, 10 μg/mL LPS, 50 nM siRNA per well). Statistical significance was calculated using One-way ANOVA tests. For FIGS. 75B-75D, n=3 biological replicates, error bar represents SEM.

### DETAILED DESCRIPTION

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Description—Bisphosphonate—Substituted Piperazine Ionizable Lipid Compounds and Bone-Targeted Lipid Nanoparticles (LNPs) Thereof

The utilization of mRNA-LNPs holds promise for the treatment of bone-related diseases; however, achieving targeted delivery remains a significant challenge. In one aspect, described herein are bone-targeting ionizable lipids featuring a robust piperazine backbone, which forms strong interactions with hydroxyapatite ([Cas(PO₄)₃OH]), a key component of mineralized tissues. These lipids, conjugated with derivatives of bisphosphonates demonstrate exceptional biocompatibility and low toxicity. The findings described herein underscore the importance of the piperazine backbone in facilitating enhanced bone delivery while minimizing adverse effects. This research offers insights into the rational design of ionizable lipids for next-generate bone-targeting delivery systems.

Bones are predominantly composed of calcium-phosphate complexes and collagen, making bisphosphonates (BPs) with structures resembling phosphates commonly utilized as therapeutics for diseases such as osteoporosis. BPs inhibit bone resorption, promote bone formation, and increase bone strength and density by enhancing calcium deposition on bone trabecular surfaces and inhibiting the activity of osteoclasts, which absorb bone tissue. Additionally, effective treatment of bone-related disorders can be achieved through gene therapy, which targets the root causes of diseases. Recently, lipid nanoparticles (LNPs) have gained attention as a therapeutic carrier for nucleotide therapeutics, notably through COVID-19 vaccine treatments. LNPs offer several advantages as a clinically approved non-viral delivery system, primarily due to their biocompatible nature, attributed to their composition of ionizable lipids, structural lipids, PEG-phospholipids, and cholesterol, often inspired by bio-inspired lipid structures. Therefore, in the treatment of bone-related diseases, leveraging these advantages, LNPs can be combined with widely used bisphosphonate compounds to target bone-related diseases. Bone-targeted LNPs, thus manufactured, enable targeted and localized interactions with cells distributed within a bone, facilitating the role of gene therapy in the cellular signaling pathways of diseases.

Building on this premise, research targeting bones using LNPs and bisphosphonates, such as ALENDRONATEL™, where bone-targeted LNPs were delivered to a bone. However, the long-term or excessive use of bisphosphonates typically leads to adverse effects such as gastrointestinal issues, decreased blood supply to bones resulting in increased fracture risks, and renal problems. Therefore, there is a need to enhance the binding affinity between bone-targeted LNPs and bone while reducing the dosage of bisphosphonates to mitigate these side effects and improve protein expression.

Described herein, to address these needs, is a novel form of piperazine-based bisphosphonate-linked ionizable lipids which was successfully developed using a robust structure called piperazine (PIP) and bisphosphonates such as ALENDRONATE™. Bone-targeted LNPs developed based on these lipids were utilized to observe cellular uptake, luminescence expression through lysosomal-endosomal escape, and binding affinity with bones. Additionally, to enhance the understanding of the conceptual phenomenon, bone-targeting ionizable lipids were categorized into five types, resulting in the construction of a vast library comprising a total of 140 lipid variants. Most of the bone-targeting ionizable lipids demonstrated high cell viability, and their strong binding affinity with bone constituents was confirmed through atomic force microscopy (AFM), laser confocal microscopy, and in vitro experiments.

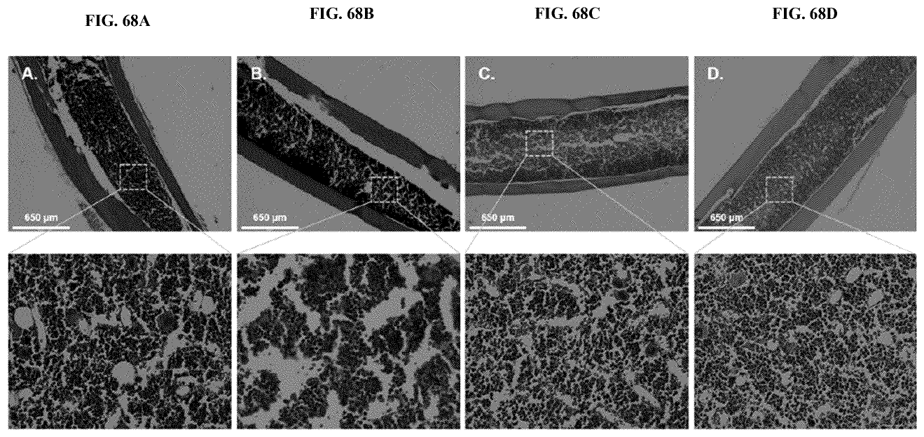

In this study, although only 10% of ‘PIP-based BP-linked’ ionizable lipid and 90% of ‘C12-200’ ionizable lipid were utilized, efficient mRNA delivery to bone cells was achieved, resulting in high luminescence expression. ‘C12-200’ ionizable lipid was prepared based on published literature. Compared to the negative control group using ‘C12-200’ ionizable lipid alone, the luminescence expression in bone cells was significantly higher, while the toxicity to cells was remarkably low (FIGS. 68A-68D and FIGS. 69A-69D). Thus, successful in vivo delivery of LNPs targeting only bones was achieved, addressing the issues of adverse effects associated with the long-term or excessive use of bisphosphonates. Furthermore, while previous studies have shown high protein expression with LNPs containing RNA based on piperazine-based ionizable lipids, the underlying reasons were not known. However, the results of this study provided a concise theoretical explanation of the influence of rigid piperazine-based ionizable lipids on the overall structure of LNPs from a morphology perspective.

Here, the targeted delivery of mRNA into bone marrow cells was controlled using the binding of a piperazine-based bisphosphonate-linked group to hydroxyapatite via modification of the ionizable lipid of lipid nanoparticles. The ionizable lipid is used for targeting instead of the PEG-phospholipids, because PEG-phospholipid constitutes only about 2% of LNPs' composition, and more importantly, modifying the terminal group of PEG-phospholipids with bisphosphonates presents challenges in controlling the orientation of these bisphosphonates on the LNP surface. Specifically, due to the flexible ether group's chemical structure, some bisphosphonate-linked PEG-phospholipids may exist on the surface of LNPs, while others may be located inside the LNPs.

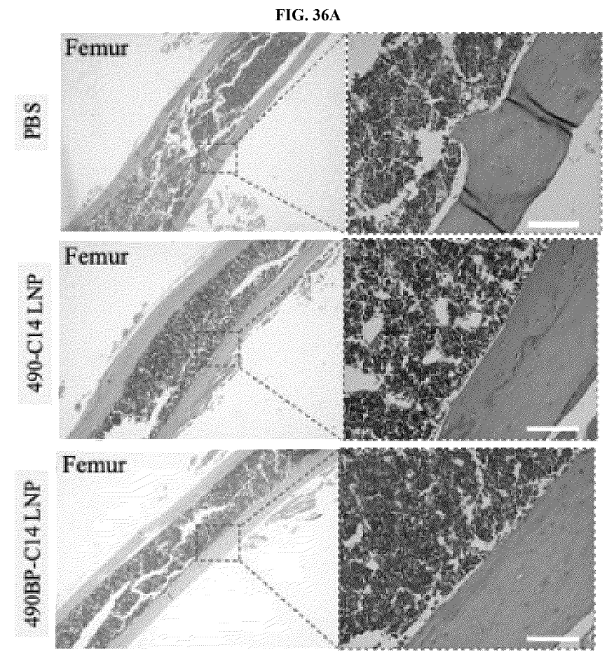

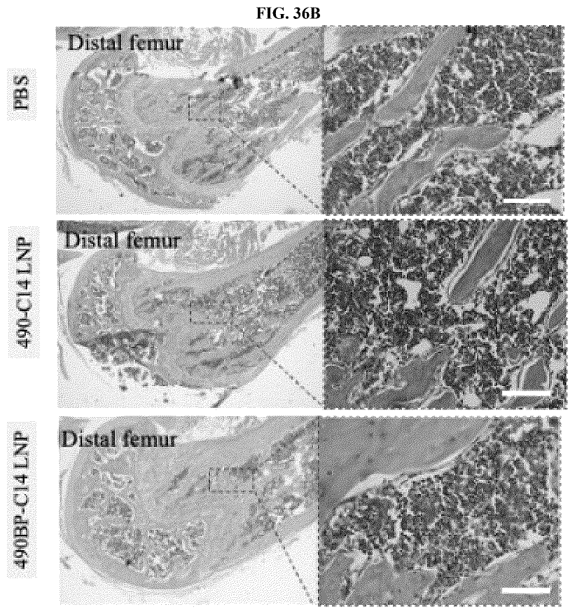

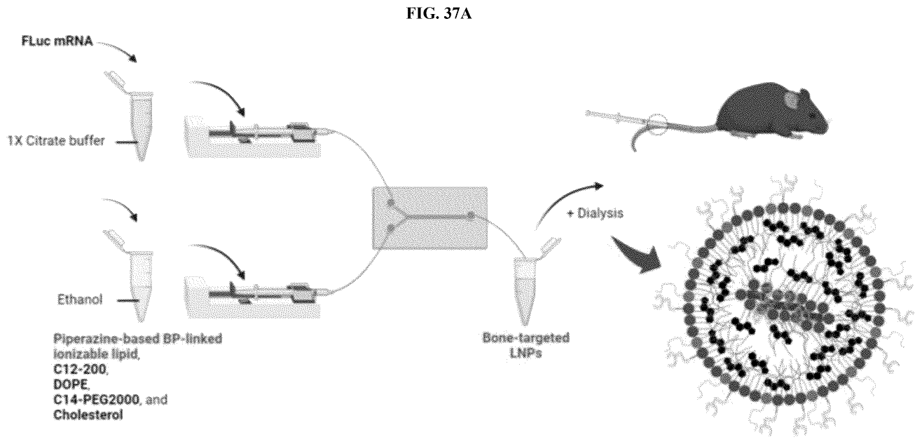

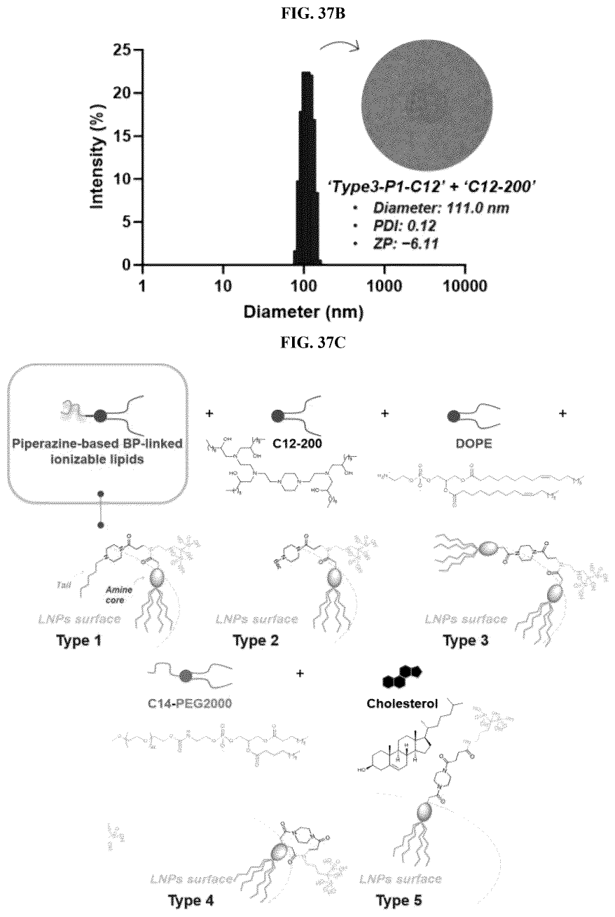

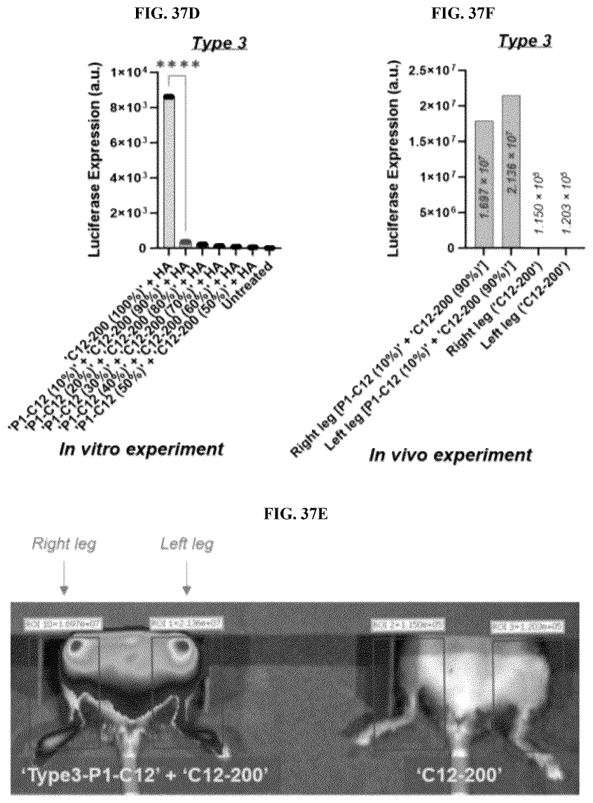

Without wishing to be limited by any theory, these piperazine-based bisphosphonate-linked (PIP-BP) ionizable lipids are distinct from previous bisphosphonate ionizable lipids in certain aspects, such as but not limited to piperazine-based linkages and incorporation of alkyl chains attached to the piperazine terminal group. In one aspect, to achieve strong binding to bones, it is crucial for the terminal group of piperazine-bisphosphonate ionizable lipids, as depicted in FIGS. 37A-37F, to maintain a chemical skeleton on the surface of LNPs. To accomplish this, a chemically structured piperazine with a chair-form configuration was introduced to immobilize the movement of bisphosphonate molecules. Specifically, inspired by the robust structure that can be formed when this piperazine ring undergoes amide bond formation with acyl groups via coupling reactions, novel ionizable lipids were synthesized. In another aspect, these formed amides play a vital role in enhancing luciferase expression by assisting in the late stage of endosomal escape of mRNA. Secondly, besides the fixation of bisphosphonate on the surface of LNPs, diverse synthesis designs of ionizable lipids were developed chemically to facilitate lysosomal-endosomal escape (Type 1 to Type 5). A comparison between Type 1 and Type 2 elucidates how alkyl chains attached to the piperazine terminal group disrupt the LNP membrane to induce mRNA release. In other words, long alkyl chains with hydrophobicity form loose associations with adjacent lipids and PEG units compared to the Boc protecting group, thereby enhancing mRNA escape capability in the late stage of endosomal escape. In contrast to Type 1, Type 2, Type 3, and Type 4 primarily focus on maintaining the skeleton on the surface of LNPs. However, the structural drawback of Type 4 lies in the ring structure formed through cyclization, which may result in the random orientation of the bisphosphonate group on the surface of LNPs, either outward or inward. Finally, Type 5 was designed and synthesized to clearly demonstrate the unique rigidity of the acyl piperazine backbone, bearing a similar skeletal structure to the ionizable lipids used in previously developed LNPs.

These PIP-BP ionizable lipids were initially formulated into LNPs using the commonly utilized molar ratios in lipid formulation, including 35% piperazine-based bisphosphonate-linked ionizable lipid, 16% DOPE phospholipid, 46.5% cholesterol, and 2.5% C14-PEG2000. Typically, most lipids with epoxide-based alkyl chains exhibit strong expression in the liver. Therefore, after the formulation of these LNPs, an initial screening was performed using the Hep-G2 cell line to prioritize selecting lipids that demonstrate top performance. Through this series of research endeavors, an understanding of the mechanism of action of PIP-BP ionizable lipids and LNPs within cells has been achieved, and in-depth studies on these fundamental principles are ongoing as an extension of this work. The PIP-BP ionizable lipids utilized in this study, characterized by their relatively large molecular weight compared to other ionizable lipids, were synthesized to enhance the stability of the resulting PIP-BP LNPs and to produce LNPs with a uniform size distribution, achieved through mixing with ‘C12-200’ ionizable lipid, which is based on a piperazine backbone structure. The most effective PIP-BP LNPs in in vivo experiments, ‘Type3-P1-C12’+‘C12-200’, maintained physicochemical properties of 111 nm in size, 0.12 polydispersity index (PDI), and a 6 value of −6.11 at the fixed molar ratio.

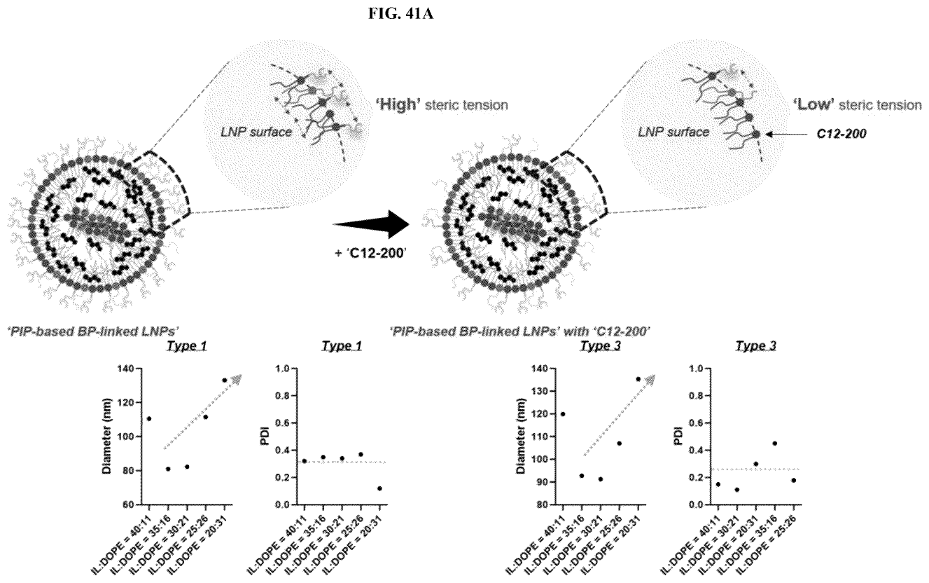

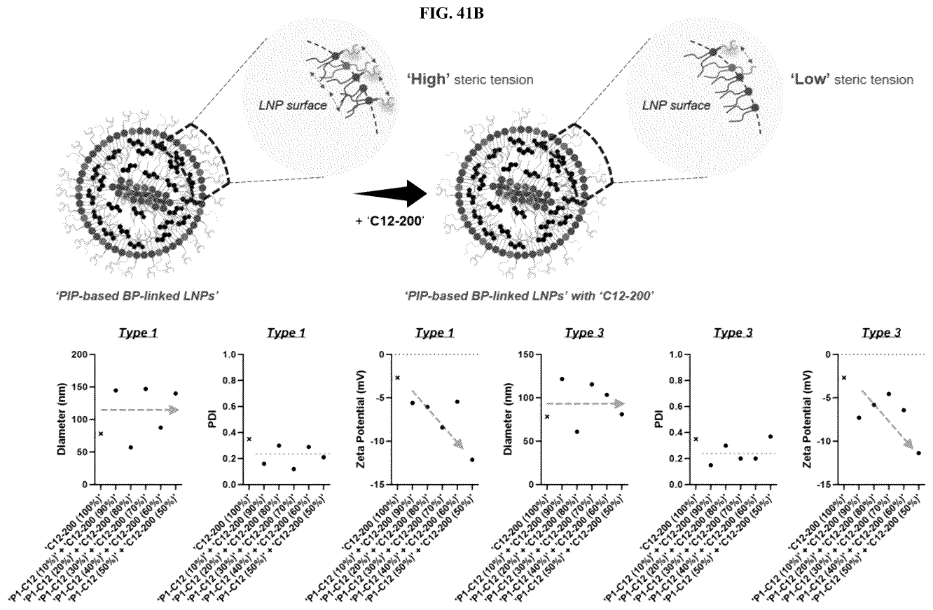

One non-limiting advantage of using the newly synthesized piperazine-based bisphosphonate ionizable lipids lies in their ease of application across various fields. In this study, bone-targeting LNPs were manufactured based on various types of PIP-BP ionizable lipids, with a fixed molar ratio (Ionizable lipid/DOPE/Cholesterol/C14-PEG2000=35/16/46.5/2.5%) maintained among LNPs formulations. All 140 types of bone-targeted LNPs exhibited consistent negative surface charges and chemically anticipated morphologies. Altering the molar ratios among the lipids in this LNP formulation enables the manufacture of bone-targeting LNPs with tunable physicochemical properties (FIG. 41A). Hence, this library of novel bone-targeting ionizable lipids is impactful for targeting the transfection of cells in mineralized tissue microenvironments.

Further, these PIP-BP LNPs can be immobilized on a hydroxyapatite surface, which enables three-dimensional shape analysis using analytical equipment such as AFM, QCM-D, and cryo-TEM. These characterizations allow accurate interpretation of LNP morphology in future work. The piperazine-based bisphosphonate-linked lipid ‘Type3-P1-C12’ used in this study exhibited very high luciferase expression in both in vitro and in vivo experiments, with significantly lower cytotoxicity compared to LNPs based on ‘C12-200’ ionizable lipid. The biological applications based on this research hold immense potential for advancement, including nucleotide delivery to bone marrow cells, bone regeneration, and dental and craniofacial tissues.

In conclusion, PIP-BP LNPs were fabricated from synthesized ionizable lipids for targeted transfection of mineralized tissues. Analysis of structural-activity relationships revealed the impact of bisphosphonate-conjugated piperazine backbone ionizable lipids on LNPs and their roles in cells. Observations of lipid interactions provided insights into LNP morphology. This study paves the way for diverse applications and the development of efficient ionizable lipids based on lipid design approaches and chemical methodologies.

### Description—Bisphosphonate Lipid Nanoparticle (BP-LNP)-Adsorbed Mineralized Tissue Compositions and Methods of Use Thereof

Mineralized tissue refers to complex structures in vertebrates such as bone, dentin, and enamel, composed of cellular components, vasculature, minerals, collagen, and extracellular proteins. Local delivery of therapeutics to mineralized tissue niches holds significant potential for enabling novel targeted treatments for diseases affecting mineralized tissues. Localized drug delivery strategies for bone repair and regeneration, craniofacial complex regeneration, and periodontal treatments are emerging, but the difficulty of obtaining high concentrations of the drug within the diseased area has led to a great demand for new drug delivery systems for the local treatment within bones and oral cavities.

Nanomedicines are recognized for their ability to improve drug delivery by enhancing targeting, increasing bioavailability, and minimizing side effects. Target nucleic acid expression (TEX) systems represent a promising class of treatments for mineralized tissues, as they modulate gene expression at specific sites by introducing exogenous nucleic acids to meet clinical needs, including messenger RNA (mRNA), plasmid DNA (pDNA), small interfering RNA (siRNA), and microRNA (miRNA). To date, lipid nanoparticles (LNPs) are among the most advanced non-viral RNA therapeutics delivery vectors, offering high biocompatibility and effective clinical performance. Notably, LNP-based RNA therapeutics, including FDA-approved siRNA therapy ONPATTRO from Alnylam Pharmaceuticals and the mRNA COVID-19 vaccines developed by Moderna and Pfizer/BioNTech, had progressed from emergency use to full approval, highlighting the translatability of RNA-LNP-based TEX systems. Hence, described herein, in one aspect, is the development of a novel Mineralized tissue Target EXpression system (MiTEX) localized in mineralized tissue niches via RNA-LNPs.

Dysregulation in the signaling pathways in cells leads to diseases that are influenced by genetic factors, but existing therapies mainly offer symptomatic relief and are often limited by clinical complications and potential side effects. Signal transducer and activator of transcription 3 (STAT3) is a phosphorylation-activated protein that translocates to the nucleus to regulate gene expression, playing a key role in a broad range of pathological processes, including immune escape, tumorigenesis, and inflammation. STAT3 functions as a common downstream effector of multiple cytokines, modulating cellular proliferation and intercellular interactions, while directly influencing disease progression through its regulation of mesenchymal stem cell differentiation, osteoclast activation, macrophage polarization, angiogenesis, and cartilage degradation. Small interfering RNA (siRNA) therapeutics are promising for reversibly silencing any gene, which is a valuable tool to treat disease by inhibiting the expression of any targeted protein implicated in disease progression. Among these, STAT3 siRNA delivered via lipid nanoparticles (LNPs) can be a potential candidate for treating inflammatory diseases within mineralized tissues by silencing STAT3 expression, thereby modulating cytokine signaling and halting disease progression.

Recent advances have focused on the rational design of novel lipid components to formulate LNPs capable of targeting specific tissues and cell types. This LNP targeting strategy avoids low-efficiency bioconjugate reactions and species-specific affinity ligands, which present difficulties for scale-up and translation of targeted nanomedicines. The effectiveness of local and systemically administered drugs is limited mainly due to poor bone and oral bioavailability which leads to the low effectiveness of the drugs in mineralized tissues. Alendronate is a bisphosphonate drug that helps prevent bone resorption and enhance bone density, making it an effective treatment for osteoporosis and other bone-related disorders. The piperazine structure improves the presentation of bisphosphonate groups on the nanoparticle surface, thereby promoting stability and affinity to bone minerals. Candidate piperazine-linked bisphosphonate ionizable lipids were identified from in vitro screening and demonstrated targeted delivery to the bone microenvironment in vivo following systemic administration. However, the interactions between bisphosphonate LNPs (BP-LNPs) within the MiTEX and cells on bone mineral interfaces, as well as local delivery approaches of MiTEX required for oral and orthopedic practices, remain unexplored.

In one aspect, the disclosure describes the design of a new gene expression system targeting mineralized tissue to restore oral and bone-related tissues, Mineralized tissue Target Expression system (MiTEX). MiTEX proposed a new approach to locally deliver RNA to mineralized tissue niches by the surface affinity and adsorption of bisphosphonate lipid nanoparticles (BP-LNPs). Bisphosphonate ionizable lipids formed stable, bone-affinitive LNPs when combined with DOPE, cholesterol, and C14-PEG2000 through pipette mixing. After performing an initial selection from in vitro screening on BJ cells, BP-200-C12 LNP was identified as the lead formulation among a series of BP-LNPs due to its piperazine core and shorter epoxide tail, which enhance its cell transfection efficiency. BP-LNPs also successfully delivered Cre-recombinase mRNA to tdROSA mMSCs, which established a basis for investigating the localized delivery of genetic-labeling mRNA-LNPs to mineralized tissue interfaces in vivo. Compared to LNP that did not incorporate bisphosphonate group (C12-200 LNP), BP-LNPs exhibited significantly greater affinity and binding efficiency to HA substrate. The adsorption of MiTEX on HA surface retained RNA delivery capacity to adjacent cells, which provided a new strategy for locally delivering RNA in mineral substrates. Subsequently, bone graft material was utilized as a MiTEX adsorption and storage substrate that successfully delivered mRNA to surrounding cells in vitro. STAT3 expression and downstream biomarker levels were significantly decreased after treating gingival fibroblasts with STAT3 siMiTEX in vitro, which can potentially be applied for anti-inflammatory therapeutics in mineralized tissues.

This study found that the surface affinity and adsorption of MiTEXs on mineral substrates provide a local reservoir for RNA loading and delivery, and these can be applied for nucleic acid based-nanomedicines localizing in mineralized tissue niches. MiTEX exhibited high affinity and efficient adsorption in mineral substrates, rendering HA-mediated transfection in surrounding cells, compared to control. This adsorption-transfection was also shown during the in vivo bone formation process. mMSCs were mixed with MiTEX into one solution before being loaded onto the bone graft scaffolds to avoid insufficient adsorption and variables in each group. Since the pre-mix time was shorter than 5 minutes prior to subcutaneous implantation, it was assumed that there was negligible transfection caused by direct contact of LNPs with the cells, and the main transfection was happening in vivo, mediated by adsorbing MiTEXs within the bone graft scaffolds. This assumption was supported by the adsorption-transfection via the MiTEX-loaded bone graft observed in vitro.

With this strategy, the potential of delivering siRNA-LNPs to modulate STAT3 pathway in the mineralized tissue microenvironment was exhibited. The binding and capture of MiTEXs by HA substrates were illustrated in previous studies, providing a foundational rationale for further investigating the mechanisms and efficiency of their adsorption onto HA, particularly in the context of targeted delivery to mineralized tissue interfaces. However, the capacity of RNA delivery after HA adsorption and local delivery approaches were first explored in this study, demonstrating a new strategy of RNA delivery via surface adsorption and uptake on mineral substrates. MiTEX targeting bone mineral interfaces provide a novel RNA delivery and gene expression platform for local treatments in mineralized tissues, which hold immense potential for clinical translation through delivering siRNA therapeutics or combining them with clinical-used biomaterials. This versatile strategy for localizing and storing MiTEXs can be applied to a wide array of biomaterials, paving the way for new advances in precision nanomedicines.

### Definitions

The term “about” as used herein can allow for a degree of variability in a value or range, for example, within 10%, within 5%, or within 1% of a stated value or of a stated limit of a range, and includes the exact stated value or range.

The term “abnormal” when used in the context of organisms, tissues, cells or components thereof, refers to those organisms, tissues, cells or components thereof that differ in at least one observable or detectable characteristic (e.g., age, treatment, time of day, and so forth) from those organisms, tissues, cells or components thereof that display the “normal” (expected) respective characteristic. Characteristics which are normal or expected for one cell or tissue type, might be abnormal for a different cell or tissue type.

The term “adjuvant” as used herein is defined as any molecule to enhance an antigen-specific adaptive immune response.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)=CH₂, —C(CH₃)═CH(CH₃), —C(CH₂CH₃)=CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

“Alkylamino” refers to a group of the formula —NHRₐ or —NRₐRₐ where each Rₐ is, independently, an alkyl, alkenyl or alkynyl group as defined above containing 1 to 20 carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl group as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl group as defined above. Unless stated otherwise specifically in the specification, an alkyl carbonyl group can be optionally substituted.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C═C(CH₂CH₃), —CH₂C≡CH, —CH₂C≡C(CH₃), and —CH₂C≡C(CH₂CH₃) among others.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms. Similarly, the terms “heteroalkylenyl”, “cycloalkylenyl”, “heterocycloalkylenyl”, and the like, as used herein, refer to a divalent radical of the moiety corresponding to the base group (e.g., heteroalkyl, cycloalkyl, and/or heterocycloalkyl). A divalent radical possesses two open valencies at any position(s) of the group, wherein each radical may be on a carbon atom or heteroatom. Thus, the divalent radical may form a single bond to two distinct atoms or groups, or may form a double bond with one atom.

As used herein, the terms “amino acid”, “amino acidic monomer”, or “amino acid residue” refer to any of the twenty naturally occurring amino acids including synthetic amino acids with unnatural side chains and including both D and L optical isomers.

The “aminoalkyl linker” or “aminoalkylenyl” as used herein refers to a bivalent, at least partially saturated, aliphatic diradical comprising at least one nitrogen atom. In certain embodiments, the nitrogen atom has a lone pair. In certain embodiments, the term may be regarded as a moiety derived from the corresponding aminoalkyl by removal of two hydrogen atoms from the same or different carbon atom and/or heteroatom(s). The terms “mono”, “di”, “tri”, “tetra”, “penta”, and the like, used in conjunction with the term “aminoalkyl linker” indicate the number of nitrogen atoms comprising the moiety (i.e., a triaminoalkyl linker comprises three nitrogen atoms).

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically.

An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, prodrug, or other variant of the reference molecule.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. □ and □ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “monovalent cation” as used herein refers to any positively charged (+1) organic or inorganic ion. Non-limiting examples include H⁺, NH₄⁺, Li⁺, Na⁺, K⁺, Cu⁺, Ag⁺, Cs⁺, and Au⁺.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cationic lipids comprise a protonatable tertiary amine (e.g., pH titratable) head group, C₁₈ alkyl chains, ether linkages between the head group and alkyl chains, and 0 to 3 double bonds. Such lipids include, e.g., DSDMA, DLinDMA, DLenDMA, and DODMA.

The term “conjugated lipid” as used herein refers to a lipid which is conjugated to one or more polymeric groups, which inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

In particular, in the case of a mRNA, and “effective amount” or “therapeutically effective amount” of a therapeutic nucleic acid as relating to a mRNA is an amount sufficient to produce the desired effect, e.g., mRNA-directed expression of an amount of a protein that causes a desirable biological effect in the organism within which the protein is expressed. For example, in some embodiments, the expressed protein is an active form of a protein that is normally expressed in a cell type within the body, and the therapeutically effective amount of the mRNA is an amount that produces an amount of the encoded protein that is at least 50% (e.g., at least 60%, or at least 70%, or at least 80%, or at least 90%) of the amount of the protein that is normally expressed in the cell type of a healthy individual. For example, in some embodiments, the expressed protein is a protein that is normally expressed in a cell type within the body, and the therapeutically effective amount of the mRNA is an amount that produces a similar level of expression as observed in a healthy individual in an individual with aberrant expression of the protein (i.e., protein deficient individual). Suitable assays for measuring the expression of an mRNA or protein include, but are not limited to dot blots, Northern blots, in situ hybridization, ELISA, immunoprecipitation, enzyme function, as well as phenotypic assays known to those of skill in the art.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “fully encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an OLIGREEN® assay. OLIGREEN® is an ultra-sensitive fluorescent nucleic acid stain for quantitating oligonucleotides and single-stranded DNA or RNA in solution (available from Invitrogen Corporation; Carlsbad, Calif). “Fully encapsulated” also indicates that the lipid particles are serum stable, that is, that they do not rapidly decompose into their component parts upon in vivo administration.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “helper lipid” as used herein refers to a lipid capable of increasing the effectiveness of delivery of lipid-based particles such as cationic lipid-based particles to a target, preferably into a cell. The helper lipid can be neutral, positively charged, or negatively charged. In certain embodiments, the helper lipid is neutral or negatively charged. Non-limiting examples of helper lipids include 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3phosphocholin (POPC) and 1,2-dioleoyl-sn-glycero-3-phosphocholine (DOPC).

The term “heteroalkyl” as used herein by itself or in combination with another term, means, unless otherwise stated, a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) (e.g., O, N, P, and S) may be placed at any interior position of the heteroalkyl group or at either terminal position at which the group is attached to the remainder of the molecule.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

“Immunogen” refers to any substance introduced into the body in order to generate an immune response. That substance can a physical molecule, such as a protein, or can be encoded by a vector, such as DNA, mRNA, or a virus.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKₐ of the protonatable group in the range of about 4 to about 7.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ—C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “immune cell,” as used herein refers to any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKₐ of the protonatable group in the range of about 4 to about 7.

The term “linker” as used herein refers to an organic moiety that connects two parts of a compound (e.g., a small molecule drug and an antibody). The linker can be, in non-limiting examples, a direct bond, a single atom (e.g., —O—), a peptide, or a substituted or unsubstituted alkylene or heteroalkylene moiety (e.g., polyethylene glycol). One skilled in the art would be apprised of the common linkers suitable for use in antibody drug conjugates and methods of preparation thereof.

The term “local delivery,” as used herein, refers to delivery of an active agent or therapeutic agent such as a messenger RNA directly to a target site within an organism. For example, an agent can be locally delivered by direct injection into a disease site such as a tumor or other target site such as a site of inflammation or a target organ such as the liver, heart, pancreas, kidney, and the like.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “conjugated lipid” as used herein refers to a lipid which is conjugated to one or more polymeric groups, which inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

As used herein, “lipid encapsulated” can refer to a lipid particle that provides an active agent or therapeutic agent, such as a nucleic acid (e.g., a protein cargo), with full encapsulation, partial encapsulation, or both. In a preferred embodiment, the nucleic acid is fully encapsulated in the lipid particle (e.g., to form an SPLP, pSPLP, SNALP, or other nucleic acid-lipid particle).

The term “lipid nanoparticle” refers to a particle having at least one dimension on the order of nanometers (e.g., 1-1,000 nm) which includes one or more lipids and/or additional agents.

The term “lipid particle” is used herein to refer to a lipid formulation that can be used to deliver an active agent or therapeutic agent, such as a nucleic acid (e.g., mRNA), to a target site of interest. In the lipid particle of the disclosure, which is typically formed from a cationic lipid, a non-cationic lipid, and a conjugated lipid that prevents aggregation of the particle, the active agent or therapeutic agent may be encapsulated in the lipid, thereby protecting the agent from enzymatic degradation.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “monovalent” as used herein refers to a substituent connecting via a single bond to a substituted molecule. When a substituent is monovalent, such as, for example, F or Cl, it is bonded to the atom it is substituting by a single bond.

The term “mRNA” or “messenger RNA” as used herein refers to a ribonucleic acid sequences which encodes a peptide or protein. In certain embodiments, the mRNA may comprise a “transcript” that is produced by using a DNA template and encodes a peptide or protein. Typically, mRNA comprises 5′-UTR, protein coding region and 3′-UTR. mRNA can be produced by in vitro transcription from a DNA template. Methods of in vitro transcription are known to those of skill in the art. For example, various in vitro transfer kits are commercially available. According to the present invention, mRNA can be modified by further stabilizing modifications and cap formation in addition to the modifications according to the invention.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (P1, PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mal. Cell. Probes, 8:91-98 (1994)).

As used herein, the term “nucleic acid” includes any oligonucleotide or polynucleotide, with fragments containing up to 60 nucleotides generally termed oligonucleotides, and longer fragments termed polynucleotides. In particular embodiments, oligonucleotides of the disclosure are from about 15 to about 60 nucleotides in length. Nucleic acid may be administered alone in the lipid particles of the disclosure, or in combination (e.g., co-administered) with lipid particles of the disclosure comprising peptides, polypeptides, or small molecules such as conventional drugs. In other embodiments, the nucleic acid may be administered in a viral vector.

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “prodrug” refers to an agent that is converted into the parent drug in vivo. For example, the term “prodrug” refers to a derivative of a known direct acting drug, which derivative has enhanced delivery characteristics and therapeutic value as compared to the drug, and is transformed into the active drug by an enzymatic or chemical process. In some embodiments, “prodrug” refers to an inactive or relatively less active form of an active agent that becomes active by undergoing a chemical conversion through one or more metabolic processes. In certain embodiments, upon in vivo administration, a prodrug is chemically converted to the biologically, pharmaceutically, or therapeutically active form of the compound. In yet other embodiments, a prodrug is enzymatically metabolized by one or more steps or processes to the biologically, pharmaceutically, or therapeutically active form of the compound. For example, the present compounds can be administered to a subject as a prodrug that includes an initiator bound to an active agent, and, by virtue of being degraded by a metabolic process, the active agent is released in its active form.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

In certain embodiments, “pseudouridine” refers, in yet other embodiments, to m¹acp³Y (1-methyl-3-(3-amino-3-carboxypropyl) pseudouridine. In yet other embodiments, the term refers to m¹Y (1-methylpseudouridine). In yet other embodiments, the term refers to Ym (2′-O-methylpseudouridine. In yet other embodiments, the term refers to m⁵D (5-methyldihydrouridine). In yet other embodiments, the term refers to m³Y (3-methylpseudouridine). In yet other embodiments, the term refers to a pseudouridine moiety that is not further modified. In yet other embodiments, the term refers to a monophosphate, diphosphate, or triphosphate of any of the above pseudouridines. In yet other embodiments, the term refers to any other pseudouridine known in the art. Each possibility represents a separate embodiment of the present disclosure.

The term “siRNA” or “small interfering RNA” as used herein refers to a small (e.g. generally less than 30 nucleotides) non-coding RNA molecule which functions in transcriptional and post-transcriptional regulation of gene expression. Generally, a siRNA specifically targets 1 nucleic acid. In general, a siRNA comprises a double-stranded RNA molecule that ranges from about 15 to about 29 nucleotides in length. In some embodiments, the siRNA may be 16, 17, 18, 19, 20, 21, 22, 23, 24, 25, 26, 27, 28 or 29 nucleotides in length. In some embodiments, the siRNA may be less than 16, 17, 18, 19, 20, 21, 22, 23, 24, 25, 26, 27, 28 or 29 nucleotides in length. In some embodiments, the siRNA may be more than 16, 17, 18, 19, 20, 21, 22, 23, 24, 25, 26, 27, 28 or 29 nucleotides in length. A siRNA may optionally further comprise one or two single-stranded overhangs, e.g., a 5′ overhang on one or both ends, a 3′ overhang on one or both ends, or a combination thereof. The siRNA may be formed from two RNA molecules that hybridize together or, alternatively, may be generated from a short hairpin RNA (shRNA). In some embodiments, the two strands of the siRNA may be completely complementary, such that no mismatches or bulges exist in the duplex formed between the two sequences. In other embodiments, the two strands of the siRNA may be substantially complementary, such that one or more mismatches and/or bulges may exist in the duplex formed between the two sequences. In certain embodiments, one or both of the 5′ ends of the siRNA may have a phosphate group, while in other embodiments one or both of the 5′ ends lack a phosphate group. In other embodiments, one or both of the 3′ ends of the siRNA may have a hydroxyl group, while in other embodiments one or both of the 5′ ends lack a hydroxyl group. Typically, siRNAs are targeted to exonic sequences of the target nucleic acid. One strand of the siRNA, which is referred to as the “antisense strand” or “guide strand,” includes a portion that hybridizes with a target nucleic acid. A target nucleic acid refers to a nucleic acid sequence expressed by a cell for which it is desired expression be disrupted. In the context of a therapeutic composition of the invention, disrupting expression of a target nucleic acid may produce a beneficial effect. Those of skill in the art are familiar with programs, algorithms, and/or commercial services that design siRNAs for target genes. For example, the Rosetta siRNA Design Algorithm (Rosetta Inpharmatics, North Seattle, Wash.), MISSION® siRNA (Sigma-Aldrich, St. Louis, Mo.) and siGENOME siRNA (Thermo Scientific) may be used.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “peptide,” “polypeptide,” and “protein” are used interchangeably herein, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “substantially” as used herein refers to a majority of, or mostly, as in at least about 50%, 60%, 70%, 80%, 90%, 95%, 96%, 97%, 98%, 99%, 99.5%, 99.9%, 99.9%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(=NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “therapeutic protein” as used herein refers to a protein or peptide which has a positive or advantageous effect on a condition or disease state of a subject when provided to the subject in a therapeutically effective amount. In one embodiment, a therapeutic protein or peptide has curative or palliative properties and may be administered to ameliorate, relieve, alleviate, reverse, delay onset of or lessen the severity of one or more symptoms of a disease or disorder. A therapeutic protein or peptide may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Exemplary therapeutic proteins include, but are not limited to, an analgesic protein, an anti-inflammatory protein, an anti-proliferative protein, an proapoptotic protein, an anti-angiogenic protein, a cytotoxic protein, a cytostatic protein, a cytokine, a chemokine, a growth factor, a wound healing protein, a pharmaceutical protein, or a pro-drug activating protein. Therapeutic proteins may include growth factors (EGF, TGF-α, TGF-β, TNF, HGF, IGF, and IL-1-8, inter alia) cytokines, paratopes, Fabs (fragments, antigen binding), and antibodies.

The terms “treat,” “treating” and “treatment,” as used herein, means reducing the frequency or severity with which symptoms of a disease or condition are experienced by a subject by virtue of administering an agent or compound to the subject.

### Bisphosphonate Lipid Compounds

### Formulae (I)-(IV)

In one aspect, the present disclosure provides a compound having the structure of Formula (I), or a racemate, enantiomer, diastereomer, pharmaceutically acceptable salt, solvate, or derivative thereof:

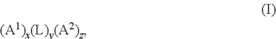

wherein: each occurrence of A¹ is independently

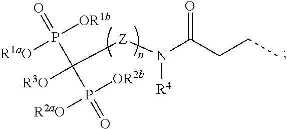

each occurrence of A² is independently

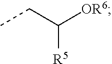

each occurrence of L is an amine linker independently selected from the group consisting of aminoalkyl linker, substituted aminoalkyl linker, diaminoalkyl linker, substituted diaminoalkyl linker, triaminoalkyl linker, substituted triaminoalkyl linker, tetraaminoalkyl linker, substituted tetraaminoalkyl linker, pentaaminoalkyl linker, substituted pentaaminoalkyl linker, polyaminoalkyl linker, substituted polyaminoalkyl linker, aminocycloalkyl linker, substituted aminocycloalkyl linker, diaminocycloalkyl linker, substituted diaminocycloalkyl linker, triaminocycloalkyl linker, substituted triaminocycloalkyl linker, tetraaminocycloalkyl linker, substituted tetraaminocycloalkyl linker, pentaaminocycloalkyl linker, substituted pentaaminocycloalkyl linker, polyaminocycloalkyl linker, substituted polyaminocycloalkyl linker, and any combination thereof;
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}-(C5-C12 cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof,
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si;
each occurrence of z′ and z″ is independently an integer represented by 0, 1, or 2;
wherein x, y, and z are independently an integer from 0 to 20;
each occurrence of n is independently an integer from 0 to 10.

In certain embodiments, L is selected from the group consisting of

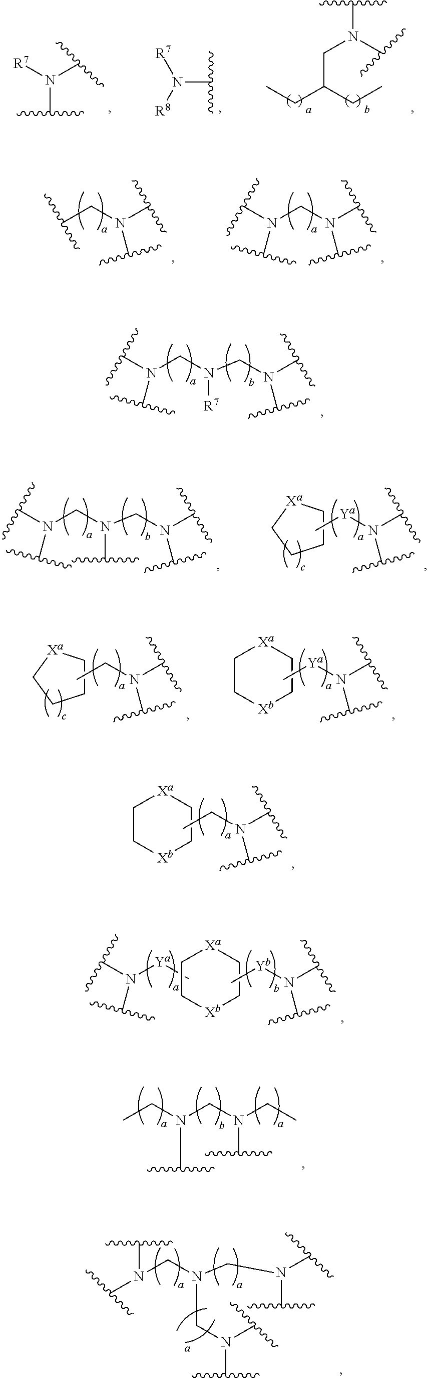

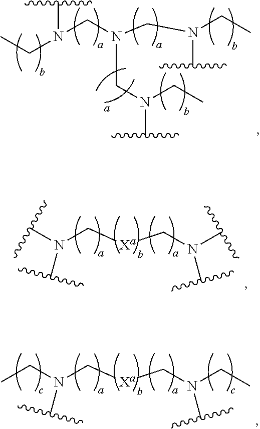

and any combination thereof,
wherein:
each occurrence of R⁷ and R⁸ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z′}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰_{z″},-ester, —Y(R⁹)_{z′}(R¹⁰)z,, —NO₂, —CN, ═O, ═S, sulfoxy, sulfonate, sulfate, sulfite, and sulfide, and any combination thereof,
each occurrence of X^{a} and X^{b} is independently selected from the group consisting of O, S, N(R⁹)_{z′}, P(R⁹)_{z′}, and any combination thereof,
each occurrence of Y^{a} and Y^{b} is independently selected from the group consisting of C1-C₁₂ alkylenyl, substituted C₁-C₁₂ alkylenyl, C₃-C₈ cycloalkylenyl, substituted C₃-C₈ cycloalkylenyl, —Y′(R⁹)_{z′}(R¹⁰)_{z″}—(C₃-C₈ cycloalkylenyl), substituted —Y′(R⁹)_{z′}(R¹⁰_{z″},—C₃-C₈ cycloalkylenyl, C₂-C₈ heterocycloalkylenyl, substituted C₂-C₈ heterocycloalkylenyl, —Y′(R⁹)_{z′}(R¹⁰)_{z″}—C₂-C₈ heterocycloalkylenyl, substituted-Y′(R⁹)_{z′}(R¹⁰)_{z″}—C₂-C₈ heterocycloalkylenyl, C₂-C₈ alkenylenyl, substituted C₂-C₈ alkenylenyl, C₅-C₁₀ cycloalkenylenyl, substituted C₅-C₁₀ cycloalkenylenyl, —Y′(R⁹)_{z′}(R¹⁰)_{z″}—C₅-C₁₀ cycloalkenylenyl, substituted —Y′(R⁹)_{z′}(R¹⁰)_{z″}—C₅-C₁₀ cycloalkenylenyl, C₂-C₈ alkynylenyl, substituted C₂-C₈ alkynylenyl, C₈-C₁₂ cycloalkynylenyl, substituted C₈-C₁₂ cycloalkynylenyl, —Y′(R⁹)_{z′}(R¹⁰)_{z″}—C₈-C₁₂ cycloalkynylenyl, substituted —Y′(R⁹)_{z′}(R¹⁰)_{z″}—C₈-C₁₂ cycloalkynylenyl, C₆-C₁₀ arylenyl, substituted C₆-C₁₀ arylenyl, —Y′(R⁹)_{z′}(R¹⁰)_{z″}—C₆-C₁₀ arylenyl, substituted —Y′(R⁹)_{z′}(R¹⁰)_{z″}—C₆-C₁₀ arylenyl, C₂-C₁₀ heteroarylenyl, substituted C₂-C₁₀ heteroarylenyl, —Y′(R⁹)_{z′}(R¹⁰)_{z″}—C₂-C₁₀ heteroarylenyl, and substituted —Y′(R⁹)_{z′}(R¹⁰_{z″}, —C₂—C₁₀ heteroarylenyl;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2;
wherein each occurrence of a, b, and c is independently an integer from 0 to 10;
each occurrence of [symbol] indicates a bond between a N atom of L and A¹ or A².

In certain embodiments, L is selected from the group consisting of

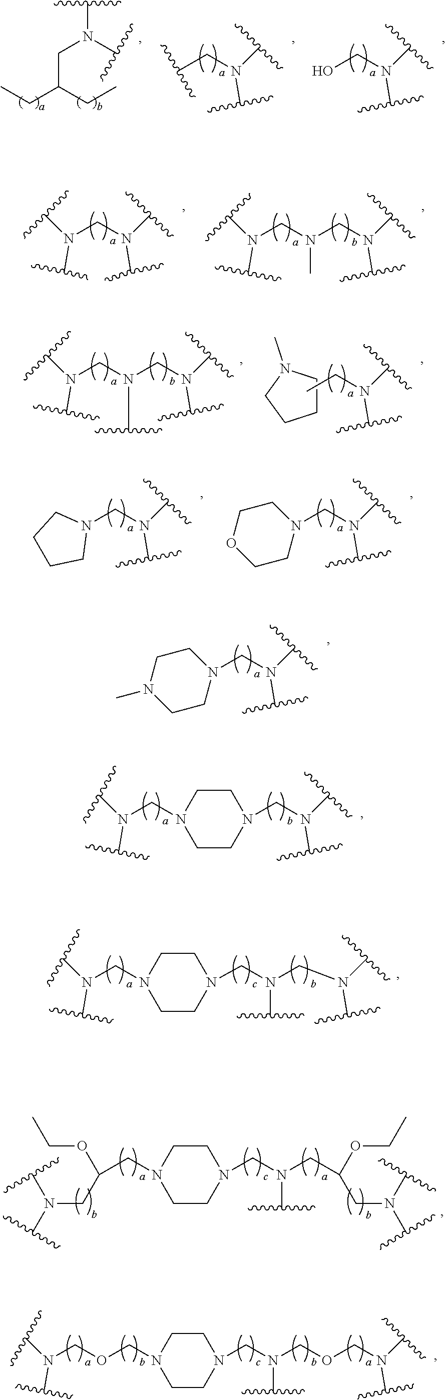

any combination thereof,
wherein:
each occurrence of a, b, and c is independently an integer from 0 to 10; and
each occurrence of

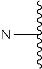 [SMILES: *N]

indicates a bond between a N atom of L and A¹ or A².

In certain embodiments, the compound having the structure of Formula (I) is

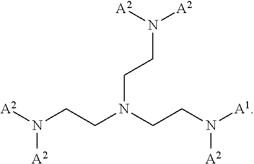

In certain embodiments, the compound having the structure of Formula (I) is

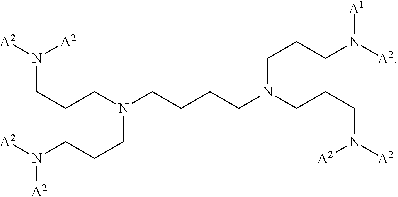

In certain embodiments, the compound having the structure of Formula (I) is

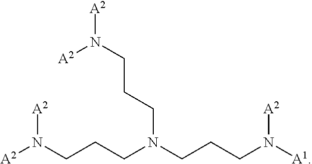

In certain embodiments, the compound having the structure of Formula (I) is

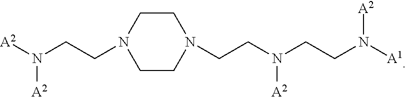

In certain embodiments, the compound having the structure of Formula (I) is

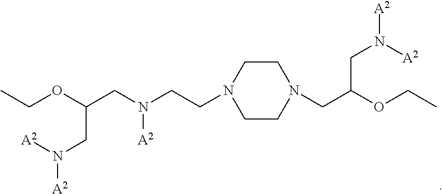

In certain embodiments, the compound having the structure of Formula (I) is

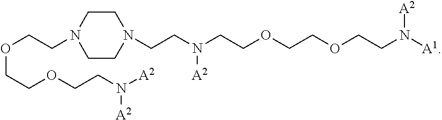

In certain embodiments, the compound having the structure of Formula (I) is

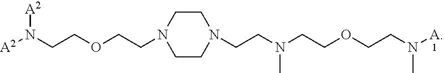

In certain embodiments, A¹ is

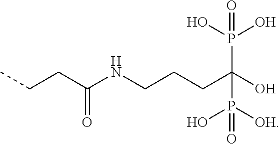

In certain embodiments, A² is

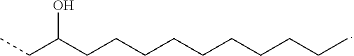

In certain embodiments, A² is

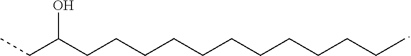

In certain embodiments, A² is

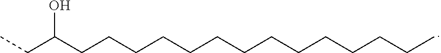

In certain embodiments, the compound having the structure of Formula (I) is a compound having the structure selected from the group consisting of:

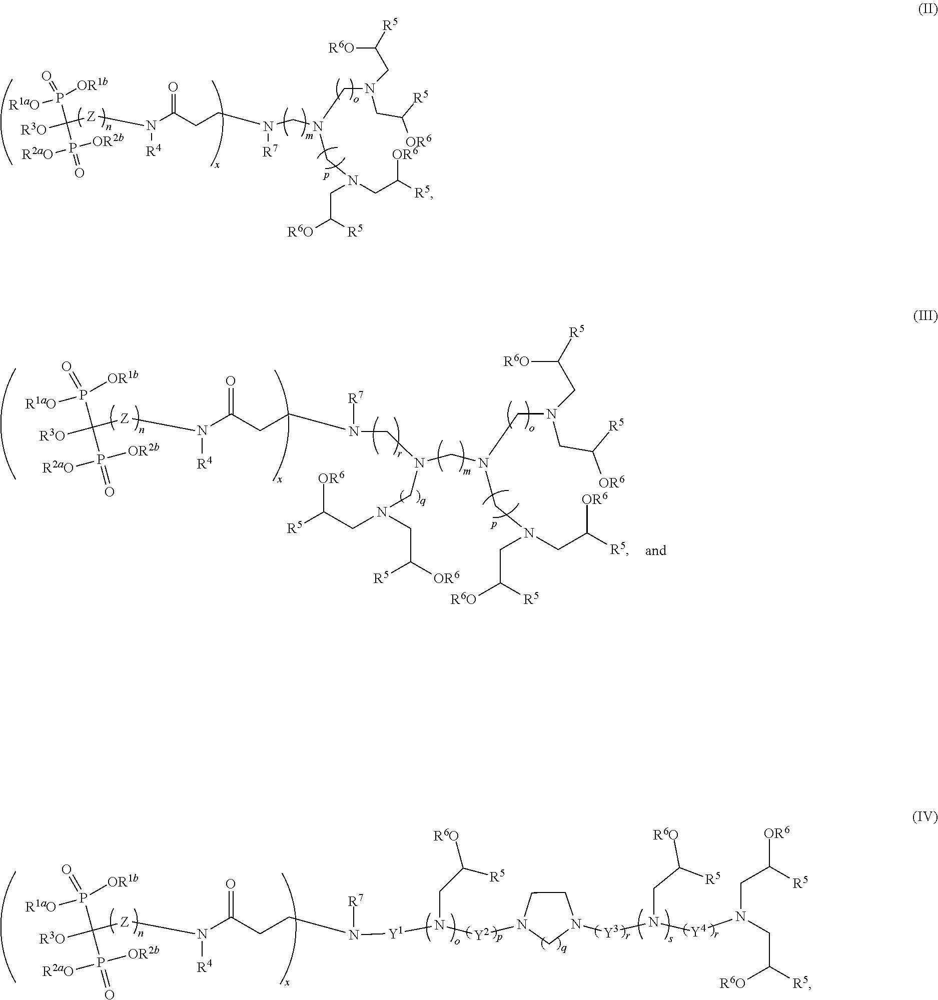

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰_{z″}, —(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰_{z″},-ester, —Y(R⁹)_{z′}(R¹⁰)_{z″}, —NO₂, —CN, ═O, ═S, sulfoxy, sulfonate, sulfate, sulfite, and sulfide, and any combination thereof,
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(Cs—C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof,
each occurrence of Y¹, Y², Y³, and Y⁴ is independently selected from the group consisting of O, S, C(R⁹)_{z′}(R¹⁰)_{z″},N(R⁹)_{z′}, P(R⁹)_{z′}, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

In certain embodiments, the compound having the structure of Formula (I) is a compound having the structure selected from the group consisting of:

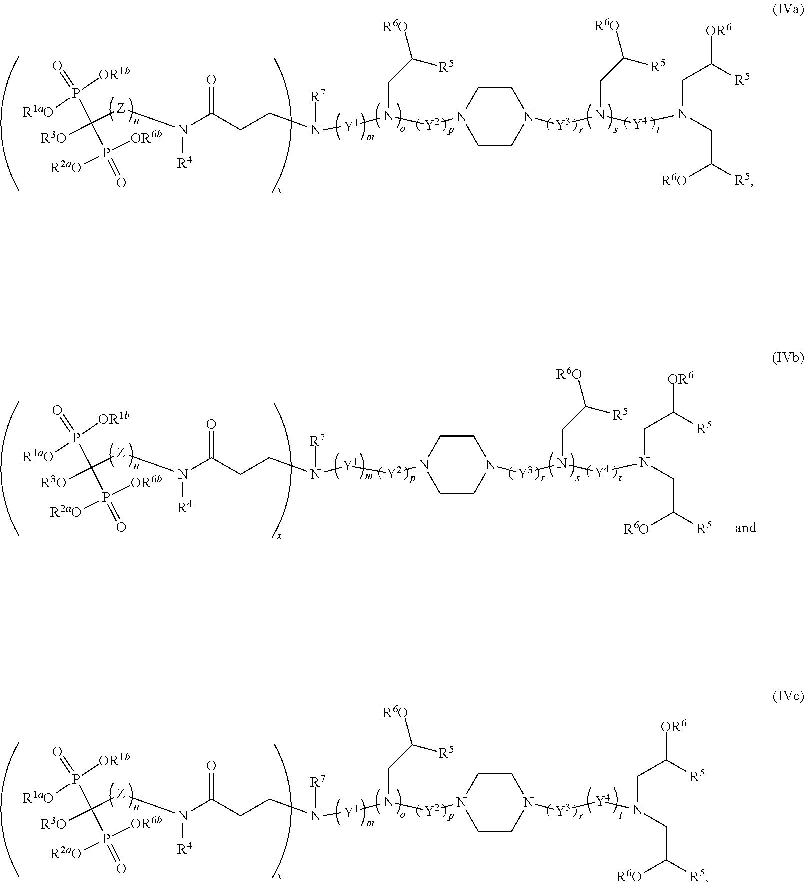

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R¹, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(Cs—C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof,
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof,
each occurrence of Y¹, Y², Y³, and Y⁴ is independently selected from the group consisting of O, S, C(R⁹)_{z′}(R¹⁰)_{z″},N(R⁹)_{z′}, P(R⁹)_{z′}, and any combination thereof; each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

In certain embodiments, the compound having the structure of Formula (I) is a compound having the structure selected from the group consisting of:

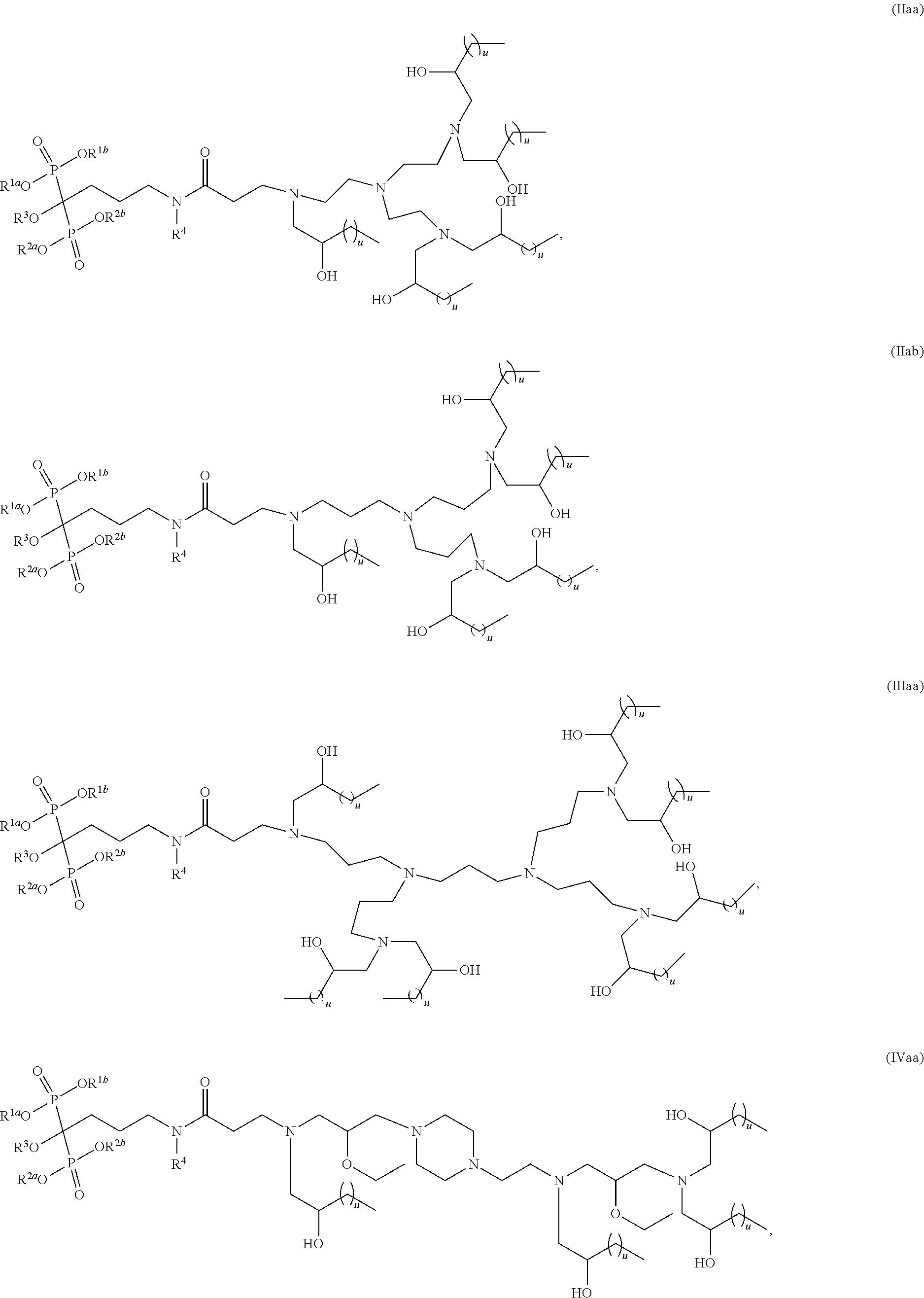

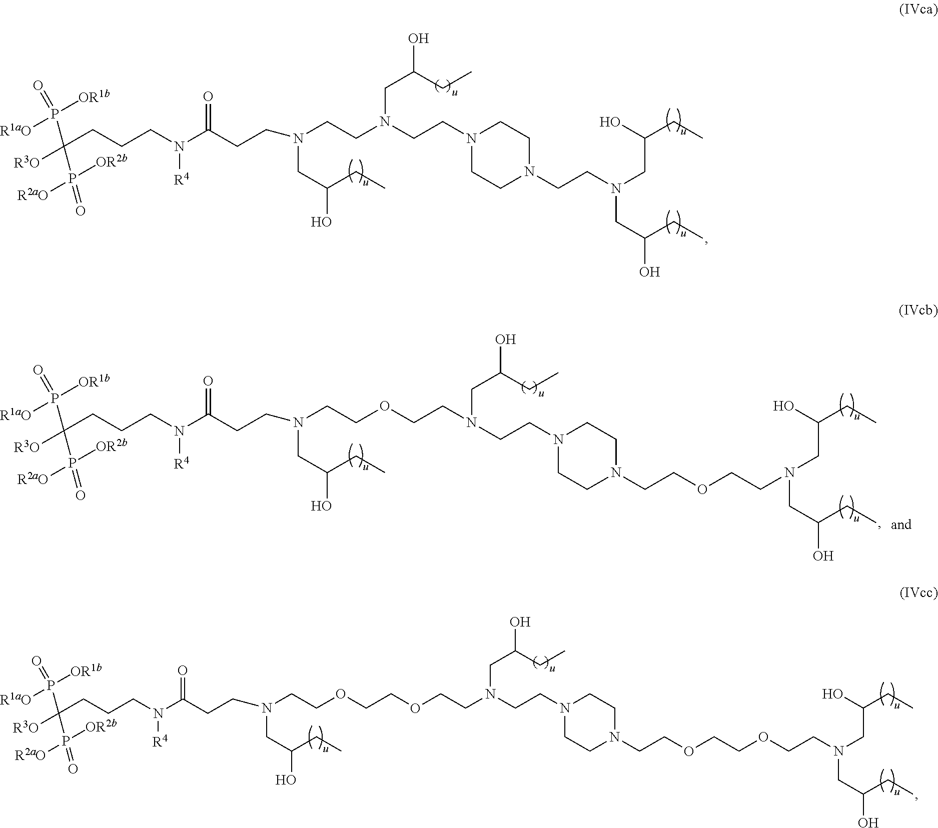

wherein.
each occurrence of R^{1a}, R^{1b}, R^{2a}, R^{2b}, R³, and R⁴ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰_{z″}, —(C₃-C₁₂ cycloalkyl), optionally substituted C₂-C₁₂ heterocycloalkyl, optionally substituted-(R⁹)_{z′}(R¹⁰)_{z′}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z′}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰_{z″}, —(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of R⁹ and R¹⁰ is independently selected from the group consisting of halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein u is an integer from 0 to 20.

In certain embodiments, the compound having the structure of Formula (I) is selected from the group consisting of

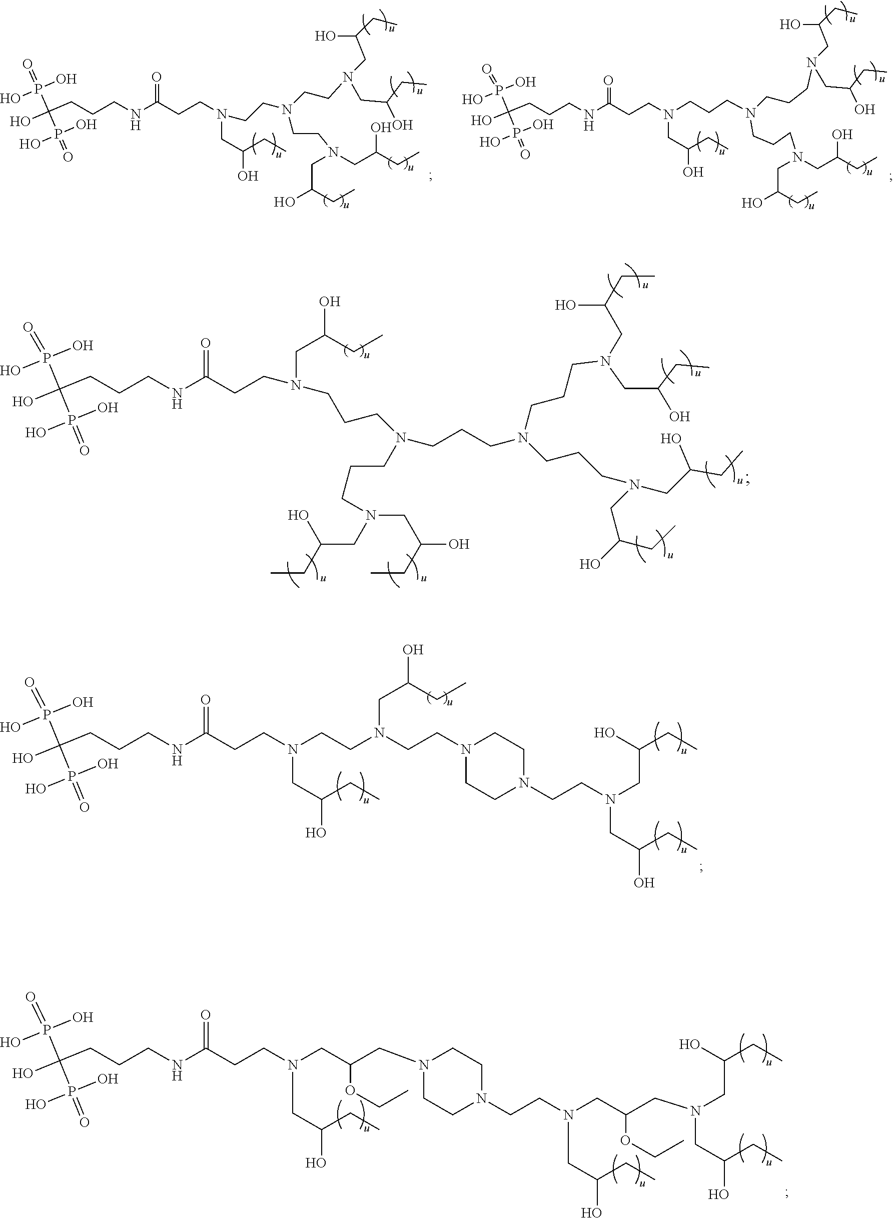

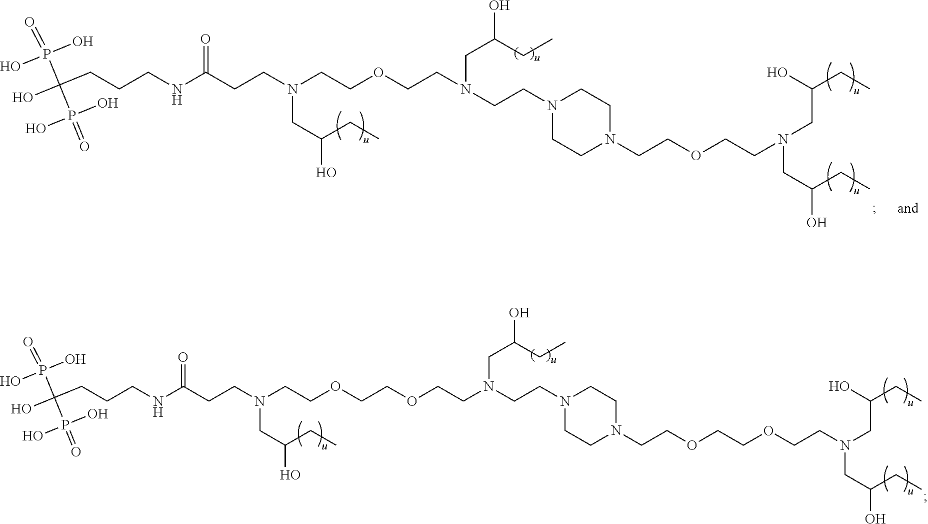

wherein u is an integer from 5 to 15.

In certain embodiments, the compound having the structure of Formula (I) is:

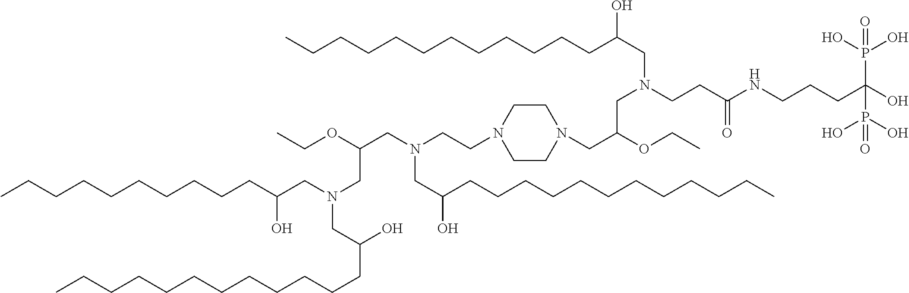 [SMILES: CCCCCCCCCCCCC(O)CN(CCC(=O)NCCCC(O)(P(=O)(O)O)P(=O)(O)O)CC(CN1CCN(CCN(CC(O)CCCCCCCCCCCC)CC(CN(CC(O)CCCCCCCCCC)CC(O)CCCCCCCCCCCC)OCC)CC1)OCC]

(4-(3-((3-(4-(2-((3-(bis(2-hydroxytetradecyl)amino)-2-ethoxypropyl)(2-hydroxytetradecyl)amino)ethyl)piperazin-1-yl)-2-ethoxypropyl)(2-hydroxytetradecyl)amino)propanamido)-1-hydroxybutane-1,1-diyl)bis(phosphonic acid).

### Formula (V)

In one aspect, the disclosure provides compound of Formula (V), or a salt, stereoisomer, or isotopologue thereof:

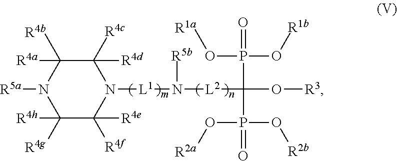

wherein:
R^{1a}, R^{1b}, R^{2a}, R^{2b}, and R³ are each independently selected from the group consisting of H, C(═O)R^{A}, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
R^{4a}, R^{4b}, R^{4c}, R^{4d}, R^{4e}, R^{4f}, R^{4g}, and R^{4h} are each independently selected from the group consisting of H, halogen, and optionally substituted C₁-C₆ alkyl;
R^{5a} and R^{5b} are each independently selected from the group consisting of H, optionally substituted C₁-C₂₄ alkyl, —C(═O)(optionally substituted C₁-C₂₄ alkyl), —C(═O)O(optionally substituted C₁-C₂₄ alkyl), and R⁶,
   wherein at least one of R^{5a} and R^{5b} is R⁶;
each occurrence of R⁶ is independently

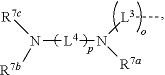

or
   two occurrences of R⁶ can combine with the atoms to which they are bound to form

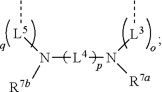

each occurrence of L¹, L², L³, L⁵, and L⁶, if present, is independently selected from the group consisting of -(optionally substituted C₁-C₃ alkylenyl)-, —C(═O)—, —O—, and —N(R^{A})—;
each occurrence of L⁴ is independently selected from the group consisting of —X—, -(optionally substituted C₁-C₁₂ alkylenyl)-, -(optionally substituted C₂-C₁₂ alkenylenyl)-, -(optionally substituted C₁-C₁₂ alkynylenyl)-, -(optionally substituted C₁-C₁₂ heteroalkylenyl)-, -(optionally substituted C₃-C₈ cycloalkylenyl)-, -(optionally substituted C₂-C₈ heterocyloalkylenyl)-, -(optionally substituted C₆-C₁₀ arylenyl)-, and -(optionally substituted C₂-C₈ heteroarylenyl)-;
each occurrence of X, if present, is independently selected from the group consisting of —N(R^{7d})—, —N(R⁸)—, —C(═O)—, and —O—;
each occurrence of R^{7a}, R^{7b}, R^{7c}, R^{7d}, R^{7c}, and R^{7f}, if present, are each independently selected from the group consisting of H, optionally substituted C₁-C₂₄ alkyl and optionally substituted C₁-C₂₄ heteroalkyl;
each occurrence of R⁸ is independently

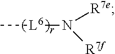

each occurrence of m and n, o, p, q, and r, if present, are each independently an integer selected from the group consisting of 1, 2, 3, 4, 5, 6, 7, 8, 9, and 10; and
each occurrence of R^{A} is independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

In certain embodiments, the compound of Formula (V) is a compound of Formula (Va):

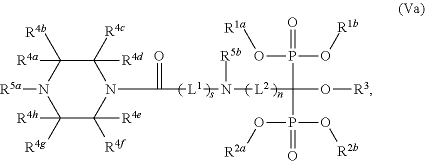

wherein s is an integer selected from the group consisting of 1, 2, 3, 4, 5, 6, 7, 8, and 9.

In certain embodiments, at least one of R^{1a}, R^{1b}, R^{2a}, R^{2b}, and R³ is H. In certain embodiments, at least two of R^{1a}, R^{1b}, R^{2a}, R^{2b}, and R³ are H. In certain embodiments, at least three of R^{1a}, R^{1b}, R^{2a}, R^{2b}, and R³ are H. In certain embodiments, at least four of R^{1a}, R^{1b}, R^{2a}, R^{2b}, and R³ are H. In certain embodiments, each of R^{1a}, R^{1b}, R^{2a}, R^{2b}, and R³ are H.

In certain embodiments, L¹ is —C(═O)—. In certain embodiments, L¹ is —(CH₂)—.

In certain embodiments, -(L¹).- is —C(═O)(CH₂)₂—. In certain embodiments, -(L¹).- is —C(═O)(CH₂)₂C(═O)—.

In certain embodiments, L² is independently —(CH₂)—.

In certain embodiments, -(L²)n- is —(CH₂)₃—.

In certain embodiments, at least one of R^{4a}, R^{4b}, R^{4c}, R^{4d}, R^{4e}, R⁴f R^{4g}, and R^{4h} is H.

In certain embodiments, at least two of R^{4a}, R^{4b}, R^{4c}, R^{4d}, R^{4e}, R⁴f R⁴⁸, and R^{4h} are H. In certain embodiments, at least three of R^{4a}, R^{4b}, R^{4c}, R^{4d}, R^{4e}, R⁴f R⁴⁸, and R⁴h are H. In certain embodiments, at least four of R^{4a}, R^{4b}, R^{4c}, R^{4d}, R^{4e}, R⁴f R⁴⁹, and R^{4h} are H. In certain embodiments, at least five of R^{4a}, R^{4b}, R^{4c}, R^{4d}, R^{4e}, R⁴f R⁴⁹, and R^{4h} are H. In certain embodiments, at least six of R^{4a}, R^{4b}, R^{4c}, R^{4d}, R^{4e}, R⁴f R^{4g}, and R^{4h} are H. In certain embodiments, at least seven of R^{4a}, R^{4b}, R^{4c}, R^{4d}, R^{4e}, R⁴f R^{4g}, and R^{4h} are H. In certain embodiments, each of R^{4a}, R^{4b}, R^{4c}, R^{4d}, R^{4e}, R^{4f} R⁴ and R^{4h} is H.

In certain embodiments, R^{5a} is H. In certain embodiments, R^{5a} is C(═O)O(optionally substituted C₁-C₁₂ alkyl). In certain embodiments, R^{5a} is optionally substituted C₁-C₁₂ alkyl. In certain embodiments, R^{5a} is R⁶.

In certain embodiments, R^{5b} is H. In certain embodiments, R^{5b} is C(═O)O(optionally substituted C₁-C₁₂ alkyl). In certain embodiments, R^{5b} is optionally substituted C₁-C₁₂ alkyl. In certain embodiments, R^{5b} is R⁶.

In certain embodiments, L³ is —C(═O)—. In certain embodiments, L³ is —(CH₂)—. In certain embodiments, -(L³)_{g} is —C(═O)(CH₂)—.

In certain embodiments, L⁵ is —C(═O)—. In certain embodiments, L⁵ is —(CH₂)—. In certain embodiments, -(L⁵)q- is —C(═O)(CH₂)—.

In certain embodiments, L⁴ is —(CH₂)₁₋₃—. In certain embodiments, L⁴ is —O—. In certain embodiments, L⁴ is —N(R^{7d})—. In certain embodiments, L⁴ is —N(R⁸)—. In certain embodiments,

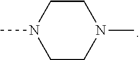

In certain embodiments, L⁶ is —(CH₂)₁₋₃—.

In certain embodiments, R⁶ is

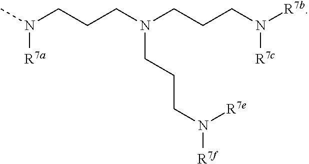

In certain embodiments R⁶ is

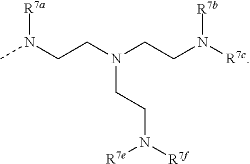

In certain embodiments, R⁶ is

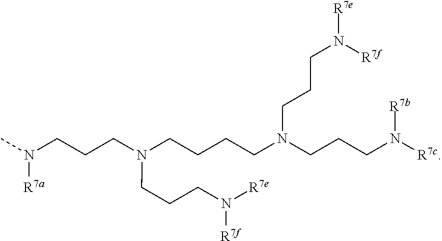

In certain embodiments, R⁶ is

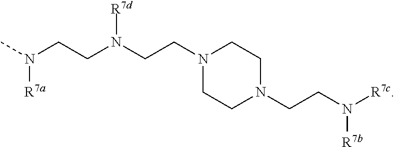

In certain embodiments, R⁶ is

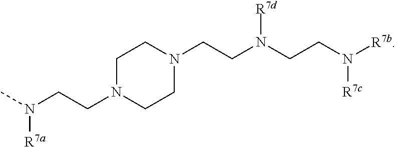

In certain embodiments, R⁶ is

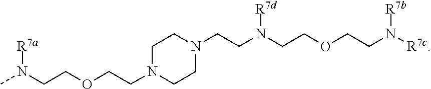

In certain embodiments, R⁶ is

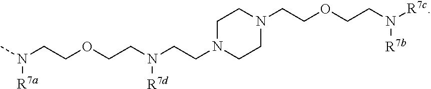

In certain embodiments, R⁶ is

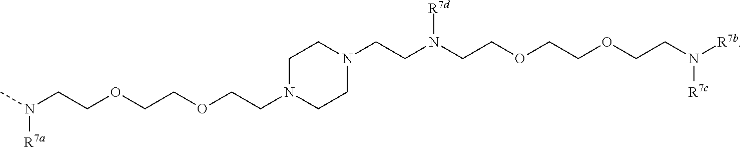

In certain embodiments, R⁶ is

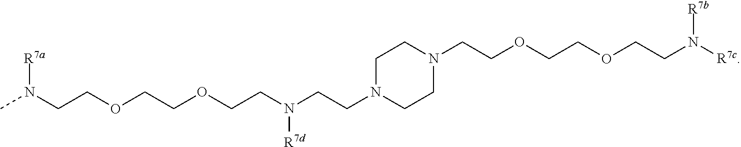

in certain embodiments, R⁶ is

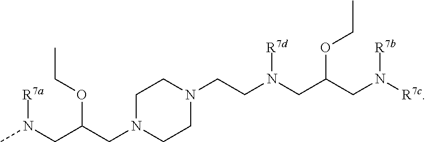

In certain embodiments, R⁶ is

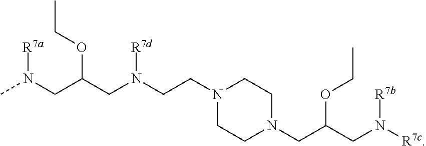

In certain embodiments, two occurrences of R⁶ combine with the atoms to which they are bound to form

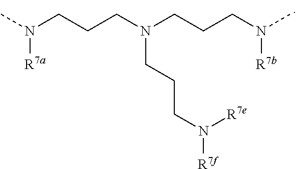

In certain embodiments, two occurrences of R⁶ combine with the atoms to which they are bound to form

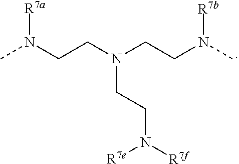

In certain embodiments, two occurrences of R⁶ combine with the atoms to which they are bound to form

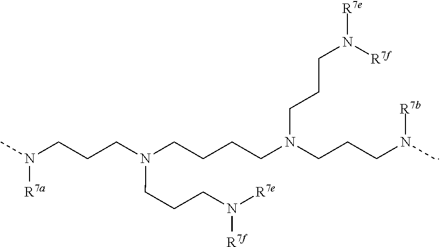

In certain embodiments, two occurrences of R⁶ combine with the atoms to which they are bound to form

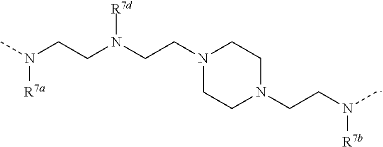

In certain embodiments, two occurrences of R⁶ combine with the atoms to which they are bound to form

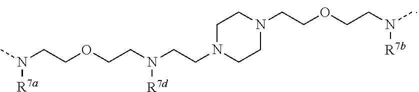

In certain embodiments, two occurrences of R⁶ combine with the atoms to which they are bound to form

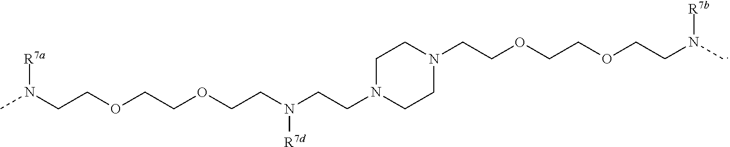

In certain embodiments, two occurrences of R⁶ combine with the atoms to which they are bound to form

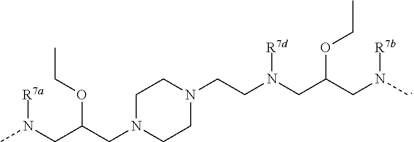

In certain embodiments, R^{7a} is —(CH₂)CH(OH)(optionally substituted C₁-C₂₂ alkyl). In certain embodiments, R⁷h is —(CH₂)CH(OH)(optionally substituted C₁-C₂₂ alkyl). In certain embodiments, R^{7c} is —(CH₂)CH(OH)(optionally substituted C₁-C₂₂ alkyl). In certain embodiments, R^{7d} is —(CH₂)CH(OH)(optionally substituted C₁-C₂₂ alkyl). In certain embodiments, R^{7c} is —(CH₂)CH(OH)(optionally substituted C₁-C₂₂ alkyl). In certain embodiments, R^{7f} is —(CH₂)CH(OH)(optionally substituted C₁-C₂₂ alkyl).

In certain embodiments, R^{7a} is

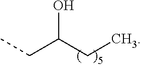

In certain embodiments, R^{7a} is

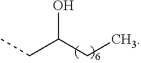

In certain embodiments, R^{7a} is

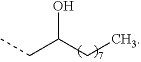

In certain embodiments, R^{7a} is

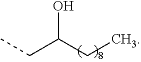

In certain embodiments, R^{7a} is

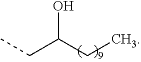

In certain embodiments, R^{7a} is

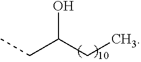

In certain embodiments, R^{7a} is

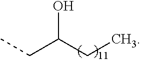

In certain embodiments, R^{7a} is

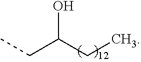

In certain embodiments, R^{7a} is

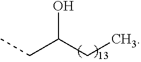

In certain embodiments, R^{7b} is

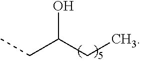

In certain embodiments, R^{7b} is

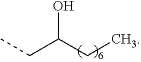

In certain embodiments, R^{7b} is

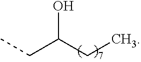

In certain embodiments, R^{7b} is

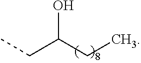

In certain embodiments, R^{7b} is

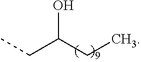

In certain embodiments, R^{7b} is

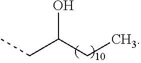

In certain embodiments, R^{7b} is

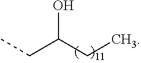

In certain embodiments, R^{7b} is

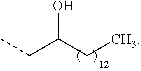

In certain embodiments, R^{7b} is

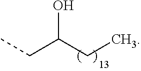

In certain embodiments, R^{7c} is

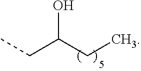

In certain embodiments, R^{7c} is

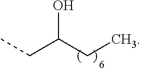

In certain embodiments, R^{7c} is

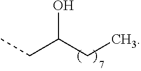

In certain embodiments, R^{7c} is

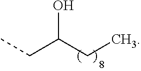

In certain embodiments, R^{7c} is

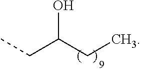

In certain embodiments, R^{7c} is

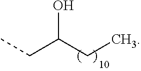

In certain embodiments, R^{7c} is

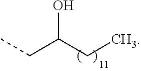

In certain embodiments, R^{7c} is

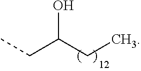

In certain embodiments, R^{7c} is

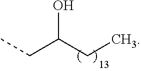

In certain embodiments, R^{7d} is

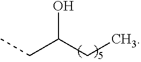

In certain embodiments, R^{7d} is

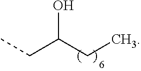

In certain embodiments, R^{7d} is

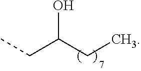

In certain embodiments, R^{7d} is

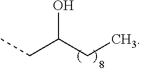

In certain embodiments, R^{7d} is

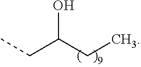

In certain embodiments, R^{7d} is

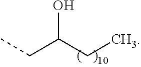

In certain embodiments, R^{7d} is

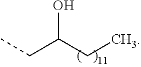

In certain embodiments, R^{7d} is

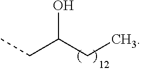

In certain embodiments, R^{7d} is

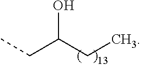

In certain embodiments, R^{7c} is

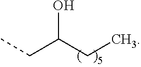

In certain embodiments, R^{7c} is

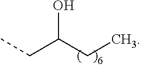

In certain embodiments, R^{7c} is

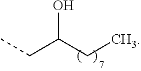

In certain embodiments, R^{7c} is

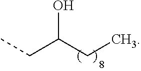

In certain embodiments, R^{7c} is

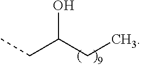

In certain embodiments, R^{7c} is

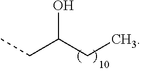

In certain embodiments, R^{7c} is

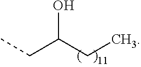

In certain embodiments, R^{7c} is

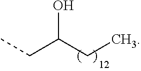

In certain embodiments, R^{7c} is

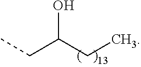

In certain embodiments, R^{7f} is

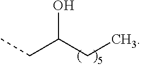

In certain embodiments, R^{7f} is

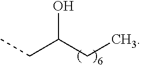

In certain embodiments, R^{7f} is

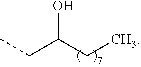

In certain embodiments, R^{7f} is

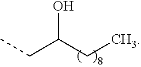

In certain embodiments, R^{7f} is

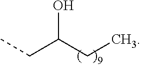

In certain embodiments, R^{7f} is

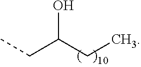

In certain embodiments, R^{7f} is

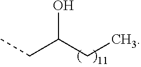

In certain embodiments, R^{7f} is

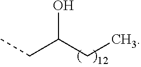

In certain embodiments, R^{7f} is

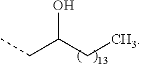

In certain embodiments, the compound is selected from the group consisting of Type1-B1-C10, Type1-B1-C12, Type1-B1-C14, Type1-B1-C16, Type1-B2-C10, Type1-B2-C12, Type1-B2-C14, Type1-B2-C16, Type1-B3-C10, Type1-B3-C12, Type1-B3-C14, Type1-B3-C16, Type1-P1-C10, Type1-P1-C12, Type1-P1-C14, Type1-P1-C16, Type1-P2-C10, Type1-P2-C12, Type1-P2-C14, Type1-P2-C16, Type1-P3-C10, Type1-P3-C12, Type1-P3-C14, Type1-P3-C16, Type1-P4-C10, Type1-P4-C12, Type1-P4-C14, Type1-P4-C16, Type2-B1-C10, Type2-B1-C12, Type2-B1-C14, Type2-B1-C16, Type2-B2-C10, Type2-B2-C12, Type2-B2-C14, Type2-B2-C16, Type2-B3-C10, Type2-B3-C12, Type2-B3-C14, Type2-B3-C16, Type2-P1-C10, Type2-P1-C12, Type2-P1-C14, Type2-P1-C16, Type2-P2-C10, Type2-P2-C12, Type2-P2-C14, Type2-P2-C16, Type2-P3-C10, Type2-P3-C12, Type2-P3-C14, Type2-P3-C16, Type2-P4-C10, Type2-P4-C12, Type2-P4-C14, Type2-P4-C16, Type3-B1-C10, Type3-B1-C12, Type3-B1-C14, Type3-B1-C16, Type3-B2-C10, Type3-B2-C12, Type3-B2-C14, Type3-B2-C16, Type3-B3-C10, Type3-B3-C12, Type3-B3-C14, Type3-B3-C16, Type3-P1-C10, Type3-P1-C12, Type3-P1-C14, Type3-P1-C16, Type3-P2-C10, Type3-P2-C12, Type3-P2-C14, Type3-P2-C16, Type3-P3-C10, Type3-P3-C12, Type3-P3-C14, Type3-P3-C16, Type3-P4-C10, Type3-P4-C12, Type3-P4-C14, Type3-P4-C16, Type4-B1-C10, Type4-B1-C12, Type4-B1-C14, Type4-B1-C16, Type4-B2-C10, Type4-B2-C12, Type4-B2-C14, Type4-B2-C16, Type4-B3-C10, Type4-B3-C12, Type4-B3-C14, Type4-B3-C16, Type4-P1-C10, Type4-P1-C12, Type4-P1-C14, Type4-P1-C16, Type4-P2-C10, Type4-P2-C12, Type4-P2-C14, Type4-P2-C16, Type4-P3-C10, Type4-P3-C12, Type4-P3-C14, Type4-P3-C16, Type4-P4-C10, Type4-P4-C12, Type4-P4-C14, Type4-P4-C16, Type5-B1-C10, Type5-B1-C12, Type5-B1-C14, Type5-B1-C16, Type5-B2-C10, Type5-B2-C12, Type5-B2-C14, Type5-B2-C16, Type5-B3-C10, Type5-B3-C12, Type5-B3-C14, Type5-B3-C16, Type5-P1-C10, Type5-P1-C12, Type5-P1-C14, Type5-P1-C16, Type5-P2-C10, Type5-P2-C12, Type5-P2-C14, Type5-P2-C16, Type5-P3-C10, Type5-P3-C12, Type5-P3-C14, Type5-P3-C16, Type5-P4-C10, Type5-P4-C12, Type5-P4-C14, and Type5-P4-C16.

### LNP Compositions

In certain embodiments, the cationic lipid is an amino lipid. Suitable amino lipids useful in the disclosure include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In certain embodiments, the lipid is a PEGylated lipid, including, but not limited to, DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16—O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). In certain embodiments, the neutral lipid is 1,2-distearoyl-sn-glycero-3-phosphocholine (DSPC).

In some embodiments, the composition comprises a neutral lipid selected from DSPC, DPPC, DSPE, SOPE, SOPC, DOTAP, DMPC, DOPC, POPC, DOPE, and SM.

A “steroid” is a compound comprising the following carbon skeleton:

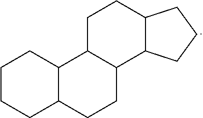 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include polyethylene glycol (PEG), maleimide PEG (mPEG), DSPE-PEG-DBCO, 1-(monomethoxy-polyethyleneglycol)-2,3-dimyristoylglycerol (PEG-s- DMG), DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

In certain embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In certain embodiments, the polyethylene glycol-lipid is N-[(methoxy poly(ethylene glycol)₂₀₀₀)carbamyl]-1,2-dimyristyloxlpropyl-3-amine (PEG-c-DMA). In certain embodiments, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(w-methoxy(polyethoxy)ethyl)butanedioate (PEG-S-DMG), a pegylated ceramide (PEG-cer), or a PEG dialkoxypropylcarbamate such as w-methoxy(polyethoxy)ethyl-N-(2,3-di(tetradecanoxy)propyl)carbamate or 2,3-di(tetradecanoxy)propyl-N-(w-methoxy(polyethoxy)ethyl)carbamate.

In certain embodiments, the additional lipid is present in the LNP in an amount from about 1 mol % to about 10 mol %. In certain embodiments, the additional lipid is present in the LNP in an amount from about 1 mol % to about 5 mol %. In certain embodiments, the additional lipid is present in the LNP in about 1 mol % or about 2.5 mol %.

The term “lipid nanoparticle” refers to a particle having at least one dimension on the order of nanometers (e.g., 1-1,000 nm) which includes one or more lipids, for example a lipid of Formula (I)—(V).

In various embodiments, the LNPs have a mean diameter of from about 10 nm to about 1500 nm, about 30 nm to about 1000 nm, about 30 nm to about 500 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 200 nm, 250 nm, 300 nm, 310 nm, 375 nm, 400 nm, 500 nm, 800 nm, 1000 nm, 1250 nm, 1400 nm, or 1500 nm. For example, in some embodiments, the LNPs have a mean diameter of from about 10 nm to about 1000 nm. In some embodiments, the LNPs have a mean diameter of from about 50 nm to about 500 nm.

In various embodiments, the lipids or the LNP of the present disclosure are substantially non-toxic.

In various embodiments, the lipids or the LNPs described herein are formulated for stability for in vivo cell targeting. For example, in some embodiments, the LNP formulated for stability for in vivo cell targeting comprises at least one compound having the structure of Formula (I) in a concentration range of about 0.1 mol % to about 99.99 mol %. In some embodiments, the at least one compound having the structure of Formula (I) is present in concentration range of about 1 mol % to about 45 mol %. In some embodiments, the at least one compound having the structure of Formula (I) is present in a concentration of about 40 mol %. In some embodiments, the at least one compound having the structure of Formula (I) is present in a concentration of about 30 mol %.

In some embodiments, the LNP formulated for stability for in vivo cell targeting comprises a phospholipid in a concentration range of about 10 mol % to about 45 mol %. In certain embodiments, the phospholipid is dioleoyl-phosphatidylethanolamine (DOPE), and the DOPE is present in a molar ratio of about 16 or at a molar percentage of about 16%.

In some embodiments, the LNP formulated for stability for in vivo a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.) targeting comprises total cholesterol lipid in a concentration range of about 5 mol % to about 50 mol %. In certain embodiments, the total cholesterol is present in a molar ratio of about 46.5, or at a molar percentage of about 46.5%.

In some embodiments, the total cholesterol comprises a substituted cholesterol lipid. In some embodiments, the total cholesterol comprises a mixture of cholesterol and one or more substituted cholesterol lipid. In certain embodiments, the LNP molecule comprises total cholesterol at a ratio of 50% cholesterol:50% substituted cholesterol. In certain embodiments, the LNP molecule comprises total cholesterol at a ratio of 75% cholesterol:25% substituted cholesterol. In certain embodiments, the LNP molecule comprises total cholesterol at a ratio of 87.5% cholesterol:12.5% substituted cholesterol. In certain embodiments, the LNP molecule comprises total cholesterol at a ratio of 0% cholesterol:100% substituted cholesterol.

Exemplary substituted cholesterol lipids that can be incorporated into the LNP of the disclosure include, but are not limited to, a hydroxy substituted cholesterol, an epoxy substituted cholesterol and a keto substituted cholesterol.

In some embodiments, the substituted cholesterol lipid is 7a-hydroxycholesterol, 70-hydroxycholesterol, 19-hydroxycholesterol, 20(S)-hydroxycholesterol, 24(S)-hydroxycholesterol, 25-hydroxycholesterol, 7-ketocholesterol, 5,6-epoxycholesterol, 3β, 5α, 6β-trihydroxycholesterol, 4β-hydroxycholesterol, 27-hydroxycholesterol or 22(R)-hydroxycholesterol.

By way of example, In certain embodiments, the LNP molecule comprises a mixture of 50% cholesterol:50% 7a-hydroxycholesterol. In certain embodiments, the LNP molecule comprises a mixture of 75% cholesterol:25% 7a-hydroxycholesterol.

In some embodiments, the LNP of the present disclosure comprises total PEG in a concentration range of about 0.5 mol % to about 12.5 mol %. In certain embodiments, the total PEG is present in a molar ratio of about 2.5, or at a molar percentage of about 2.5%.

In some embodiments, the PEG comprises a mixture of PEG maleimide PEG (mPEG).

In various embodiments, the LNP of the present disclosure comprises at least one compound having the structure of Formula (I), phospholipid, total cholesterol, and a polymer-conjugated lipid (e.g., PEG-conjugated lipid), wherein the at least one compound having the structure of Formula (I): phospholipid:total cholesterol: polymer-conjugated lipid are present in a molar ratio of about 1-80: 5-45:5-55:0.5-12.5 or at a molar percentage of about 1-⁸0%: 5-45%:5-55%:0.5-12.5%. In certain embodiments, the LNP comprises at least one compound having the structure of Formula (I), phospholipid, total cholesterol and polymer-conjugated lipid, wherein the at least one compound having the structure of Formula (I): phospholipid:total cholesterol: polymer-conjugated lipid are present in a molar ratio of about 35-45: 5-20:40-55: 1-2.5 or at a molar percentage of about 35-45%:5-20%:40-55%:1-2.5%. In certain embodiments, the LNP comprises at least one compound having the structure of Formula (I), phospholipid, total cholesterol and polymer-conjugated lipid, wherein the at least one compound having the structure of Formula (I): phospholipid:total cholesterol: polymer-conjugated lipid are present in a molar ratio of about 30-35:16: 46.5:2.5 or at a molar percentage of about 35%:16%:46.5%:2.5%. In certain embodiments, the LNP comprises at least one compound having the structure of Formula (I), phospholipid, total cholesterol and polymer-conjugated lipid, wherein the at least one compound having the structure of Formula (I): phospholipid:total cholesterol: polymer-conjugated lipid are present in a molar ratio of about 35:16: 46.5:2.5 or at a molar percentage of about 30-35%:16%: 46.5%:2.5%.

For example, In certain embodiments, the LNP comprises at least one compound having the structure of Formula (I), DOPE, total cholesterol, and PEG, wherein the at least one compound having the structure of Formula (I):DOPE:total cholesterol:PEG are present in a molar ratio of about 1-80: 5-45:5-55:0.5-12.5 or at a molar percentage of about 1-⁸0%: 5-45%:5-55%:0.5-1²0.5%. In certain embodiments, the LNP comprises at least one compound having the structure of Formula (I), DOPE, total cholesterol and PEG, wherein the at least one compound having the structure of Formula (I):DOPE:total cholesterol:PEG are present in a molar ratio of about 35-45: 5-20:40-55: 1-2.5 or at a molar percentage of about 35-45%:5-20%:40-55%:1-2.5%. In certain embodiments, the LNP comprises at least one compound having the structure of Formula (I), DOPE, total cholesterol and PEG, wherein the at least one compound having the structure of Formula (I):DOPE:total cholesterol:PEG are present in a molar ratio of about 30-35:16: 46.5:2.5 or at a molar percentage of about 35%: 16%:46.5%:2.5%. In certain embodiments, the LNP comprises at least one compound having the structure of Formula (I), DOPE, total cholesterol and PEG, wherein the at least one compound having the structure of Formula (I):DOPE:total cholesterol:PEG are present in a molar ratio of about 35:16: 46.5:2.5 or at a molar percentage of about 30-35%:16%: 46.5%:2.5%.

In various embodiments, the LNP targets at least one cell of interest. For example, in some embodiments, the LNP targets at least one stem cell, HSC, bone cell, bone marrow cell, or any combination thereof. In some embodiments, the LNP targets at least one stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, or any combination thereof.

In one aspect, the LNP comprises at least one cargo. In various aspects, the disclosure is not limited to any particular cargo or otherwise agent for which the LNP is able to carry or transport. Rather, the disclosure includes any agent that can be carried by the LNP. For example, agents that can be carried by the LNP of the disclosure include, but are not limited to, diagnostic agents, detectable agents, and therapeutic agents. Thus, in various embodiments, the LNP comprises at least one agent. In other embodiments, the LNP encapsulates at least one agent.

In some embodiments, the LNP comprises, or encapsulates, at least one agent. In some embodiments, the weight ratio of (a): the at least one agent is between about 1:1 to about 10:1. In some embodiments, the LNP comprises, or encapsulates, at least one agent. In some embodiments, the weight ratio of (a): the at least one agent is between about 2:1 to about 10:1. In some embodiments, the LNP comprises, or encapsulates, at least one agent. In some embodiments, the weight ratio of (a): the at least one agent is between about 3:1 to about 10:1. In some embodiments, the LNP comprises, or encapsulates, at least one agent. In some embodiments, the weight ratio of (a): the at least one agent is between about 4:1 to about 10:1. In some embodiments, the LNP comprises, or encapsulates, at least one agent. In some embodiments, the weight ratio of (a): the at least one agent is between about 5:1 to about 10:1. In some embodiments, the LNP comprises, or encapsulates, at least one agent. In some embodiments, the weight ratio of (a): the at least one agent is between about 6:1 to about 10:1. In some embodiments, the LNP comprises, or encapsulates, at least one agent. In some embodiments, the weight ratio of (a): the at least one agent is between about 7:1 to about 10:1. In some embodiments, the LNP comprises, or encapsulates, at least one agent. In some embodiments, the weight ratio of (a): the at least one agent is between about 8:1 to about 10:1. In some embodiments, the LNP comprises, or encapsulates, at least one agent. In some embodiments, the weight ratio of (a): the at least one agent is between about 9:1 to about 10:1. In some embodiments, the LNP comprises, or encapsulates, at least one agent. In some embodiments, the weight ratio of (a): the at least one agent is between about 9.5:1 to about 10:1.

Thus, in various embodiments, the LNP is suitable for delivering at least one cargo to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.).

In one aspect, the present disclosure relates to a composition comprising at least one compound or LNP of the present disclosure. In one aspect, the present disclosure relates to a composition comprising at least one compound or LNP of the present disclosure that selectively targets at least one cell of interest. For example, in some embodiments, the composition targets at least one bone cell and/or bone marrow cell (e.g., a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.).

In one aspect, the composition of the present disclosure comprises one or more LNP formulated for targeted delivery of an agent to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.). Examples of such agents include, but are not limited to, a therapeutic agent, diagnostic agent, detectable agent, small molecule, peptide, polypeptide, amino acid molecule, nucleic acid molecule, drug, pro-drug, label, or any combination thereof.

For example, in some embodiments, the composition of the present disclosure comprises at least one therapeutic agent. In certain embodiments, the therapeutic agent is a hydrophobic therapeutic agent. In certain embodiments, the therapeutic agent is a hydrophilic therapeutic agent. Examples of such therapeutic agents include, but are not limited to, one or more drugs, proteins, amino acids, peptides, antibodies, antibiotics, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In certain embodiments, the therapeutic agent is one or more non-therapeutic moieties. In some embodiments, the nanoparticle comprises one or more therapeutic moieties, one or more non-therapeutic moieties, or any combination thereof. In certain embodiments, the therapeutic moiety targets cancer. In some embodiments, the composition comprises folic acid, peptides, proteins, aptamers, antibodies, small RNA molecules, miRNA, shRNA, siRNA, poorly water-soluble therapeutic agents, anti-cancer agents, or any combinations thereof.

In certain embodiments, the therapeutic agent may be an anti-cancer agent. Any suitable anti-cancer agent may be used in the compositions and methods of the present disclosure. The selection of a suitable anti-cancer agent may depend upon, among other things, the type of cancer to be treated and the nanoparticle compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but is not limited to, chemotherapeutic agents, antiproliferative agents, anti-tumor agents, checkpoint inhibitors, and anti-angiogenic agents. For example, in certain embodiments, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene). Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the disclosure can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In certain embodiments, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In some embodiments, the LNP further comprises one or more gene components, such as siRNA or therapeutic DNA fragments. In some embodiments, the gene component is encapsulated in the LNP. In some embodiments, the gene component is on the surface of the LNP, for example, attached to or within the coating material.

In some embodiments, the LNP further comprises a biocompatible metal. Examples of biocompatible metals include, but are not limited to, copper, copper sulfide, iron oxide, cobalt and noble metals, such as gold and/or silver. One of ordinary skill in the art will be able to select of a suitable type of LNP taking into consideration at least the type of imaging and/or therapy to be performed.

### Lipid Nanoparticles (LNPs)

### Bisphosphonate- Substituted Piperazine Ionizable Lipid Compounds and Bone-Targeted Lipid Nanoparticles (LNPs) Thereof

In another aspect, the disclosure provides a lipid nanoparticle (LNP) composition. In certain embodiments, the LNP composition comprises at least one ionizable lipid, wherein the at least one ionizable lipid comprises at least one compound of formula (V). In certain embodiments, the LNP composition comprises at least one neutral lipid. In certain embodiments, the LNP composition comprises at least one cholesterol lipid and/or a modified derivative thereof. In certain embodiments, the LNP composition comprises at least one polymer-conjugated lipid and/or a modified derivative thereof.

In certain embodiments, the at least one ionizable lipid compound comprises less than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 mol % of the LNP. In certain embodiments, the at least one ionizable lipid compound comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 mol % of the LNP. In certain embodiments, the at least one ionizable lipid compound comprises greater than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 mol % of the LNP.

In certain embodiments, the at least one ionizable lipid compound comprises less than about 35 mol % of the LNP. In certain embodiments, the at least one ionizable lipid compound comprises about 35 mol % of the LNP. In certain embodiments, the at least one ionizable lipid compound comprises greater than about 35 mol % of the LNP.

In certain embodiments, the ionizable lipid further comprises C12-200. In certain embodiments, the at least one ionizable lipid compound of formula (V) and the C12-200 have a ratio ranging from about 10:1 to about 1:10 (formula (V):C12:200). In certain embodiments, the at least one ionizable lipid compound of formula (V) and the C12-200 have a ratio of about 2:3 (40% formula (V)).

In certain embodiments, the at least one neutral lipid comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 mol % of the LNP. In certain embodiments, the at least one neutral lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 mol % of the LNP. In certain embodiments, the at least one neutral lipid comprises greater than about 1,2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 mol % of the LNP.

In certain embodiments, the at least one neutral lipid comprises less than about 16 mol % of the LNP. In certain embodiments, the at least one neutral lipid comprises about 16 mol % of the LNP. In certain embodiments, the at least one neutral lipid comprises greater than about 16 mol % of the LNP.

In certain embodiments, the neutral lipid comprises or consists essentially of at least one neutral lipid selected from the group consisting of dioleoylphosphatidylethanolamine (DOPE), distearoylphosphatidylcholine (DSPC), and dioleoylphosphatidylcholine (DOPC).

In certain embodiments, the neutral lipid comprises or consists essentially of dioleoylphosphatidylethanolamine (DOPE).

In certain embodiments, the at least one cholesterol lipid and/or modified derivative thereof comprises less than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or about 75 mol % of the LNP. In certain embodiments, the at least one cholesterol lipid and/or modified derivative thereof comprises about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or about 75 mol % of the LNP. In certain embodiments, the at least one cholesterol lipid and/or modified derivative thereof comprises greater than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or about 75 mol % of the LNP.

In certain embodiments, the at least one cholesterol lipid and/or modified derivative thereof comprises less than about 46.5 mol % of the LNP. In certain embodiments, the at least one cholesterol lipid and/or modified derivative thereof comprises about 46.5 mol % of the LNP. In certain embodiments, the at least one cholesterol lipid and/or modified derivative thereof comprises greater than about 46.5 mol % of the LNP.

In certain embodiments, the at least one cholesterol lipid and/or modified derivative thereof comprises or consists essentially of cholesterol.

In certain embodiments, the at least one polymer-conjugated lipid comprises less than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7. 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, or about 15 mol % of the LNP. In certain embodiments, the at least one polymer-conjugated lipid comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7. 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, or about 15 mol % of the LNP. In certain embodiments, the at least one polymer-conjugated lipid comprises greater than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7. 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, or about 15 mol % of the LNP.

In certain embodiments, the at least one polymer-conjugated lipid comprises less than about 2.5 mol %. In certain embodiments, the at least one polymer-conjugated lipid comprises greater than about 2.5 mol %.

In certain embodiments, the at least one polymer-conjugated lipid comprises or consists essentially of C14-PEG2000.

In certain embodiments, the LNP has a molar ratio of (a): (b): (c): (d) of about 35:16:46.5:2.5.

In certain embodiments, the LNP further comprises at least one cargo selected from the group consisting of a nucleic acid molecule and a therapeutic agent. In certain embodiments, the therapeutic agent is at least one selected from the group consisting of a small molecule, a protein, and an antibody.

In certain embodiments, the LNP comprises a nucleic acid molecule. In certain embodiments, the nucleic acid molecule is a DNA molecule or an RNA molecule. In certain embodiments, the nucleic acid molecule is selected from the group consisting of cDNA, mRNA, miRNA, siRNA, modified RNA, antagomir, antisense molecule, and a targeted nucleic acid, or any combination thereof. In certain embodiments, the nucleic acid molecule encodes a chimeric antigen receptor (CAR). In certain embodiments, the CAR is specific for binding to a surface antigen of a pathogenic cell. In certain embodiments, the nucleic acid molecule encodes at least one selected from the group consisting of mRNA and sgRNA, optionally wherein the ionizable lipid and mRNA have a weight ratio of about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1. In certain embodiments, the mRNA encodes a therapeutic protein, optionally wherein the therapeutic protein is a CRISPR-associated protein, and optionally wherein the CRISPR-associated protein is CRISPR-associated protein 9 (Cas9). In certain embodiments, the therapeutic agent is a CRISPR-associated protein, optionally wherein the CRISPR-associated protein is CRISPR-associated protein 9 (Cas9).

Ionizable Lipids and/or Cationic Lipids

The scope of ionizable lipids contemplated for use in the present disclosure is not limited to ionizable lipids of Formula (V). In the lipid nanoparticles of the disclosure, the cationic lipid or ionizable lipid may comprise, e.g., one or more of the following: (6Z,9Z,28Z,31Z)-heptatriaconta-6,9,28,31-tetraen-19-yl 4-(dimethylamino)butanoate (DLinMC3DMA), [(4-hydroxybutyl)azanediyl]di(hexane-6,1-diyl) bis(2-hexyldecanoate) (ALC-0315), heptadecan-9-yl 8-{(2-hydroxyethyl)[6-oxo-6-(undecyloxy)hexyl]amino}octanoate (SM-102), 1,1′-[[2-[4-[2-[[2-[bis(2-hydroxydodecyl)amino]ethyl](2-hydroxydodecyl)amino]ethyl]-1-piperazinyl]ethyl]imino]bis-2-dodecanol (C12-200), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-45 dimethylaminopropyl)- 1,3]-dioxolane (D Lin-K-C3-D MA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dili-noleyl-4-dimethylaminomethyl-[1,3]-dioxolane (DLin-KDMA), 1,2-dilinoleylcarbamoyloxy-3-dimethylaminopropane (D Lin-C-DAP), 1,2-dilinoleyoxy-3-(dimethylaminoacetoxypropane (DLin-DAC), 1-2dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (D Lin-MPZ), 3-(N,N-dilinoleylamino)-1,2-propanediol (D LinAP), 3-(N,N-dioleylamino)-1,2-propanedio (DOAP), 1,2-dilinoleyloxo-3-(2-N,N-dimethylamino)ethoxypropane (D Lin-EG-DMA), N,N-dioleyl-N,N-dimethylanrmonium chloride (DODAC), 1,2-dioleyloxy-N,N-dimethylaminopropane (DODMA), 1,2-distearyloxy-N,N-dimethylaminopropane (DSD MA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N, N-trimethylammonium chloride (DOTAP), 3-(N—(N′,N′dimethylaminoethane)-carbamoyl)cholesterol (DC-Chol), N-(1,2-dimyristyloxyprop-3-yl)-N,N-dimethyl-N-hydroxyethyl anrmonium bromide (DMRIE), 2,3-dioleyloxy-N-[2 (spermine-carboxamidoethyl]-N,N-dimethy 1-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy) propane (CpLinDMA), N,N-dimethyl-3,4-dioleyloxybenzylamine (DMOBA), 1,2-N,N′dioleylcarbamyl-3-dimethylaminopropane (DOcarbDAP), 1,2-N,N′-dilinoleylcarbamyl-3-dimethylaminopropane (DLincarbDAP), or mixtures thereof. In certain embodiments, the cationic lipid is DLinDMA, DLin-K-C2-DMA (“XTC2”), or mixtures thereof. The ionizable lipids are not limited to those recited herein, and can further include ionizable lipids known to those skilled in the art, or described in PCT Application No. PCT/US2020/056255 and/or PCT Application No. PCT/US2020/056252, the disclosures of which are herein incorporated by reference in its entirety.

The synthesis of cationic lipids such as DLin-K-C2-DMA (“XTC2”), DLin-K-C3-DMA, DLin-K-C4-DMA, DLin-K6-DMA, and DLin-K-MPZ, as well as additional cationic lipids, is described in U.S. Application Publication No. US 2011/0256175, the disclosure of which is herein incorporated by reference in its entirety for all purposes. The synthesis of cationic lipids such as DLin-K-DMA, DLin-CDAP, DLin-DAC, DLin-MA, DLinDAP, DLin-S-DMA, DLin-2-DMAP, DLin-TMA.Cl, DLin-TAP.Cl, DLin-MPZ, DLinAP, DOAP, and DLin-EG-DMA, as well as additional cationic lipids, is described in PCT Application No. PCT/US08/88676, filed Dec. 31, 2008, the disclosure of which is herein incorporated by reference in its entirety for all purposes. The synthesis of cationic lipids such as CLinDMA, as well as additional cationic lipids, is described in U.S. Patent Publication No. US20060240554, the disclosure of which is herein incorporated by reference in its entirety for all purposes.

### Non-Cationic Lipid

In the nucleic acid-lipid particles of the present disclosure, the non-cationic lipid may comprise, e.g., one or more anionic lipids and/or neutral lipids. In some embodiments, the non-cationic lipid comprises one of the following neutral lipid components: (1) cholesterol or a derivative thereof (2) a phospholipid; or (3) a mixture of a phospholipid and cholesterol or a derivative thereof.

Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The synthesis of cholesteryl-2′-hydroxyethyl ether is known to one skilled in the art and described in U.S. Pat. Nos. 8,058,069, 8,492,359, 8,822,668, 9,364,435, 9,504,651, and 11,141,378, all of which are hereby incorporated herein in their entireties for all purposes.

Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), ioleoylphosphatidylethanolamine (DOPE), palmitoyloleoylphosphatidylcholine (POPC), palmitoyloleoylphosphatidylethanolamine (POPE), palmitoyloleyolphosphatidylglycerol (POPG), dioleoylphosphatidylethanolamine 4-(N-maleimidomethyl)-cyclohexane-1-carboxylate DOPE-mal), dipalmitoylphosphatidylethanolamine (DPPE), dimyristoylphosphatidylethanolamine (DMPE), distearoylphosphatidylethanolamine (DSPE), monomethylphosphatidylethanolamine, dimethylphosphatidylethanolamine, dielaidoylphosphatidylethanolamine (DEPE), stearoyloleoylphosphatidylethanolamine (SOPE), lysophosphatidylcholine, dilinoleoylphosphatidylcholine, and mixtures thereof.

Other diacylphosphatidylcholine and diacylphosphatidylethanolamine phospholipids can also be used. The acyl groups in these lipids can be, for example, acyl groups derived from fatty acids having C₁₀-C₂₄ carbon chains, e.g., lauroyl, myristoyl, palmitoyl, stearoyl, or oleoyl. Additional examples of non-cationic lipids include sterols such as cholesterol and derivatives thereof such as cholestanol, cholestanone, cholestenone, coprostanol, cholesteryl-2′-hydroxyethyl ether, cholesteryl-4′-hydroxybutyl ether, and mixtures thereof. In certain embodiments, the phospholipid is DPPC, DSPC, or mixtures thereof.

### Conjugated Lipid

In the nucleic acid-lipid particles of the present disclosure, the conjugated lipid that inhibits aggregation of particles may comprise, e.g., one or more of the following: a polyethyleneglycol (PEG) lipid conjugate, a polyamide (ATTA)-lipid conjugate, a cationic-polymer-lipid conjugates (CPLs), or mixtures thereof. In some embodiments, the nucleic acid-lipid particles comprise either a PEG-lipid conjugate or an ATTA-lipid conjugate.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEGOH), monomethoxypolyethylene glycolsuccinate (MePEGS), monomethoxypolyethylene glycolsuccinimidyl succinate (MePEG-S—NHS), monomethoxypolyethylene glycolamine (MePEG-NH₂), monomethoxypolyethylene glycoltresylate (MePEG-TRES), and monomethoxypolyethylene glycolimidazolylcarbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present disclosure. The disclosures of these patents are herein incorporated by reference in their entirety for all purposes. In addition, monomethoxypolyethyleneglycolacetic acid (MePEG-CH₂COOH) is particularly useful for preparing PEG-lipid conjugates including, e.g., PEG-DAA conjugates.

In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEGDAA conjugate may be PEG-dilauryloxypropyl (C₁₂), a PEG-dimyristyloxypropyl (C₁₄), a PEG-dipalmityloxypropyl (C₁₆), a PEG-distearyloxypropyl (C₁₈), or mixtures thereof.

Additional PEG-lipid conjugates suitable for use in the disclosure include, but are not limited to, mPEG2000-1,2-diO-alkyl-sn3-carbomoylglyceride (PEG-C-DOMG). The synthesis of PEG-C-DOMG is described in PCT Application No. PCT/US08/88676, filed Dec. 31, 2008, the disclosure of which is herein incorporated by reference in its entirety for all purposes. Yet additional PEG-lipid conjugates suitable for use in the disclosure include, without limitation, 1-[8′-(1,2-dimyristoyl-3-propanoxy)-carboxamido-3′,6′-dioxaoctanyl]carbamoyl-methyl-poly(ethylene glycol) (2 KPEG-DMG). The synthesis of 2 KPEG-DMG is described in U.S. Pat. No. 7,404,969, the disclosure of which is herein incorporated by reference in its entirety for all purposes.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In some embodiments, the PEG moiety has an average molecular weight of about 2,000 daltons or about 750 daltons.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the particles (e.g., LNP) of the present disclosure can further comprise cationic poly(ethylene glycol) (PEG) lipids or CPLs (e.g., Chen et al., Bioconj. Chem., 11:433-437 (2000)). Suitable SPLPs and SPLP-CPLs for use in the present disclosure, and methods of making and using SPLPs and SPLP-CPLs, are disclosed, e.g., in U.S. Pat. No. 6,852,334 and PCT Publication No. WO 00/62813, the disclosures of which are herein incorporated by reference in their entirety for all purposes.

In certain instances, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle.

In the lipid nanoparticles of the present disclosure, the active agent or therapeutic agent may be fully encapsulated within the lipid portion of the particle, thereby protecting the active agent or therapeutic agent from enzymatic degradation. In some embodiments, a nucleic acid-lipid particle comprising a nucleic acid such as a messenger RNA (i.e., mRNA) is fully encapsulated within the lipid portion of the particle, thereby protecting the nucleic acid from nuclease degradation. In certain instances, the nucleic acid in the nucleic acid-lipid particle is not substantially degraded after exposure of the particle to a nuclease at 37° C. for at least about 20, 30, 45, or 60 minutes. In certain other instances, the nucleic acid in the nucleic acid-lipid particle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the active agent or therapeutic agent (e.g., nucleic acid such as siRNA) is complexed with the lipid portion of the particle. One of the benefits of the formulations of the present disclosure is that the lipid particle compositions are substantially non-toxic to mammals such as humans.

### Small Molecule

In various embodiments, the agent is a small molecule. In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis, and in vitro translation systems, using methods well known in the art. In certain embodiments, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the disclosure, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the disclosure, the therapeutic agent is synthesized via small library synthesis.

The small molecule and small molecule compounds described herein may be present as salts even if salts are not depicted, and it is understood that the disclosure embraces all salts and solvates of the therapeutic agents depicted here, as well as the non-salt and non-solvate form of the therapeutic agents, as is well understood by the skilled artisan. In some embodiments, the salts of the therapeutic agents of the disclosure are pharmaceutically acceptable salts.

Where tautomeric forms may be present for any of the therapeutic agents described herein, each and every tautomeric form is intended to be included in the present disclosure, even though only one or some of the tautomeric forms may be explicitly depicted. For example, when a 2-hydroxypyridyl moiety is depicted, the corresponding 2-pyridone tautomer is also intended.

The disclosure also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the disclosure, such as crystalline or non-crystalline forms of the therapeutic agent. Compositions comprising a therapeutic agents of the disclosure are also intended, such as a composition of substantially pure therapeutic agent, including a specific stereochemical form thereof, or a composition comprising mixtures of therapeutic agents of the disclosure in any ratio, including two or more stereochemical forms, such as in a racemic or non-racemic mixture.

The disclosure also includes any or all active analog or derivative, such as a prodrug, of any therapeutic agent described herein. In certain embodiments, the therapeutic agent is a prodrug. In certain embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present disclosure can be used to treat a disease, disorder, or condition.

In certain embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Molecule

In other related aspects, the agent is a nucleic acid molecule. In various embodiments, the agent is an isolated nucleic acid. Thus, in certain embodiments, an isolated nucleic acid, including for example a DNA oligonucleotide and a RNA oligonucleotide can be incorporated in the composition of the disclosure. In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In certain embodiments, the isolated nucleic acid molecule encodes a therapeutic peptide such a thrombomodulin, endothelial protein C receptor (EPCR), anti-thrombotic proteins including plasminogen activators and their mutants, antioxidant proteins including catalase, superoxide dismutase (SOD) and iron-sequestering proteins. In some embodiments, the therapeutic agent is an siRNA, miRNA, shRNA, or an antisense molecule, which inhibits a targeted nucleic acid including those encoding proteins that are involved in aggravation of the pathological processes.

In certain embodiments, the nucleic acid comprises a promoter/regulatory sequence such that the nucleic acid is capable of directing expression of the nucleic acid. Thus, the disclosure encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216.

In one aspect, the disclosure includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a delivery vehicle of the disclosure. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the disclosure is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present disclosure to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the disclosure or the gene construct of the disclosure can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In certain embodiments, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, p-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like.

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl- methyl-, thio- and other modified forms of adenine, cytidine, guanine, thymine, and uridine.

In certain embodiments of the disclosure, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the disclosure may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the disclosure include oligonucleotide derivatives known in the art which have improved biological activity compared to unmodified oligonucleotides (see U.S. Pat. No. 5,023,243).

In certain embodiments of the disclosure, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In certain embodiments, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In certain embodiments, the therapeutic agent comprises a gRNA or a nucleic acid molecule encoding a gRNA. In certain embodiments, the therapeutic agent comprises a Cas peptide or a nucleic acid molecule encoding a Cas peptide.

In certain embodiments, the agent comprises a miRNA or a mimic of a miRNA. In certain embodiments, the agent comprises a nucleic acid molecule that encodes a miRNA or mimic of a miRNA. miRNAs are small non-coding RNA molecules that are capable of causing post-transcriptional silencing of specific genes in cells by the inhibition of translation or through degradation of the targeted mRNA. A miRNA can be completely complementary or can have a region of non-complementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre- miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre -microRNA, mature or hairpin form. In other embodiments, a combination of oligonucleotides comprising a sequence of one or more disease-associated miRNAs, any pre -miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell -type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single- stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene- bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In certain embodiments, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the ICSQ. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest- ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,61 1, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In certain embodiments, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In certain embodiments, the oligonucleotide shares identity with endogenous miRNA or miRNA precursor nucleobase sequences. An oligonucleotide selected for inclusion in a composition of the present disclosure may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present disclosure encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In the sense used in this description, a nucleotide sequence is “substantially homologous” to any of the nucleotide sequences describe herein when its nucleotide sequence has a degree of identity with respect to the nucleotide sequence of at least 60%, advantageously of at least 70%, preferably of at least 85%, and more preferably of at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

### Polypeptide

In other related aspects, the agent is a polypeptide. In various embodiments, the agent is an isolated polypeptide. In other related aspects, the therapeutic agent includes an isolated polypeptide. For example, in certain embodiments, the polypeptide of the disclosure inhibits or activates a target directly by binding to the target thereby modulating the normal functional activity of the target. In certain embodiments, the polypeptide of the disclosure modulates the target by competing with endogenous proteins. In certain embodiments, the polypeptide of the disclosure modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present disclosure, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

In one aspect, the disclosure includes an ionizable LNP molecule comprising or encapsulating one or more agent (e.g., a nucleic acid molecule) for targeted in vivo delivery of the encapsulated agent to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.). In certain embodiments, the nucleic acid molecule is a mRNA molecule.

In some embodiments, the mRNA molecule comprises a nucleotide sequence that can alternatively comprise sequence variations with respect to the original nucleotide sequences, for example, substitutions, insertions and/or deletions of one or more nucleotides, with the condition that the resulting polynucleotide encodes a polypeptide according to the disclosure.

As used herein, an amino acid sequence is “substantially homologous” to any of the amino acid sequences described herein when its amino acid sequence has a degree of identity with respect to the amino acid sequence of at least 60%, advantageously of at least 70%, preferably of at least 85%, and more preferably of at least 95%. The identity between two amino acid sequences is preferably determined by using the BLASTN algorithm.

In certain embodiments, the composition comprises a plurality of constructs, each construct encoding one or more antigens. In certain embodiments, the composition comprises 1 or more, 2 or more, 5 or more, 10 or more, 15 or more, or 20 or more constructs. In certain embodiments, the composition comprises a first construct, comprising a nucleotide sequence encoding an antigen; and a second construct, comprising a nucleotide sequence encoding an adjuvant.

In certain embodiments, the construct comprises a plurality of nucleotide sequences encoding a plurality of antigens. In certain embodiments, the construct encodes 1 or more, 2 or more, 5 or more, 10 or more, 15 or more, or 20 or more antigens. In certain embodiments, the disclosure relates to a construct, comprising a nucleotide sequence encoding an adjuvant. For example, in certain embodiments, the construct comprises a first nucleotide sequence encoding an antigen and a second nucleotide sequence encoding an adjuvant.

In another particular embodiment, the construct is operatively bound to a translational control element. The construct can incorporate an operatively bound regulatory sequence for the expression of the nucleotide sequence of the disclosure, thus forming an expression cassette.

### Peptides

In certain embodiments, the agent is a peptide. Thus, in one aspect, a peptide can be incorporated into the LNP. Thus, in certain embodiments, the agent is a peptide. The peptide of the present disclosure may be made using chemical methods. For example, peptides can be synthesized by solid phase techniques, cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present disclosure may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present disclosure, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present disclosure includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm.

The peptides of the disclosure can be post-translationally modified. For example, post-translational modifications that fall within the scope of the present disclosure include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the disclosure may include unnatural amino acids formed by post-translational modification or by introducing unnatural amino acids during translation.

### Antibodies

In certain embodiments, the agent is an antibody. Thus, in various embodiments, the composition of the disclosure comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al., U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Chimeric Antigen Receptor (CAR) Agents

In certain embodiments, the agent comprises a nucleic acid sequence encoding a chimeric antigen receptor (CAR). In certain embodiments, the agent comprises an mRNA molecule encoding a CAR. In certain embodiments, the agent comprises a modified nucleoside mRNA molecule encoding a CAR.

In certain embodiments, a CAR comprises an extracellular domain capable of binding an antigen, including a tumor or pathogen antigen.

Targets of antigen-specific targeting regions of CARs may be of any kind. In some embodiments, the antigen-specific targeting region of the CAR targets antigens specific for cancer, inflammatory disease, neuronal-disorders, diabetes, cardiovascular disease, infectious diseases or a combination thereof. Examples of antigens that may be targeted by the CARs include but are not limited to antigens expressed on B-cells, antigens expressed on carcinomas, sarcomas, lymphomas, leukemia, germ cell tumors, blastomas, antigens expressed on various immune cells, and antigens expressed on cells associated with various hematologic diseases, autoimmune diseases, and/or inflammatory diseases. The CARs of the disclosure may be capable of redirecting the effector function of the expressing-cells to the target antigen(s).

Antigens that may be targeted by the CARs of the disclosure include but are not limited to any one or more of 4-IBB, 707-AP, 5T4, adenocarcinoma antigen, alpha-fetoprotein, BAFF, β-lymphoma cell, C242 antigen, CA-125, carbonic anhydrase 9 (CA-IX), C-MET, ART-4, BAGE, b-catenin/m, bcr-abl, CAMEL, CAP-1, CCR4, CD 152, CD7, CD 19, CD₂O, CD200, CD22, CD221, CD23 (IgE receptor), CD28, CD30 (TNFRSF8), CD33, CD4, CD38, CD40, CD44 v6, CD44v7/8, CD51, CD52, CD56, CD74, CD80, CD93, CD123, CD171, CEA, CLPP, CNT0888, CTLA-4, carcinoembryonic antigen, EGP2, EGP40, DR5, ErbB2, ErbB3/4, EGFR, EpCAM, EPV-E6, CD3, CASP-8, CD109, CDK/4, CDC-27, Cyp-B, DAM-8, DAM-10, ELV-M2, ETV6, FAP, fibronectin extra domain-B, folate receptor 1, GAGE, GD2, GD3 ganglioside, glycoprotein 75, GPNMB, G250, Gp100, HAGE, HER2/neu, HGF, HMW-MAA, human scatter factor receptor kinase, hTERT, IGF-1 receptor, IGF-I, IgGI, —I-CAM, IL-13, IL-6, insulin-like growth factor I receptor, integrin a5p1, integrin avP3, Kappa or light chain, LAGE, Lewis Y, G250/CAIX, Glypican-3, MAGE, MCi-R, mesothelin, MORAb-009, MS4A1, MUC1, MUC16, mucin CanAg, N-glycolylneuraminic acid, NPC-1C, PDGF-R a, PDL192, phosphatidylserine, PSC1, PSMA, NKG2D ligands, RANKL, RON, ROR1, SAGE, SCH 900105, SDC1, SLAMF7, TAG-72, TEL/AML, tenascin C, TGF beta 2, TGF-β, TRAIL-R¹, TRAIL-R², tumor antigen CTAA16.88, VEGF-A, VEGFR-1, VEGFR2, vimentin, B7-H6, IL-13 receptor a2, IL-11 receptor Rₐ, 8H9, NCAM, Fetal AchR, iCE, MART-1, tyrosinase, WT-1, TEM-1, TEM-2, TEM-3, TEM-4, TEM-5, TEM-6, TEM-7, TEM-8, ROBO-4, and so forth. Other antigens specific for cancer will be apparent to those of skill in the art and may be used in connection with alternate embodiments of the disclosure.

Particular examples of target antigens include but are not limited to surface proteins found on cancer cells in a specific or amplified fashion (e.g. the IL-14 receptor, CD 19, CD20 and CD40 for B-cell lymphoma, the Lewis Y and CEA antigens for a variety of carcinomas, the Tag72 antigen for breast and colorectal cancer, EGF-R for lung cancer, folate binding protein and the HER-2 protein that is often amplified in human breast and ovarian carcinomas), or viral proteins (e.g. gp120 and gp41 envelope proteins of HIV, envelope proteins from the Hepatitis B and C viruses, the glycoprotein B and other envelope glycoproteins of human cytomegalovirus, the envelope proteins from oncoviruses such as Kaposi's sarcoma-associated Herpes virus). Other targets of the CARs of the disclosure include CD4, where the ligand is the HIV gp120 envelope glycoprotein, and other viral receptors, for example ICAM, which is the receptor for the human rhinovirus, and the related receptor molecule for poliovirus.

In some embodiments, the bispecific chimeric antigen receptors target and bind at least two different antigens. Examples of pairings of at least two antigens bound by the bispecific CARs of the disclosure include but are not limited to any combination with HER2, CD 19 and CD₂O, CD 19 and CD22, CD20 and —I-CAM, —I-CAM and GD2, EGFR and —I-CAM, EGFR and C-MET, EGFR and HER2, C-MET and HER2 and EGFR and ROR1. Other pairings of antigens specific for cancer will be apparent to those of skill in the art and may be used in connection with alternate embodiments of the disclosure. In yet other embodiments, the bispecific chimeric antigen receptor targets CD 19 and CD20.

Antigens specific for inflammatory diseases that may be targeted by the CARs of the disclosure include but are not limited to any one or more of AOC3 (VAP-1), CAM-3001, CCL 11 (eotaxin-1), CD125, CD147 (basigin), CD154 (CD40L), CD2, CD₂O, CD23 (IgE receptor), CD25 (a chain of IL-2 receptor), CD3, CD4, CD5, IFN-α, IFN-7, IgE, IgE Fc region, IL-1, IL-12, IL-23, IL-13, IL-17, IL-17A, IL-22, IL-4, IL-5, IL-5, IL-6, IL-6 receptor, integrin a4, integrin a407, Lama glama, LFA-1 (CD11a), MEDI-528, myostatin, OX-40, rhuMAb (37, scleroscin, SOST, TGF beta 1, TNF-α or VEGF-A. Other antigens specific for inflammatory diseases will be apparent to those of skill in the art and may be used in connection with alternate embodiments of the disclosure.

Antigens specific for neuronal disorders that may be targeted by the CARs of the disclosure include but are not limited to any one or more of beta amyloid or MABT5102A. Other antigens specific for neuronal disorders will be apparent to those of skill in the art and may be used in connection with alternate embodiments of the disclosure.

Antigens specific for diabetes that may be targeted by the CARs of the disclosure include but are not limited to any one or more of L-43 or CD3. Other antigens specific for diabetes or other metabolic disorders will be apparent to those of skill in the art and may be used in connection with alternate embodiments of the disclosure.

Antigens specific for cardiovascular diseases which may be targeted by the CARs of the disclosure include but are not limited to any one or more of C5, cardiac myosin, CD41 (integrin alpha-lib), fibrin II, beta chain, ITGB2 (CD 18) and sphingosine-1-phosphate. Other antigens specific for cardiovascular diseases will be apparent to those of skill in the art and may be used in connection with alternate embodiments of the disclosure.

Antigens specific for infectious diseases that may be targeted by the CARs of the disclosure include but are not limited to any one or more of anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus and TNF-α. Other antigens specific for infectious diseases will be apparent to those of skill in the art and may be used in connection with alternate embodiments of the disclosure.

Additional targets of the CARs of the disclosure include antigens involved in B-cell associated diseases. Yet further targets of the CARs of the disclosure will be apparent to those of skill in the art and may be used in connection with alternate embodiments of the disclosure.

Other antigens specific for cancer will be apparent to those of skill in the art and may be used in connection with alternate embodiments of the disclosure.

In certain embodiments, the CAR comprises an antigen binding domain. In a particular non-limiting embodiment, the antigen-binding domain is an scFv specific for binding to a surface antigen of a target cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.).

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et a1., Curr. Opin. Immunol. (2009); Hollyman et al., J. Immunother. 32:169-180 (2009)).

“First generation” CARs for use in the disclosure comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3ξ-chain, which is the primary transmitter of signals from endogenous T cell receptors (TCRs). “First generation” CARs can provide de novo antigen recognition and cause activation of both CD4+ and CD8+ T cells through their CD3ξ chain signaling domain in a single fusion molecule, independent of HLA-mediated antigen presentation.

“Second-generation” CARs for use in the disclosure comprise an antigen binding domain, for example, a single-chain variable fragment (scFv), fused to an intracellular signaling domain capable of activating T cells and a co-stimulatory domain designed to augment T cell potency and persistence. CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ξ signaling domain. Preclinical studies have indicated that “Second Generation” CARs can improve the anti-tumor activity of cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL).

“Third generation” CARs provide multiple co-stimulation, for example, by comprising both CD28 and 4-1 in domains, and activation, for example, by comprising a CD3ξ activation domain.

“Fourth generation” CARs provide co-stimulation, for example, by CD28 or 4-1 in domains, and activation, for example, by a CD3ξ signaling domain in addition to a constitutive or inducible chemokine component.

“Fifth generation” CARs provide co-stimulation, for example, by CD28 or 4-1 in domains, and activation, for example, by a CD3ξ signaling domain, a constitutive or inducible chemokine component, and an intracellular domain of a cytokine receptor, for example, IL-2RP.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above.

### Adjuvant

In certain embodiments, the agent is an adjuvant. Thus, in various embodiments, the composition comprises an adjuvant. In certain embodiments, the composition comprises a nucleic acid molecule encoding an adjuvant. In certain embodiments, the adjuvant-encoding nucleic acid molecule is IVT RNA. In certain embodiments, the adjuvant-encoding nucleic acid molecule is nucleoside-modified mRNA.

Exemplary adjuvants include, but is not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHIC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R², TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, INK, interferon response genes, NFkB, Bax, TRAIL, TRAILrec, TRAILrecDRC5, TRAIL-R³, TRAIL-R⁴, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

Alternatively, one or more of the agents below is delivered for therapeutic purposes as a sole agent and is not intended to function as an adjuvant to a co-administered compound. For example, the coding sequence for a gene therapy (e.g., replacement) of a desired protein may an agent delivered via an LNP as provided herein. Additionally, the coding sequence for a gene editing enzyme may be delivered. In these and other instances, the LNP may be formulated to minimize any immune response to the agent.

### Nucleoside-Modified RNA

In certain embodiments, the agent is a nucleoside-modified RNA. Thus, in one aspect, the composition comprises a nucleoside-modified RNA. Thus, in certain embodiments, the agent is a nucleoside-modified RNA In certain embodiments, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present disclosure is further described in U.S. Pat. No. 8,278,036, which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days. The amount of mRNA required to exert a physiological effect is small and that makes it applicable for human therapy.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable.

It has been demonstrated that the presence of modified nucleosides, including pseudouridines in RNA suppress their innate immunogenicity. Further, protein-encoding, in vitro-transcribed RNA containing pseudouridine can be translated more efficiently than RNA containing no or other modified nucleosides. Subsequently, it is shown that the presence of pseudouridine improves the stability of RNA and abates both activation of PKR and inhibition of translation. A preparative HPLC purification procedure has been established that was critical to obtain pseudouridine-containing RNA that has superior translational potential and no innate immunogenicity. Administering HPLC-purified, pseudourine-containing RNA coding for erythropoietin into mice and macaques resulted in a significant increase of serum EPO levels, thus confirming that pseudouridine-containing mRNA is suitable for in vivo protein therapy.

The present disclosure encompasses RNA, oligoribonucleotide, and polyribonucleotide molecules comprising pseudouridine or a modified nucleoside. In certain embodiments, the composition comprises an isolated nucleic acid encoding an antigen or antigen binding molecule, wherein the nucleic acid comprises a pseudouridine or a modified nucleoside. In certain embodiments, the composition comprises a vector, comprising an isolated nucleic acid encoding an antigen, an antigen binding molecule, an adjuvant, or combination thereof, wherein the nucleic acid comprises a pseudouridine or a modified nucleoside.

In certain embodiments, the nucleoside-modified RNA of the disclosure is IVT RNA. For example, in certain embodiments, the nucleoside-modified RNA is synthesized by T7 phage RNA polymerase. In certain embodiments, the nucleoside-modified mRNA is synthesized by SP6 phage RNA polymerase. In certain embodiments, the nucleoside-modified RNA is synthesized by T3 phage RNA polymerase.

In certain embodiments, the modified nucleoside is m¹acp³Ψ (1-methyl-3-(3-amino-3-carboxypropyl) pseudouridine. In certain embodiments, the modified nucleoside is m¹Ψ -methylpseudouridine). In certain embodiments, the modified nucleoside is Ψm (2′-O-methylpseudouridine. In certain embodiments, the modified nucleoside is m⁵D (5-methyldihydrouridine). In certain embodiments, the modified nucleoside is m³Ψ (3-methylpseudouridine). In certain embodiments, the modified nucleoside is a pseudouridine moiety that is not further modified. In certain embodiments, the modified nucleoside is a monophosphate, diphosphate, or triphosphate of any of the above pseudouridines. In certain embodiments, the modified nucleoside is any other pseudouridine-like nucleoside known in the art.

In certain embodiments, the modified nucleoside of the present disclosure is m⁵C (5-methylcytidine). In certain embodiments, the modified nucleoside is m⁵U (5-methyluridine). In certain embodiments, the modified nucleoside is m⁶A (N⁶-methyladenosine). In certain embodiments, the modified nucleoside is s²U (2-thiouridine). In certain embodiments, the modified nucleoside is Ψ (pseudouridine). In certain embodiments, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); fVC (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²²G (N²,N²-dimethylguanosine); m²Gm (N²,2′—O-dimethylguanosine); m²²Gm (N²,N²,2′—O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQo (7-cyano-7-deazaguanosine); preQi (7-aminomethyl-7-deazaguanosine); G+(archaeosine); D (dihydrouridine); m⁵Um (5,2′-O-dimethyluridine); s⁴U (4-thiouridine); m⁵s2U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm^{S}U (5-methoxycarbonylmethyluridine); mcm^{S}Um (5-methoxycarbonylmethyl-2′-O-methyluridine); mcm⁵s2U (5-methoxycarbonylmethyl-2-thiouridine); nm⁵s2U (5-aminomethyl-2-thiouridine); mnm⁵U (5-methylaminomethyluridine); mnm⁵s2U (5-methylaminomethyl-2-thiouridine); mnm⁵se²U (5-methylaminomethyl-2-selenouridine); ncm⁵U (5-carbamoylmethyluridine); nCm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s2U (5-carboxymethylaminomethyl-2-thiouridine); m⁶²A (N⁶,N⁶-dimethyladenosine); Im (2′-O-methylinosine); m⁴C (N⁴-methylcytidine); m⁴Cm (N^{4,2}′—O-dimethylcytidine); hm⁵C (5-hydroxymethylcytidine); m³U (3-methyluridine); cm⁵U (5-carboxymethyluridine); m⁶Am (N⁶,2′—O-dimethyladenosine); m⁶²Am (N⁶,N⁶,0-2′-trimethyladenosine); m²,7G (N²,7-dimethylguanosine); m²,2,⁷G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); Tm⁵U (5-taurinomethyluridine); im⁵s2U (5-taurinomethyl-2-thiouridine)); imG-14 (4-demethylwyosine); imG2 (isowyosine); or ac⁶A (N⁶-acetyladenosine).

In certain embodiments, a nucleoside-modified RNA of the present disclosure comprises a combination of 2 or more of the above modifications. In certain embodiments, the nucleoside-modified RNA comprises a combination of 3 or more of the above modifications. In certain embodiments, the nucleoside-modified RNA comprises a combination of more than 3 of the above modifications.

In certain embodiments, between 0.1% and 100% of the residues in the nucleoside-modified of the present disclosure are modified (e.g. either by the presence of pseudouridine or a modified nucleoside base). In certain embodiments, 0.1% of the residues are modified. In certain embodiments, the fraction of modified residues is 0.2%. In certain embodiments, the fraction is 0.3%. In certain embodiments, the fraction is 0.4%. In certain embodiments, the fraction is 0.5%. In certain embodiments, the fraction is 0.6%. In certain embodiments, the fraction is 0.8%. In certain embodiments, the fraction is 1%. In certain embodiments, the fraction is 1.5%. In certain embodiments, the fraction is 2%. In certain embodiments, the fraction is 2.5%. In certain embodiments, the fraction is 3%. In certain embodiments, the fraction is 4%. In certain embodiments, the fraction is 5%. In certain embodiments, the fraction is 6%. In certain embodiments, the fraction is 8%. In certain embodiments, the fraction is 10%. In certain embodiments, the fraction is 12%. In certain embodiments, the fraction is 14%. In certain embodiments, the fraction is 16%. In certain embodiments, the fraction is 18%. In certain embodiments, the fraction is 20%. In certain embodiments, the fraction is 25%. In certain embodiments, the fraction is 30%. In certain embodiments, the fraction is 35%. In certain embodiments, the fraction is 40%. In certain embodiments, the fraction is 45%. In certain embodiments, the fraction is 50%. In certain embodiments, the fraction is 60%. In certain embodiments, the fraction is 70%. In certain embodiments, the fraction is 80%. In certain embodiments, the fraction is 90%. In certain embodiments, the fraction is 100%.

In certain embodiments, the fraction is less than 5%. In certain embodiments, the fraction is less than 3%. In certain embodiments, the fraction is less than 1%. In certain embodiments, the fraction is less than 2%. In certain embodiments, the fraction is less than 4%. In certain embodiments, the fraction is less than 6%. In certain embodiments, the fraction is less than 8%. In certain embodiments, the fraction is less than 10%. In certain embodiments, the fraction is less than 12%. In certain embodiments, the fraction is less than 15%. In certain embodiments, the fraction is less than 20%. In certain embodiments, the fraction is less than 30%. In certain embodiments, the fraction is less than 40%. In certain embodiments, the fraction is less than 50%. In certain embodiments, the fraction is less than 60%. In certain embodiments, the fraction is less than 70%.

In certain embodiments, 0.1% of the residues of a given nucleoside (i.e., uridine, cytidine, guanosine, or adenosine) are modified. In certain embodiments, the fraction of the given nucleotide that is modified is 0.2%. In certain embodiments, the fraction is 0.3%. In certain embodiments, the fraction is 0.4%. In certain embodiments, the fraction is 0.5%. In certain embodiments, the fraction is 0.6%. In certain embodiments, the fraction is 0.8%. In certain embodiments, the fraction is 1%. In certain embodiments, the fraction is 1.5%. In certain embodiments, the fraction is 2%. In certain embodiments, the fraction is 2.5%. In certain embodiments, the fraction is 3%. In certain embodiments, the fraction is 4%. In certain embodiments, the fraction is 5%. In certain embodiments, the fraction is 6%. In certain embodiments, the fraction is 8%. In certain embodiments, the fraction is 10%. In certain embodiments, the fraction is 12%. In certain embodiments, the fraction is 14%. In certain embodiments, the fraction is 16%. In certain embodiments, the fraction is 18%. In certain embodiments, the fraction is 20%. In certain embodiments, the fraction is 25%. In certain embodiments, the fraction is 30%. In certain embodiments, the fraction is 35%. In certain embodiments, the fraction is 40%. In certain embodiments, the fraction is 45%. In certain embodiments, the fraction is 50%. In certain embodiments, the fraction is 60%. In certain embodiments, the fraction is 70%. In certain embodiments, the fraction is 80%. In certain embodiments, the fraction is 90%. In certain embodiments, the fraction is 100%.

In certain embodiments, the fraction of the given nucleotide that is modified is less than 8%. In certain embodiments, the fraction is less than 10%. In certain embodiments, the fraction is less than 5%. In certain embodiments, the fraction is less than 3%. In certain embodiments, the fraction is less than 1%. In certain embodiments, the fraction is less than 2%. In certain embodiments, the fraction is less than 4%. In certain embodiments, the fraction is less than 6%. In certain embodiments, the fraction is less than 12%. In certain embodiments, the fraction is less than 15%. In certain embodiments, the fraction is less than 20%. In certain embodiments, the fraction is less than 30%. In certain embodiments, the fraction is less than 40%. In certain embodiments, the fraction is less than 50%. In certain embodiments, the fraction is less than 60%. In certain embodiments, the fraction is less than 70%.

In certain embodiments, a nucleoside-modified RNA of the present disclosure is translated in the cell more efficiently than an unmodified RNA molecule with the same sequence. In certain embodiments, the nucleoside-modified RNA exhibits enhanced ability to be translated by a target cell. In certain embodiments, translation is enhanced by a factor of 2-fold relative to its unmodified counterpart. In certain embodiments, translation is enhanced by a 3-fold factor. In certain embodiments, translation is enhanced by a 5-fold factor. In certain embodiments, translation is enhanced by a 7-fold factor. In certain embodiments, translation is enhanced by a 10-fold factor. In certain embodiments, translation is enhanced by a 15-fold factor. In certain embodiments, translation is enhanced by a 20-fold factor. In certain embodiments, translation is enhanced by a 50-fold factor. In certain embodiments, translation is enhanced by a 100-fold factor. In certain embodiments, translation is enhanced by a 200-fold factor. In certain embodiments, translation is enhanced by a 500-fold factor. In certain embodiments, translation is enhanced by a 1000-fold factor. In certain embodiments, translation is enhanced by a 2000-fold factor. In certain embodiments, the factor is 10-1000-fold. In certain embodiments, the factor is 10-100-fold. In certain embodiments, the factor is 10-200-fold. In certain embodiments, the factor is 10-300-fold. In certain embodiments, the factor is 10-500-fold. In certain embodiments, the factor is 20-1000-fold. In certain embodiments, the factor is 30-1000-fold. In certain embodiments, the factor is 50-1000-fold. In certain embodiments, the factor is 100-1000-fold. In certain embodiments, the factor is 200-1000-fold. In certain embodiments, translation is enhanced by any other significant amount or range of amounts.

In certain embodiments, the nucleoside-modified antigen-encoding RNA of the present disclosure induces significantly more adaptive immune response than an unmodified in vitro-synthesized RNA molecule with the same sequence. In certain embodiments, the modified RNA molecule exhibits an adaptive immune response that is 2-fold greater than its unmodified counterpart. In certain embodiments, the adaptive immune response is increased by a 3-fold factor. In certain embodiments the adaptive immune response is increased by a 5-fold factor. In certain embodiments, the adaptive immune response is increased by a 7-fold factor. In certain embodiments, the adaptive immune response is increased by a 10-fold factor. In certain embodiments, the adaptive immune response is increased by a 15-fold factor. In certain embodiments the adaptive immune response is increased by a 20-fold factor. In certain embodiments, the adaptive immune response is increased by a 50-fold factor. In certain embodiments, the adaptive immune response is increased by a 100-fold factor. In certain embodiments, the adaptive immune response is increased by a 200-fold factor. In certain embodiments, the adaptive immune response is increased by a 500-fold factor. In certain embodiments, the adaptive immune response is increased by a 1000-fold factor. In certain embodiments, the adaptive immune response is increased by a 2000-fold factor. In certain embodiments, the adaptive immune response is increased by another fold difference.

In certain embodiments, “induces significantly more adaptive immune response” refers to a detectable increase in an adaptive immune response. In certain embodiments, the term refers to a fold increase in the adaptive immune response (e.g., 1 of the fold increases enumerated above). In certain embodiments, the term refers to an increase such that the nucleoside-modified RNA can be administered at a lower dose or frequency than an unmodified RNA molecule with the same species while still inducing an effective adaptive immune response. In certain embodiments, the increase is such that the nucleoside-modified RNA can be administered using a single dose to induce an effective adaptive immune response.

In certain embodiments, the nucleoside-modified RNA of the present disclosure exhibits significantly less innate immunogenicity than an unmodified in vitro-synthesized RNA molecule with the same sequence. In certain embodiments, the modified RNA molecule exhibits an innate immune response that is 2-fold less than its unmodified counterpart. In certain embodiments, innate immunogenicity is reduced by a 3-fold factor. In certain embodiments, innate immunogenicity is reduced by a 5-fold factor. In certain embodiments, innate immunogenicity is reduced by a 7-fold factor. In certain embodiments, innate immunogenicity is reduced by a 10-fold factor. In certain embodiments, innate immunogenicity is reduced by a 15-fold factor. In certain embodiments, innate immunogenicity is reduced by a 20-fold factor. In certain embodiments, innate immunogenicity is reduced by a 50-fold factor. In certain embodiments, innate immunogenicity is reduced by a 100-fold factor. In certain embodiments, innate immunogenicity is reduced by a 200-fold factor. In certain embodiments, innate immunogenicity is reduced by a 500-fold factor. In certain embodiments, innate immunogenicity is reduced by a 1000-fold factor. In certain embodiments, innate immunogenicity is reduced by a 2000-fold factor. In certain embodiments, innate immunogenicity is reduced by another fold difference.

In certain embodiments, “exhibits significantly less innate immunogenicity” refers to a detectable decrease in innate immunogenicity. In certain embodiments, the term refers to a fold decrease in innate immunogenicity (e.g., 1 of the fold decreases enumerated above). In certain embodiments, the term refers to a decrease such that an effective amount of the nucleoside-modified RNA can be administered without triggering a detectable innate immune response. In certain embodiments, the term refers to a decrease such that the nucleoside-modified RNA can be repeatedly administered without eliciting an innate immune response sufficient to detectably reduce production of the recombinant protein. In certain embodiments, the decrease is such that the nucleoside-modified RNA can be repeatedly administered without eliciting an innate immune response sufficient to eliminate detectable production of the recombinant protein.

In various embodiments, the composition comprises an in vitro transcribed (IVT) RNA molecule. For example, in certain embodiments, the composition of the disclosure comprises an IVT RNA molecule, which encodes an agent. In certain embodiments, the IVT RNA molecule of the present composition is a nucleoside-modified mRNA molecule.

In certain embodiments, the composition comprises at least one RNA molecule encoding a combination of at least two agents. In certain embodiments, the composition comprises a combination of two or more RNA molecules encoding a combination of two or more agents.

In certain embodiments, the present disclosure provides a method for gene editing of a cell of interest of a subject (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.). For example, the method can be used to provide one or more component of a gene editing system (e.g., a component of a CRISPR system) to a cell of interest of a subject. In some embodiments, the method comprises administering to the subject a composition comprising one or more ionizable LNP molecule formulated for targeted delivery comprising one or more nucleoside-modified RNA molecule for gene editing.

In certain embodiments, the method comprises administration of the composition to a subject. In certain embodiments, the method comprises administering a plurality of doses to the subject. In certain embodiments, the method comprises administering a single dose of the composition, where the single dose is effective in delivery of the target therapeutic agent.

In certain embodiments, the LNPs provided herein include a coding sequence for an editing enzyme encapsulated therein. Editing enzymes include various types of nucleases that are used to cut nucleic acid molecules. Such enzymes include zinc finger nucleases, Transcription activator-like effector nucleases (TALENs), meganucleases, clustered regularly interspaced short palindromic repeats (CRISPR) associated protein (CAS, e.g., CAS9), OMEGA enzymes (IscB), etc.

In certain embodiments, the nuclease is naturally occurring. In other embodiments, the nuclease is non-naturally occurring, i.e., engineered in the DNA-binding domain and/or cleavage domain. For example, the DNA-binding domain of a naturally-occurring nuclease may be altered to bind to a selected target site (e.g., CAS9 nuclease, a meganuclease that has been engineered to bind to site different than the cognate binding site). In other embodiments, the nuclease comprises heterologous DNA-binding and cleavage domains (e.g., zinc finger nucleases; TAL-effector nucleases; meganuclease DNA-binding domains with heterologous cleavage domains).

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms and serve as a prominent tool in the field of genome editing. In cretain embodiments, the coding sequence encodes a zinc finger.

Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). In certain embodiments, the coding sequence encodes a transcription activator-like (TAL) effector nuclease (TALEN).

In certain embodiments, the coding sequence encodes a CRISPR-associated nuclease (Cas9). “Cas9” (CRISPR associated protein 9) refers to family of RNA-guided DNA endonucleases which is characterized by two signature nuclease domains, RuvC (cleaves non-coding strand) and HNH (coding strand). Suitable bacterial sources of Cas9 include Staphylococcus aureus (SaCas9), Streptoococcus pyogenes (SpCas9), and Neisseria meningitides (KM Estelt et al., Nat Meth, 10: 1116-1121 (2013), incorporated herein by reference). The wild-type coding sequences may be utilized in the constructs described herein. Alternatively, the bacterial codons are optimized for expression in humans, e.g., using any of a variety of known human codon optimizing algorithms. Alternatively, these sequences may be produced synthetically, either in full or in part. Other endonucleases with similar properties may optionally be substituted. See, e.g., the public CRISPR database (db) accessible at crispr.u-psud.fr/crispr.

In certain embodiments, the compositions described herein include coding sequences for editing enzymes, particularly nucleases, which are useful targeting a gene for the insertion of a transgene. In certain situations, for example, for applications that do not require precise targeting of an existing gene or locus (e.g., to introduce or modify an endogenous gene, allele, or regulatory element), a common strategy is to target transgene integration to one of a small number of genomic “safe harbor” sites (SHS) for expression, presumably without disrupting the expression of adjacent or more distant genes. These putative SHS play an increasingly important role in developing effective gene therapies; in the investigation of gene structure, function, and regulation; and in cell-based biotechnology.

Certain SHS are known in the art, or may be discovered. Known SHS include the AAVS1 site on chromosome 19q, CCR5 chemokine receptor gene, ROSA26, PCKS9, and TTR. See, e.g., Monnat et al., New Human Chromosomal Sites with “Safe Harbor” Potential for Targeted Transgene Insertion, Hum Gene Ther. 2019 Jul. 1; 30(7): 814-828, which is incorporated by reference. In certain embodiments, the editing enzyme targets a SHS.

In certain embodiments, the editing enzyme is a nuclease that is specific for Proprotein convertase subtilisin/kexin type 9 (PCSK9). In other embodiments, the editing enzyme is a nuclease that is specific for transthyretin (TTR). See, e.g., Conway et al., Non-viral Delivery of Zinc Finger Nuclease mRNA Enables Highly Efficient In Vivo Genome Editing of Multiple Therapeutic Gene Targets, Molecular Therapy, 27(4):866-877 (April 2019), which is incorporated herein by reference. In certain embodiments, the nuclease is a meganuclease such as that described, e.g., in International Patent Publication No. WO 2018/195449.

In some embodiments, the LNP further includes sequences which direct the nuclease to a target site in the target locus. As used herein, the term “target site” or “target sequence” refers to the specific nucleotide sequence that is recognized by the editing enzyme, or its guide sequence. The “target locus” or “target gene locus” is any site in the gene coding region where insertion of the heterologous transgene is desired. For example, in certain embodiments, the target PCSK9 locus is in Exon 7 of the PCSK9 coding sequence located on chromosome 1.

In certain embodiments, such as a meganuclease specific for PCSK9 or TTR, no further sequences are required to direct the nuclease to the target site. However, in the case, for example, of Cas9, an additional sequence, called a “single guide RNA” or “sgRNA” is provided, which is specific for the target sequence. As used herein, the sgRNA has at least a 20-base sequence (or about 24-28 bases, sometimes called the seed region) for specific DNA binding (i.e., homologous to the target DNA), in combination with the gRNA scaffold. Transcription of sgRNAs should start precisely at the 5′ end. When targeting the template DNA strand, the base-pairing region of the sgRNA has the same sequence identity as the transcribed sequence. When targeting the non-template DNA strand, the base-pairing region of the sgRNA is the reverse-complement of the transcribed sequence. Optionally, the LNP may contain more than one sgRNA. The sgRNA is 5′ to a protospacer-adjacent motif (PAM) which is specifically recognized by the Cas9 (or Cpf1) enzyme. Typically, the sgRNA is “immediately” 5′ to the PAM sequence, i.e., there are no spacer or intervening sequences. Suitable sgRNAs can be designed by the person of skill in the art.

The sgRNA includes at least 20 nucleotides and specifically binds to a target site in the target gene, said target site being 5′ to a protospacer-adjacent motif (PAM) that is specifically recognized by the Cas9. The seed region in some embodiments shares 100% complementarity with the target site in the target gene. In other embodiments, the seed region contains 1, 2, 3, 4, or 5 mismatches as compared to the target site.

In other embodiments, for example, wherein the nuclease is a Cas9, the gene editing vector further includes one or more nuclear localization signal (NLSs). In certain embodiments, the NLSs flank the coding sequence for the Cas9.

In certain embodiments, the cargo is a DNA molecule or an RNA molecule. In certain embodiments, the cargo is a cDNA or mRNA molecule. In various embodiments, the composition comprises an in vitro transcribed (IVT) RNA molecule. For example, in certain embodiments, the composition comprises an IVT RNA molecule, which encodes an editing enzyme. In certain embodiments, the IVT RNA molecule is a nucleoside-modified mRNA molecule. In certain embodiments, where the nuclease coding sequence is provided as messenger RNA (mRNA). An mRNA may include a 5′ untranslated region, a 3′ untranslated region, and/or a coding or translating sequence.

An mRNA may be a naturally or non-naturally occurring mRNA. An mRNA may include one or more modified nucleobases, nucleosides, or nucleotides. In some embodiments, the mRNA in the compositions comprise at least one modification which confers increased or enhanced stability to the nucleic acid, including, for example, improved resistance to nuclease digestion in vivo. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present disclosure, and particularly with respect to the mRNA, refer to increased or enhanced resistance to degradation by, for example nucleases (i.e., endonucleases or exonucleases) which are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. Also contemplated by the terms “modification” and “modified” as such terms related to the mRNA of the present disclosure are alterations which improve or enhance translation of mRNA nucleic acids, including for example, the inclusion of sequences which function in the initiation of protein translation (e.g., the Kozak consensus sequence).

In some embodiments, the mRNA described herein have undergone a chemical or biological modification to render them more stable. Exemplary modifications to an mRNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring mRNA, for example, covalent modifications such as the introduction of modified nucleotides, (e.g., nucleotide analogs, or the inclusion of pendant groups which are not naturally found in such mRNA molecules).

In some embodiments, the number of C and/or U residues in an mRNA sequence is reduced. In certain embodiments, the number of C and/or U residues is reduced by substitution of one codon encoding a particular amino acid for another codon encoding the same or a related amino acid. Contemplated modifications to the mRNA nucleic acids of the present disclosure also include the incorporation of pseudouridines pseudouridine (W) or 5-methylcytosine (m5C). Substitutions and modifications to the mRNA of the present disclosure may be performed by methods readily known to one or ordinary skill in the art.

In certain embodiments, the mRNA includes a 5′ cap structure, a chain terminating nucleotide, a stem loop, a polyA sequence, and/or a polyadenylation signal. A cap structure or cap species is a compound including two nucleoside moieties joined by a linker and may be selected from a naturally occurring cap, a non-naturally occurring cap or cap analog, or an anti-reverse cap analog. An mRNA may instead or additionally include a chain terminating nucleoside.

In certain embodiments, the mRNA includes a stem loop, such as a histone stem loop. A stem loop may include 1, 2, 3, 4, 5, 6, 7, 8, or more nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

In certain embodiments, the mRNA includes a polyA sequence. A polyA sequence may be comprised entirely or mostly of adenine nucleotides or analogs or derivatives thereof. In certain embodiments, the polyA sequence is a tail located adjacent to a 3′ untranslated region of an mRNA.

In certain embodiments, the present disclosure provides a method for gene editing of a cell of interest of a subject (e.g., a bone cell and/or bone marrow cell). For example, the method can be used to provide one or more component of a gene editing system (e.g., a component of a CRISPR system) to a cell of interest of a subject. In certain embodiments, a second nucleic acid molecule is provided that encodes a transgene of interest, or an expression cassette containing the transgene coding sequence. In some embodiments, the transgene is a therapeutic agent.

Examples of suitable transgenes for delivery include, e.g., those associated with familial hypercholesterolemia (e.g., VLDLr, LDLr, ApoE, see, e.g., WO 2020/132155, WO 2018/152485, WO 2017/100682, which are incorporated herein by reference), muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB—P51608), Amyotrophic Lateral Sclerosis (ALS), Duchenne Type Muscular dystrophy, Friedrichs Ataxia (e.g., frataxin), progranulin (PRGN) (associated with non-Alzheimer's cerebral degenerations, including, frontotemporal dementia (FTD), progressive non-fluent aphasia (PNFA) and semantic dementia), among others. Other useful gene products include, carbamoyl synthetase I, ornithine transcarbamylase (OTC), arginosuccinate synthetase, arginosuccinate lyase (ASL) for treatment of arginosuccinate lyase deficiency, arginase, fumarylacetate hydrolase, phenylalanine hydroxylase, alpha-1 antitrypsin, rhesus alpha- fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding p-glucuronidase (GUSB)). Examples of suitable transgene for delivery may include human frataxin delivered in an AAV vector as described, e.g., PCT/US20/66167, Dec. 18, 2020, U.S. Provisional Patent Application No. 62/950,834, filed Dec. 19, 2019, and U.S. Provisional Application No. 63/136,059 filed on Jan. 11, 2021 which are incorporated herein by reference. Another example of suitable transgene for delivery may include human acid-a-glucosidase (GAA) delivered in an AAV vector as described, e.g., PCT/US20/30493, Apr. 30, 2020, now published as WO2020/223362A1, PCT/US20/30484, Apr. 20, 2020, now published as WO 2020/223356 A1, U.S. Provisional Patent Application No. 62/840,911, filed Apr. 30, 2019, US Provisional Application No. 62.913,401, filed Oct. 10, 2019, U.S. Provisional Patent Application No. 63/024,941, filed May 14, 2020, and U.S. Provisional Patent Application No. 63/109,677, filed Nov. 4, 2020 which are incorporated herein by reference. Also, another example of suitable transgene for delivery may include human a-L-iduronidase (IDUA) delivered in an AAV vector as described, e.g., PCT/US2014/025509, Mar. 13, 2014, now published as WO 2014/151341, and U.S. Provisional Patent Application No. 61/788,724, filed Mar. 15, 2013 which are incorporated herein by reference.

In certain embodiments, the transgene may be operably linked to regulatory sequences that direct the expression thereof. In some embodiments, the transgene cassette includes a promoter, the transgene coding sequence, and a poly A sequence. In other embodiments, a transgene is provided without a promoter, and is inserted in the genome downstream of a native promoter, e.g., the PCSK9 promoter.

In addition to a promoter, the transgene cassette may contain one or more appropriate “regulatory elements” or “regulatory sequences”, which comprise but are not limited to an enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others. These control sequences or the regulatory sequences are operably linked to the nuclease coding sequences or transgene coding sequence.

In addition to the transgene cassette, in certain embodiments, the LNP composition described herein also includes homology-directed recombination (HDR) arms 5′ and 3′ to the transgene cassette, to facilitate homology directed recombination of the transgene into the endogenous genome. The homology arms are directed to the target locus and can be of varying length. In some embodiments, the HDR arms are from about 100 bp to about 1000 bp in length. In other embodiments, the HDR arms are from about 130 bp to about 500 bp. In other embodiments, the HDR arms are from about 100 bp to about 300 bp. In certain embodiments, the HDR arm is 130 bp. In other embodiments, the HDR arms are about 130 bp to 140 bp. In certain embodiments, the HDR arms are about 500 bp. In certain embodiments, the HDR arms are absent. The HDR arms ideally share a high level of complementarity with the target locus, although it need not be 100% complementarity. In some embodiments, 1, 2, 3, 4, 5, 6, 7, 8, 9, 10, 11, 12, 13, 14, 15, 16, 17, 18, 19, 20, or more mismatches are permitted in each HDR arm.

The ratio of ionizable lipid to nucleic acid may be varied in the LNP in a range from about 1:1 to about 10:1 by weight. In certain embodiments, the lipid:nucleic acid ratio is about 5:1. In certain embodiments, the lipid:nucleic acid ratio is about 6:1. In certain embodiments, the lipid:nucleic acid ratio is about 7:1. In certain embodiments, the lipid:nucleic acid ratio is about 8:1. In certain embodiments, the lipid:nucleic acid ratio is about 9:1. In certain embodiments, the lipid:nucleic acid ratio is about 10:1.

In embodiments where the composition includes a coding sequence for a Cas9 (e.g., mRNA encoding Cas9), the mRNA to sgRNA ratio can be present in a range of from about 1:5 to about 5:1 by weight. In certain embodiments, the mRNA:sgRNA ratio is about 1:5. In certain embodiments, the mRNA:sgRNA ratio is about 1:4. In certain embodiments, the mRNA:sgRNA ratio is about 1:3. In certain embodiments, the mRNA:sgRNA ratio is about 1:2. In certain embodiments, the mRNA:sgRNA ratio is about 1:1. In certain embodiments, the mRNA:sgRNA ratio is about 2:1. In certain embodiments, the mRNA:sgRNA ratio is about 3:1. In certain embodiments, the mRNA:sgRNA ratio is about 4:1. In certain embodiments, the mRNA:sgRNA ratio is about 5:1. Other ratios within this range can be utilized.

LNP formation and encapsulation of cargo may be accomplished using techniques known in the art. See, e.g., Jeffs, et a1 (March 2005). A Scalable, Extrusion-Free Method for Efficient Liposomal Encapsulation of Plasmid DNA. Pharmaceutical Research, 22(3), 362-372, and Kulkarni et al., On the Formation and Morphology of Lipid Nanoparticles Containing Ionizable Cationic Lipids and siRNA, ACS Nano, 12:4787-4795 (April 2018) both of which are incorporated herein by reference. For example, in brief, LNP-mRNA compositions are generated by rapid mixing of lipids in ethanol with mRNA in aqueous buffer (pH 4.0), followed by dialysis to remove ethanol and to raise the pH to 7.4.

### Combinations

In certain embodiments, the composition of the present disclosure comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, in certain embodiments, the composition comprises a 1:1 ratio of two individual agents. However, the combination is not limited to any particular ratio. Rather any ratio that is shown to be effective is encompassed.

### Treatment Methods

The present disclosure provides methods of delivering an agent to a cell of interest of a target subject. Exemplary cells that can be targeted using the LNP compositions of the disclosure include, but are not limited to, a bone cell and/or bone marrow cell (e.g., a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.). Thus, in various embodiments, the disclosure relates to methods delivering an agent to a bone, bone marrow, or a combination thereof.

In some embodiments, the agent is a diagnostic agent to detect at least one marker associated with a disease, disorder, or condition. In some embodiments, the agent is a therapeutic agent for the treatment or prevention of a disease, disorder, or condition. In some embodiments, the agent is an editing enzyme for gene editing. In some embodiments, the agent replaces at least one protein in a subject in need thereof. Therefore, in some embodiments, the disclosure provides methods for diagnosing, treating, or preventing a disease, disorder, or condition comprising administering an effective amount of the LNP composition comprising one or more diagnostic or therapeutic agents, one or more adjuvants, or a combination thereof.

For example, in certain embodiments, the disease, disorder, or condition is a bone disease or disorder, bone marrow disease or disorder, condition associated with bone, condition associated with bone marrow, bone fracture, bone defect, root canal, nonunion fracture, cancer, leukemia, lymphoma, myeloma, other blood cancers, myeloma, sickle cell disease, inflammatory disease or disorder, or any combination thereof.

In some embodiments, the disclosure relates to methods of treating or preventing bone diseases, disorders, or conditions in subjects in need thereof, the method comprising administering the LNP composition of the disclosure. Exemplary bone diseases, disorders, or conditions that can be treated using the LNP compositions and methods of the disclosure include, but are not limited to, osteoporosis, fracture, bone defect, bone cavity, root canal, nonunion fracture, scoliosis, Paget's disease, osteoarthritis, rheumatoid arthritis, gout, bursitis, solid tumor cancer that metastasizes to bone, leukemia, lymphoma, myeloma, other blood cancers, or any combination thereof.

For example, in certain embodiments, bone diseases, disorders, or conditions that can be treated using the LNP compositions and methods of the is a fracture, bone defect, bone cavity, root canal, nonunion fracture, or any combination thereof. Thus, in some embodiments, the disclosure relates to methods of bone regeneration in subjects in need thereof, the method comprising administering the LNP composition of the disclosure.

In some embodiments, the disclosure relates to methods of treating or preventing bone marrow diseases, disorders, or conditions in subjects in need thereof, the method comprising administering the LNP composition of the disclosure. Exemplary bone marrow diseases, disorders, or conditions that can be treated using the LNP compositions and methods of the disclosure include, but are not limited to, leukemia, myelodysplastic syndrome (MDS), myeloproliferative disorders (MPD), aplastic anemia, iron deficiency anemia, disorders of plasma cells, plasma cell dyscrasia, lymphomas, thrombotic thrombocytopenic purpura, sickle cell disease, myeloma, a disease, disorder, or condition arising from hematopoietic stem cells, or any combination thereof.

In some embodiments, the disclosure relates to methods of treating or preventing cancer and diseases or disorders associated therewith in subjects in need thereof, the method comprising administering the LNP composition of the disclosure.

In some embodiments, the present disclosure provides a method for inducing an immune response in subjects in need thereof, the method comprising administering the LNP composition of the disclosure. For example, in certain embodiments, the method for inducing an immune response in subjects in need thereof is a cancer immunotherapy comprising administering the LNP comprising CAR to the subject to induce an immune response against cancer.

Exemplary cancers that can be treated using the LNP compositions and methods of the disclosure include, but are not limited to, acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

In various embodiments, the disease, disorder, or condition is a disease, disorder, or condition associated with at least one cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.). For example, in certain embodiments, the disease, disorder, or condition associated with at least one cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.) is a bone disease, disorder, or condition, bone marrow disease, disorder, or condition, cancer, or any combination thereof.

In certain embodiments, the method comprises administering a LNP composition of the disclosure comprising one or more nucleic acid molecules for treatment or prevention of a disease, disorder, or condition (e.g., a bone disease, disorder, or condition, bone marrow disease, disorder, or condition, cancer, or any combination thereof). In certain embodiments, the one or more nucleic acid molecules encode a therapeutic agent for the treatment of the disease, disorder, or condition (e.g., a bone disease, disorder, or condition, bone marrow disease, disorder, or condition, cancer, or any combination thereof).

In certain embodiments, the compositions of the disclosure can be administered in combination with one or more additional therapeutic agent, an adjuvant, or a combination thereof. For example, in certain embodiments, the method comprises administering an LNP composition comprising a nucleic acid molecule encoding one or more agent for targeted administration to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.) and a second LNP comprising a nucleic acid molecule encoding one or more adjuvants. In certain embodiments, the method comprises administering a single LNP composition comprising a nucleic acid molecule encoding one or more agent for targeted administration to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.) and a nucleic acid molecule encoding one or more adjuvants.

In certain embodiments, the method comprises administering to subject a plurality of LNPs of the disclosure comprising nucleoside-modified nucleic acid molecules encoding a plurality of agents to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.), adjuvants, or a combination thereof.

In certain embodiments, the method comprises administering the LNP of the disclosure comprising nucleoside-modified RNA, which provides stable expression of a nucleic acid encoded agent (e.g., a therapeutic agent encoded by a nucleoside modified mRNA molecule) described herein to a cell of interest (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.).

Administration of the compositions of the disclosure in a method of treatment can be achieved in a number of different ways, using methods known in the art. In certain embodiments, the method of the disclosure comprises systemic administration of the subject, including for example enteral or parenteral administration. In certain embodiments, the method comprises intradermal delivery of the composition. In certain embodiments, the method comprises intravenous delivery of the composition. In some embodiments, the method comprises intramuscular delivery of the composition. In certain embodiments, the method comprises subcutaneous delivery of the composition. In certain embodiments, the method comprises inhalation of the composition. In certain embodiments, the method comprises intranasal delivery of the composition.

It will be appreciated that the composition of the disclosure may be administered to a subject either alone, or in conjunction with another agent.

The therapeutic and prophylactic methods of the disclosure thus encompass the use of pharmaceutical compositions comprising at least one LNP of the disclosure comprising an agent (e.g., an mRNA molecule) described herein, to practice the methods of the disclosure. The pharmaceutical compositions useful for practicing the disclosure may be administered to deliver a dose of from 0.001 ng/kg/day and 100 mg/kg/day. For example, in some embodiments, the pharmaceutical compositions useful for practicing the disclosure may be administered to deliver a dose of from 0.005 mg/kg/day and 5 mg/kg/day. In certain embodiments, the disclosure envisions administration of a dose which results in a concentration of the compound of the present disclosure from 10 nM and 10 μM in a mammal.

Typically, dosages which may be administered in a method of the disclosure to a mammal, preferably a human, range in amount from 0.01 g to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 0.1 g to about 10 mg per kilogram of body weight of the mammal. More preferably, the dosage will vary from about 1 g to about 5 mg per kilogram of body weight of the mammal. For example, in some embodiments, the dosage will vary from about 0.005 mg to about 5 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of a composition of the present disclosure may be performed by single administration or boosted by multiple administrations.

In certain embodiments, the disclosure includes a method comprising administering a combination of LNP compositions described herein. In certain embodiments, the combination has an additive effect, wherein the overall effect of the administering the combination is approximately equal to the sum of the effects of administering each LNP composition. In other embodiments, the combination has a synergistic effect, wherein the overall effect of administering the combination is greater than the sum of the effects of administering each LNP composition.

In some aspects of the disclosure, the method provides for delivery of compositions for gene editing or genetic manipulation to a target cell (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.) of a subject to treat or prevent a disease, disorder, or condition (e.g., a bone cell and/or bone marrow cell, such as a stem cell, HSC, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, etc.).

### Therapy

In one aspect, the therapeutic compounds or compositions of the disclosure may be administered prophylactically (i.e., to prevent disease, disorder, or condition, such as a bone disease, disorder, or condition, bone marrow disease, disorder, or condition, cancer, or any combination thereof) or therapeutically (i.e., to treat disease, disorder, or condition, such as a bone disease, disorder, or condition, bone marrow disease, disorder, or condition, cancer, or any combination thereof) to subjects suffering from or at risk of (or susceptible to) developing the disease, disorder, or condition (e.g., a bone disease, disorder, or condition, bone marrow disease, disorder, or condition, cancer, or any combination thereof). Such subjects may be identified using standard clinical methods.

In the context of the present disclosure, prophylactic administration occurs prior to the manifestation of overt clinical symptoms of disease, disorder, or condition (e.g., a bone disease, disorder, or condition, bone marrow disease, disorder, or condition, cancer, or any combination thereof), such that the disease, disorder, or condition is prevented or alternatively delayed in its progression. In the context of the field of medicine, the term “prevent” encompasses any activity which reduces the burden of mortality or morbidity from a disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The composition of the disclosure can be useful in combination with therapeutic, anti-cancer, and/or radiotherapeutic agents. Thus, the present disclosure provides a combination of the present LNP with therapeutic, anti-cancer, and/or radiotherapeutic agents for simultaneous, separate, or sequential administration. The composition of the disclosure and the other anticancer agent can act additively or synergistically.

The therapeutic agent, anti-cancer agent, and/or radiation therapy can be administered according to therapeutic protocols well known in the art. It will be apparent to those skilled in the art that the administration of the therapeutic agent, anti-cancer agent, and/or radiation therapy can be varied depending on the disease being treated and the known effects of the anti-cancer agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse effects.

### Bisphosphonate- Substituted Piperazine Ionizable Lipid Compounds and Bone-Targeted Lipid Nanoparticles (LNPs) Thereof

In another aspect, the disclosure provides a method of delivering an agent to a bone of a subject. In certain embodiments, the method comprises administering to the subject at least one LNP of the disclosure or at least one pharmaceutical composition of the disclosure.

In certain embodiments, the therapeutic cargo is delivering to a bone cell, bone tissue, or bone marrow of the subject. In certain embodiments, the agent is selected from the group consisting of a nucleic acid molecule, a small molecule, a protein, an antibody, a therapeutic agent, and any combination thereof.

In another aspect, the disclosure provides a method of treating, ameliorating, and/or preventing at least one disease, disorder, or condition in a subject in need thereof. In certain embodiments, the method comprises administering to the subject at least one LNP of the disclosure or at least one pharmaceutical composition of the disclosure.

In certain embodiments, the disease, disorder, or condition is selected from the group consisting of bone disease or disorder, bone marrow disease or disorder, condition associated with bone, condition associated with bone marrow, bone fracture, bone defect, cancer, leukemia, lymphoma, myeloma, other blood cancers, myeloma, sickle cell disease, inflammatory disease or disorder, and any combination thereof.

In another aspect, the disclosure provides a method of inducing a bone regeneration in a subject in need thereof. In certain embodiments, the method comprises administering to the subject at least one LNP of the disclosure or at least one pharmaceutical composition of the disclosure.

### Bisphosphonate Lipid Nanoparticle (BP-LNP)-Adsorbed Mineralized Tissue Compositions and Methods of Use Thereof

In another aspect, the disclosure provides a method for treating, preventing, and/or ameliorating an orthopedic or dental disease in a subject in need thereof. In certain embodiments, the method comprises contacting a mineralized tissue of the subject with the LNP-adsorbed mineral tissue composition of the disclosure under conditions effective to graft the mineralized tissue of the composition to the mineralized tissue of the subject.

In another aspect, the disclosure provides a method for delivering a nucleic acid molecule and/or therapeutic agent to a mineralized tissue of a subject in need thereof. In certain embodiments, the method comprises contacting the mineralized tissue of the subject with the LNP-adsorbed mineral tissue composition of the disclosure under conditions effective to graft the mineralized tissue of the composition to the mineralized tissue of the subject.

In another aspect, the disclosure provides a method for repairing, restoring, or reducing degradation of a mineralized tissue in a subject in need thereof. In certain embodiments, the method comprises contacting the mineralized tissue of the subject with the LNP-adsorbed mineral tissue composition of the disclosure under conditions effective to graft the mineralized tissue of the composition to the mineralized tissue of the subject.

In certain embodiments, the mineralized tissue comprises hydroxyapatite. In certain embodiments, wherein the mineralized tissue is selected from the group consisting of bone, dentin, enamel, cementum, and a calcified cartilage. In certain embodiments, the mineralized tissue surface is an allograft, autograft, xenograft, or synthetic graft.

In certain embodiments, the composition enhances osteointegration, bone deposition, remineralization, or cellular uptake of the nucleic acid molecule and/or therapeutic agent.

In certain embodiments, the orthopedic or dental disease is selected from the group consisting of a bone injury (e.g., bone fracture, stress fracture, non-union or bone defect, or compression injury), bone degeneration (e.g., osteoporosis, osteopenia, osteomalacia, or avascular necrosis), inflammation (e.g., osteoarthritis, rheumatoid arthritis, synovitis, periostitis, tendinitis, or bursitis), tooth or periodontal tissue damage (e.g., caries, enamel demineralization, dentin hypersensitivity, enamel erosion, or cracked tooth), periodontal and gingival disease (e.g., periodontitis, gingivitis, peri-implant mucositis, or peri-implantitis), inflammation (e.g., pulpitis, apical periodontitis, periapical abscesses, osteomyelitis, or inflammatory resorption).

In certain embodiments, the subject is a mammal. In certain embodiments, the mammal is a human.

### LNP-Adsorbed Mineral Tissue Composition

In one aspect, the disclosure provides a composition comprising a mineralized tissue and the lipid nanoparticle (LNP) of the disclosure. In certain embodiments, the LNP is adsorbed to a surface of the mineralized tissue.

In certain embodiments, the mineralized tissue comprises hydroxyapatite.

In certain embodiments, the mineralized tissue is selected from the group consisting of bone, dentin, enamel, cementum, and a calcified cartilage.

In certain embodiments, the LNP further comprises at least one cargo selected from the group consisting of a nucleic acid molecule and a therapeutic agent.

In certain embodiments, the nucleic acid molecule is selected from the group consisting of cDNA, mRNA, miRNA, siRNA, modified RNA, antagomir, antisense molecule, and a targeted nucleic acid, or any combination thereof. In certain embodiments, the siRNA comprises STAT3 siRNA.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 99.99% (w/w) active ingredient.

In addition to the active ingredient, a pharmaceutical composition of the disclosure may further comprise one or more additional pharmaceutically active agents.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a 5 sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65 F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences (1985, Genaro, ed., Mack Publishing Co., Easton, PA), which is incorporated herein by reference.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of therapeutic (i.e., composition) necessary to achieve a therapeutic effect may vary according to factors such as the activity of the particular therapeutic employed; the time of administration; the rate of excretion of the composition; the duration of the treatment; other drugs, compounds or materials used in combination with the composition; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic composition of the disclosure is from about 0.01 mg/kg to 100 mg/kg of body weight/per day of active agent (i.e., nucleic acid). One of ordinary skill in the art would be able to study the relevant factors and make the determination regarding the effective amount of the therapeutic composition without undue experimentation.

The composition may be administered to an animal as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic composition to produce the desired therapeutic effect in association with the required pharmaceutical vehicle. The dosage unit forms of the disclosure are dictated by and directly dependent on (a) the unique characteristics of the therapeutic composition and the particular therapeutic effect to be achieved, and (b) the limitations inherent in the art of compounding/formulating such a therapeutic composition for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

The amount of active agent of the composition(s) of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of active agent (i.e., nucleic acid) present in the composition of the disclosure is from about 0.5 μg and about 5,000 mg. In some embodiments, a dose of active agent present in the composition of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of the composition of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Experimental Examples

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present disclosure and practice the claimed methods. The following working examples therefore, specifically point out the preferred embodiments of the present disclosure, and are not to be construed as limiting in any way the remainder of the disclosure.

### Bisphosphonate Lipids, Lipid Nanoparticle Compositions Comprising the Same, and Methods of Use Thereof for Targeted Delivery

### Materials and Methods

### Chemicals and Reagents for Synthesis

1,2-Epoxy-dodecan (C12, 90%, Sigma-Aldrich), 1,2-epoxytetradecan (C14, technical grade, 85%, Sigma-Aldrich), 1,2-epoxyhexadecan (C16, technical grade, 85%, Sigma-Aldrich), acryloyl chloride (97%, Sigma-Aldrich), N-hydroxysuccinimide (NHS, 98%, TCI), 6-(p-toluidinyl)naphthalene-2-sulfonic acid (TNS, Sigma-Aldrich), triethylamine (TEA, anhydrous, 99.5%, Sigma-Aldrich), diisopropylethylamine (DIPEA, Sigma-Aldrich), tris(2-aminoethyl)amine (Core 110, 97%, Alfa Aesar), tris(3-aminopropyl)amine (Core T3A, 97%, TCI), bis(3-aminopropyl)({4-[bis(3-aminopropyl)amino]butyl})amine (Core 197, Enamine), N1-(2-(4-(2-aminoethyl)piperazin-1-yl)ethyl)ethane-1,2-diamine (Core 200, Enamine), {2-[2-(2-aminoethoxy)ethoxy]ethyl}[2-(4-{2-[2-(2-aminoethoxy)ethoxy]ethyl}piperazin-1-yl)ethyl]amine (Core 488, Enamine), 3-(4-{2-[(3-amino-2-ethoxypropyl)amino]ethyl}piperazin-1-yl)-2-ethoxypropan-1-amine (Core 490, Enamine), 2-{2-[4-(2-{[2-(2-aminoethoxy)ethyl]amino}ethyl)piperazin-1-yl]ethoxy}ethan-1-amine (Core 494, Enamine), 1,2-dioleoyl-sn-glycero-3-phosphoethanolamine (DOPE, AvantiPolarLipids), cholesterol (Sigma-Aldrich) and 1,2-dimyristoyl-sn-glycero-3-phosphoethanolamine-N-[methoxy(polyethyleneglycol)-2000](C14-PEG2000, AvantiPolarLipids), hydroxyapatite (HA) nanoparticles (Sigma-Aldrich), benzoxazolium, 3-octadecyl-2-[3-(3-octadecyl-2(3H)-benzoxazolylidene)-1-propenyl]-, perchlorate (DiO, ThermoFisher), 1,1′-dioctadecyl-3,3,3′,3′-tetramethylindotricarbocyanine iodide (DiR, ThermoFisher) and alendronate sodium (Ald, Santa Cruz) were used as received. Organic solvents were purchased from Fisher Scientific. Deuterium oxide (D₂O, 99.8%) and chloroform-d (CDCl₃) were purchased from Acros Organics.

### Nucleic Acids and Other Reagents for Biological Assays

Luciferase 1000 Assay System (Ref E4550) and CellTiter-Glo Luminescent Cell Viability (Ref G7572) were purchased from Promega Corporation. Alanine Transaminase (ALT) Colorimetric Activity Assay Kit (Item. 700260) and Aspartate Aminotransferase (AST) Colorimetric Activity Assay Kit (Item. 701640) were purchased from Cayman Chemical. Mouse BMP2 ELISA Kit (Ref. ab119582) was purchased from abcam. Anti-mouse CD11b antibody (PE-AF610), CD45R/B220 antibody (Percp), CD 117 antibody (Brilliant Violet 711), CD3 antibody (PE), Ly6c antibody (AF700), CD31 antibody (APC), Ly6G antibody (Brilliant Violet 650) and CD19 antibody (Brilliant Violet 421) were purchased from Biolegend.

### Cell Culture

Dulbecco's Modified Eagle Medium (DMEM) was purchased from Gibco containing high glucose, L-glutamine, pherol red, and without sodium pyruvate and HEPES. Trypsin-EDTA (0.25%), penicillin streptomycin (P/S) were purchased from Gibco. Fetal bovine serum (FBS) was purchased from Sigma-Aldrich. HeLa cells were cultured in DMEM supplemented with 10% FBS and 1% P/S.

### Animal Studies

C57BL/6J mice were purchased from Jackson Laboratory.

### Instruments

¹H NMR spectrum were performed on a NEO 400 MHz spectrometer. LC-MS was performed on an Agilent LCMS system equipped with UV-Vis and evaporative light scattering detectors (ELSD). Flash chromatography was performed on a Teledyne Isco CombiFlash Rf-200i chromatography system equipped with UV-Vis and evaporative light scattering detectors (ELSD). LNPs were formulated by a Pump33DS syringe pump (Harvard Apparatus, Holliston, MA). Particle size and zeta potentials were measured by Dynamic Light Scattering (DLS) with Malvern Zetasizer Nano ZS. Particle morphology was measured by Cryo-TEM. LNP binding efficiency to the bone surface was imaged using Leica DiM8. Flow cytometry was performed using an LSRB machine. In vitro luminescent intensity, cell viability, ALT qualification, AST qualification, TNS assay and ex vivo LNPs binding to the bone surface was quantified using an Infinite M Plex plate reader (Tecan, Morrisville, NC).

### Synthesis of N-acryloxysuccinimide (NAS)

N-Hydroxysuccinimide (NHS, 5.75 g, 50 mmol, 1.0 equiv) and triethylamine (TEA, 8.36 mL, 60 mmol, 1.2 equiv) were dissolved in anhydrous dichloromethane (DCM; 50 mL) and the mixture was cooled to 0° C. on an ice bath. Acryloyl chloride (5.43 g, 60 mmol, 1.2 equiv) was added dropwise under vigorous stirring. The reaction was then allowed to warm to room temperature overnight. The supernatant was washed with HCl (1%; 2×50 mL), brine (2×50 mL), saturated sodium bicarbonate (2×50 mL) and brine (2×50 mL). The organic layer was collected, dried over Na₂SO₄, and concentrated in vacuo. The final pure monomer was further purified by flash chromatographic (silica gel, DCM/hexane=1/1) as a white powder.

¹H NMR (400 MHz, CDCl₃), δ 6.72-6.65 (d, 1H), 6.37-6.26 (q, 1H), 6.20-6.14 (d, 1H), 2.86 (s, 4H). LC-MS (m/z): Calcd for [M+H]⁺: 170.1, Found: 170.1.

### Synthesis of Alendronate Acrylamide

Briefly, a flame-dried 50 mL round-bottom flask was charged with NAS (101.48 mg, 0.6 mmol, 1.2 equiv), alendronate sodium (135 mg, 0.5 mmol, 1.0 equiv) and diisopropylethylamine (DIPEA, 129.24 mg, 1 mmol, 2.0 equiv), deionized water and ethanol. The reaction was conducted under 40° C. overnight. Then, the solution was concentrated in vacuo and rinsed with DCM. The obtained solid was filtered off and dissolved again in water. The final product was afforded by precipitating in acetone twice, and was used without further purification. ¹H NMR (400 MHz, D₂O), δ 6.39-6.09 (d, 2H), 5.73-5.68 (q, 1H), 6.20-6.14 (d, 1H), 3.29-3.23 (m, 2H), 2.04-1.76 (m, 4H). LC-MS (m/z): Calcd for [M+H]⁺: 304.1, Found: 304.3.

### General Synthesis of Alendronate Amide-Bearing Lipid Cores

Taking synthesis of bis(2-aminoethyl)amine-1-aminoethyl-alendronate (110BP) as an example, alendronate acrylamine (100 mg, 0.307 mmol, 1 equiv) and Core 110 (224.8 mg, 1.538 mmol, 5 equiv) were added in a glass vial equipped with a stir bar dissolved in deionized water (300 L). The reaction was processed overnight at room temperature. Then the solution was concentrated and precipitated in acetone. The resulting oil phase was collected by centrifugation, which was dissolved in water and precipitated in acetone again. The final product was afforded after centrifugation and drying, and was used without further purification. ¹H NMR (400 MHz, D₂O), δ 3.18-3.10 (m, 2H), 2.98-2.81 (m, 6H), 2.74-2.54 (m, 10H), 1.93-1.67 (m, 4H). LC-MS (m/z): Calcd for [M+H]⁺: 450.4, Found: 450.7.

Below are the ¹H NMR shifts and mass-to-charge ratio (m/z) for amide-bearing lipid cores.

T3ABP. ¹H NMR (400 MHz, D₂O), 63.17-3.10 (m, 2H), 2.90-2.74 (m, 6H), 2.65-2.38 (m, 10H), 1.90-1.58 (m, 10H). LC-MS (m/z): Calcd for [M+H]⁺: 492.4, Found: 492.7.

197BP. ¹H NMR (400 MHz, D₂O), δ 3.21-3.09 (m, 2H), 2.96-2.30 (m, 22H), 2.19-2.10 (m, 2H), 1.97-1.30 (m, 16H). LC-MS (m/z): Calcd for [M+H]⁺: 620.7, Found: 620.6.

200BP. ¹H NMR (400 MHz, D₂O), δ 3.18-3.11 (m, 2H), 2.99-2.69 (m, 12H), 2.65-2.36 (m, 12H), 1.94-1.67 (m, 4H). LC-MS (m/z): Calcd for [M+H]⁺: 519.5, Found: 519.7.

488BP. ¹H NMR (400 MHz, D₂O), δ 3.67-3.57 (m, 18H), 3.18-3.11 (m, 2H), 3.09-2.93 (m, 6H), 2.92-2.71 (m, 8H), 2.61-2.42 (m, 8H), 2.02-1.68 (m, 8H). LC-MS (m/z): Calcd for [M+H]⁺: 695.7, Found: 695.6.

490BP. ¹H NMR (400 MHz, D₂O), δ 3.71-3.52 (m, 6H), 3.20-3.10 (m, 2H), 3.08-2.98 (m, 2H), 2.96-2.26 (m, 24H), 2.19-2.09 (m, 2H), 1.98-1.63 (m, 4H), 1.17-1.05 (m, 6H). LC-MS (m/z): Calcd for [M+H]⁺: 635.6, Found: 635.6.

494BP. ¹H NMR (400 MHz, D₂O), δ 3.70-3.50 (m, 10H), 3.15-3.10 (m, 2H), 3.04-2.90 (m, 4H), 2.84-2.71 (m, 6H), 2.62-2.36 (m, 12H), 2.03-1.61 (m, 4H). LC-MS (m/z): Calcd for [M+H]⁺: 607.6, Found: 607.6.

### Synthesis for Alendronate-Bearing Ionizable Lipid Libraries

An alendronate-bearing ionizable lipid library (21 ionizable lipids) was prepared by Michael addition reaction between the above described seven different BP-lipids and three different epoxide tails [1,2-epoxy-dodecane (C12), 1,2-epoxytetradecane (C14), 1,2-epoxyhexadecane (C16)]. Taking synthesis of 490BP-C14 as an example, 490BP (9.85 mg, 0.015 mmol, 1 equiv) and C14 (15.29 mg, 0.072 mmol, 4.8 equiv) were added in a glass vial equipped with a stir bar dissolved in ethanol/water (1/1). The reaction was stirred at 80° C. for three days. Then the solution was concentrated and precipitated in acetone. The crude product was afforded by removing the solvents and was used to screen the library for Luc mRNA delivery without further purification. 490BP-C14. ¹H NMR (400 MHz, CD₃OD), δ 3.71-3.52 (m, 10H), 3.27-3.13 (m, 2H), 3.05-2.31 (m, 18H), 2.16-1.88 (m, 2H), 1.59-1.45 (m, 8H), 1.44-1.26 (m, 80H), 1.25-1.18 (m, 6H), 0.97-0.88 (m, 12H). LC-MS (m/z): Calcd for [M+H]⁺: 1485.2, Found: 1485.3.

### BP-Free LNP Library

To evaluate the effects of BPs in enhancing bone-targeting, a similar library without BPs was synthesized using a similar protocol.

Formulation of BP Lipid-Like Materials into Lipid Nanoparticles (LNPs)

All LNPs used in this study were prepared as follows. An ethanol phase containing all lipids and an aqueous phase containing mRNA (Luc mRNA, EGFP mRNA or BMP-2 mRNA) were mixed using a microfluidic device to formulate LNPs. The ethanol phase contained alendronate-bearing ionizable lipid or normal ionizable lipid, 1,2-dioleoyl-sn-glycero-3-phosphoethanolamine (DOPE), cholesterol and 1,2-dimyristoyl-sn-glycero-3-phosphoethanolamine-N-[methoxy(polyethyleneglycol)-2000](C14-PEG2000) with a fixed molar ratio of 35%, 16%, 46.5% and 2.5%, respectively. Aqueous phase was composed of mRNA dissolved in 10 mM citrate buffer. The ethanol and aqueous phases were mixed at a flow rate of 1.8 ml/min and 0.6 ml/min (3:1) using Pump33DS syringe pumps. LNPs were dialyzed in 1×PBS using a microdialysis cassette (20,000 MWCO, Thermo Fisher Scientific, Waltham, MA) for 2 h and then filtered through a 0.22 μm filter. Zetasizer Nano was used to measure the Z-average diameters, polydispersity index (PDI) and Zeta potential. mRNA concentration and encapsulation efficiency in each LNP formulation were measured using a modified Quant-iT R^{1b}oGreen (ThermoFisher) assay on a plate reader.

In Vitro Luc mRNA LNP Library Screening

In a white transparent 96-well plate, HeLa cells were seeded at a density of 5×10³ cells per well in 100 μL growth medium (DMEM, 10% FBS, 1% P/S), and were incubated at 37° C. in 5% CO₂. The medium was exchanged for fresh growth medium, and then LNPs were treated at a dose of 10 ng Luc mRNA per well. Firefly luciferase expression was measured 24 h after LNP transfection using a Luciferase Assay System (Promega) according to the manufacturer's protocol. The luminescent signal was normalized to PBS treated cells. Cell viability was measured using a CellTiter-Glo Luminescent Cell Viability Assay (Promega), in which the luminescence was normalized to PBS treated cells according to the manufacturer's protocol.

In vitro Bone Targeting of LNPs via Hydroxyapatite Binding

The affinity of BP-LNPs and LNPs in the absence of BPs toward bone mineral was investigated. DiO labelled LNP formulations were incubated with hydroxyapatite nanoparticles (HA) in Eppendorf tubes and shaking for varying lengths of time (0, 30, 60, 120, 360 and 1440 min). At the end of incubation time, HA nanoparticles and the LNPs bound to them were pelleted when the Eppendorf tubes were spun at 85×g for 5 min. The binding affinity of BP-LNPs and LNPs was determined by measuring the decrease in the relative concentration (by measuring the fluorescence intensity) of targeted BP-LNPs in the supernatant compared to LNPs in the absence of BPs. Experiment was performed in triplicate.

Ex vivo Bone Affinity of LNPs

Specific binding of DiO labelled 490BP-C14 LNPs to bone fragment ex vivo (femur, mice) was investigated by imaging using a fluorescent microscope. DiO labelled 490-C14 LNPs and DiO fluorescent molecules were used controls. Bone fragments were incubated with the above LNP formulation (mass concentration: 0.2 mg/mL) with different DiO concentrations (0.2% and 0.4%) or DiO solution (0.2%) for 240 min, with constant shaking. After incubation, bone was washed three times with PBS, air dried in dark, and then subjected for imaging. Representative images are shown from experiments performed in triplicate.

In vivo Luciferase mRNA LNP Delivery

All animal procedures were performed on female C57BL/6J mice aged 6-8 weeks approved by the Institutional Animal Care & Use Committee (IACUC) of University of Pennsylvania. Mice were administered a single intravenous DiR labelled Luc mRNA at a dosage of 0.5 mg/kg via tail vein injection. The luciferase expression was evaluated using an IVIS Spectrum imaging system (Caliper Life Sciences) 12 h post-injection. Mice were then injected D-luciferin (PerkinElmer) at a dose of 150 mg/kg by intraperitoneal injection (IP). After 10 min incubation under anesthesia, bioluminescence and fluorescence intensity were quantified by measuring photon flux in the region of interest where signal emanated using Living IMAGE Software provided by Caliper. Ex vivo imaging was performed on heart, liver, spleen, lung, kidney, left leg, spine and right leg after resection.

Flow Cytometry of EGFP mRNA Transfected Diverse Cell Types in Bone Marrow

Mice were administered a single intravenous DiR labelled EGFP mRNA 490BP-C14 LNPs at a dosage of 0.5 mg/kg via tail vein injection. After 12 h, mice were euthanized by cervical dislocation and their femur and tibia were resected then collected in 1×PBS. Bone marrow was collected by flushing femurs and tibia in DMEM medium (10 mL), the obtained suspension was then centrifuged (5 min, 0.5 xg) and lysed by ACK lysis buffer (ThermoFisher) (1 mL) for 10 min. Afterwards, single-cell suspensions were obtained by centrifugation (5 min, 0.5 xg) and resuspended in 1×PBS (200 L). The following fluorochrome antibodies specific to mouse were used: CD11b (PE-AF610), CD45R/B220 (Percp), CD 117 (Brilliant Violet 711), CD3 (PE), Ly6c (AF700), CD31 (APC), Ly6G (Brilliant Violet 650) and CD19 (Brilliant Violet 421). The obtained single-cell suspensions were stained at 4° C. for 1 h by each of the above antibodies (3 L), and afterwards were centrifuged, washed, centrifuged and resuspended in 1×PBS (1 mL). To define transfection efficiency of 490BP-C14 LNPs, cells from 490-C14 LNPs in the absence of BPs and PBS treated mice were used as control. Data was acquired using LSRB flow machine and analyzed with FlowJo software.

mRNA LNP-Mediated Bone Morphogenetic Protein-2 (BMP-2) Secretion in Bone Marrow

Mice were administered a single intravenous injection of 490BP-C14 LNPs encapsulating mRNA encoding for BMP-2 at dosages ranging from 0.5-1.5 mg/kg via tail vein injection. After 12 h, mice were sacrificed and their legs and spine were collected. Bone marrow was flushed with 1 mL DMEM media solution. BMP-2 was extracted from the bone surface after incubating with 4M Guanidine hydrochloride solution overnight. BMP-2 levels on the bone surface and in the bone marrow were measured using individual assay kit according to manufacturer's protocols.

In vivo LNP Toxicity Measurements

To evaluate the in vivo toxicity of 490BP-C14 and 490-C14 LNPs, mouse blood was drawn and serum was isolated at 12 h after injection. Alanine aminotransferase (ALT) and aspartate aminotransferase (AST) levels were measured using individual assay kits according to manufacturer's protocols.

### Example 1: Bisphosphonate Lipid-Like Materials for mRNA Delivery to Bone Microenvironment

One class of therapeutics that were of particular interest for delivery to bone were nucleic acid therapeutics, which regulated therapeutic gene expression within targeted sites to treat diseases by delivering exogenous nucleic acids, such as plasmid DNA (pDNA), small interfering RNA (siRNA), messenger RNA (mRNA), and micro RNA (miRNA). Currently, the most clinically advanced non-viral delivery vectors for RNA therapeutics were lipid nanoparticles (LNPs), a four-component formulation consisting of ionizable lipids, cholesterol, phospholipids, and polyethylene glycol (PEG)-conjugated lipids, which were used in Alnylam Pharmaceutical's siRNA therapeutic ONPATTRO and Pfizer/BioNTech's and Moderna's COVID-19 mRNA vaccines. These nanocarriers were biocompatible, reproducibly manufactured, encapsulated a range of nucleic acids, and had physical characteristics suitable for various administration routes. Recently, LNPs encapsulating siRNA were systemically delivered to bone marrow cells to silence genes in the inflammatory monocyte subset and endothelial cells to selectively inhibit migration of these cells and their progeny. Despite this encouraging progress, passive diffusion of LNPs still makes them extremely challenging to specifically deliver mRNA to the bone microenvironment and there was a critical unmet need to develop a targeted LNP technology for mRNA delivery to bone.

Although previous studies have also demonstrated that ligand substitution is a promising approach for targeted mRNA-LNPs therapeutic delivery, for instance, Peer and co-workers demonstrated that primary lymphocytes re robustly silenced by attaching a pan leukocyte selective targeting ligand onto normal lipid nanoparticles formulations (Ramishetti, S., et al., 2020, Advanced Materials, 32(12):21906128), there remained a critical unmet need to develop a targeted LNP technology for mRNA delivery to bone.

Therefore, approaches incorporating ligands with affinity for bone enhance LNP targeting and accumulation in the bone microenvironment were investigated. Among various targeting ligands, bisphosphonate (BP), such as alendronate, are analogs of inorganic pyrophosphate and chelate strongly with the calcium ion (Ca²⁺) of hydroxyapatite (HA), which is the main inorganic component of bone, endowing bisphosphonate with strong affinity and rapid adsorption to the bone surface. After administration of BP-LNPs, strong interactions between the bone-targeting ligand on the LNP surface and HA improve retention and accumulation of LNPs in the bone microenvironment.

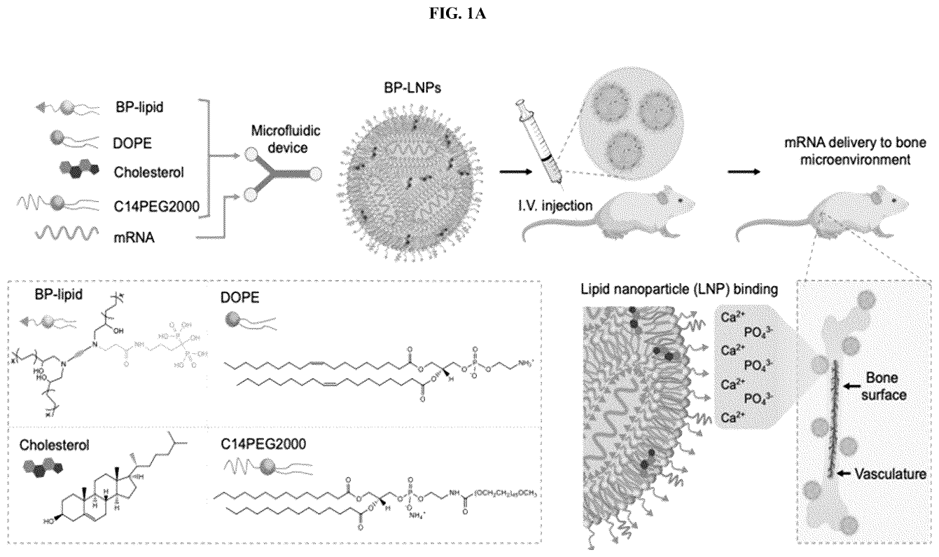

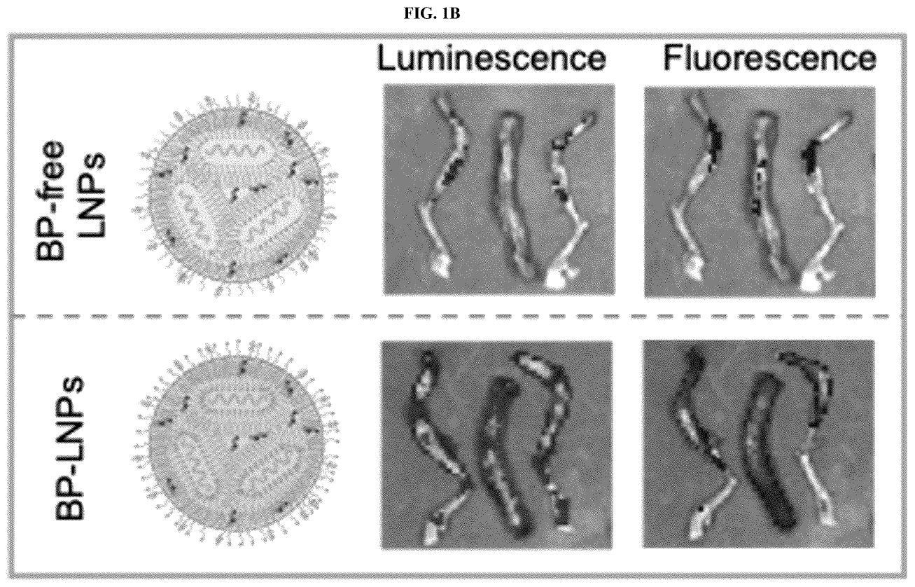

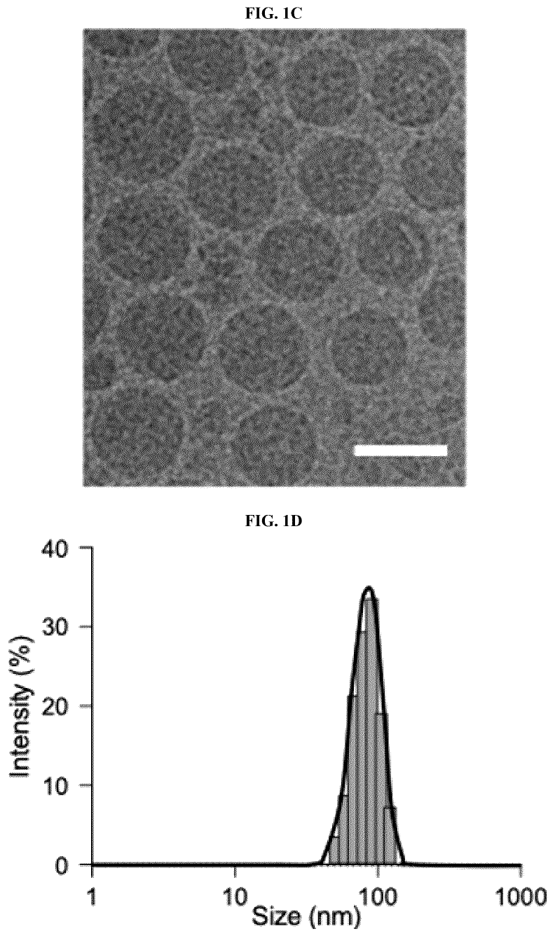

The present example describes the rational design and synthesis of a series of BP-conjugated ionizable lipid-like materials and the formulated library of BP-LNPs for enhanced systemic delivery of mRNA to the bone microenvironment (FIGS. 1A-1). A library of twenty-one BP-functionalized ionizable lipids were investigated to formulate a library of BP-LNPs with a narrow size distribution (FIGS. 1Cs1D). Following an initial in vitro HeLa cell screening with firefly luciferase-encoding mRNA, 490BP-C14 LNP was identified as the lead formulation, which showed higher protein expression (2.5-fold) compared to LNPs in the absence of BPs (490-C14 LNP). These BP-functionalized LNPs not only displayed significantly higher affinity to both HA and bone surfaces in vitro and ex vivo, but also showed enhanced accumulation and transfection efficiency in the bone microenvironment following systemic administration in vivo (FIG. 1). Additionally, 490BP-C14 LNPs exhibited enhanced transfection and uptake in a range of cell types in bone marrow, ranging from B cells, T cells, monocytes, granulocytes, to endothelial cells. Furthermore, BP-LNPs mediated the secretion of bone morphogenetic protein-2 (BMP-2) in the bone microenvironment (both on the bone surface and in the bone marrow) following delivery of BMP-2 mRNA via intravenous injection, demonstrating utility in therapeutic applications in bone regeneration and fracture healing. Due to the facile and versatile preparation of BP-lipid like materials, additional studies focus on applying these materials towards a range of mRNA LNP therapeutic applications in bone, including regenerative medicine, protein replacement, and gene editing therapies.

### Example 2: Rational Design and Synthesis of BP-Functionalized Ionizable Lipids and Incorporation into LNP Formulations

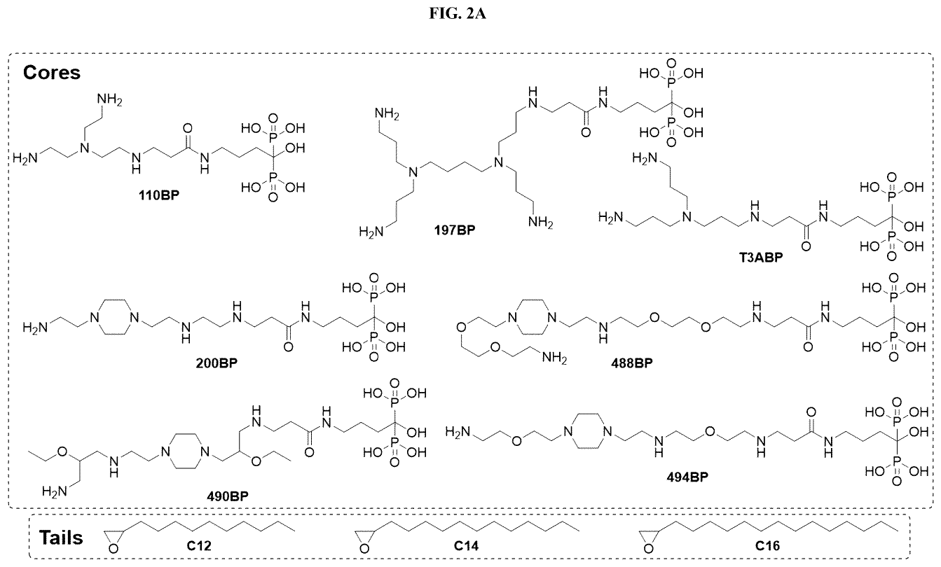

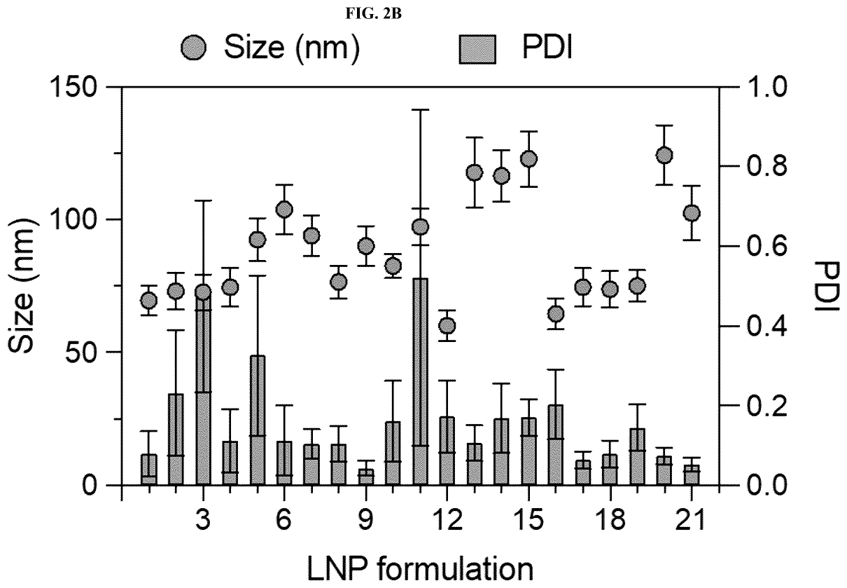

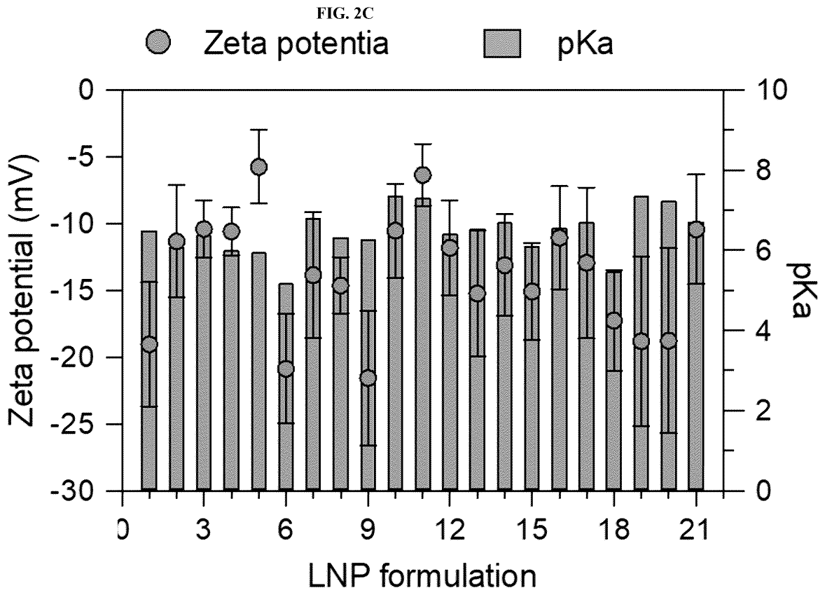

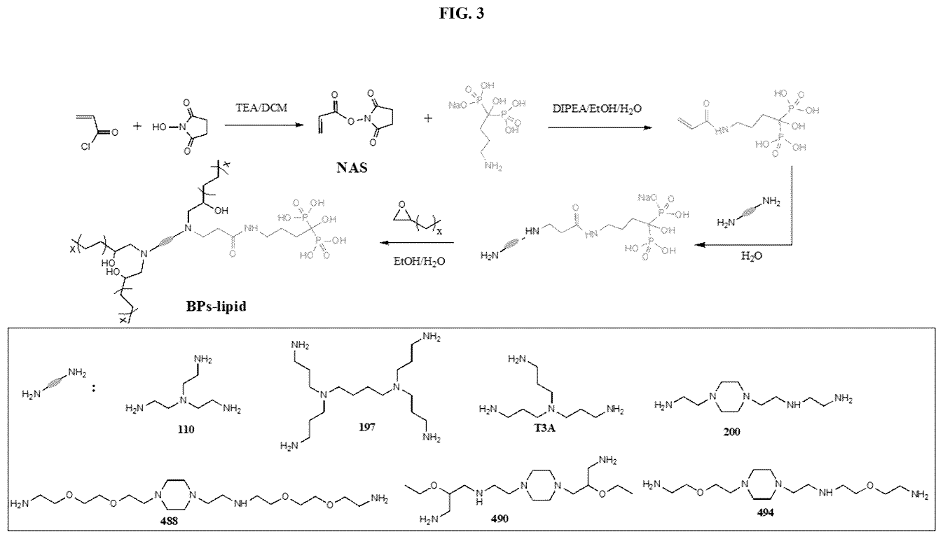

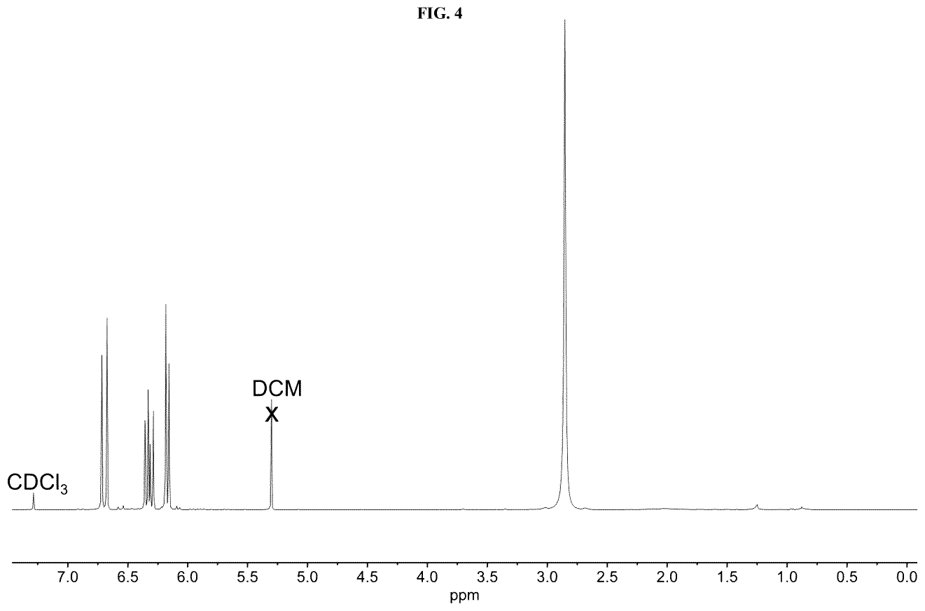

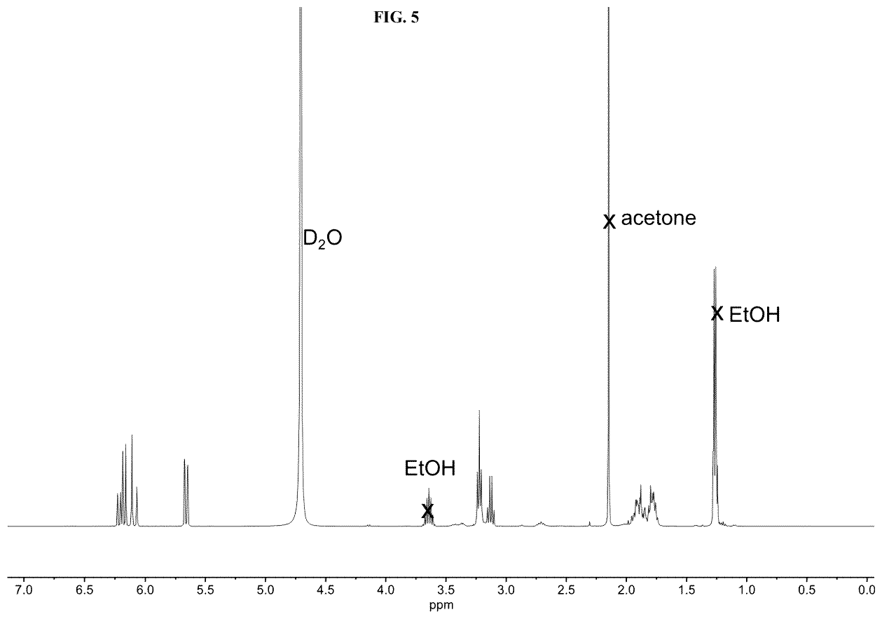

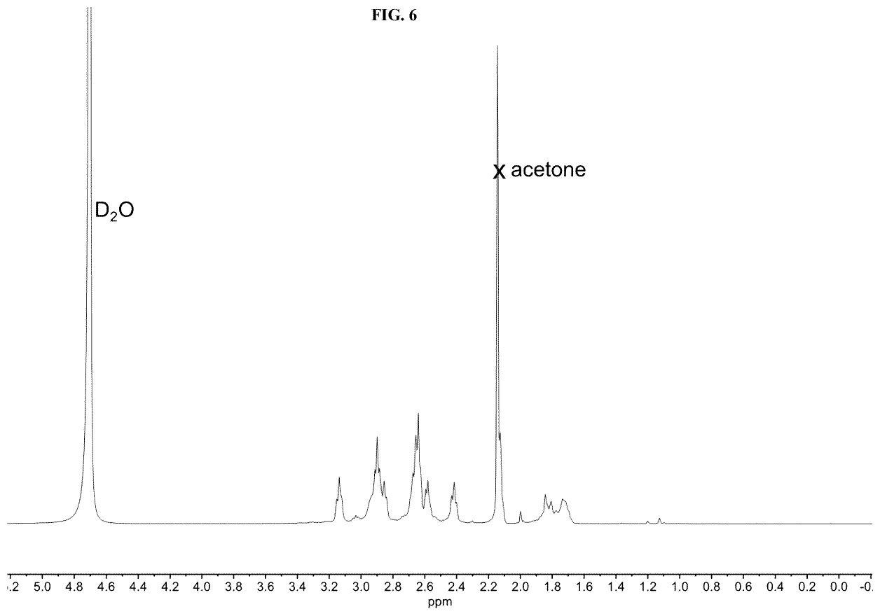

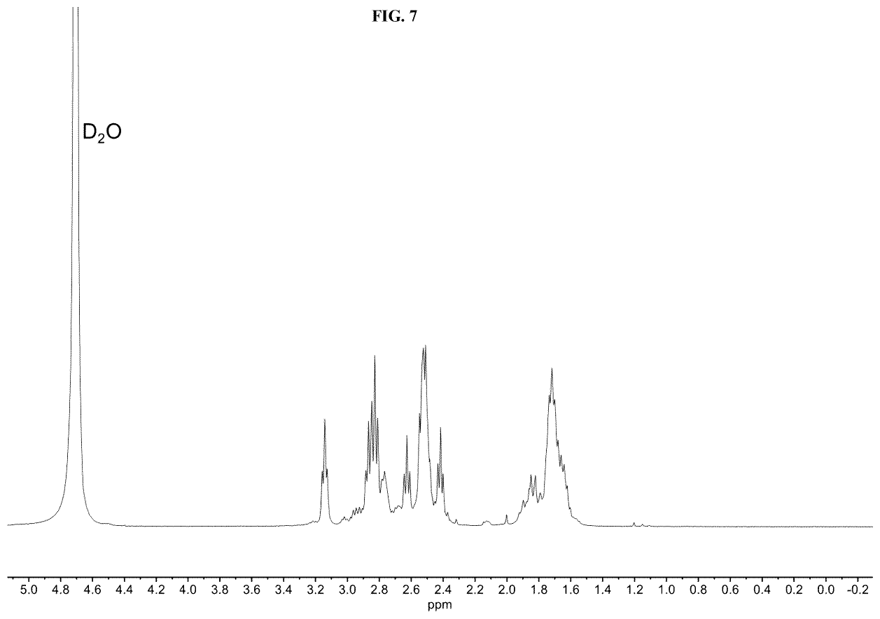

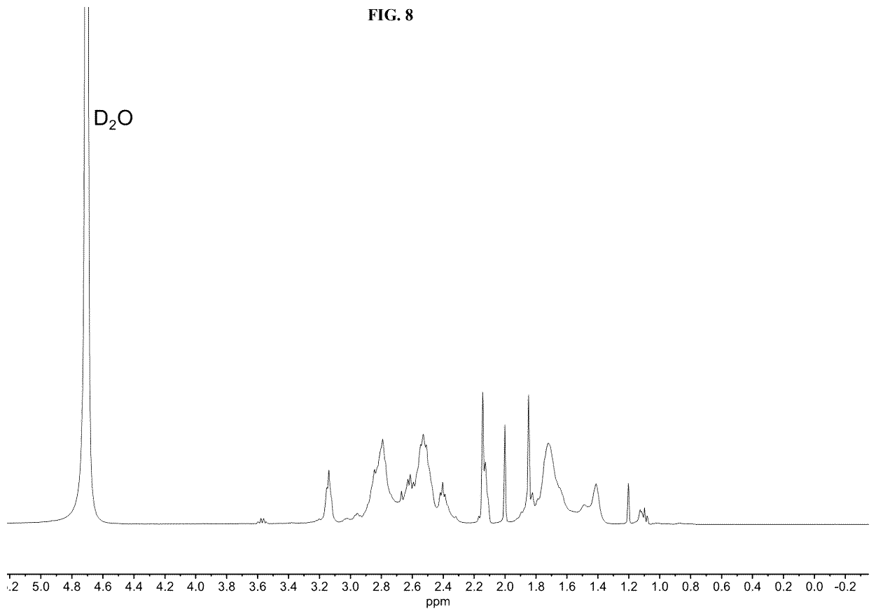

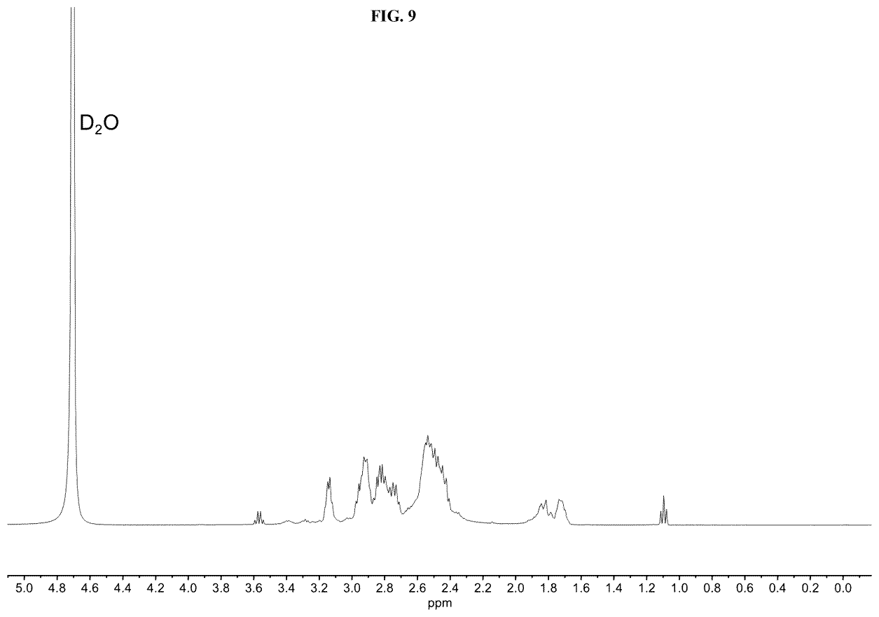

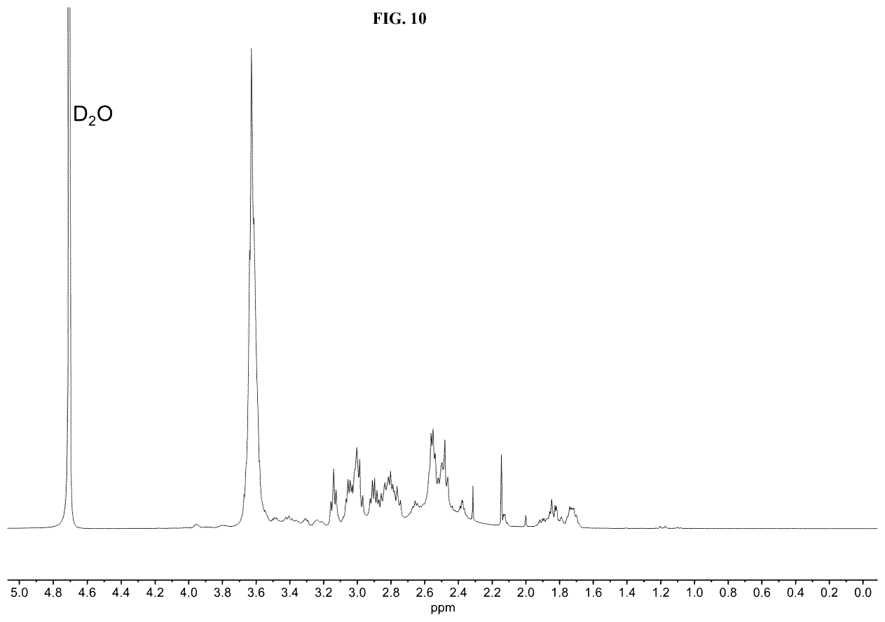

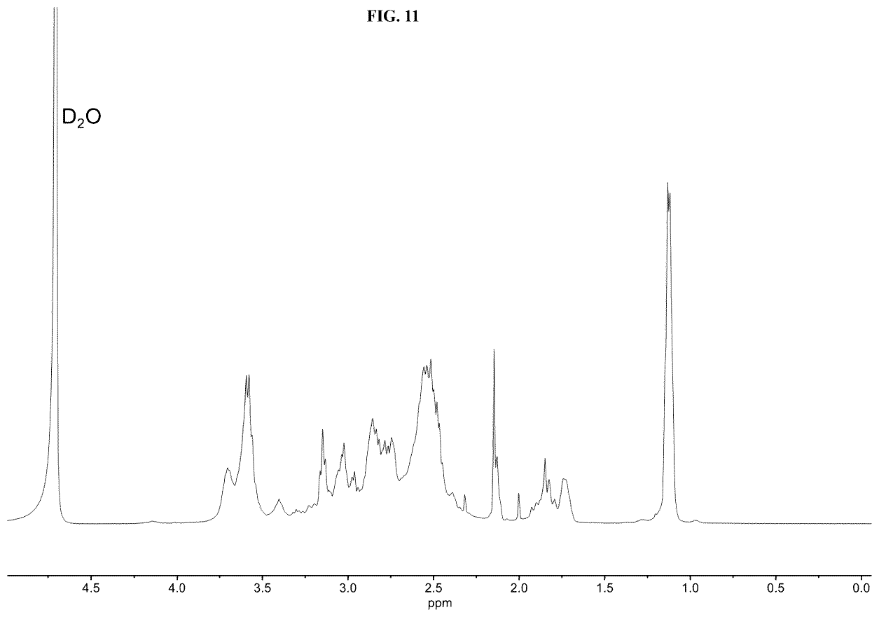

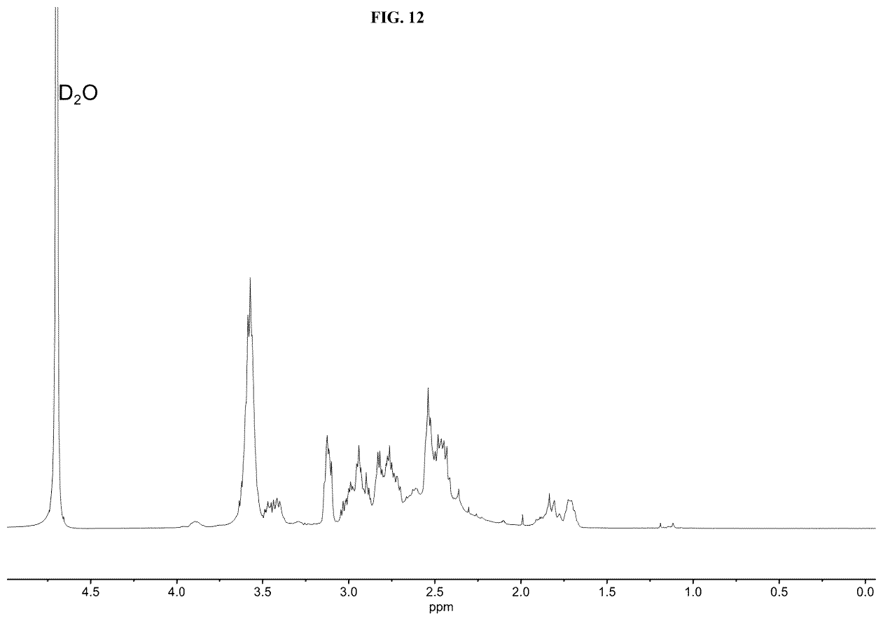

To enable bone microenvironment targeting of LNP formulations, a series of BP lipid-like materials consisting of alendronate, a type of BP, were designed and synthesized (FIG. 2A and FIG. 3). First, alendronate acrylamide was synthesized by reacting alendronate sodium with N-acryloxysuccinimide (FIGS. 3-5). The sodium salt was removed under acidic environments. Then, alendronate acrylamide was conjugated onto amine cores through a Michael addition reaction with a feeding molar ratio of 1:5, yielding alendronate-bearing amine cores. These structures were characterized by ¹H NMR spectrum and LC-MS analysis (FIGS. 5-12), demonstrating that although each amine of the core structures had the potential to conjugate alendronate, there was only one alendronate molecule featured on each core structure (FIG. 2A).

Following synthesis of alendronate-bearing amine cores, a library of twenty-one BP-lipids were synthesized by reacting the alendronate-bearing amine cores with epoxide-terminated alkyl chains (FIG. 2A and FIGS. 13A-13B). These BP-lipids were then mixed with the phospholipid DOPE, cholesterol, lipid-anchored polyethylene glycol (C14PEG2000), and mRNA via perfusion through a microfluidic mixing device that was designed with herringbone features to induce chaotic mixing. The naming of LNP formulations represented both the unique alendronate-conjugated amine core component (labeled as 110BP, 197BP, T3ABP, 200BP, 488BP, 490BP and 494BP) and the alkyl chain length (C12, C14 and C16) (FIG. 2A). In these LNPs, the ionizable lipid enabled cellular uptake and endosomal escape to deliver encapsulated mRNA into the cytosol. DOPE and cholesterol enhanced LNP stability, improved mRNA encapsulation, and assisted in endosomal escape. To demonstrate increased delivery to bone after the introduction of alendronate molecules, another LNP library without BPs modification was formulated and used as control (FIG. 14).

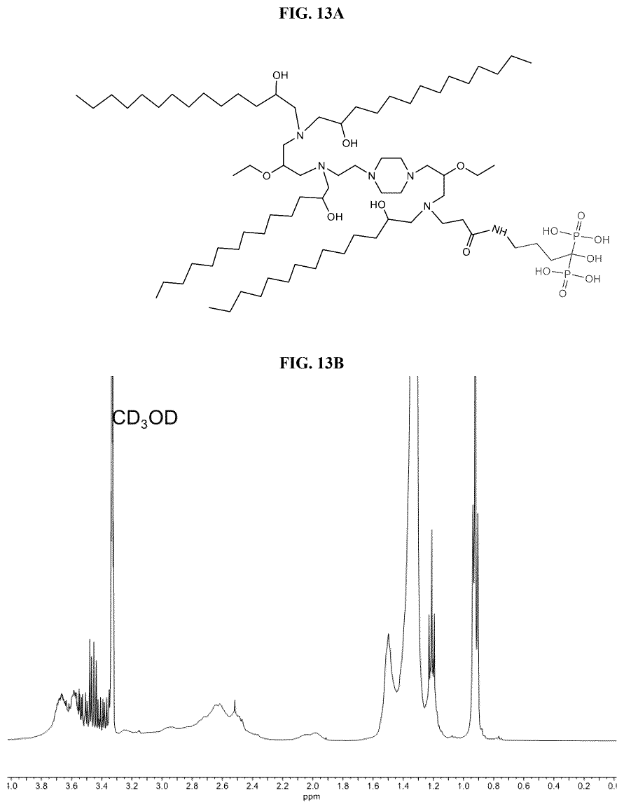

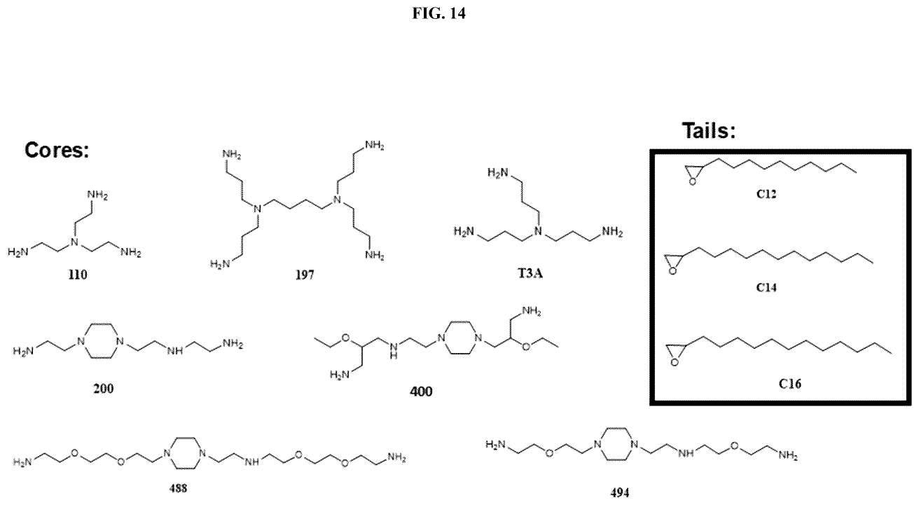

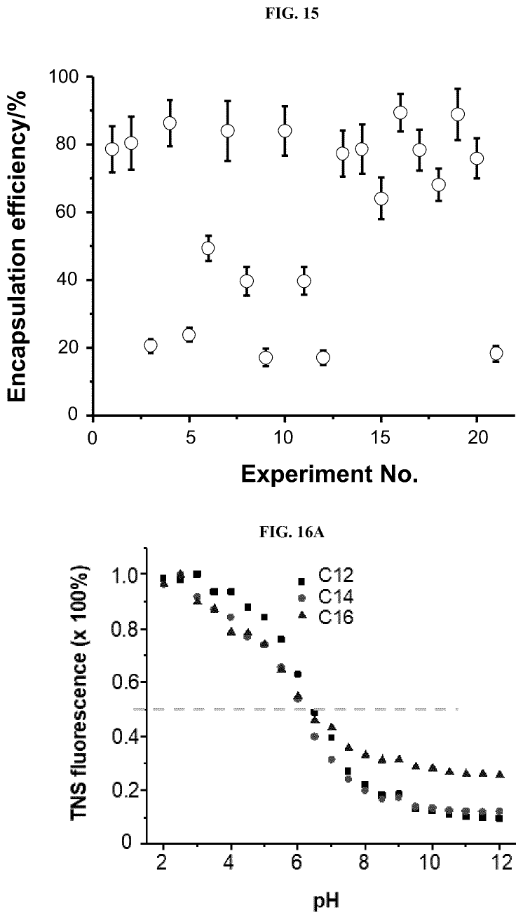

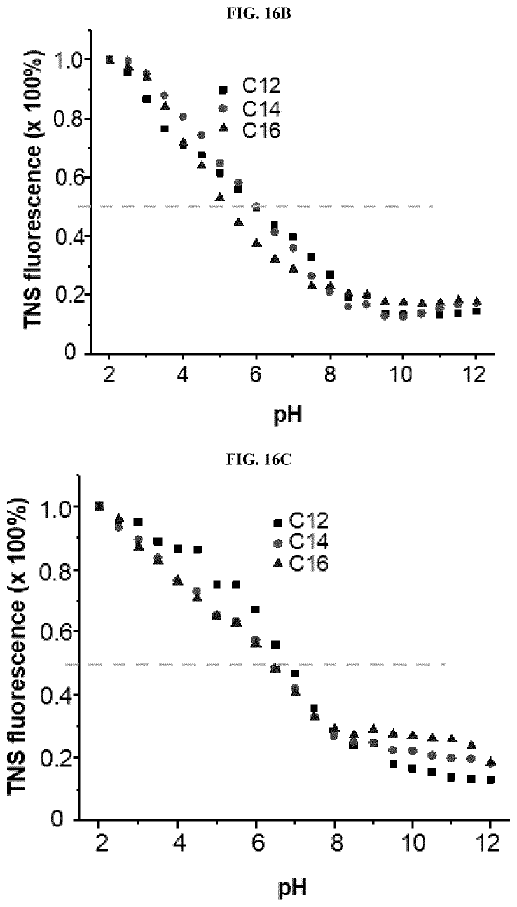

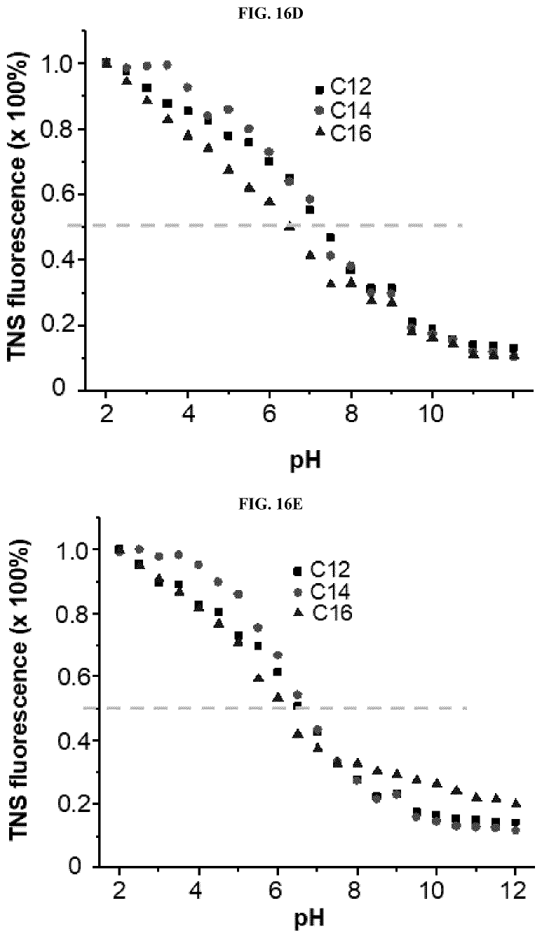

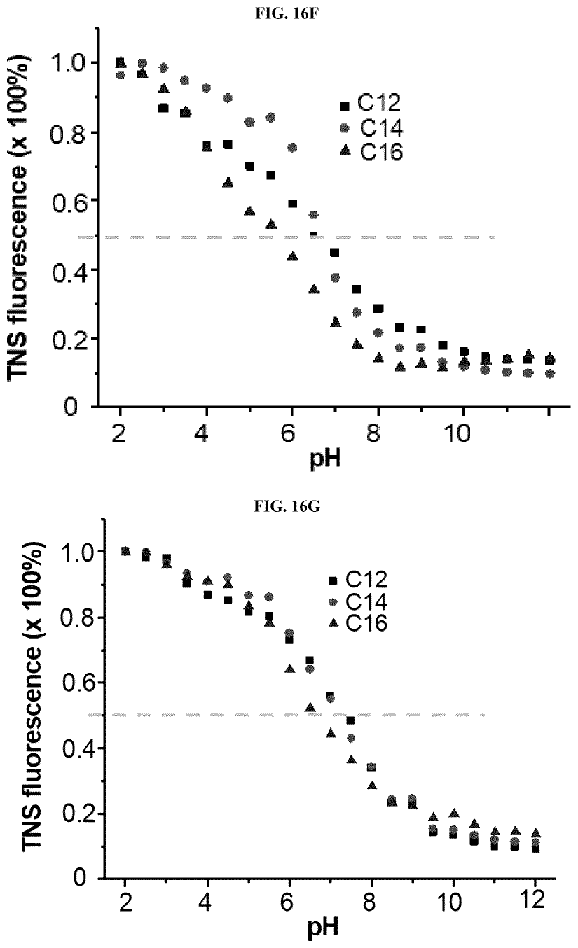

The resulting BP-LNPs were characterized to evaluate particle size, polydispersity index (PDI), zeta potential, pKa, and mRNA encapsulation efficiency. The hydrodynamic diameter for all BP-LNP formulations ranged from 60.1 to 124.2 nm by intensity measurements using dynamic light scattering (DLS) (FIG. 2B). Most BP-LNP formulations (>80%) were highly monodisperse, with a PDI value less than 0.2 (FIG. 2B). Owing to the incorporation of negatively charged alendronate molecules, BP-LNPs had a negative zeta potential (FIG. 2C). The mRNA encapsulation efficiency of BP-LNPs was assessed by a fluorescent R^{1b}oGreen assay, where most efficiencies ranged from 60 to 90%, while some LNPs containing BP lipid-like materials with a C16 alkyl chain did not formulate stable LNPs for mRNA loading due to their weak solubility during formulation (FIG. 15). Additionally, BP-LNPs were evaluated for their pKa, which indicated the pH at which BP-LNPs were 50% protonated (FIG. 2C and FIGS. 16A-16G). In previous studies, pKa values between 6 and 7 were most commonly reported for successful nucleic acid delivery as LNPs can become protonated in acidic endosomes within this range and can induce fusion with endosomal membranes to release mRNA into the cytosol. pKa values of BP-LNP formulations ranged from 5.18 to 7.35 (FIG. 2C), demonstrating that >70% of the LNPs were within the desired range (pKa 6.0-7.0) for endosomal escape and mRNA delivery.

### Example 3: BP-LNP In Vitro Screening, In Vitro Binding with Hydroxyapatite and Ex Vivo Affinity to Bone

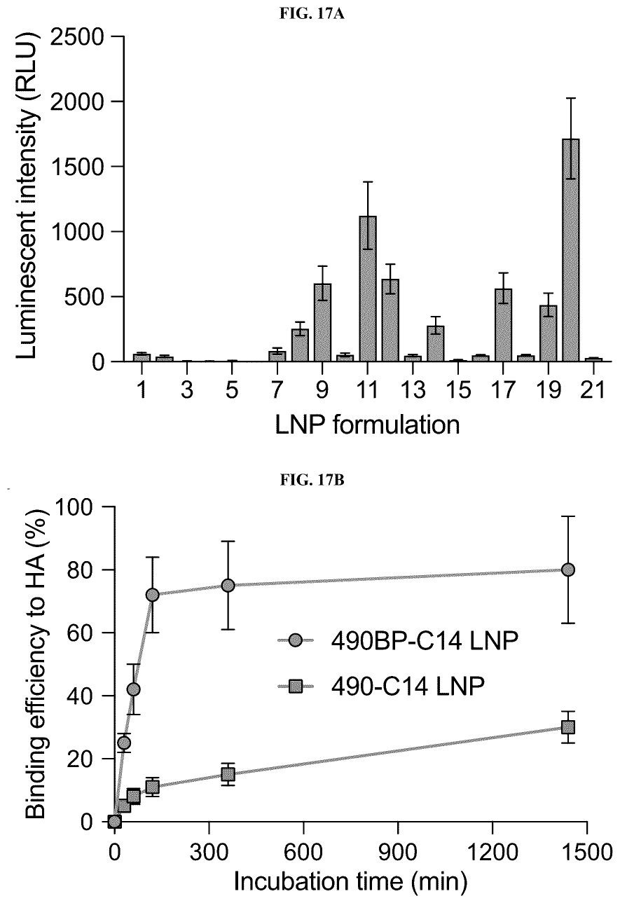

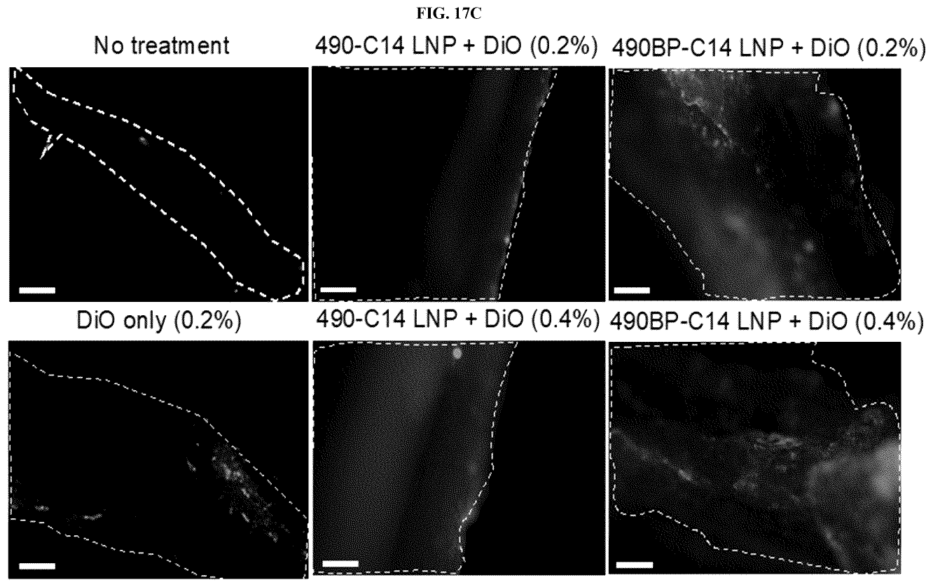

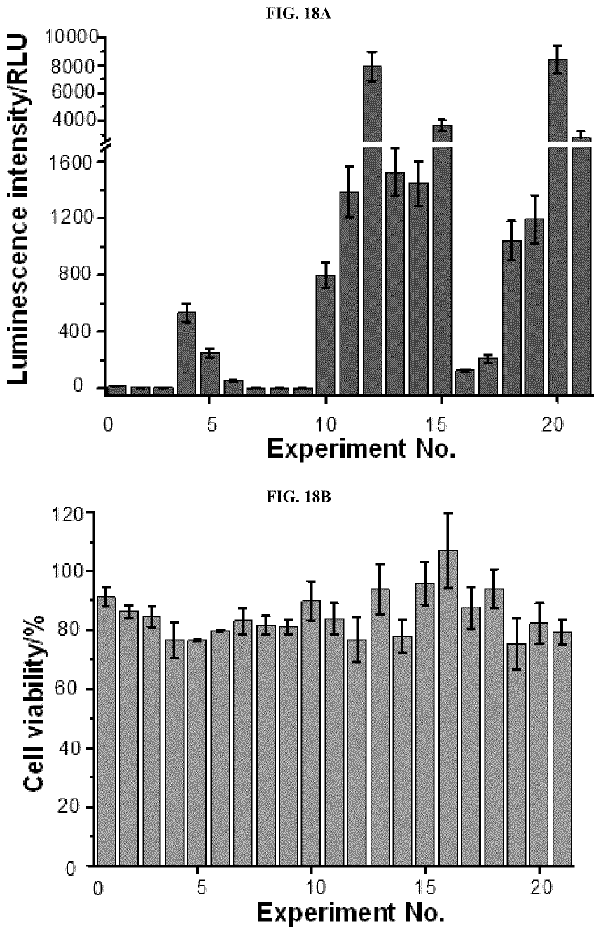

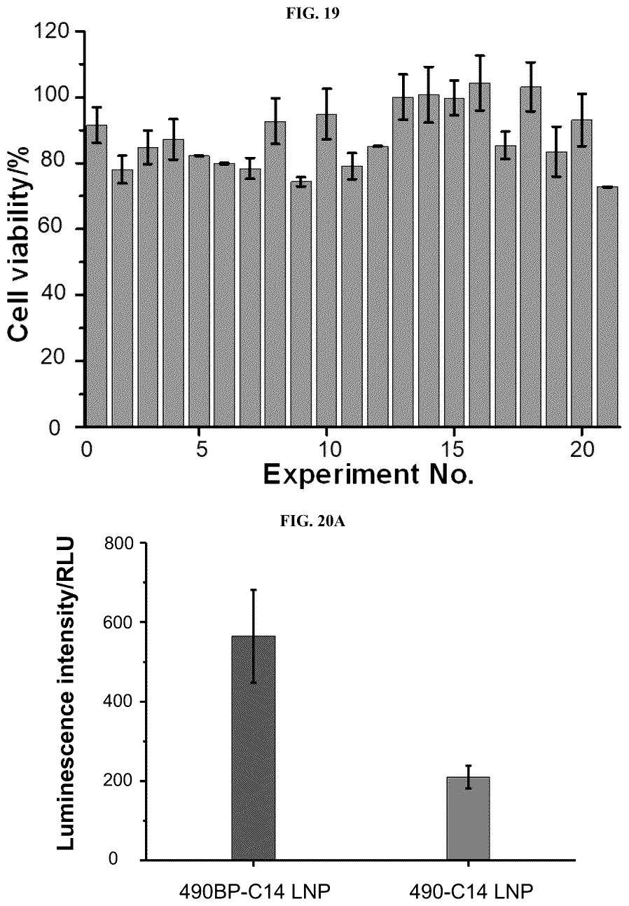

To evaluate BP-LNPs for their ability to deliver mRNA, mRNA encoding for luciferase was chosen as a reporter gene. Firefly luciferase (FLuc) mRNA with N1-Methyl-PseudoU modifications were used for screening in vitro and in vivo. These modifications have been shown to improve mRNA encapsulation efficiency, reduce overall immunogenicity, and enhance delivery of mRNA. In the present example, BP-LNP-mediated delivery of FLuc mRNA was assessed in HeLa cells, a cell line utilized for rapid in vitro screening of LNP formulations, for FLuc expression and cell viability. After 12 h treatment by BP-LNPs at a concentration of 10 ng/5000 cells, luciferase expression was evaluated via luminescence measurements, which were normalized to an untreated control group. These results allowed for determination of structure-activity relationships (SARs) with respect to epoxide-terminated alkyl tails and alendronate-amine core chemical structures, where five BP-LNPs resulted in similar or even higher luciferase expression compared to a standard LNP formulation using C12-200 as the ionizable lipid (FIG. 17A and FIGS. 18A-18B). All LNP formulations demonstrated low cytotoxicity; three formulations had cell viability between 70%-80%, while the remaining LNP formulations were >80% (FIG. 19).

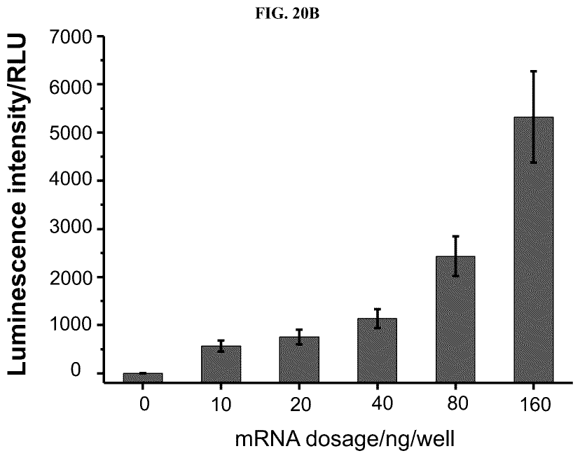

To further evaluate how the BP affects the ability of LNPs to deliver mRNA, a series of LNPs that did not incorporate BPs were formulated as controls (FIG. 14 and FIGS. 18A-18B). Among all of the LNP formulations examined in the initial screening, 490BP-C14 LNP was identified as the top formulation, as it demonstrated nearly 3 times higher transfection compared to 490-C14 LNPs in the absence of BPs (FIG. 20A). 490BP-C14 LNPs also demonstrated mRNA dose-dependent transfection of HeLa cells, showing increased luminescence intensity with increasing FLuc mRNA dosage (FIG. 20B). Therefore, 490BP-C14 was selected as the lead LNP formulation to further evaluate its ability to target bone for enhanced delivery of mRNA in vitro and in vivo.

To better explore and visualize the bone-binding efficiency of these BP-LNPs, a DiO fluorescence molecule was incorporated into both BP-LNPs and LNPs in the absence of BPs. As the bone microenvironment is rich in HA, first an HA binding assay of 490BP-LNPs in comparison to 490-C14 LNPs that did not contain BPs was performed (FIG. 17B). After incubation with HA nanoparticles, 490BP-C14 LNPs showed fast and efficient binding to HA (FIG. 17B), owing to the strong chelation of BPs on the LNP surface with HA. After a 2 h incubation, HA binding of BP-LNPs increased to 80%, significantly higher than LNPs without BPs (10%). After this time course, the binding efficiency plateaued with increased incubation time. The affinity of BP-LNPs to HA in bone fragments was further confirmed by fluorescence microscopy (FIG. 17C). Minimal fluorescence was detected on DiO-treated bone samples, indicating low binding between free DiO molecules and the bone surface. After treatment with DiO labelled 490-C14 LNPs, very weak fluorescence signal was generated, which resulted from passive accumulation of particles on the bone surface. This weak fluorescence signal was improved by increasing the DiO concentration on 490-C14 LNPs. However, 490BP-C14 LNPs exhibited the strongest binding affinity to bone fragments, indicating that the incorporation of BPs enabled LNP targeting to bone. These results demonstrated the impact of the design of targeted BP-LNPs in bone mineral binding, and the differential binding of targeted BP-LNPs that facilitated the following in vivo studies.

### Example 4: In Vivo Transfection and Biodistribution Studies of BP-LNPs

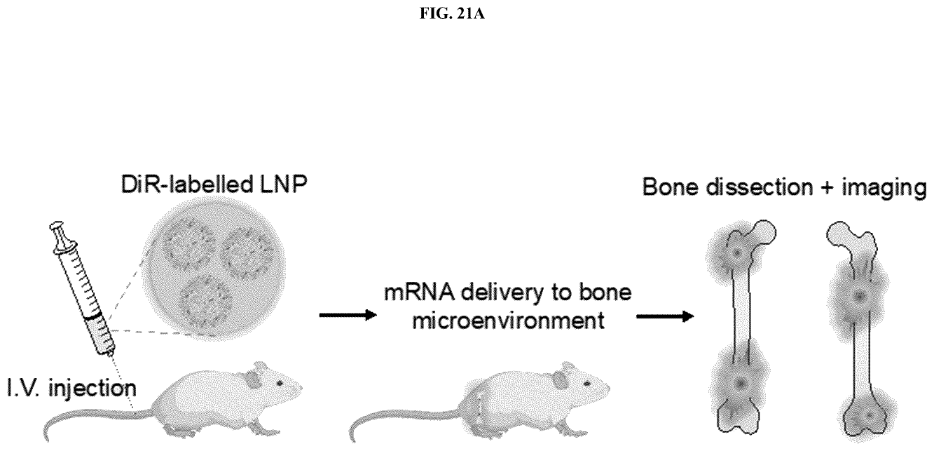

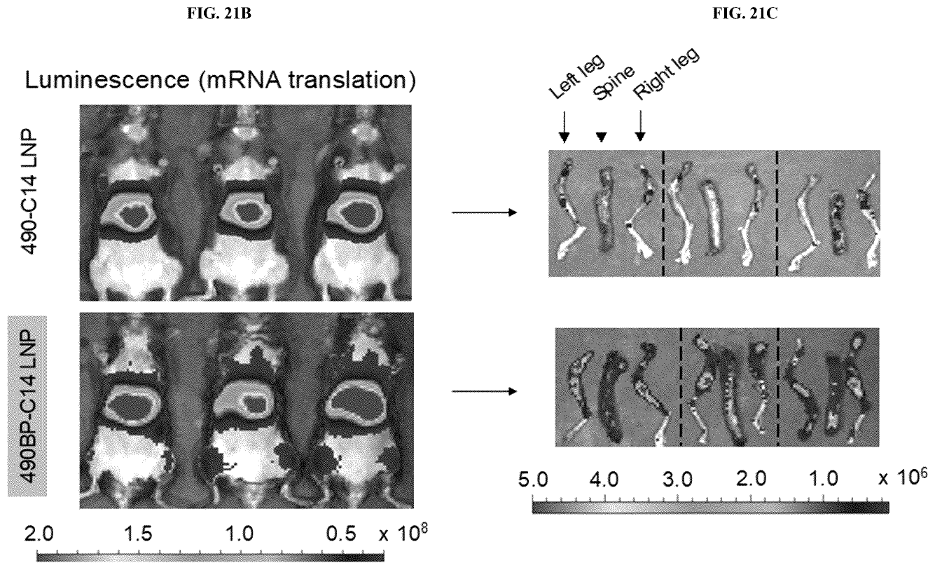

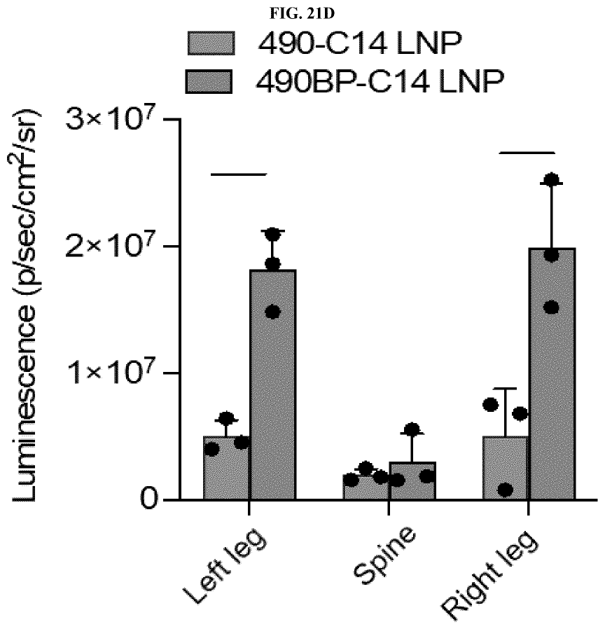

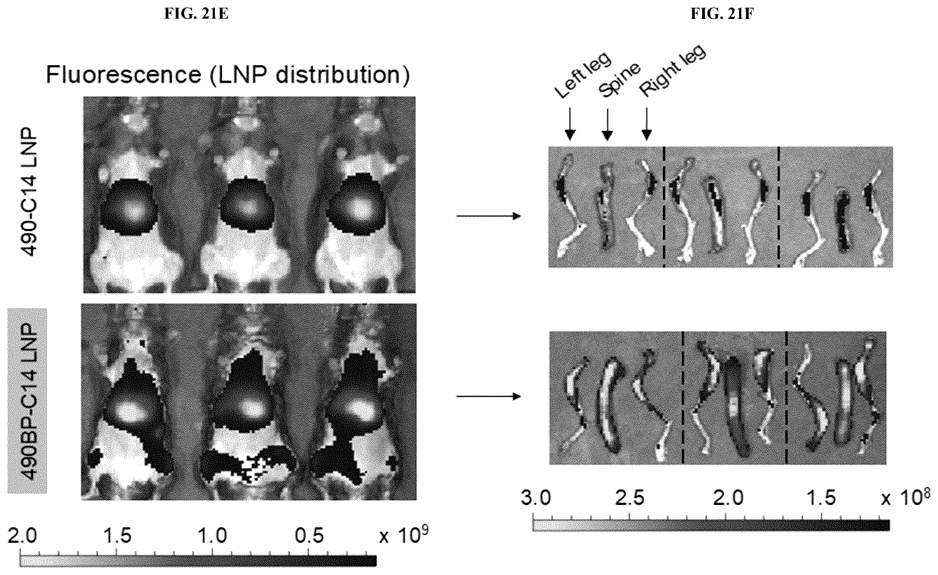

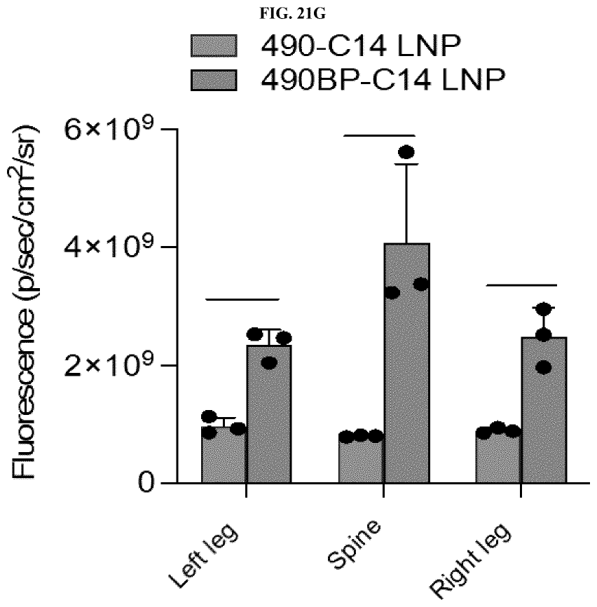

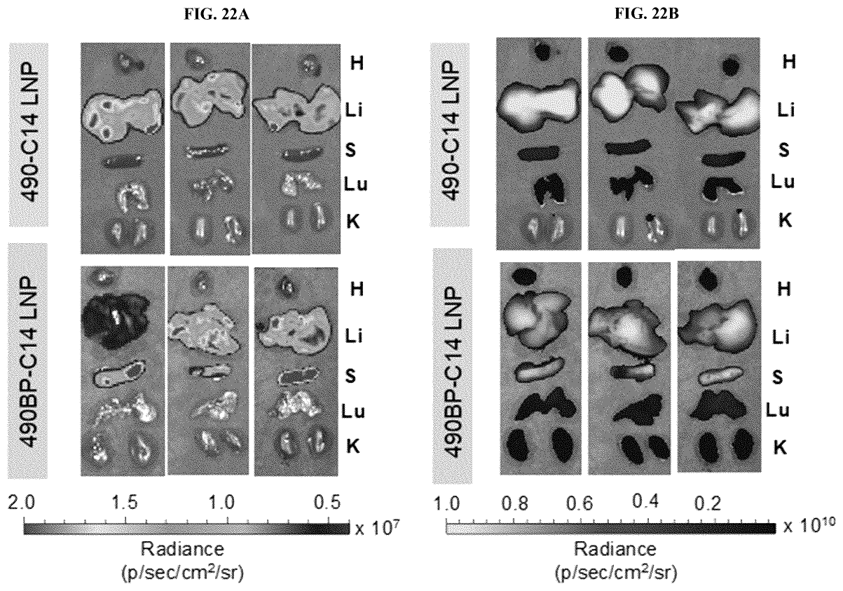

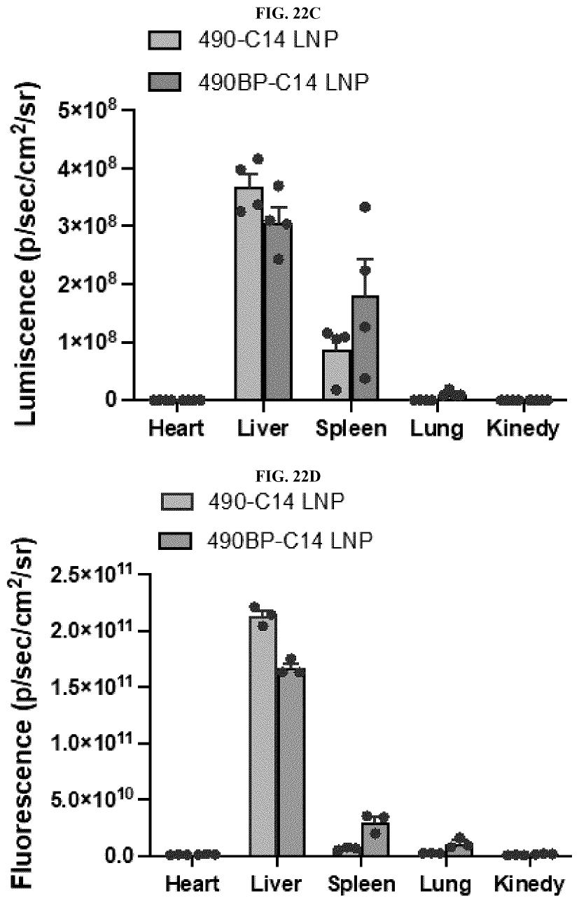

Following confirmation of the in vitro and ex vivo bone-targeting ability of BP-conjugated LNPs, in vivo transfection and biodistribution studies to investigate the bone-homing ability of BP-LNPs were performed using C57BL/6J mice following intravenous injection of 0.5 mg/kg FLuc mRNA. Fluorescent DiR was used to track LNP biodistribution using an in vivo imaging system (IVIS) (FIG. 21A). After 12 h, functional delivery of FLuc mRNA was observed in the liver and bones (FIGS. 21B-21D and FIG. 22). The luminescent intensity of the hind limbs was significantly higher following delivery of BP-LNPs (FIG. 21B). Bones from their hind limbs and spines of mice were then dissected to evaluate luminescence intensity by quantifying radiance efficiency (FIG. 21C), where LNPs without BPs showed very low transfection in the dissected bones, while BP-LNPs transfected cells on these bone surfaces, especially in the hind limbs, such as the femur and tibia (FIG. 21D). The mean transfection efficiency of left leg, spine, and right leg increased 3.7, 1.8, and 3.7 times, respectively, with BP-LNPs compared to LNPs without BPs.

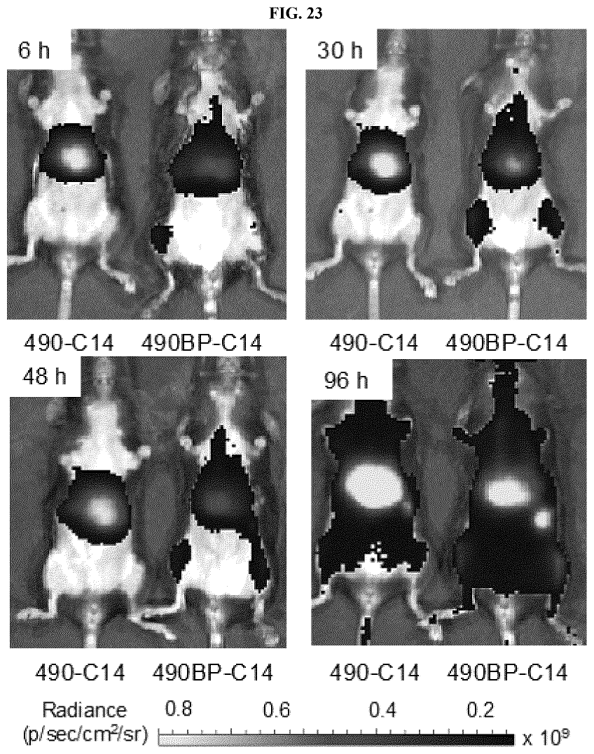

The biodistribution of BP-LNPs was then investigated (FIGS. 21E-21G). While LNPs without BPs mainly distributed in the liver, BP-LNP biodistribution was more widespread in liver/spleen and bone-dense regions (FIG. 21E). By observing the biodistribution of BP-LNPs over time, the fluorescence intensity of the whole body of mice increased, implying the adsorption of BP-LNPs during circulation (FIG. 23). For further examination of bone-targeting, dissected legs and spine were evaluated by quantifying the fluorescent intensity (FIG. 21F). All BP-LNP treated bone samples exhibited significantly increased fluorescence (FIG. 21G), with approximately 2.5, 4.5, 2.4-fold increases in the left leg, spine and right leg, respectively, compared to LNPs without BPs. Thus, in vivo transfection and biodistribution studies complement the in vitro and ex vivo high bone binding efficiency of BP-LNPs, highlighting their utility for bone-targeted mRNA delivery.

### Example 5: In Vivo BP-LNP Transfection and Distribution of Diverse Cell Types in Bone and Bone Marrow

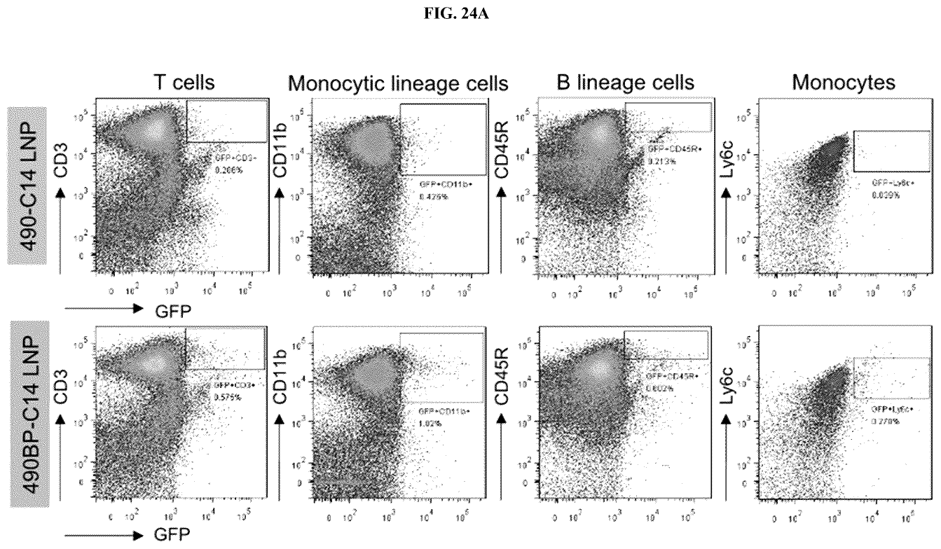

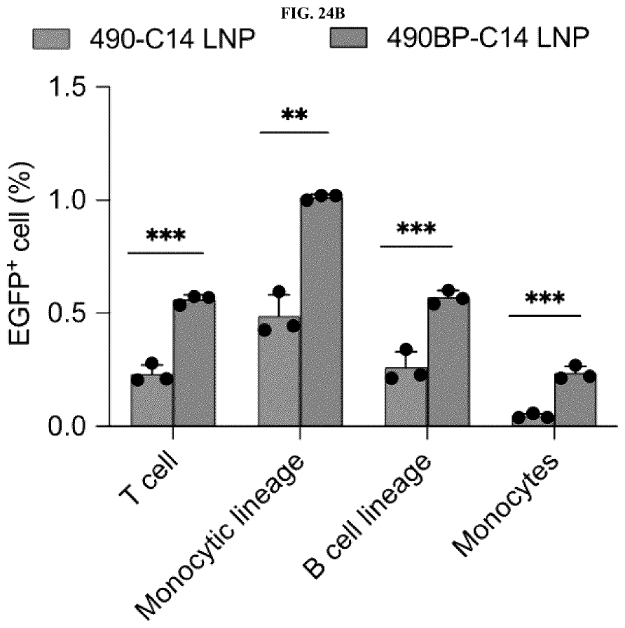

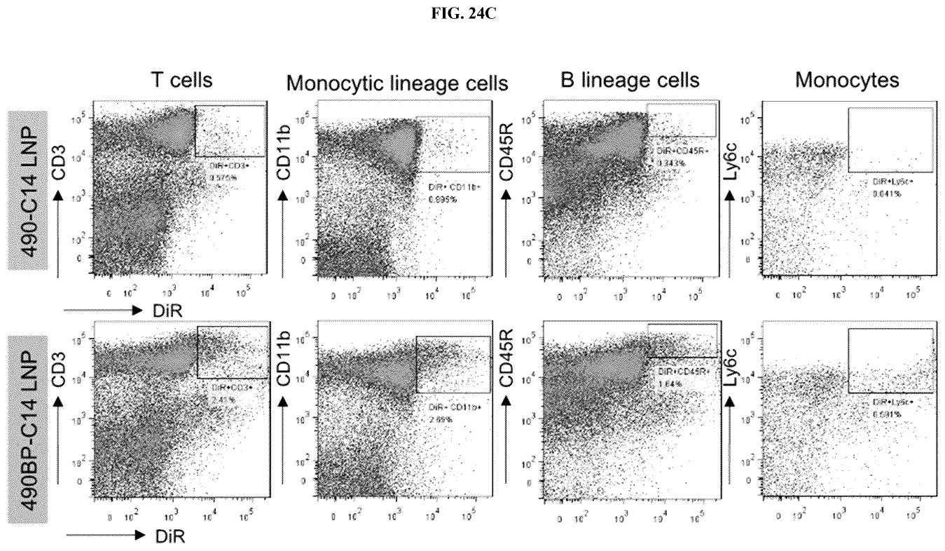

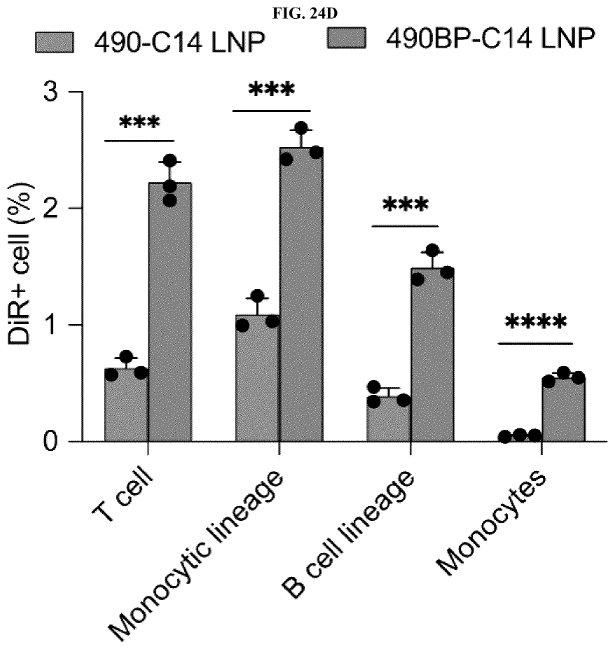

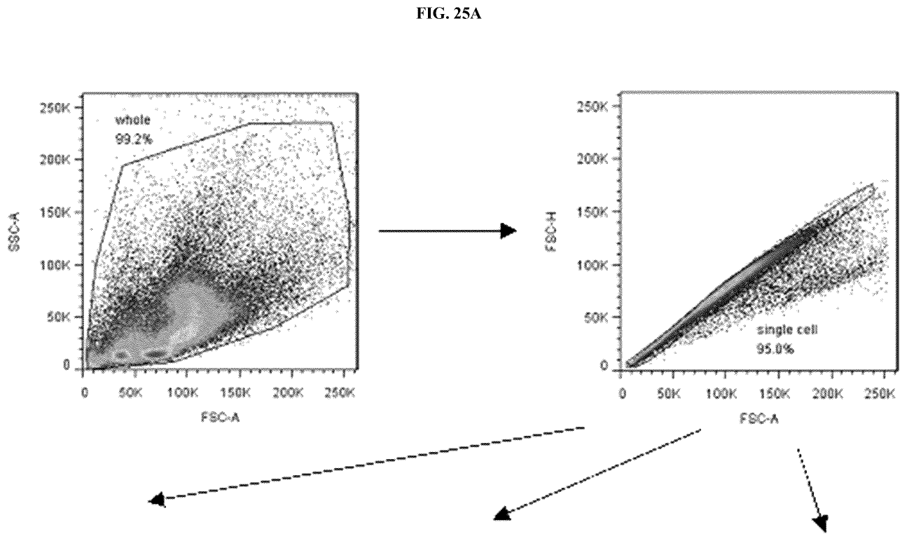

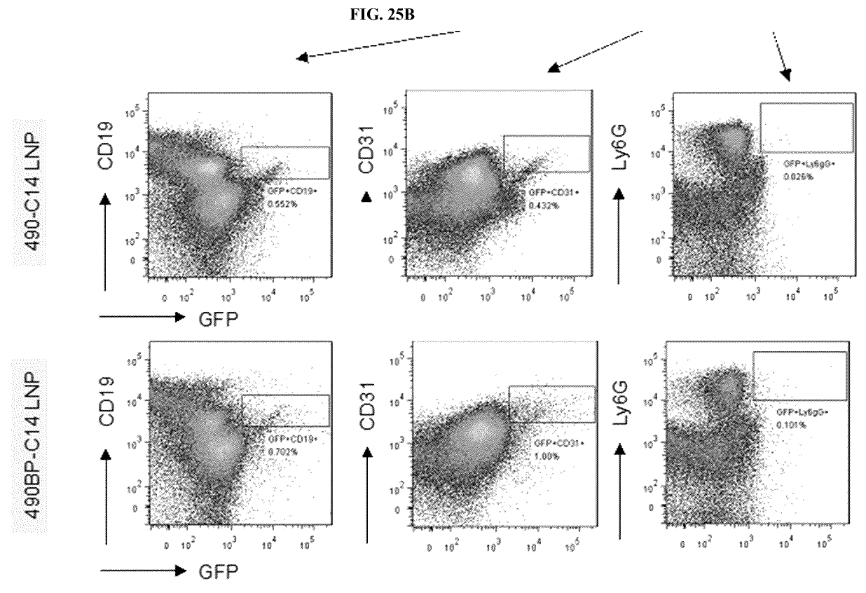

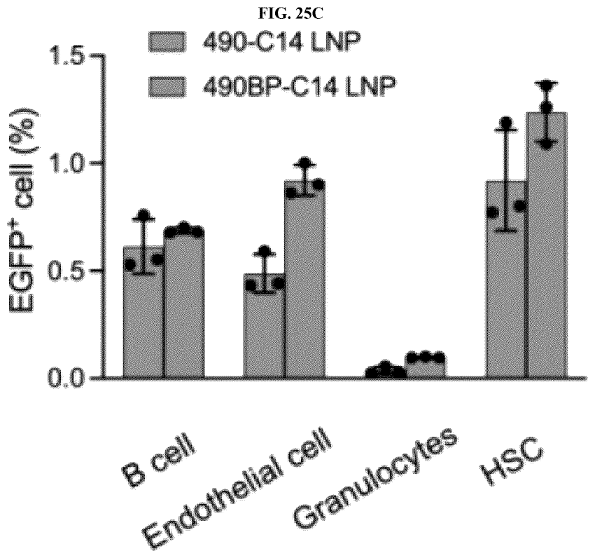

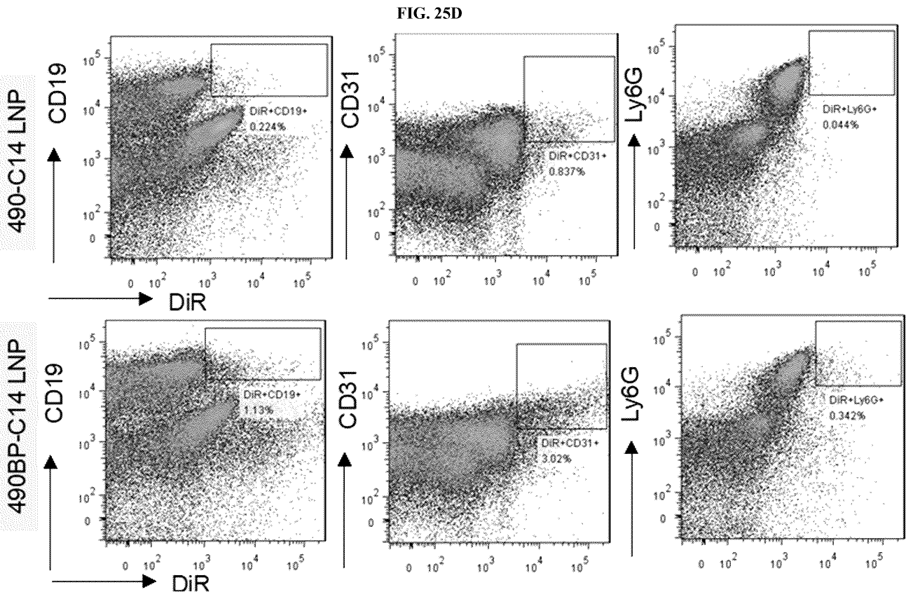

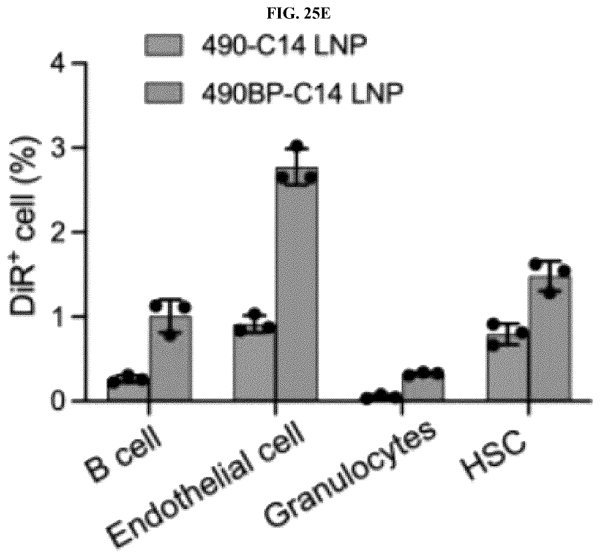

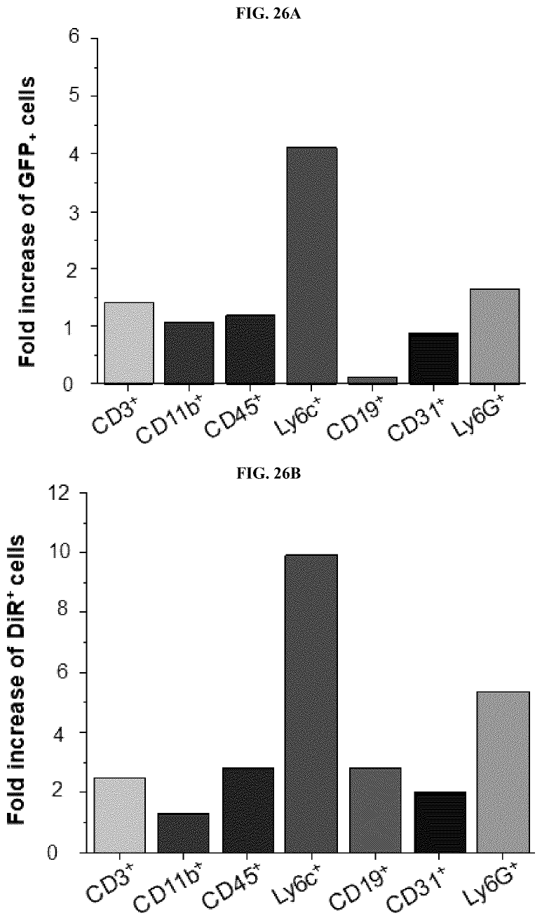

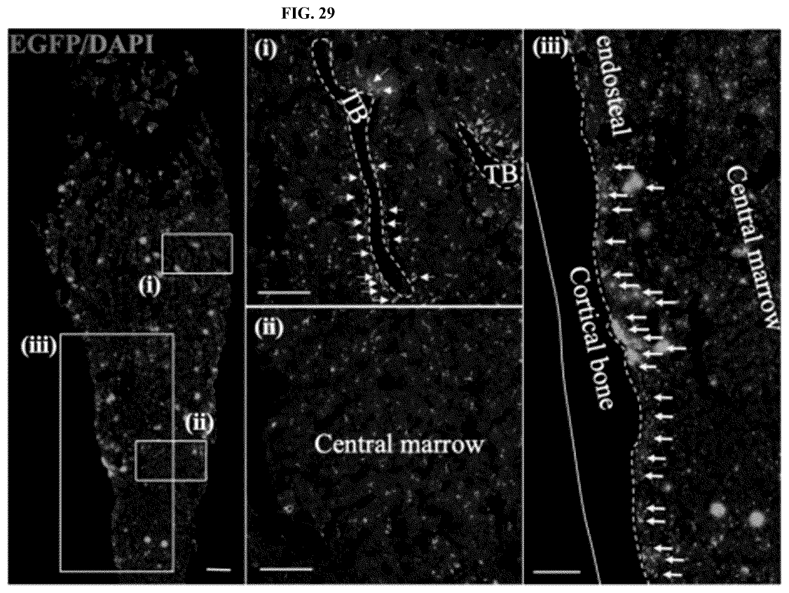

Having established that 490BP-C14 LNPs increased mRNA delivery to the bone microenvironment, the targeting of specific cell types in the bone marrow was evaluated, largely because bone marrow is the primary hematopoietic organ and residence site of diverse cell types. To demonstrate this, enhanced green fluorescent protein (EGFP)-encoding mRNA was administered to mice and flow cytometry was performed on cells isolated from bone marrow to identify transfection of different cell types. DiR labeled 490BP-C14 LNPs encapsulating EGFP mRNA were formulated and administered intravenously at 0.5 mg/kg, alongside PBS and 490-C14 LNP control groups (FIG. 29). After 12 h, mice were sacrificed, bone marrow was isolated, red blood cells were lysed, and the remaining cells were analyzed using flow cytometry. Flow cytometry analysis was conducted on diverse cell populations: monocytic lineage cells (CD11b⁺), B lineage cells (CD45R/B220⁺), T cells (CD3⁺), monocytes (Ly6c⁺), endothelial cells (CD31⁺), granulocytes (Ly6G+) and B cells (CD19⁺) (FIGS. 24A-24D and FIGS. 25A-25E). Compared to the 490-C14 LNP treated group, EGFP transfection efficiency was enhanced in T cells, monocytic lineage cells and B lineage cells as well as a small increase in B cells, endothelial cells and granulocytes following delivery of BP-LNPs (FIGS. 24A-24B and FIGS. 25A-25E). Interestingly, 490BP-C14 LNPs exhibited over 4 times higher transfection efficiency to monocytes than 490-C14 LNPs in the absence of BPs (FIGS. 26A-26B), indicating their utility in applications in inflammatory associated diseases. In addition to quantification of mRNA functional delivery, LNP biodistribution to bone marrow cells was investigated. BP-LNPs increased accumulation in diverse cell types, such as T cells, monocytic lineage cells, B lineage cells and monocytes (FIGS. 24C-24D).

The increased uptake was in agreement with the results from EGFP transfection efficiency (FIGS. 26A-26B), in which BP-LNP uptake by monocytes increased over 10 times compared with LNPs in the absence of BPs. Differing from the populations of EGFP⁺ cells, endothelial cells exhibited the highest BP-LNPs uptake ability, as a portion of BP-LNPs are not able to not pass through the blood-bone barrier after intravenous administration (FIGS. 25A-25E).

### Example 6: BMP-2 Secretion after Delivery of BMP-2 mRNA to Bone Microenvironment Using BP-LNPs

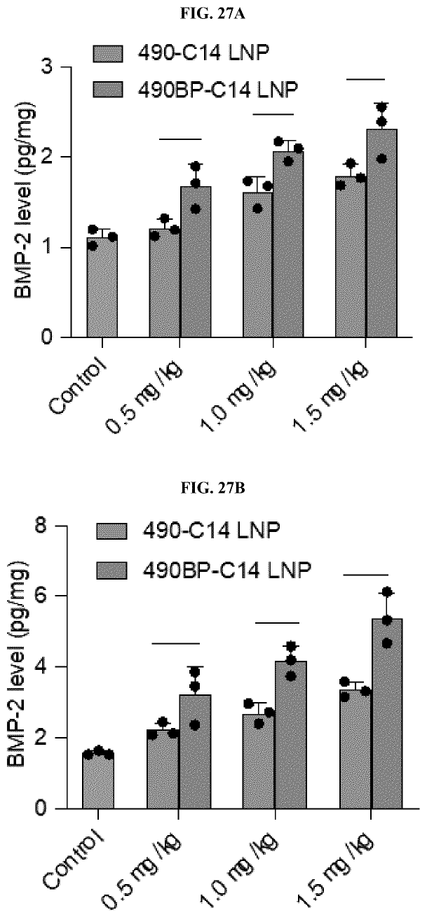

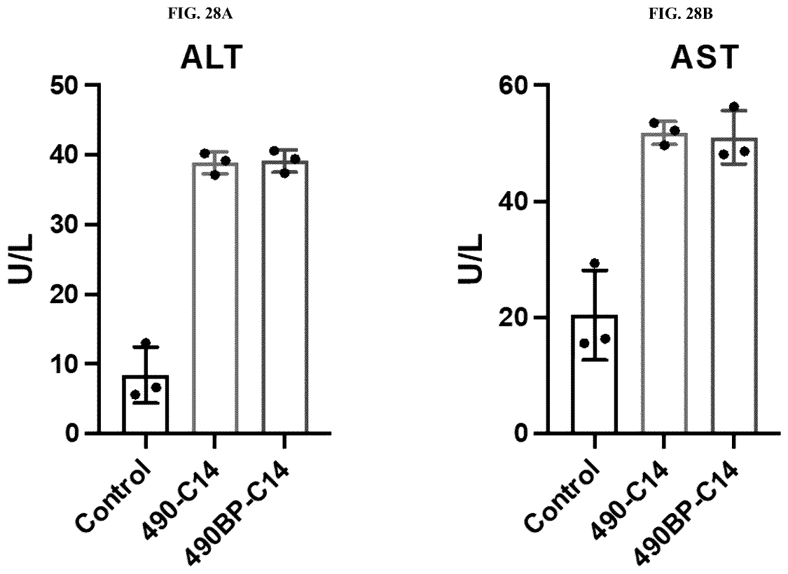

Having established BP-LNPs for enhanced systemic delivery of mRNA into the bone microenvironment, the delivery of mRNA encoding functional BMP-2 was then evaluated. BMP-2 is a multi-functional growth factor belonging to the transforming growth factor-beta (TGF-β) superfamily, which plays an important role in the process of bone induction that stimulates osteogenic differentiation. Therefore, increased expression of BMP-2 in the bone microenvironment leads to new applications for a variety of therapeutic interventions, such as bone defects, nonunion fractures, osteoporosis treatment, and root canal therapy. After BP-LNPs incorporating mRNA encoding BMP-2 were delivered by intravenous injection, BMP-2 levels on the bone surface and in the bone marrow were measured. It was noted that BMP-2 is normally secreted in the bone microenvironment, and thus BMP-2 secretion was observed both on the bone surface and in the bone marrow from the PBS treated control group. Although LNPs without BPs increased BMP-2 secretion in the bone microenvironment to some extent, BP-LNPs significantly increased BMP-2 expression (FIGS. 27A-27B). Furthermore, BMP-2 levels in the bone microenvironment increased with increasing BMP-2 mRNA dosage (FIGS. 27A-27B), reaching as high as 2.3 pg/mg and 5.3 pg/mg on the bone surface and in the bone marrow, respectively, at a mRNA dosage of 1.5 mg/kg, indicating great effectiveness for bone regeneration and fracture healing. Additionally, hematological analysis showed normal liver function (assessed by aspartate aminotransferase (AST) and alanine aminotransferase (ALT) activities) in mice treated with BP-LNPs, demonstrating that BP-LNPs did not cause notable toxicity in vivo (FIGS. 28A-28B).

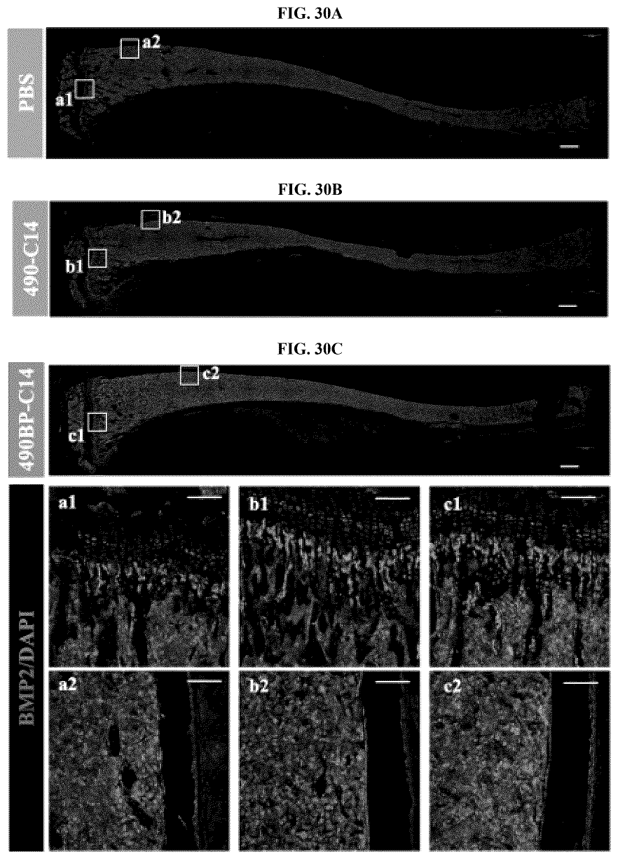

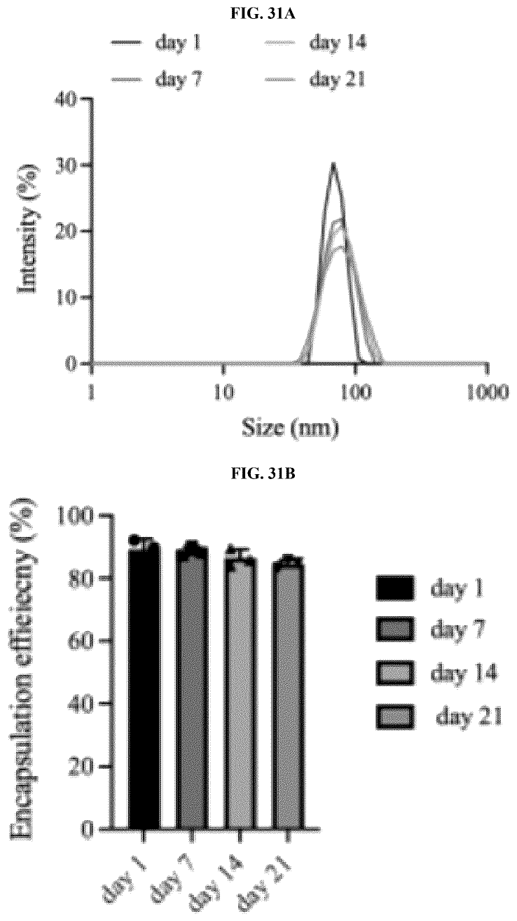

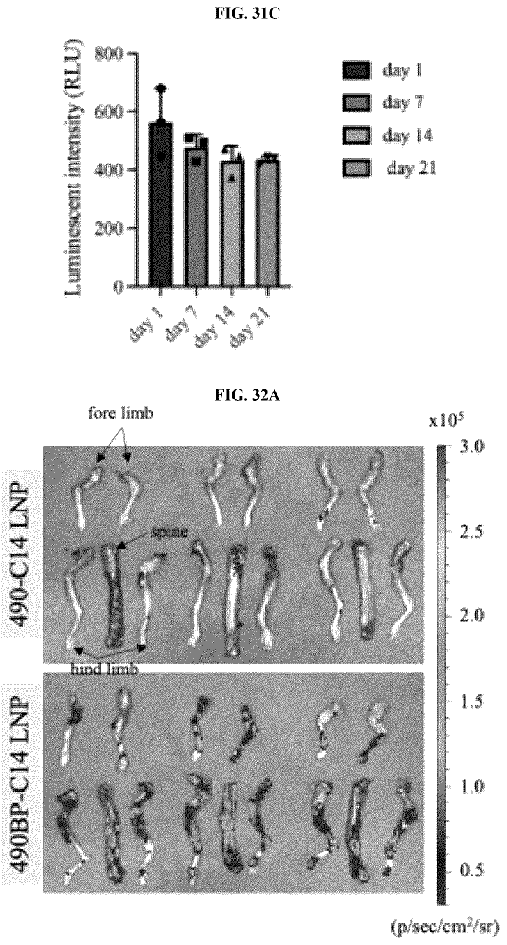

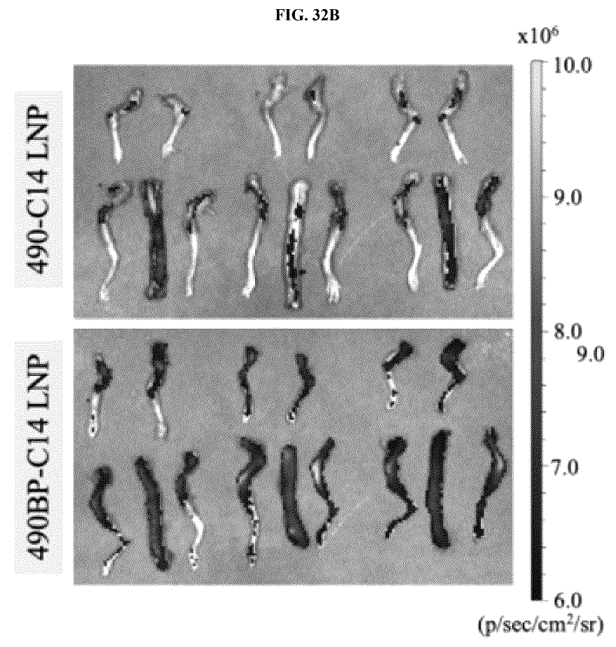

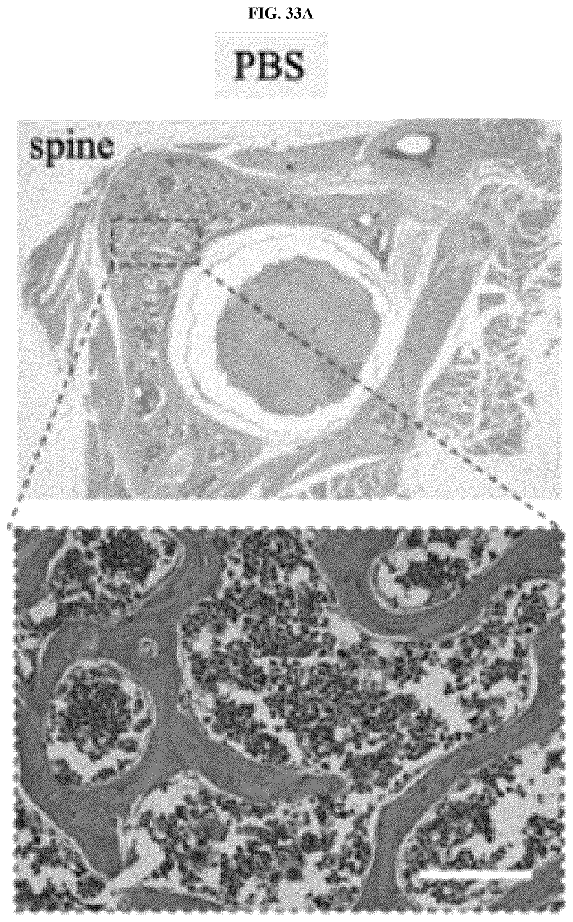

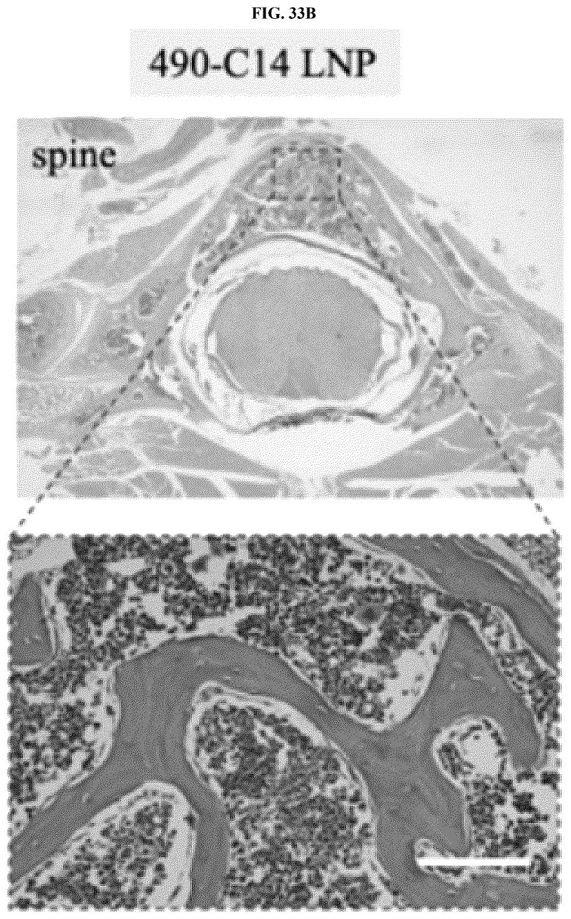

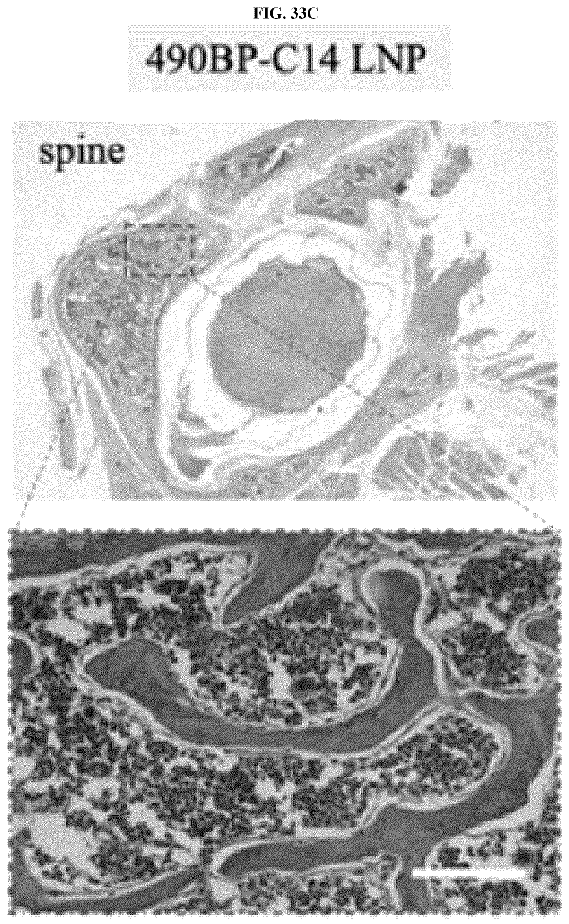

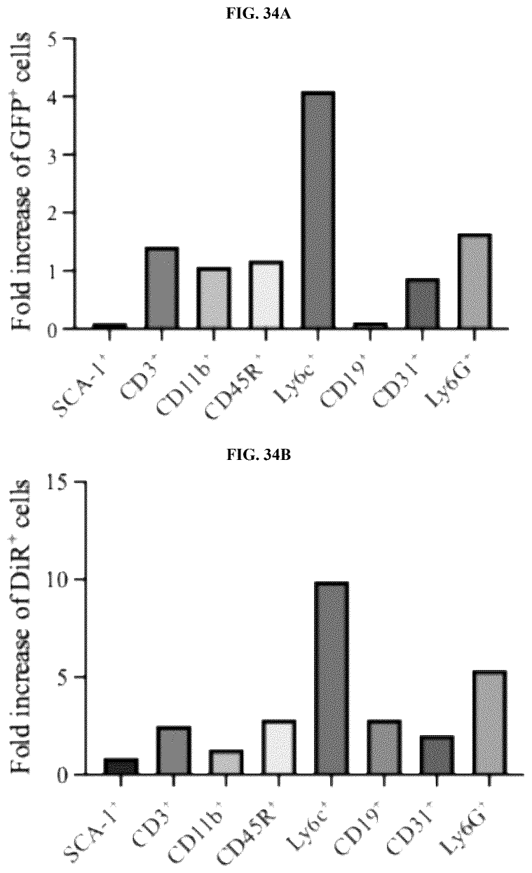

To further characterize BMP-2 expression, whole tibia bones from mice were then dissected for immunofluorescence (IF) analysis. Compared to low BMP-2 expression in mice treated by PBS (FIG. 30A), 490-C14 LNP delivering BMP-2 mRNA demonstrated relatively higher BMP-2 expression on both the trabecular and cortical bone surfaces (FIG. 30B). In addition, 490BP-C14 LNPs delivering BMP-2 mRNA demonstrated significantly higher BMP-2 expression in the metaphyseal and endosteal marrow (FIG. 30C). Representative BMP-2 expression regions, such as the growth plate, primary spongiosa, metaphyseal bone marrow (FIGS. 30A-30C; see a1, b1, and c1), and endosteal region (FIGS. 30A-30C; see a2, b2, and c2) of the medial anterior tibia were chosen to show the targeted delivery and expression of BMP-2. As shown in the magnified regions (FIGS. 30A-30C), the increase in BMP-2 expression in the metaphyseal and endosteal bone marrow for the 490BP-C14 LNP group illustrates the importance of BP lipid-like materials for mRNA delivery to bone and a potential disease treatment strategy based on BMP-2 expression.

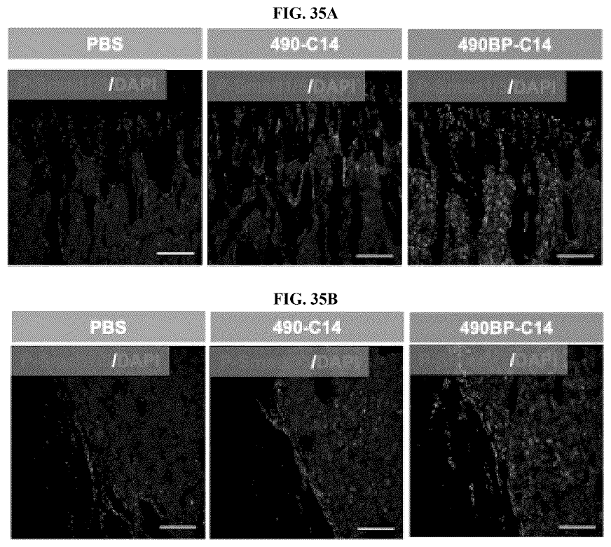

However, it is still difficult to characterize the cell populations which express BMP-2 because of (1) the diverse and complex cell populations in bone marrow and (2) the secretion of BMP-2 protein that may influence their internal distribution. To investigate whether proper signaling pathways are activated after BMP-2 mRNA delivery, IF staining of Smad1/5 was performed, which is phosphorylated during BMP-2 expression. A noticeable increase in phosphorylated Smad1/5 was detected in cells on the trabecular bone surface (FIG. 35A), on the cortical bone surface (FIG. 35B), and in bone marrow (FIGS. 35A-35B) after 490BP-C14 LNP delivery of BMP-2 mRNA, indicating efficient BMP-2 mRNA delivery to the bone microenvironment and subsequent signaling pathway activation.

In summary, a facile and versatile approach to engineer a library of BP lipid-like materials that were formulated into LNPs (BP-LNPs) for systemic delivery of mRNA to the bone microenvironment was developed. The bone-targeting BP ligand alendronate was readily conjugated onto amine cores to construct a series of novel BP lipid-like materials, which then formed stable bone-homing LNPs when combined with DOPE, cholesterol, and C14-PEG2000 through microfluidic mixing. After an initial assessment from in vitro screening, 490BP-C14 LNP was identified as the lead formulation in this library. Compared to LNPs that did not incorporate BP-lipids, 490BP-C14 LNPs showed increased higher hydroxyapatite binding in vitro and affinity to bone fragments ex vivo. Following systemic delivery, LNP homing and mRNA transfection in the bone microenvironment significantly increased with LNPs incorporating BP lipid-like materials. In addition, 490BP-C14 LNPs showed increased transfection efficiency in diverse cell types in the bone marrow, especially monocytes (4 times higher). BMP-2 secretion on the bone surface and in the bone marrow significantly increased after systemic BMP-2 mRNA delivery. The incorporation of BP lipid-like materials into LNPs demonstrated their effectiveness in targeted mRNA delivery to bone for applications in regenerative medicine, protein replacement, and gene editing therapies for bone and bone marrow. Additional studies focus on utilizing this generalizable approach to engineer targeted LNPs for enhanced site-specific targeting described here to develop expanded wealth of additional lipid-like materials for targeted mRNA therapeutics.

In conclusion, the development of LNP formulations for targeting the bone microenvironment holds significant utility for nucleic acid therapeutic applications, including bone regeneration, cancer, and hematopoietic stem cell therapies. However, therapeutic delivery to bone remained a significant challenge due to several biological barriers, such as low blood flow in bone, blood-bone marrow barriers, and low affinity between drugs and bone minerals, which led to unfavorable therapeutic dosages in the bone microenvironment. The present studies disclosed a series of BP lipid-like materials possessing high affinity to bone minerals, as a means to overcome biological barriers to deliver mRNA therapeutics efficiently to the bone microenvironment in vivo. Following in vitro screening of BP lipid-like materials formulated into LNPs, a lead BP-LNP formulation, 490BP-C14, was identified with enhanced mRNA expression and localization in the bone microenvironment of mice in vivo compared to 490-C14 LNPs in the absence of BPs. Moreover, BP-LNPs enhanced mRNA delivery and secretion of therapeutic bone morphogenetic protein-2 from the bone microenvironment upon intravenous administration. These results demonstrated the effectiveness of BP-LNPs for delivery to the bone microenvironment, which are utilized for a range of mRNA therapeutic applications, including regenerative medicine, protein replacement, and gene editing therapies.

Moreover, the materials of the present disclosure are useful in delivering nucleic acids into bones. LNPs commonly go to liver via IV injection and extra-hepatic delivery remains a daunting challenge. The bone marrow microenvironment is rich in genetic targets, such as hematopoietic stem cells (HSCs), and cancer cells that metastasize to bone. Furthermore, bone regeneration is a major challenge that mRNA therapeutics of the present disclosure address. The herein described bone-targeted ionizable lipids are also able to deliver nucleic acids to HSCs for gene editing therapies, to cancer cells for immunotherapy or gene therapies, and are also able to produce proteins to promote bone regeneration.

### Bisphosphonate-Substituted Piperazine Ionizable Lipid Compounds and Bone-Targeted Lipid Nanoparticles (LNPs) Thereof

### Materials and Methods

### Chemical Reagents

1-Boc-piperazine (98%) was purchased from Oakwood Chemical. Acryloyl chloride (98%), chloroacetyl chloride (97%), succinyl chloride (95%), N-hydroxysuccinimide (98%), triethylamine (≥99%), dichloromethane (≥99.8%), ethyl acetate (ACS reagent, >99.5%), n-hexane (≥95%), and cholesterol (≥99%) were purchased from Sigma-Aldrich. Alendronate sodium trihydrate (98%), 1,2-epoxyhexadecane (C16, 98%), and N1-(2-(4-(2-aminoethyl)piperazin-1-yl)ethyl)ethane-1,2-diamine were purchased from AmBeed Chemical. Potassium carbonate (anhydrous, 99%), sodium hydroxide (white pellets), Benzoxazolium, 3-octadecyl-2-[3-(3-octadecyl-2(3H)-benzoxazolylidene)-1-propenyl]-, perchlorate (DiO, 97% at 490 nm), and 1,1′-Dioctadecyl-3,3,3′,3′-tetramethylindodicarbocyanine (DiD, 99% at 640 nm) were purchased from ThermoFisher Scientific. N,N-Diisopropylethylamine (≥99.5%) was purchased from Acros Organics Chemicals. Trifluoroacetic acid (≥99.0%), acetonitrile (≥99.8%), and tris(2-aminoethyl)amine (B1, 97%) were purchased from Alfa Aesar Chemicals. Ethyl alcohol (200 proof, 100%) was purchased from Decon Labs. 1,2-Epoxydecane (C10, >97.0%), 1,2-epoxydodecane (C12, ≥95.0%), 1,2-epoxytetradecane (C14, ≥95.0%), and tris(3-aminopropyl)amine (B3, 97%) were purchased from TCI Chemicals. Bis(3-aminopropyl)({4-[bis(3-aminopropyl)amino]butyl})amine (B2, 95%, 2-(4-{2-[(2-aminoethyl)amino]ethyl}piperazin-1-yl)ethan-1-amine (P1, 95%), 3-(4-{2-[(3-amino-2-ethoxypropyl)amino]ethyl}piperazin-1-yl)-2-ethoxypropan-1-amine (P2, 95%), 2-{2-[4-(2-{[2-(2-aminoethoxy)ethyl]amino}ethyl)piperazin-1-yl]ethoxy}ethan-1-amine (P3, 95%), and {2-[2-(2-aminoethoxy)ethoxy]ethyl}[2-(4-{2-[2-(2-aminoethoxy)ethoxy]ethyl}piperazin-1-yl)ethyl]amine (P4, 95%) were purchased from Enamine. 1,2-dioleoyl-sn-glycero-3-phosphoethanolamine (DOPE, >99%) and 1,2-dimyristoyl-sn-glycero-3-phosphoethanolamine-N-[methoxy(polyethyleneglycol)-2000](C12-PEG2000, >99%) were purchased from Avanti Polar Lipids. Hydroxyapatite disc was purchased from Clarkson Chromatography Products. Deuterium oxide (D2O, 99.8%), dimethyl sulfoxide-d₆ (DMSO-d₆, 99.8%), and chloroform-d (CDCl₃) were purchased from Acros Organics Chemicals. All the chemicals were used without further purification.

Biological assays

Firefly Luciferase (FLuc) mRNA (5-methoxyuridine) (Ref L-7202) was purchased from TriLink Biotechnologies. Quant-it™ R^{1b}oGreen™ RNA reagent and kit were purchased from ThermoFisher Scientific. Luciferase 1000 assay system (Ref E4550) and CellTiter-Glo luminescent cell viability (Ref. G7572) were purchased from Promega Corporation.

Cell culture

Dulbecco's Modified Eagle Medium (DMEM) with high glucose, L-glutamine, phenol red, and without sodium pyruvate and HEPES was purchased from Gibco Scientific. Trypsin-EDTA (0.25%) and penicillin-streptomycin (P/S) were purchased from Gibco Scientific. Fetal bovine serum (FBS) was purchased from Sigma-Aldrich. Hep-G2 cells were cultured in DMEM supplemented with 10% FBS and 1% P/S. BJ cells were cultured in DMEM supplemented with 10% FBS.

Animal studies

C57BL/6J mice were purchased from Jackson Laboratory. All experimental procedures were approved by the Institutional Animal Care & Use Committee (IACUC) of the University of Pennsylvania and complied with relevant local, state, and federal regulations.

### Instruments

FT-IR spectra were recorded with a Thermo Scientific™ Nicolet™ STM 5 FT-IR spectrometer, equipped with an iD7 ATR diamond. ¹H and ¹³C NMR spectra were acquired using a Bruker NEO NMR spectrometer at 400 MHz and 101 MHz (or 600 MHz and 125 MHz), respectively. ³¹P NMR spectra were also acquired using the same instrument operating at 162 MHz (or 243 MHz). All the NMR measurements were conducted at room temperature in D2O, DMSO-d6, or CDC13. NMR data were processed using MNova 14 software. Chemical shifts (6) are reported in parts per million (ppm). The resonance patterns in the 1H NMR spectra are denoted as “s” (singlet), “d” (doublet), “t” (triplet), “q” (quartet), “m” (multiplet), and “br” (broad resonance). The residual protic solvent of D2O (¹H, δ 4.79 ppm), DMSO-d6 (¹H, δ 2.50 ppm; ¹³C, δ 39.52 ppm), CDC13 (¹H, δ 7.26 ppm; ¹³C, δ 77.16 ppm) and tetramethylsilane (TMS, δ 0 ppm) served as the internal reference for ¹H and ¹³C NMR spectra. The structural frameworks of all compounds were conclusively determined by two-dimensional nuclear magnetic resonance spectroscopy (2D NMR), including ¹H-¹H COSY, ¹H-¹³C HSQC, and ¹H-¹³C HMBC. LC-MS analyses were conducted on a Waters™ Chromatography Column equipped with a UV-Vis detector. The analysis was carried out using a solution consisting of a mixture of water (50%) and acetonitrile (50%) at room temperature. Compound detection was performed through UV absorbance at X=254 nm. A PerSeptive Biosystem-Voyager-DE MALDI-ToF spectrometer (Framingham, MA) equipped with a nitrogen laser (337 nm) and operating in linear mode was utilized for characterizing the ionizable lipids. Angiotensin II and Bombesin served as reference standards for calibration. In order to prepare the sample solution, each compound was initially dissolved in THF at a concentration of 5-10 mg/mL. Subsequently, a matrix solution of 2,5-dihydroxybenzoic acid was prepared in THF at a concentration of 10 mg/mL, and the two solutions were combined at a ratio of 1/5 (v/v, compound solution to matrix solution). LNPs were formulated using a Pump33DS syringe pump (Harvard Apparatus, Holliston, MA). Particle size and zeta potential were measured by Dynamic Light Scattering (DLS) using a DynaPro™ Plate Reader III or Malvern Zetasizer Nano ZS. Particle morphology was assessed using Cryo-TEM. The AFM images were acquired using a Bruker Dimension Icon AFM operating in Tapping Mode, employing MikroMasch 325 kHz cantilevers. The surface of the HA discs was examined using a confocal laser scanning microscope (Zeiss LSM800) equipped with a 20× water-dipping objective (numerical aperture=1.0). Sequential scanning was conducted using diode lasers (488 nm for DiO and 640 nm for DiD), and the emitted signal was collected using optimized emission wavelength filter sets. A 405-nm laser in reflection mode was used to visualize the HA surface. Z-stack datasets (0.260-μm pixel size and 0.850 μm/z step) were acquired and processed (maximum intensity projection) using ZEN lite software (Zeiss). The DiO fluorescence dye is excited at 490 nm and emits at 506 nm. The DiD fluorescence dye is excited at 649 nm and emits at 668 nm. All computed conformers were generated using the Gaussian 16 software package. Initial chemical structures were optimized using the Universal Force Field (UFF) molecular mechanics method, followed by single-point energy calculations. The conformers were analyzed and visualized using the GaussView 6 and Jmol software programs.

Biological assays
LNP formulation

The LNPs utilized in this investigation were synthesized through the following procedure: An ethanol phase comprising all lipid constituents and an aqueous phase containing FLuc mRNA were combined using a microfluidic device to fabricate LNPs. The ethanol phase consisted of either ALENDRONATE™-bearing ionizable lipid or standard ionizable lipid, along with DOPE, cholesterol, and C14-PEG2000 in fixed molar ratios of 35%, 16%, 46.5%, and 2.5%, respectively. The aqueous phase contained mRNA dissolved in a 10 mM citrate buffer. Subsequently, the ethanol and aqueous phases were blended at flow rates of 1.8 ml/min and 0.6 ml/min (in a 3:1 ratio) using Pump33DS syringe pumps. The resulting LNPs underwent dialysis in 1×PBS using a microdialysis cassette (MWCO=20,000, ThermoFisher Scientific, Waltham, MA) for 2 hours followed by filtration through a 0.22 μm filter. The Z-average diameters, polydispersity index (PDI), and Zeta potential were assessed using a DynaPro Plate Reader III or Malvern Zetasizer Nano ZS instrument. Additionally, the mRNA concentration and encapsulation efficiency in each LNP formulation were determined via a modified Quant-iT R^{1b}oGreen assay (ThermoFisher Scientific) conducted on a plate reader.

FLuc mRNA in vitro delivery screening

Hep-G2 or BJ cells were seeded at a density of 5,000 cells per well in 96-well plates with transparent bottoms and white walls. Each well contained 100 μL of growth medium, consisting of DMEM supplemented with 10% FBS and 1% P/S for Hep-G2 cells, and DMEM supplemented with 10% FBS for BJ cells. The plates were then incubated at 37° C. in a 5% CO₂ atmosphere. Following this, the growth medium was replaced with fresh medium, and LNPs were administered at a dosage of 5 ng FLuc mRNA per well. After 24 hours of post-LNP transfection, firefly luciferase expression was quantified using the Luciferase Assay System (Promega) as per the manufacturer's instructions. The luminescent signal was normalized against cells treated with growth medium alone. Cell viability was assessed using the CellTiter-Glo Luminescent Cell Viability Assay (Promega), following the manufacturer's protocol.

Characterization of bone-targeted LNPs-HA interaction in vitro

Cells were seeded at a density of 20,000 cells per well in a 12-well plate, with each well containing 400 μL of growth medium consisting of 10% FBS in DMEM. The plate was then incubated at 37° C. in a 5% CO2 atmosphere. Similarly, each chamber was filled with 200 μL of growth medium consisting of 10% FBS in DMEM and placed into a separate 12-well plate. These chambers in the plate were also incubated at 37° C. with a 5% CO₂ concentration. Subsequently, the growth medium in the 12-well plate was replaced with fresh medium, while the medium in the chambers was replaced with fresh medium containing LNPs at a concentration of 40 ng FLuc mRNA. After 24 hours of post-LNP transfection, the chambers were removed, and firefly luciferase expression in the adherent cells at the bottom of the 12-well plate was quantified using the Luciferase Assay System (Promega) according to the manufacturer's instructions. The luminescent signal was normalized against cells treated with growth medium alone. Cell viability was evaluated using the CellTiter-Glo Luminescent Cell Viability Assay (Promega), following the manufacturer's protocol.

FLuc mRNA in vivo delivery screening

All experimental procedures involving female C57BL/6J mice aged 6-8 weeks were conducted under the guidelines approved by the Institutional Animal Care & Use Committee (IACUC) of the University of Pennsylvania. Mice received a single intravenous injection of FLuc mRNA LNPs at a dosage of 0.5 mg/kg via the tail vein. The expression of luciferase was assessed using an IVIS Spectrum imaging system (Caliper Life Sciences) 12 hours post-injection. Subsequently, mice were injected with D-luciferin (PerkinElmer) at a dose of 150 mg/kg via intraperitoneal injection (IP). After a 10-minute incubation under anesthesia, bioluminescence intensity was quantified by measuring photon flux in the region of interest using Living IMAGE Software provided by Caliper. Ex vivo imaging was conducted on the heart, liver, spleen, lung, kidney, forelimbs, and legs after resection.

### Example 7: Ionizable Lipid Synthesis

### Product 1 (tert-Butyl 4-acryloylpiperazine-1-carboxylate)

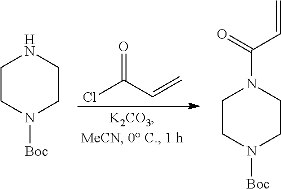

After dissolving 1-boc-piperazine (5 g, 26.84 mmol, 1 eq.) in acetonitrile (50 mL, 0.5 M to ‘1-boc-piperazine’) containing potassium carbonate (11.13 g, 80.53 mmol, 3 eq.), the mixture was stirred rapidly at room temperature for 15 minutes. Subsequently, the reaction mixture was cooled to 0° C. using an ice bath, and acryloyl chloride (2.62 mL, 32.21 mmol, 1.2 eq.) was added dropwise while maintaining the temperature at 0° C. The reaction mixture was stirred at 0° C. for 1 hour. After filtration to remove potassium carbonate, the solutions of supernatant were completely removed under reduced pressure to give the product (6.19 g, 96%). White solid. FT-IR (ATR mode): 1682 and 1633 [v(R₂N—(C=O)—)], 1618 [v(—C═C—)], 1251 and 1167 [v(—C—O-)]cm⁻¹. ¹H NMR (400 MHz, CDCl₃): δ 6.50 (m, 1H, R₂N—(C═O)—CH═CH₂), 6.23 and 5.66 (dd, 2H, R₂N—(C═O)—CH═CH₂, J=16.8, 1.9 Hz), 3.70-3.30 (m, 8H, —RN—(CH₂CH₂)₂—NR—), 1.41 (s, 9H, —(C═O)—O—C(CH₃)₃) ppm. ¹³C{H}NMR (101 MHz, CDCl₃): δ 165.6 (R₂N—(C═O)—CH═CH₂), 154.5 (—(C═O)—O—(CH₃)₃), 128.3 (R₂N—(C═O)—CH═CH₂), 127.3 (R₂N—(C═O)—CH═CH₂), 80.3 (—(C═O)—O—C(CH₃)₃), 45.5, 43.5, and 41.8 (—RN—(CH₂CH₂)₂—NR—), 28.3 (—(C═O)—O—C(CH₃)₃) ppm. MS (ES+ve) m/z (abundance %) for C₁₂H₂₀N₂O₃: calculated [M+H]⁺ 241.3068, found [M+H]⁺ 241.2650 (100).

### Product 2 (4-((3-(4-(tert-Butoxycarbonyl)piperazin-1-yl)-3-oxopropyl)amino)-1-hydroxybutane-1,1-diyl)bis(phosphonic acid)

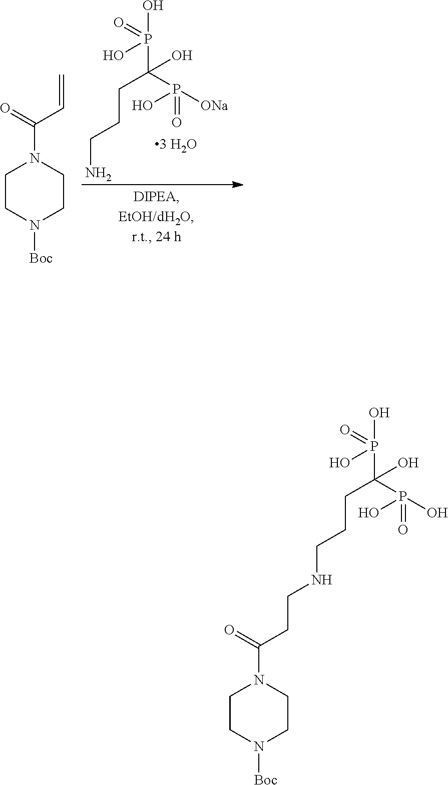

‘Product 1’ (5.32 g, 22.14 mmol, 1.2 eq.) was dissolved in ethanol (184.5 mL, 0.12 M to ‘Product 1’) and N,N-diisopropylethylamine (DIPEA) (9.64 mL, 55.35 mmol, 3 eq.) was added at room temperature. In another vial, Alendronate (6 g, 18.45 mmol, 1 eq.) was dissolved in deionized water (dH₂0) (123.0 mL, 0.15 M to ‘Alendronate™’) at room temperature and the resulting solution was added dropwise to the round-bottom flask containing ‘Product 1’. The reaction mixture was stirred at room temperature for 24 hours. The reaction solution was then completely removed under reduced pressure. Subsequently, acetone was added to induce precipitation of ‘Product 2’ (8.13 g, 90%), and no additional separations were conducted for the next reactions. White hygroscopic solid. Due to the hydrophobic alkyl chains and bisphosphonate group, the compound exhibits very low solubility in both aqueous and organic solvents. FT-IR (ATR mode): 2974 [v(—NH—)], 1692 and 1637 [v(R₂N—(C═O)—)], 1234 and 1162 [v(—C—O—)], 1053 [v(—(P=O)—)], 535 and 453 [v(—(P—O)-)]cm⁻¹. ¹H NMR (400 MHz, D₂O): 6 3.69-3.63 (m, 2H, R₂N—(C═O)—CH₂—CH₂—NMR), 3.61-3.45 (m, 8H, —RN—(CH₂CH₂)₂—NR—), 3.40-3.26 (m, 2H, R₂N—(C═O)—CH₂—CH₂—NMR), 3.11-3.03 (m, 2H, —NH—CH₂—CH₂—CH₂—), 2.16-1.93 (m, 4H, —NH—CH₂—CH₂—CH₂), 1.48 (s, 9H, —(C═O)—O—C(CH₃)₃) ppm. ¹³C{H}NMR (101 MHz, D₂O): 6 170.4 (R₂N—(C═O)—CH₂—CH₂—NHR), 156.3 (—(C═O)—O—C(CH₃)₃), 82.2 (—(C═O)—O—C(CH₃)₃), 73.5 (—C(OH)—(HPO₃)₂), 54.4 (R₂N—(C═O)—CH₂—CH₂—NHR), 45.6, 42.5, and 41.6 (—RN—(CH₂CH₂)₂—NR—), 44.9 (R₂N—(C═O)—CH₂—CH₂—NHR), 40.0 (—NH—CH₂—CH₂—CH₂—), 30.7 (—NH—CH₂—CH₂—CH₂—), 27.6 (—(C═O)—O—C(CH₃)₃), 22.3 (—NH—CH₂—CH₂—CH₂—) ppm. ³¹P{H}NMR (162 MHz, D₂O): δ 17.9 and 17.8 ppm. MS (ES+ve) m/z (abundance %) for C₁₆H₃₉N₃O₁₃P₂: calculated [M+H₂O+H]⁺, found [M+H₂O+H]⁺562.7176 (50).

### Product 3 (4-(N-(3-(4-(tert-Butoxycarbonyl)piperazin-1-yl)-3-oxopropyl)-2-chloroacetamido)-1-hydroxybutane-1,1-diyl)bis(phosphonic acid)

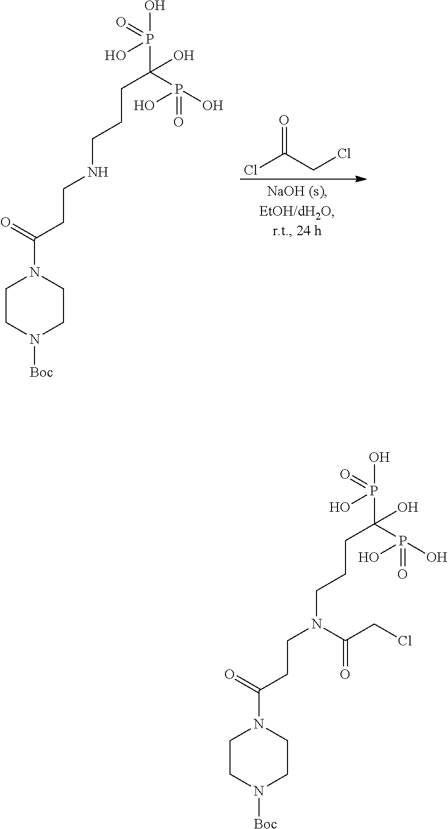 [SMILES: CC(C)(C)OC(=O)N1CCN(C(=O)CCNCCCC(O)(P(=O)(O)O)P(=O)(O)O)CC1 ; O=C(Cl)CCl ; CC(C)(C)OC(=O)N1CCN(C(=O)CCN(CCCC(O)(P(=O)(O)O)P(=O)(O)O)C(=O)CCl)CC1]

Product 2′ (4 g, 8.17 mmol, 1 eq.) was dissolved in a solution containing 33% dH₂0 and 67% ethyl acetate (EA) (21.5 mL, 0.38 M to ‘Product 2’). Subsequently, a finely ground powder of sodium hydroxide (3 eq.) was added, and the mixture was stirred at room temperature for 15 minutes. Chloroacetyl chloride (0.78 mL, 9.80 mmol, 1.2 eq.) was then added dropwise to the reaction solution, which was stirred at room temperature for 24 hours. The reaction solution was completely removed under reduced pressure to give the product (4.3 g, 93%), and no additional separations were conducted for the next reactions. White solid. Due to the hydrophobic alkyl chains and bisphosphonate group, the compound exhibits very low solubility in both aqueous and organic solvents. FT-IR (ATR mode): 1693 and 1626 [v(R₂N—(C═O)—)], 1560 [v(R₂N—(C═O)—CH₂—C1)], 1240 and 1166 [v(—C—O—)], 1054 [v(—(P═O)—)], 647 [v(—CH₂—C1)], 542 and 463 [v(—(P—O)-)]cm-1. ¹H NMR (400 MHz, D₂O): δ 4.49 (s, 2H, R₂N—(C═O)—CH₂—C1), 3.68-3.44 (m, 10H, R₂N—(C═O)—CH₂—CH₂—NHR and —RN—(CH₂CH₂)₂—NR—), 3.41-3.20 (m, 2H, R₂N—(C═O)—CH₂—CH₂—NHR), 3.14-3.02 (m, 2H, —NH—CH₂—CH₂—CH₂—), 2.19-1.97 (m, 4H, —NH—CH₂—CH₂—CH₂—), 1.50 (s, 9H, —(C═O)—O—C(CH₃)₃) ppm. ¹³C{H}NMR (101 MHz, D₂O): δ 175.0 (R₂N—(C═O)—CH₂—CH₂—NHR), 170.5 (R₂N—(C═O)—CH₂—C1), 156.4 (—(C═O)—O—C(CH₃)₃), 82.3 (—(C═O)—O—C(CH₃)₃), 73.7 (—C(OH)—(HPO₃)₂), 63.3 (R₂N—(C═O)—CH₂—C1), 53.2 (R₂N—(C═O)—CH₂—CH₂—NHR), 49.5, 45.0, and 41.7 (—RN—(CH₂CH₂)₂—NR—), 43.9 (R₂N—(C═O)—CH₂—CH₂—NHR), 38.1 (—NH—CH₂—CH₂—CH₂—), 30.3 (—NH—CH₂—CH₂—CH₂—), 27.7 (—(C═O)—O—C(CH₃)₃), 20.3 (—NH—CH₂—CH₂—CH₂—) ppm. ³¹P{H}NMR (162 MHz, D₂O): δ 18.4 and 18.0 ppm. MS (ES+ve) m/z (abundance %) for C18H40ClN3O14P2: calculated [M+2Na−H]⁺619.9222, found [M+2Na−H]⁺665.0854 (15).

### Product 4 (4-(2-Chloro-N-(3-(4-(2-chloroacetyl)piperazin-1-yl)-3-oxopropyl)acetamido)-1-hydroxybutane-1,1-diyl)bis(phosphonic acid)

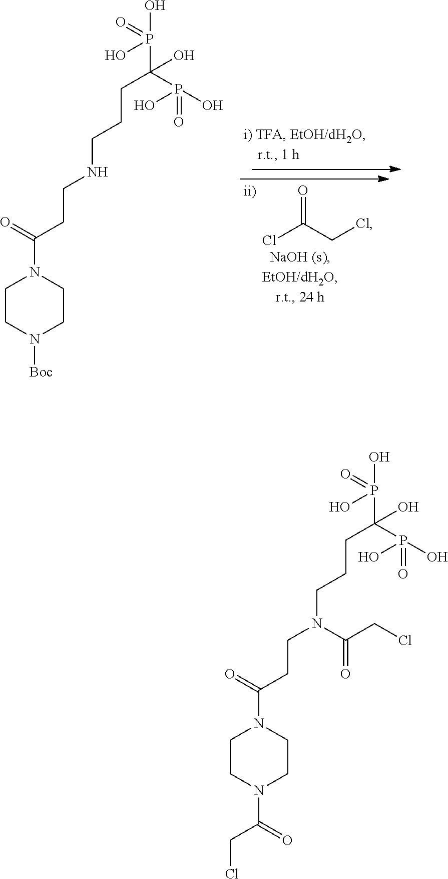 [SMILES: CC(C)(C)OC(=O)N1CCN(C(=O)CCNCCCC(O)(P(=O)(O)O)P(=O)(O)O)CC1 ; O=C(Cl)CCl ; O=C(CCl)N(CCCC(O)(P(=O)(O)O)P(=O)(O)O)CCC(=O)N1CCN(C(=O)CCl)CC1]

Product 2′ (4 g, 8.17 mmol 1 eq.) was dissolved in a solution containing 60% dH₂0 and 40% ethanol (21.5 mL, 0.38 M to ‘Product 2’). Trifluoroacetic acid (TFA) was then added dropwise at 30% of the volume of ‘Product 2’ at 0° C. using an ice bath, and the mixture was stirred at room temperature for 1 hour. After completion of the reaction, the reaction solution was removed under reduced pressure. Subsequently, any remaining TFA in the reaction vessel was completely removed using a rotary evaporator with two cycles of ethanol and dichloromethane (DCM) injections. The resulting residue was dissolved in a solution containing 33% dH₂0 and 67% EA (1 M to the ‘resulting residue’). A finely ground powder of sodium hydroxide (0.98 g, 24.5 mmol, 3 eq.) was then added, and the mixture was stirred at room temperature for 15 minutes. Following this, chloroacetyl chloride (1.56 mL, 19.61 mmol, 2.4 eq.) was added dropwise to the reaction solution, which was stirred at room temperature for 24 hours. The reaction solution was completely removed under reduced pressure to give the product (4.16 g, 94%), and no additional separations were conducted for the next reactions. White solid. Due to the hydrophobic alkyl chains and bisphosphonate group, the compound exhibits very low solubility in both aqueous and organic solvents. FT-IR (ATR mode): 1678 [v(R₂N—(C═O)—)], 1634 [v(R₂N—(C═O)—CH₂—C1)], 1183 and 1132 [v(—(P═O)—)], 719 [v(—CH₂—C1)], 521 and 446 [v(—(P—O)-)]cm 1. ¹H NMR (400 MHz, D₂O): δ 4.35 (s, 4H, 2×R₂N—(C═O)—CH₂—C1), 4.04-3.59 (m, 8H, —RN—(CH₂CH₂)₂—NR—), 3.55-3.28 (m, 2H, R₂N—(C═O)—CH₂—CH₂—NHR), 3.25-3.13 (m, 2H, —NH—CH₂—CH₂—CH₂—), 2.31-2.00 (m, 6H, R₂N—(C═O)—CH₂—CH₂—NHR and —NH—CH₂—CH₂—CH₂—) ppm. 1 ¹³C{H}NMR (101 MHz, D₂O): δ 168.2 (R₂N—(C═O)—CH₂—CH₂—NHR), 163.2 and 162.92 (2×R₂N—(C═O)—CH₂—C1), 77.2 (—C(OH)—(HPO₃)₂), 50.0, 45.1, and 41.5 (—RN—(CH₂CH₂)₂—NR—), 42.2, 42.1 (2×R₂N—(C═O)—CH₂—C1 and R₂N—(C═O)—CH₂—CH₂—NHR), 41.2 (R₂N—(C═O)—CH₂—CH₂—NHR), 40.1 (—NH—CH₂—CH₂—CH₂—), 30.6 (—NH—CH₂—CH₂—CH₂—), 20.7 (—NH—CH₂—CH₂—CH₂—) ppm. ³¹P{H}NMR (162 MHz, D₂O): δ 18.8 and 18.2 ppm. MS (ES+ve) m/z (abundance %) for C15H27C12N3010P2: calculated [M+2MeCN+H]⁺625.3539, found [M+2MeCN+H]+625.5791 (20).

### Product 5 (tert-Butyl 4-(4-((2,5-dioxopyrrolidin-1-yl)oxy)-4-oxobutanoyl)piperazine-1-carboxylate)

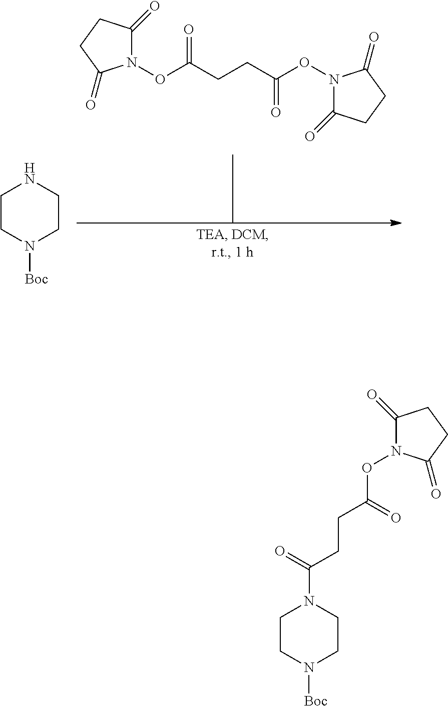 [SMILES: O=C(CCC(=O)ON1C(=O)CCC1=O)ON1C(=O)CCC1=O ; CC(C)(C)OC(=O)N1CCNCC1 ; CC(C)(C)OC(=O)N1CCN(C(=O)CCC(=O)ON2C(=O)CCC2=O)CC1]

1-Boc-piperazine (2.14 g, 11.53 mmol, 1.2 eq.) was fully dissolved in anhydrous DCM (160.2 mL, 0.06 M to ‘bis(2,5-dioxopyrrolidin-1-yl) succinate), followed by the gradual addition of triethylamine (4.02 mL, 28.83 mmol, 3 eq.). Bis(2,5-dioxopyrrolidin-1-yl) succinate (3 g, 9.61 mmol, 1 eq.) was then added dropwise at room temperature, and the mixture was stirred for 1 hour. After completion of the reaction, DCM was completely removed under reduced pressure to afford the product. No additional separations were conducted for the subsequent reactions, which were carried out continuously due to the product's rapid chemical reactivity in the presence of moisture. Brown solid. FT-IR (ATR mode): 1706 [v(—(C═O)—)], 1475 and 1395 [v(—O—NR₂)], 1212 and 1173 [v(—C—O-)]cm⁻¹.

### Product 6 (4-(4-(4-(tert-Butoxycarbonyl)piperazin-1-yl)-4-oxobutanamido)-1-hydroxybutane-1,1-diyl)bis(phosphonic acid)

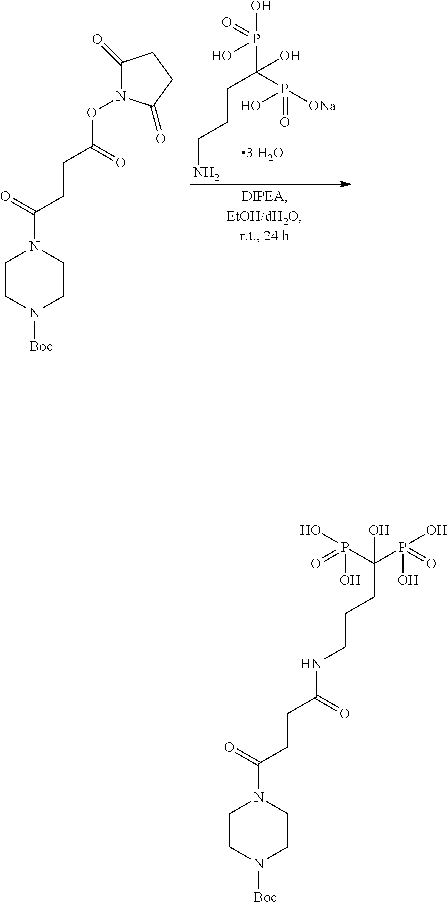

‘Product 5’ (2.83 g, 7.38 mmol, 1.2 eq.) was dissolved in ethanol (68.3 mL, 0.09 M to Alendronate) at room temperature, followed by the addition of DIPEA (3 eq.). In another vial, Alendronate (2 g, 6.15 mmol, 1 eq.) was dissolved in dH₂O (61.5 mL, 0.1 M to Alendronate) at room temperature, and the resulting reaction mixture was added dropwise to the round-bottom flask containing ‘Product 5’, followed by stirring at room temperature for 24 hours. The reaction solution was then completely removed under reduced pressure. Subsequently, acetone was added to induce precipitation of ‘Product 6’ (3.44 g, 90%), and no additional separations were conducted for the next reactions. Pale-beige solid. Due to the hydrophobic alkyl chains and bisphosphonate group, the compound exhibits very low solubility in both aqueous and organic solvents. FT-IR (ATR mode): 2978 [v(—NH—)], 1735 and 1670 [v(—NR₂—(C═O)—)], 1211 [v(—(P═O)—)], 1201 and 1157 [v(—C—O—)], 541 and 441 [v(—(P—O)-)]cm⁻¹. ¹H NMR (400 MHz, D₂O): δ 3.90-3.18 (m, 12H, R₂N—(C═O)—CH₂—CH₂—(C═O)—NR₂ and —RN—(CH₂CH₂)₂—NR—), 3.13-3.00 (m, 2H, —NH—CH₂—CH₂—CH₂—), 2.12-1.91 (m, 4H, —NH—CH₂—CH₂—CH₂—), 1.49 (s, 9H, —(C═O)—O—C(CH₃)₃) ppm. 13C{H}NMR (101 MHz, D₂O): δ 180.8 and 179.6 (R₂N—(C═O)—CH₂—CH₂—(C═O)—NR₂), 155.8 (—(C═O)—O—C(CH₃)₃), 82.7 (—(C═O)—O—C(CH₃)₃), 73.6 (—C(OH)—(HPO3)₂, 54.43 (R₂N—(C═O)—CH₂—CH₂—(C═O)—NR₂), 52.2, 43. 0, and 42.6 (—RN—(CH₂CH₂)₂—NR—), 40.0 (NH—CH₂—CH₂—CH₂—), 30.2 (—NH—CH₂—CH₂—CH₂—), 27.5 (—(C═O)—O—C(CH₃)₃), 22.4 (—NH—CH₂—CH₂—CH₂—) ppm. 31p{H}NMR (162 MHz, D₂O): δ 17.9 ppm. MS (ES+ve) m/z (abundance %) for C17H33N3011P2: calculated [M+Na]⁺540.3948, found [M+Na]⁺540.6430 (30).

### Product 7 (4-(4-(4-(2-Chloroacetyl)piperazin-1-yl)-4-oxobutanamido)-1-hydroxybutane-1,1-diyl)bis(phosphonic acid)

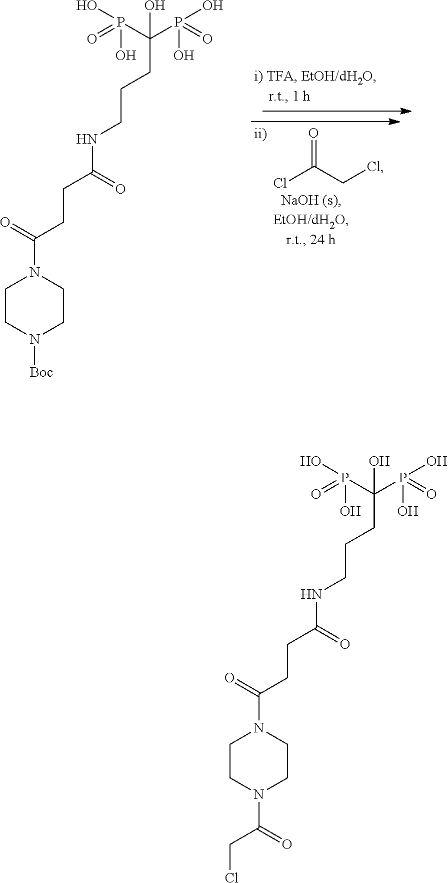 [SMILES: CC(C)(C)OC(=O)N1CCN(C(=O)CCC(=O)NCCCC(O)(P(=O)(O)O)P(=O)(O)O)CC1 ; O=C(Cl)CCl ; O=C(CCC(=O)N1CCN(C(=O)CCl)CC1)NCCCC(O)(P(=O)(O)O)P(=O)(O)O]

‘Product 6’ (3 g, 5.80 mmol, 1 eq.) was dissolved in a solution of 60% dH₂0 and 40% ethanol (15.3 mL, 0.38 M to ‘Product 6’). Subsequently, TFA was added dropwise to the solution at 30% of the volume of ‘Product 6’ at 0° C. using an ice bath, followed by stirring at room temperature for 1 hour. After the reaction was complete, the reaction solution was completely removed under reduced pressure. Ethanol or DCM was then added to the reaction vessel twice each, and the remaining TFA in the reaction vessel was completely removed using a rotavapor. The resulting residue was dissolved in a solution of 33% dH₂O and 67% EA (1 M to the ‘resulting residue’). A finely ground powder of sodium hydroxide (0.70 g, 17.4 mmol, 3 eq.) was then added, and the mixture was stirred at room temperature for 15 minutes. Subsequently, chloroacetyl chloride (0.94 mL, 11.6 mmol, 1.2 eq.) was added dropwise to the reaction mixture, and this was stirred at room temperature for 24 hours. The reaction solution was then completely removed under reduced pressure to give the product (2.63 g, 92%), and no additional separations were conducted for the next reactions. White solid. Due to the hydrophobic alkyl chains and bisphosphonate group, the compound exhibits very low solubility in both aqueous and organic solvents. FT-IR (ATR mode): 1680 and 1631 [v(R₂N—(C═O)—)], 1561 [v(R₂N—(C═O)—CH₂—C1)], 1155 and 1101 [v(—(P=O)—)], 890 [v(—CH₂—Cl)], 542 and 447 [v(—(P—O)-)]cm 1. ¹H NMR (600 MHz, D₂O): δ 3.97 (s, 2H, R₂N—(C═O)—CH₂—Cl), 4.05-3.03 (m, 14H, R₂N—(C═O)—CH₂—CH₂—(C═O)—NR₂, —RN—(CH₂CH₂)₂—NR—, and —NH—CH₂—CH₂—CH₂—), 2.20-2.02 (m, 4H, —NH—CH₂—CH₂—CH₂—) ppm. ¹³C{H}NMR (101 MHz, D₂O): δ 175.1 and 174.2 (R₂N—(C═O)—CH₂—CH₂—(C═O)—NR₂), 162.8 (R₂N—(C═O)—CH₂—Cl), 73.6 (—C(OH)—(HPO₃)₂), 52.2, 44.0, and 40.2 (—RN—(CH₂CH₂)₂—NR—), 40.1 (—NH—CH₂—CH₂—CH₂—), 30.9 (—NH—CH₂—CH₂—CH₂—), 22.3 (—NH—CH₂—CH₂—CH₂—) ppm. ³¹P{H}NMR (162 MHz, D₂O): δ 18.5 and 18.2 ppm. MS (ES+ve) m/z (abundance %) for C₁₄H₂₆ClN₃O₁₀P₂: calculated [M+MeCN+H]⁺535.8306, found [M+MeCN+H]⁺536.1517 (25).

### Synthetic Protocol for the Bone-Targeting Ionizable Lipid ‘Type 1’

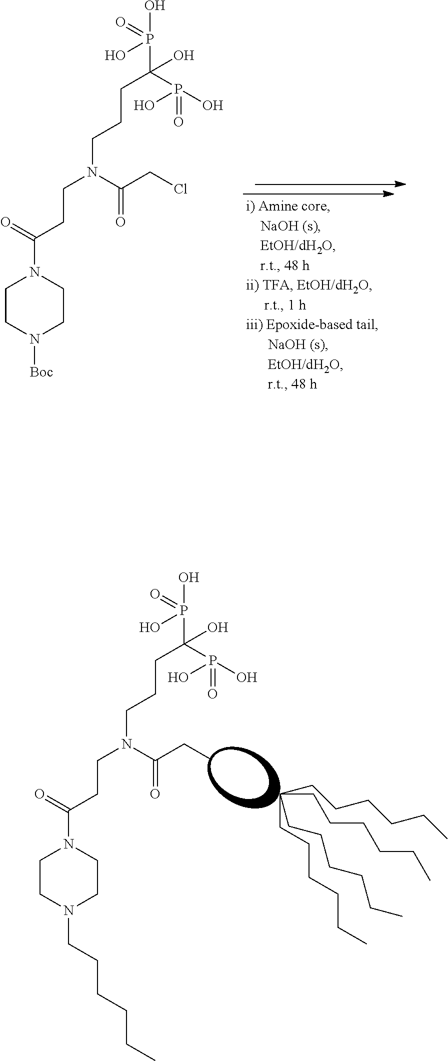

A finely ground powder of sodium hydroxide (3 eq.) was added to a solution containing 60% dH₂0 and 40% ethanol (0.12 M to ‘Product 3’). After adding the amine (1.1 eq.) to the solution, vigorous stirring was carried out at room temperature for 30 minutes. ‘Product 3’ (1 eq.) was then added dropwise to the reaction mixture at room temperature, and stirring was continued for 48 hours. Upon completion of the reaction, the reaction solution was completely removed, and acetone was added to wash out any unreacted amine through a filtration method. The obtained product was dissolved in a solution consisting of 60% dH₂0 and 40% ethanol (0.12 M to ‘Product 3’). Subsequently, the solution was cooled to 0° C. on an ice bath, and TFA was added dropwise until the pH reached 2-3. This was stirred for an additional hour at room temperature. After the reaction was complete, ethanol or DCM was added to the reaction vessel twice each, and the remaining TFA in the reaction vessel was completely removed using a rotavapor. The precipitated residue was dissolved in a solution containing 60% dH₂0 and 40% ethanol (0.12 M to ‘Product 3’), and a finely ground powder of sodium hydroxide was added and stirred at room temperature for 30 minutes to raise the pH of the reaction mixture to above 12. The ‘epoxide-based tail’ (1.2 eq. to one primary or secondary amine) was then added dropwise to the reaction mixture at room temperature, and the reaction was allowed to proceed at 80° C. for 2 days. After completion of the reaction, the reaction solution was completely removed under reduced pressure. Subsequently, acetone was added to induce precipitation of the product, and the solution was decanted to remove the liquid. n-Hexane was then added to the remaining mixture to remove unreacted epoxide-based tails. Afterward, acetone was added again to induce precipitation of the product, and the product was filtered to obtain pure product.

### Synthetic Protocol for the Bone-Targeting Ionizable Lipid ‘Type 2’

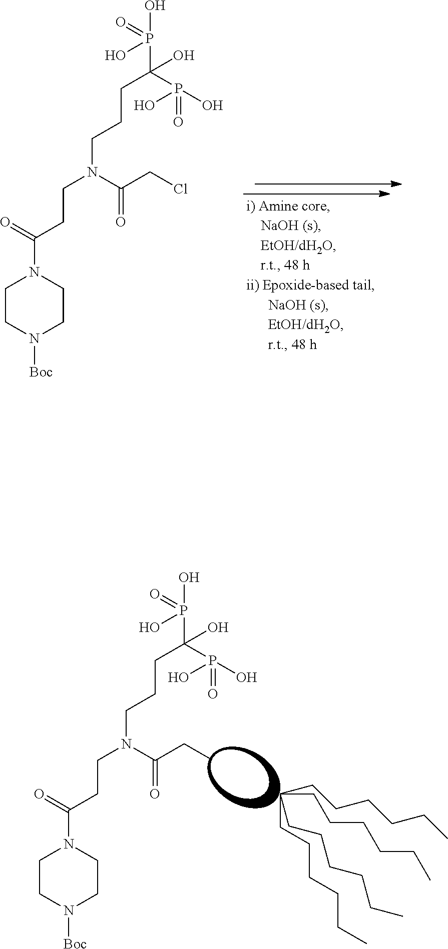

A finely ground powder of sodium hydroxide (3 eq.) was added to a solution containing 60% dH₂O and 40% ethanol (0.12 M to ‘Product 3’). After adding the amine (1.1 eq.) to the solution, vigorous stirring was carried out at room temperature for 30 minutes. ‘Product 3’ (1 eq.) was then added dropwise to the reaction mixture at room temperature, and stirring was continued for 48 hours. Upon completion of the reaction, the reaction solution was completely removed, and acetone was added to wash out any unreacted amine through a filtration method. Subsequently, the precipitated residue was dissolved in a solution containing 60% dH₂O and 40% ethanol (0.12 M to ‘Product 3’), and a finely ground powder of sodium hydroxide was added and stirred at room temperature for 30 minutes to raise the pH of the reaction mixture to 12. The ‘epoxide-based tail’ (1.2 eq. to one primary or secondary amine) was then added dropwise to the reaction mixture at room temperature, and the reaction was allowed to proceed at 80° C. for 2 days. After completion of the reaction, the reaction solution was completely removed under reduced pressure. Subsequently, acetone was added to induce precipitation of the product, and the solution was decanted to remove the liquid. n-Hexane was then added to the remaining mixture to remove unreacted epoxide-based tails. Afterward, acetone was added again to induce precipitation of the product, and the product was filtered to obtain pure product.

Synthetic protocol for the bone-targeting ionizable lipid ‘Type 3’

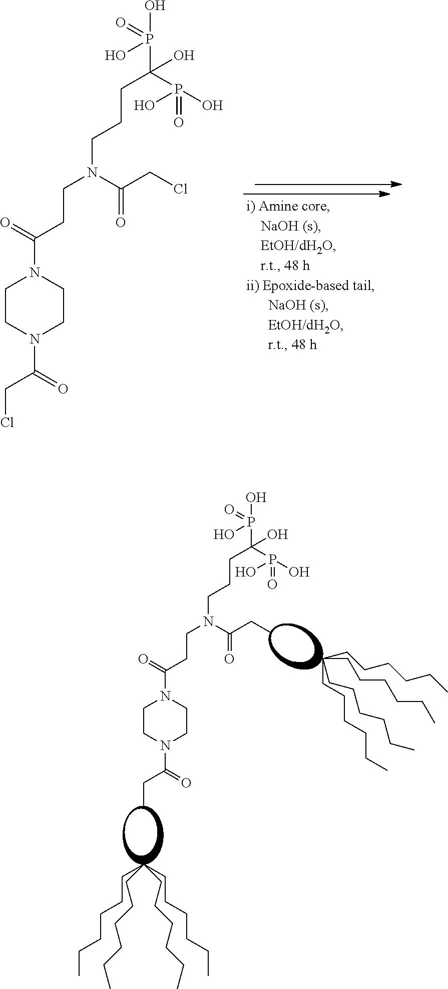

A finely ground powder of sodium hydroxide (3 eq.) was added to a solution containing 60% dH₂0 and 40% ethanol (0.12 M to ‘Product 4’). After adding the amine (1.1 eq.) to the solution, vigorous stirring was carried out at room temperature for 30 minutes. ‘Product 4’ (1 eq.) was then added dropwise to the reaction mixture at room temperature, and stirring was continued for 48 hours. The solution was then vigorously stirred at room temperature for 30 minutes after adding amine. It was stirred at room temperature for an additional 48 hours. Upon completion of the reaction, the reaction solution was completely removed, and acetone was added to wash out any unreacted amine through a filtration method. The precipitated residue was dissolved in a solution containing 60% dH₂0 and 40% ethanol (0.12 M to the ‘precipitated residue’), and a finely ground powder of sodium hydroxide was added and stirred at room temperature for 30 minutes to adjust the pH of the reaction mixture to above 12. Subsequently, ‘epoxide-based tail’ (1.2 eq. to one primary or secondary amine) was added dropwise at room temperature, and the reaction was allowed to proceed at 80° C. for 2 days. After completion of the reaction, the reaction solution was completely removed under reduced pressure. Subsequently, acetone was added to induce precipitation of the product, and the solution was decanted to remove the liquid. n-Hexane was then added to the remaining mixture to remove unreacted epoxide-based tails. Afterward, acetone was added again to induce precipitation of the product, and the product was filtered to obtain pure product.

Synthetic protocol for the bone-targeting ionizable lipid ‘Type 4’

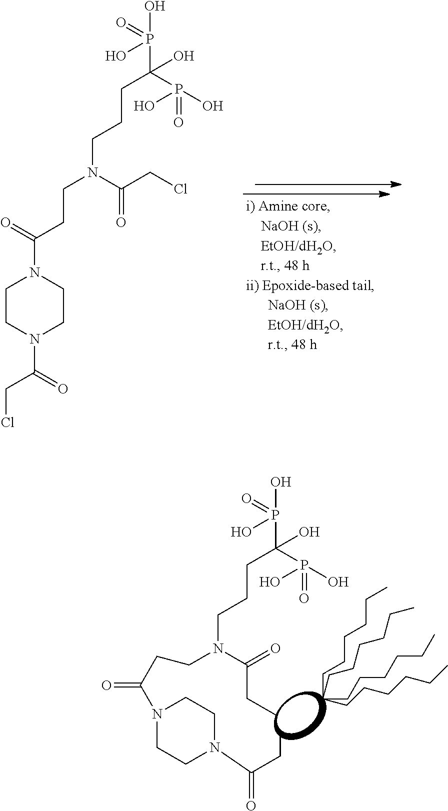

A finely ground powder of sodium hydroxide (3 eq.) was added to a solution containing 60% dH₂0 and 40% ethanol (0.12 M to ‘Product 4’). After adding the amine (1.1 eq.) to the solution, vigorous stirring was carried out at room temperature for 30 minutes. ‘Product 4’ (1 eq.) was then added dropwise to the reaction mixture at room temperature, and stirring was continued for 48 hours. The solution was then vigorously stirred at room temperature for 30 minutes after adding amine. It was stirred at room temperature for an additional 48 hours. Upon completion of the reaction, the reaction solution was completely removed, and acetone was added to wash out any unreacted amine through a filtration method. The precipitated residue was dissolved in a solution containing 60% dH₂0 and 40% ethanol (0.12 M to the ‘precipitated residue’), and a finely ground powder of sodium hydroxide was added and stirred at room temperature for 30 minutes to adjust the pH of the reaction mixture to above 12. Subsequently, ‘epoxide-based tail’ (1.2 eq. to one primary or secondary amine) was added dropwise at room temperature, and the reaction was allowed to proceed at 80° C. for 2 days. After completion of the reaction, the reaction solution was completely removed under reduced pressure. Subsequently, acetone was added to induce precipitation of the product, and the solution was decanted to remove the liquid. n-Hexane was then added to the remaining mixture to remove unreacted epoxide-based tails. Afterward, acetone was added again to induce precipitation of the product, and the product was filtered to obtain pure product.

Synthetic protocol for the bone-targeting ionizable lipid ‘Type 5’

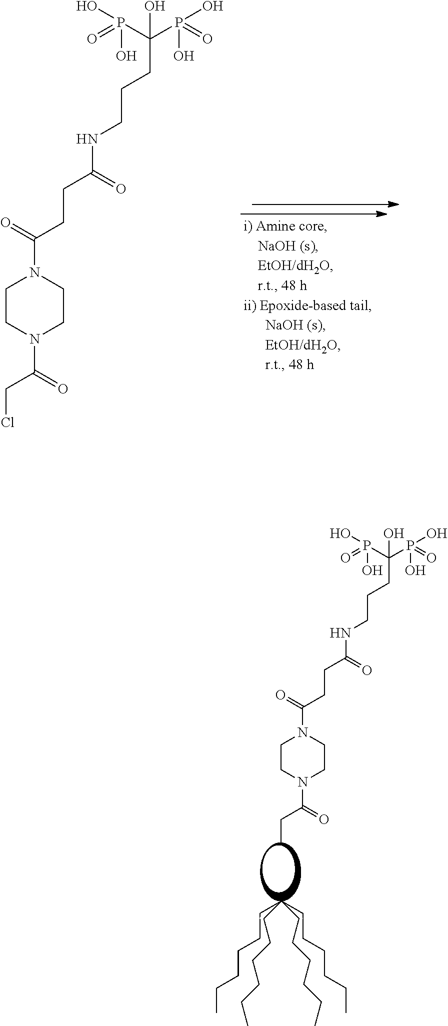

A finely ground powder of sodium hydroxide (3 eq.) was added to a solution containing 60% dH₂0 and 40% ethanol (0.12 M to ‘Product 7′). After adding the amine (1.1 eq.) to the solution, vigorous stirring was carried out at room temperature for 30 minutes. ‘Product 7′ (1 eq.) was then added dropwise to the reaction mixture at room temperature, and stirring was continued for 48 hours. Upon completion of the reaction, the reaction solution was completely removed, and acetone was used to wash out any unreacted amine through a filtration method. The precipitated residue was dissolved in a solution of 60% dH₂0 and 40% ethanol (0.12 M to the ‘precipitated residue’), followed by the addition of a finely ground sodium hydroxide powder and stirring at room temperature for 30 minutes to adjust the pH of the reaction mixture to above 12. Subsequently, the ‘epoxide-based tail’ (1.2 eq. to one primary or secondary amine) was added dropwise at room temperature, and the reaction was allowed to proceed at 80° C. for 2 days. After completion of the reaction, the reaction solution was completely removed under reduced pressure. Subsequently, acetone was added to induce precipitation of the product, and the solution was decanted to remove the liquid. n-Hexane was then added to the remaining mixture to remove unreacted epoxide-based tails. Afterward, acetone was added again to induce precipitation of the product, and the product was filtered to obtain pure product.

Structural characterization of ‘Type1-P1-C12’

White hygroscopic solid. The purity and yield of the compound exceeded 95%. Due to its very low solubility in both aqueous and organic solvents, the structure of the compound was elucidated using two different NMR solvents D₂0 and DMSO-d6. FT-IR (ATR mode): 3410 and 3316 [v(—OH)], 2954, 2915, and 2848 [v(—CH₂— and —CH₃)], 1676 [v(R₂N—(C═O)—)], 1465 [v(—CH₂— and CH₃)], 1335 [v(—OH)], 1144 [v(R₂HC—O—)], 1097 and 1070 [v(—(P=O)—)], 592 and 519 [v(—(P—O)-)]cm⁻¹. ¹H NMR (600 MHz, DMSO-d6): δ 4.62-4.34 (m, 10H, —OH), 1.43-1.33 and 1.29-1.16 (m, 90H, —CH₂-(epoxide-based alkyl chains)), 0.85 (t, 24H, —CH₃, J=6.9 Hz) ppm. ¹H NMR (600 MHz, D₂O): δ 4.00-2.50 (m, 51H, —CH₂-(main structural framework)), 1.98 (m, 4H, —NH—CH₂—CH₂—CH₂—), 1.76-0.94 (m, 90H, —CH₂-(epoxide-based alkyl chains)), 0.86-0.69 (m, 15H, —CH₃) ppm. ¹³C{H}NMR (151 MHz, DMSO-d6): δ 175.5, 166.8, and 158.1 (—(C═O)—), 75.0 (—C(OH)—(HPO₃)₂), 71.2, 68.9, 66.1, and 65.8 (R₂C(OH)—), 56.2, 51.7, and 46.2 (—CH₂—(C═O)—N(CH₂CH₂)₂N—(C═O)—, —(CH₂)₂NCH₂CH₂N(CH₂—)CH₂CH₂—N(CH₂CH₂)₂NCH₂CH₂N(CH₂)₂—, and —CH₂N(—(C═O)—)CH₂CH₂CH₂C(OH)—(HPO₃)₂), 39.5 (—NH—CH₂—CH₂—CH₂—), 33.9, 33.5, 31.4, 29.4, 29.3, 29.2, 29.1, 29.0, 28.9, 28.8, 25.3, 25.2, and 25.1 (—CH₂-(epoxide-based alkyl chains)), 31.4 (—NH—CH₂—CH₂—CH₂—), 22.2 (—NH—CH₂—CH₂—CH₂—), 15.2 (—CH₂—C(OH)—(HPO₃)₂), 14.1 (—CH₃) ppm. MS (MALDI+ve) m/z (abundance %) for C83H170N8O14P2: calculated [M+4-MeCN+2H]⁺866.2195, found [M+4-MeCN+2H]⁺866.9426 (100).

Structural characterization of ‘Type3-PI—C12’

White hygroscopic solid. The purity and yield of the compound exceeded 95%. Due to its very low solubility in both aqueous and organic solvents, the structure of the compound was elucidated using two different NMR solvents D₂0 and DMSO-d6. FT-IR (ATR mode): 3407 and 3319 [v(—OH)], 2954, 2915, and 2848 [v(—CH₂— and —CH₃)], 1679 [v(R₂N—(C═O)—)], 1464 [v(—CH₂— and CH₃)], 1336 [v(—OH)], 1143 [v(R₂HC—O—)], 1099 and 1070 [v(—(P=O)—)], 592 and 517 [v(—(P—O)-)]cm⁻¹. ¹H NMR (600 MHz, DMSO-d6): δ 4.50-4.28 (m, 13H, —OH), 1.44-1.14 (m, 144H, —CH₂-(epoxide-based alkyl chains)), 0.85 (t, 24H, —CH₃, J=7.0 Hz) ppm. ¹H NMR (600 MHz, D₂O): δ 4.00-2.50 (m, 82H, —CH₂-(main structural framework)), 2.03 (m, 4H, —NH—CH₂—CH₂—CH₂—), 1.62-1.06 (m, 144H, —CH₂-(epoxide-based alkyl chains)), 0.90-0.76 (m, 24H, —CH₃) ppm. ¹³C{H}NMR (151 MHz, DMSO-d6): δ 175.9, 166.8, and 158.2 (—(C═O)—), 75.0 (—C(OH)—(HPO₃)₂), 71.1, 68.9, 66.0, and 65.7 (R₂C(OH)—), 56.1, 51.6, and 46.1 (—CH₂—(C═O)—N(CH₂CH₂)₂N—(C═O)—, —(CH₂)₂NCH₂CH₂N(CH₂—)CH₂CH₂—N(CH₂CH₂)₂NCH₂CH₂N(CH₂)₂—, and —CH₂N(—(C═O)—)CH₂CH₂CH₂C(OH)—(HPO₃)₂), 39.5 (—NH—CH₂—CH₂—CH₂—), 33.8, 33.7, 33.4, 31.9, 31.3, 29.3, 29.2, 29.1, 29.0, 28.9, 28.8, 25.6, 25.2, and 25.1 (—CH₂-(epoxide-based alkyl chains)), 31.3 (—NH—CH₂—CH₂—CH₂—), 22.1 (—NH—CH₂—CH₂—CH₂—), 15.2 (—CH₂—C(OH)—(HPO₃)₂), 14.0 (—CH₃) ppm. MS (MALDI+ve) m/z (abundance %) for C131H267N13O18P2: calculated [M+H₂O+3-Na]+820.5102, found [M+H₂O+3-Na]⁺820.8733 (100).

### Example 8: The rationale design, synthesis, and initial in vitro screening of bone-targeting ionizable lipids

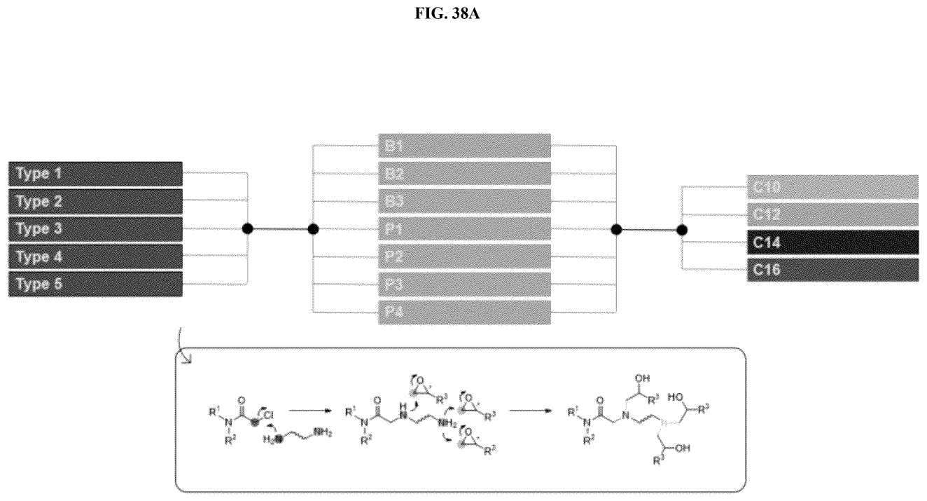

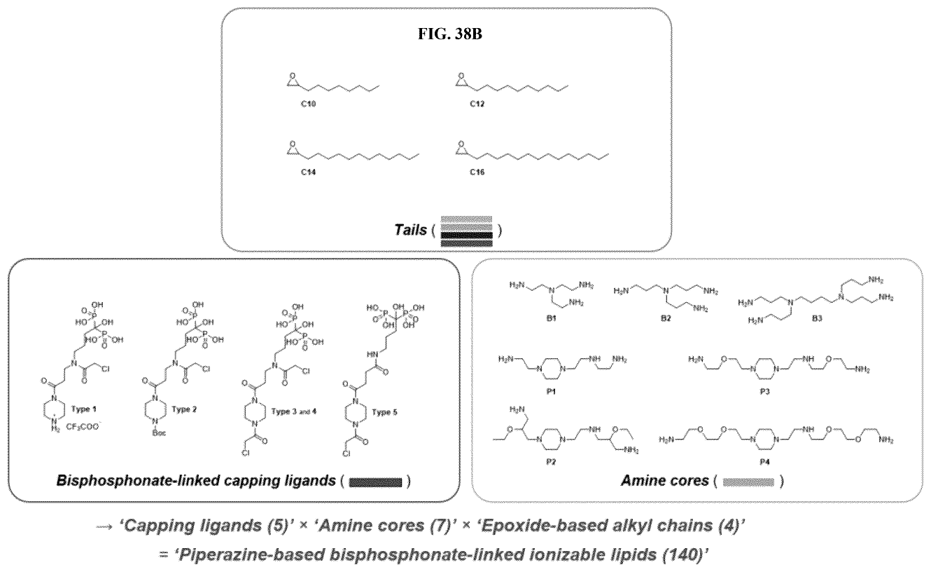

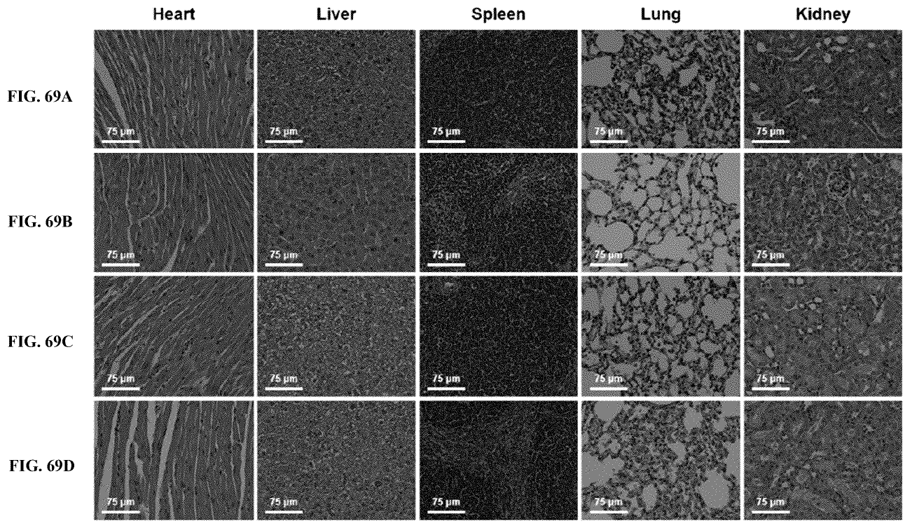

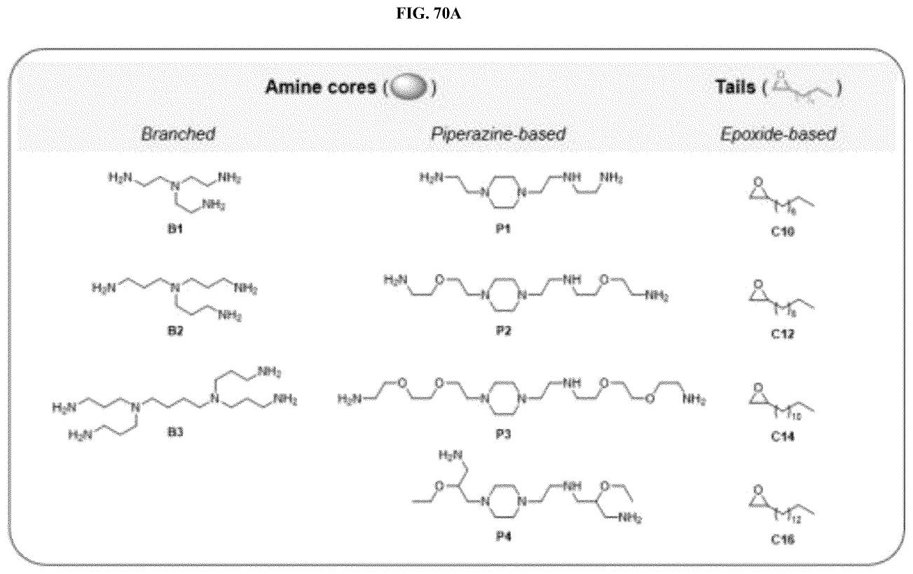

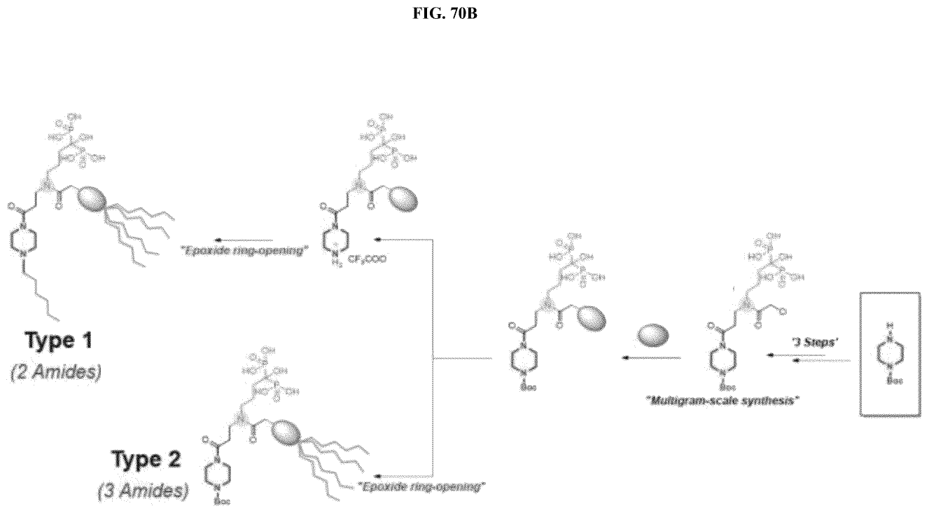

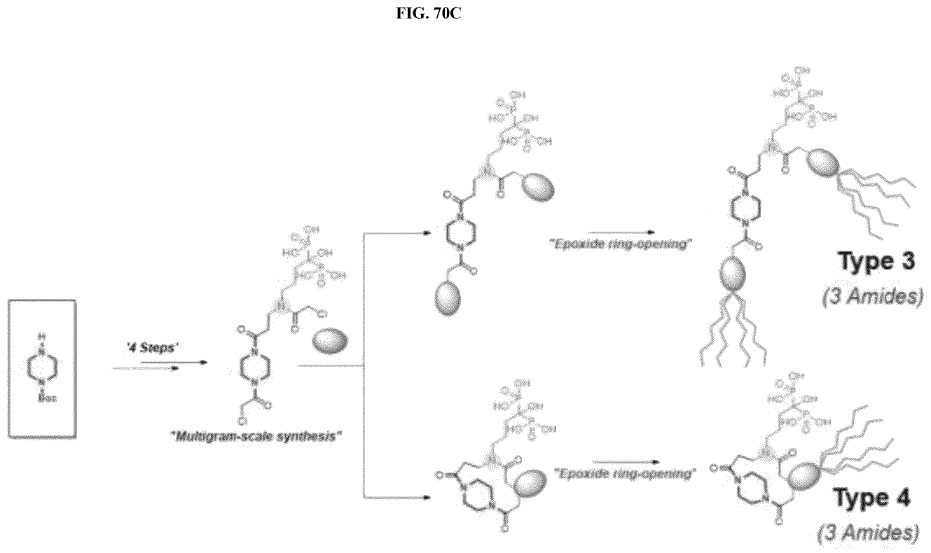

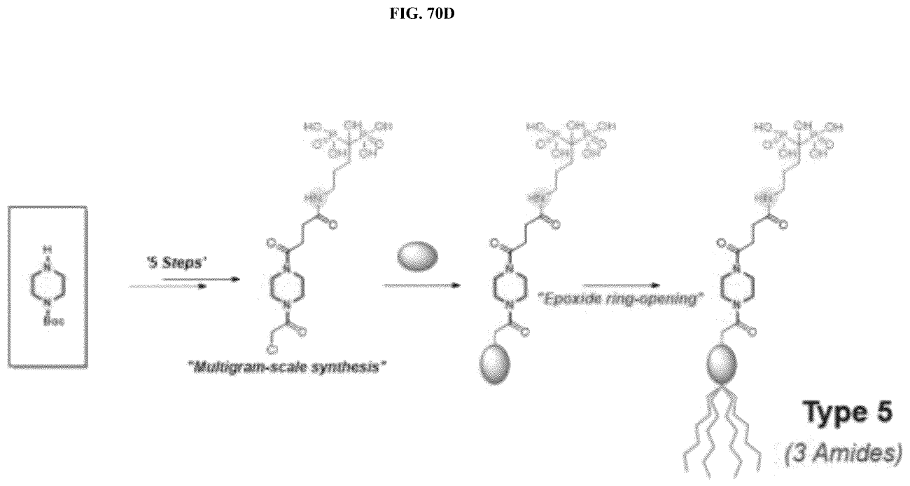

As depicted in FIGS. 38A-38B, the synthesis of PIP-based BP-linked ionizable lipids is divided into three main components. The first crucial part involves bisphosphonate-linked capping ligands based on piperazine. These ligands are chemically designed to enable the generation of new PIP-based BP-linked ionizable lipids through chemical bonding with various forms of amines. Precursors used for the synthesis of PIP-based BP-linked ionizable lipids from Type 1 to Type 5 were synthesized on a multi-gram scale via this route. The other components, amine cores, and epoxide-based tails, are commercially available, enabling the design of a synthetic pathway for the synthesis of novel forms of bone-targeting ionizable lipids through chemical bonding with bisphosphonate-linked capping ligands (FIGS. 70A-70D). Notably, in the chemical synthesis, commercially available bisphosphonates such as Alendronate™, which are only soluble in water, were successfully synthesized using co-solvent systems, allowing for the successful production of target molecules. Furthermore, most of the synthesized compounds were purified without using column-based physical purification methods. Instead, compound separation was achieved through extraction and recrystallization methods utilizing differences in solubility, leveraging the physicochemical properties of the materials. The overall yield of the final products following this synthetic route(s) and purification procedure is at a minimum of 60%, with a purity of at least 90%. Therefore, the designed synthetic pathways allow for immediate industrial application of the chemical processes. Currently, a total of 140 new PIP-BP ionizable lipids have been synthesized using this synthetic route.

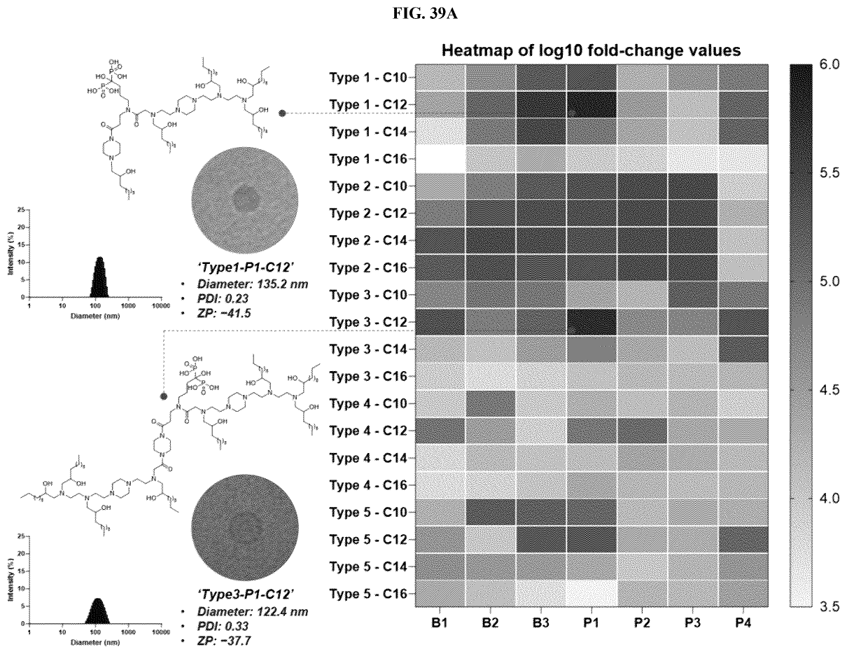

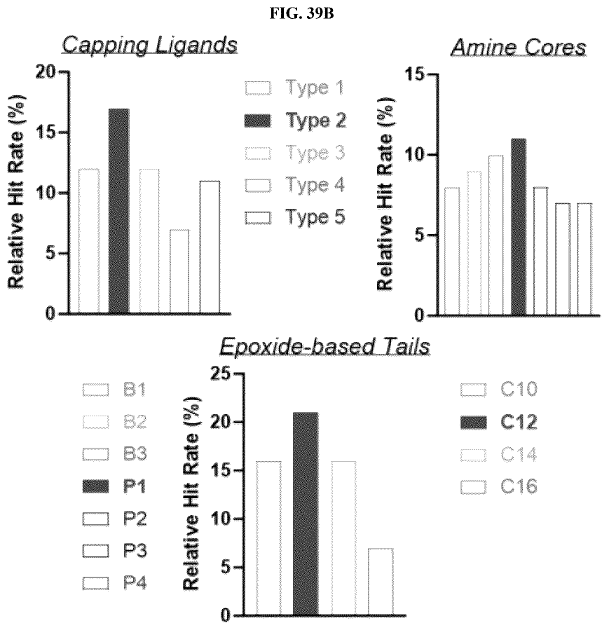

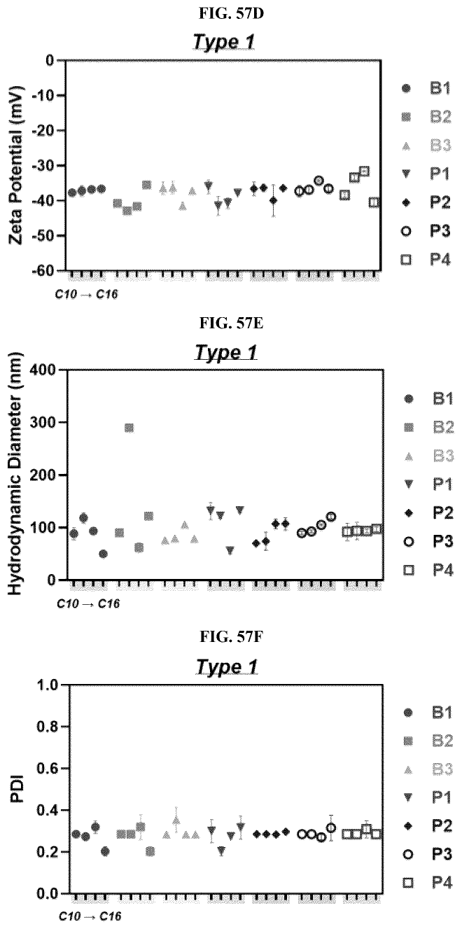

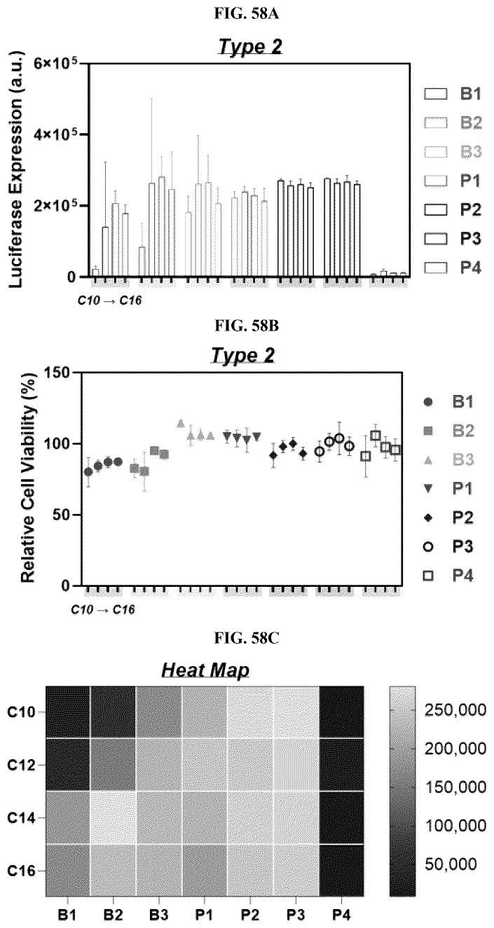

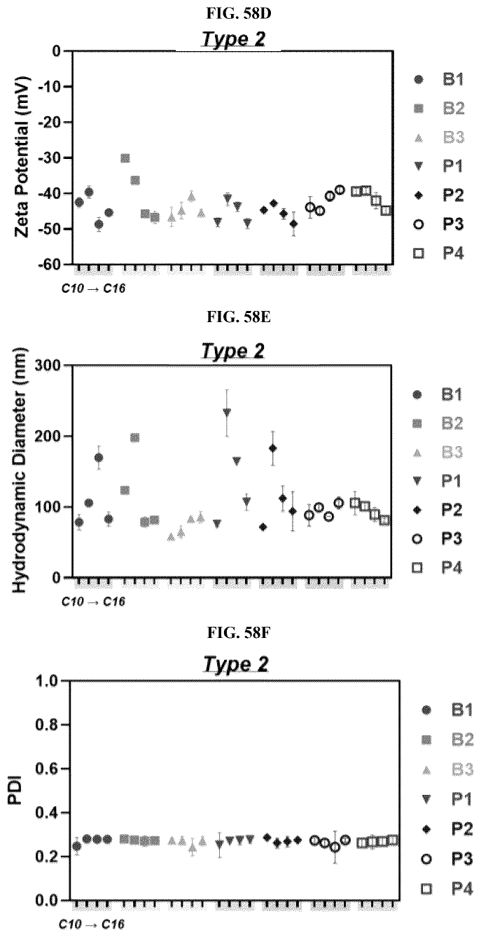

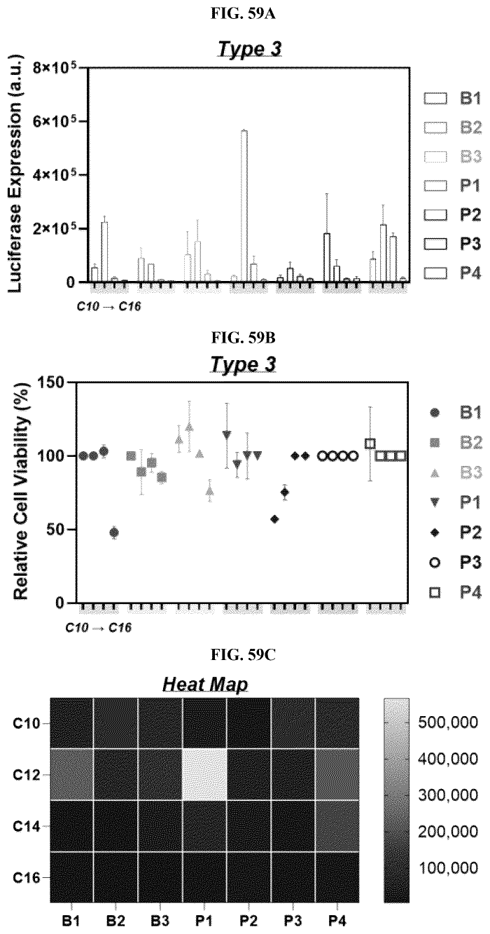

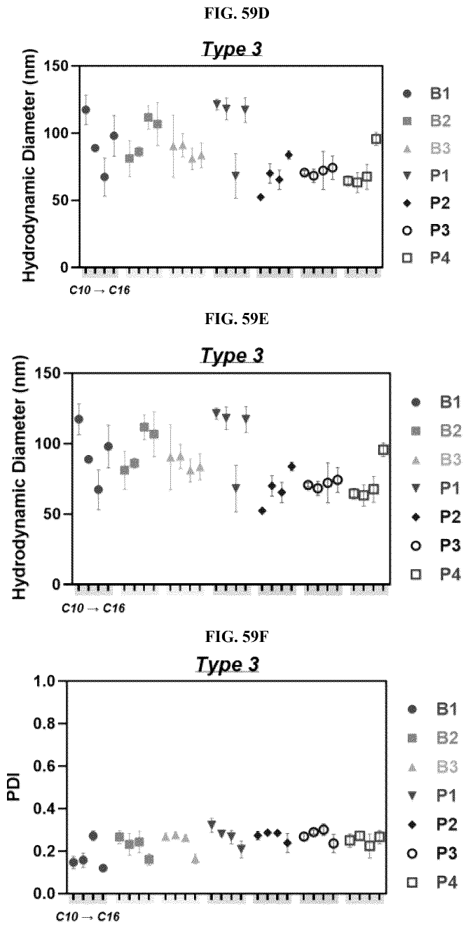

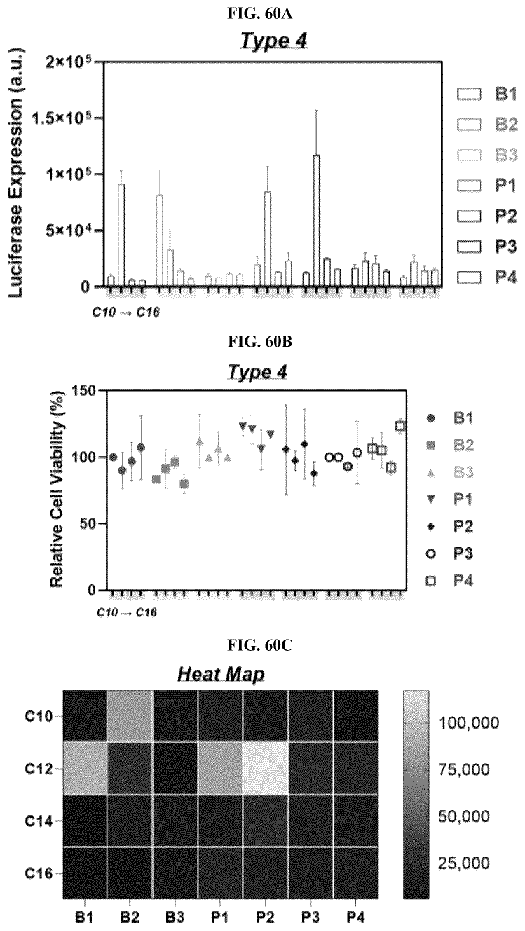

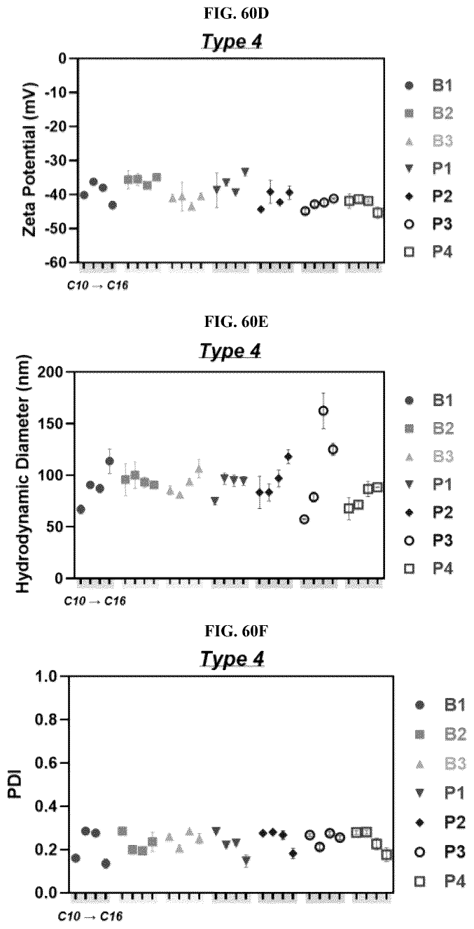

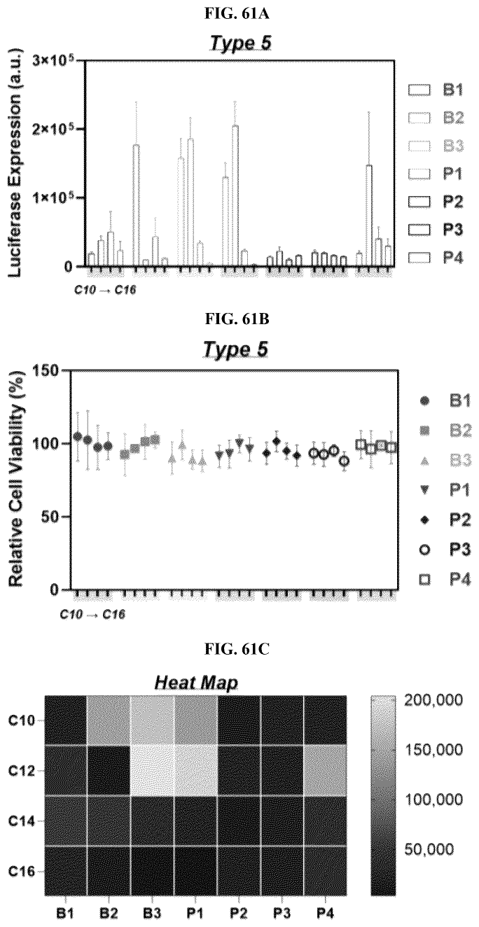

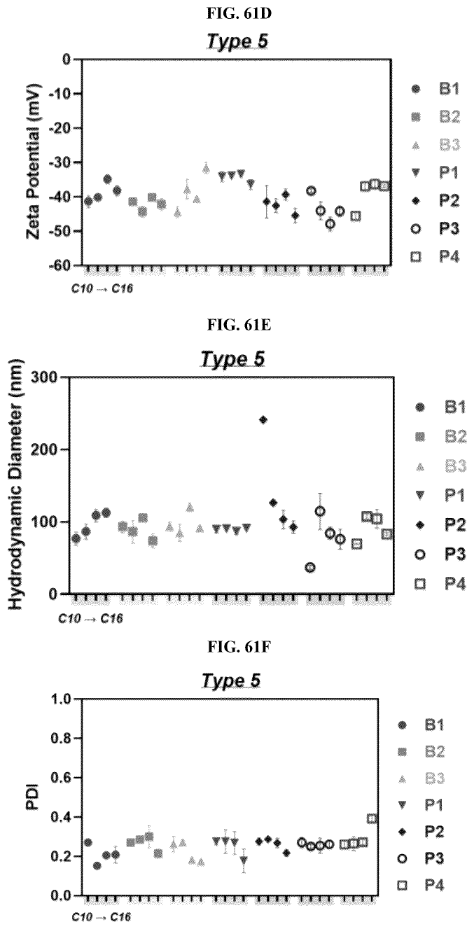

The PIP-BP LNPs were prepared using newly synthesized bone-targeting ionizable lipids at specific lipid formulation parameters (Ionizable lipid/DOPE/Cholesterol/C14-PEG2000=35/16/46.5/2.5%, molar ratios). The encapsulation efficiency of mRNA in these LNPs and the concentration of mRNA were measured using the R^{1b}oGreen assay. Screening both in vitro and in vivo utilized firefly luciferase (FLuc) mRNA substituted with 5-methoxy-U. To evaluate the structure-activity relationship of the PIP-BP lipid library for mRNA delivery, LNPs containing FLuc mRNA were formulated and employed for transfection of Hep-G2 cells. The results of the initial screening of these PIP-BP LNPs in the Hep-G2 cell line are presented in FIGS. 39A-39B, FIGS. 57A-57F, FIGS. 58A-58F, FIGS. 59A-59F, FIGS. 60A-60F, and FIGS. 61A-61F. Luciferase expression values were normalized to the untreated group, and a concentration of 10 ng/5000 cells per well was applied in this in vitro experiment using a 96-well microplate. A heatmap was generated to evaluate the influence of various PIP-BP structural parameters on in vitro mRNA delivery by calculating a relative hit rate. Among the PIP-BP LNPs generated with the fixed molar ratios, LNPs based on ‘Type1-P1-C12’ and ‘Type3-P1-C12’ ionizable lipids exhibited the highest efficiencies (FIGS. 39A-39B). A brief structure-activity reveals that overall, LNPs with Type 2 capping ligands bearing P1 amine cores and a 12-carbon epoxide-based alkyl chain showed superior transfection in the Hep-G2 cell line (FIGS. 39C-39E). This could be attributed to the simple chemical structure of capping ligands similar to Type 2, excluding the 1-acyl-4-boc piperazine structure, which allows direct binding of Alendronate™ to the amine cores, providing favorable conditions for interactions among adjacent lipids and lysosomal-endosomal escape in the Hep-G2 cell line. From the perspective of amine cores, the analysis suggests that the packing among adjacent lipids facilitated by amines contributes to favorable transfection, analogous to the analysis of the relationship between chemical structure and luciferase expression through capping ligands.

Comparing amine cores between the B series and P1, it is evident that transfection occurs more efficiently when a rigid backbone like piperazine is present. Additionally, comparing amine cores within the P series, the simpler structure of the piperazine-based core P1 appears to favor transfection. Lastly, considering the relationship between tail length and transfection, it can be inferred that shorter tails are advantageous for transfection when the molecular size of the capping ligand and amine core is large, facilitating interactions among adjacent lipids. However, C12 is preferred over C10 because transfection seems to be determined by the shape, number, and size of the amine core. Furthermore, except for Type 3 capping ligands, which bind to one amine, the molecular size of the ionizable lipid center is relatively small in other Types, indicating that epoxide-based alkyl chains with slightly longer lengths, such as 12 carbons, are generally advantageous for transfection.

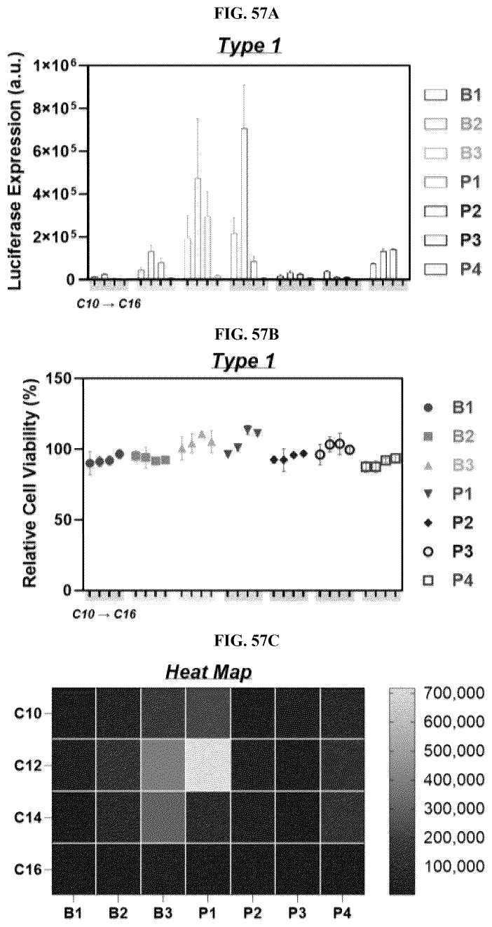

To gain a fundamental understanding of the relationship between chemical structure and luciferase expression, theoretical interpretations of interactions among adjacent lipids leading to packing and morphological analyses are essential. Both low toxicity in cells and high luciferase expression were observed (FIGS. 57A-57F, FIGS. 58A-58F, FIGS. 59A-59F, FIGS. 60A-60F, FIGS. 61A-61F, and FIGS. 62-64).

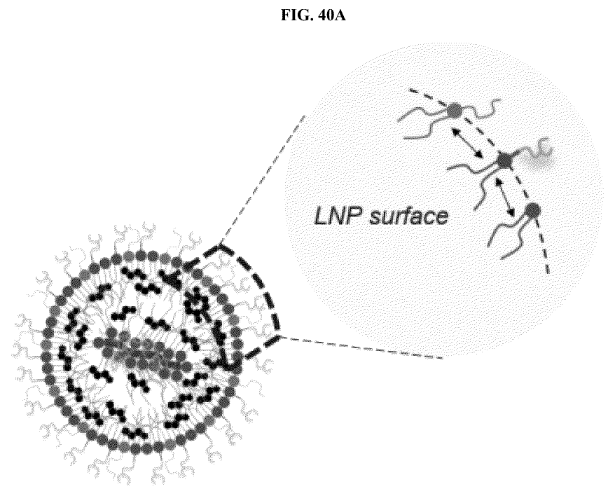

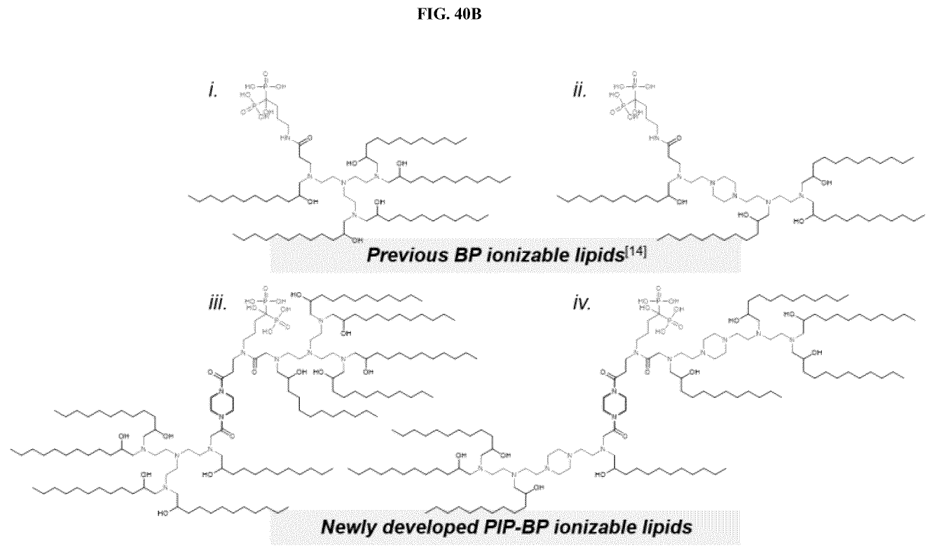

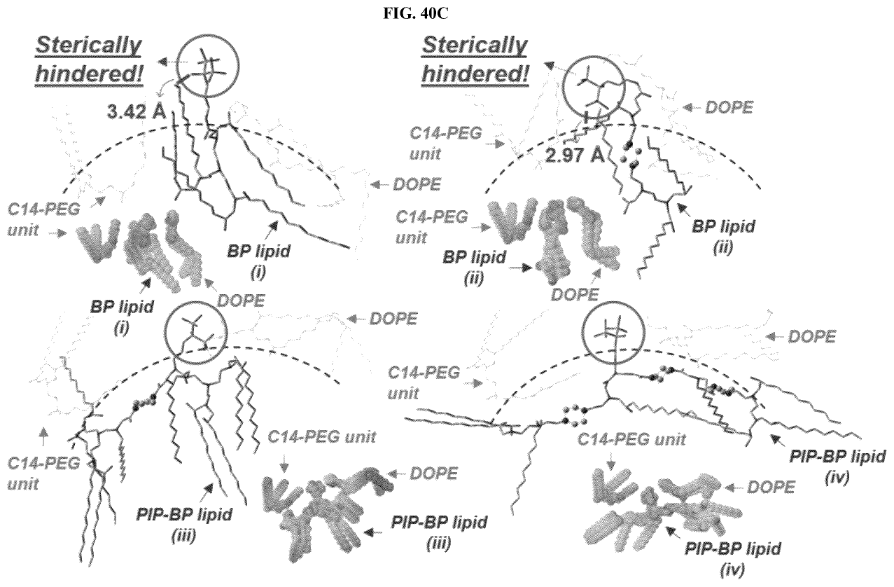

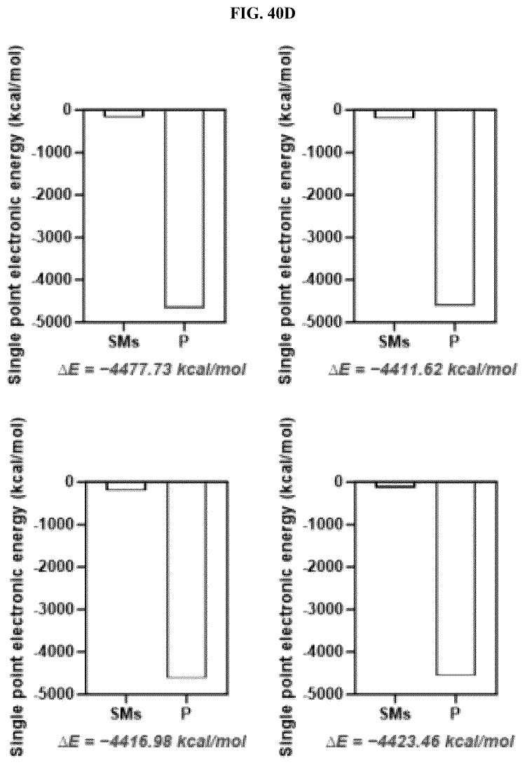

In order to further analyze this from a chemical structural perspective, simulations were conducted using a molecular dynamic program commonly employed in computational chemistry. The aim was to discuss the distinctiveness of the newly developed PIP-BP LNPs compared to conventional ones. As illustrated in FIG. 40A and FIG. 40C, the focus was primarily on understanding the interactions between lipids that led to the formation of LNPs.

The calculated molecular structures showed that the thermodynamic energy was more stable (approximately -4,400 kcal/mol) when lipids interacted with each other rather than existing individually (FIG. 40D). This indicates a tendency for the lipids to aggregate. Additionally, the electrostatic potential map revealed that the amine groups exhibited electron- deficient regions (blue-colored region), facilitating easy binding with mRNA. Conversely, the negatively charged bisphosphonate groups (red-colored circles) posed challenges for mRNA binding (FIG. 40C).

Through chemical structural interpretation based on the computed molecular structures, it was clearly discerned that the newly developed PIP-based BP-linked ionizable lipids were more exposed on the LNPs' surface. This suggests that they could interact more actively with the bone microenvironment ex vivo and/or in vivo (FIG. 40C). In contrast, the chemical structure of conventional bone-targeting ionizable lipids exhibited lower rigidity within LNPs (FIG. 40C), making them more susceptible to being shielded by surrounding lipids or experiencing interference.

Upon examining the computational results, it appears that increasing the rigidity of the overall chemical structure when developing targeting lipids could facilitate the smooth functioning of terminal group functions, typically found in active sites, leading to favorable outcomes in both in vitro and in vivo experiments. Experimentally, it was also observed that the surface charges of these bone-targeted LNPs are all around −30 mV, indicating that the bisphosphonate group, Alendronate™, remains exposed while maintaining the rigidity of the ionizable lipid on the surface of LNPs (FIGS. 57A-57F, FIGS. 58A-58F, FIGS. 59A-59F, FIGS. 60A-60F, and FIGS. 61A-61F).

### Example 9: Efficient Formulation of PIP-Based BP-Linked LNPs

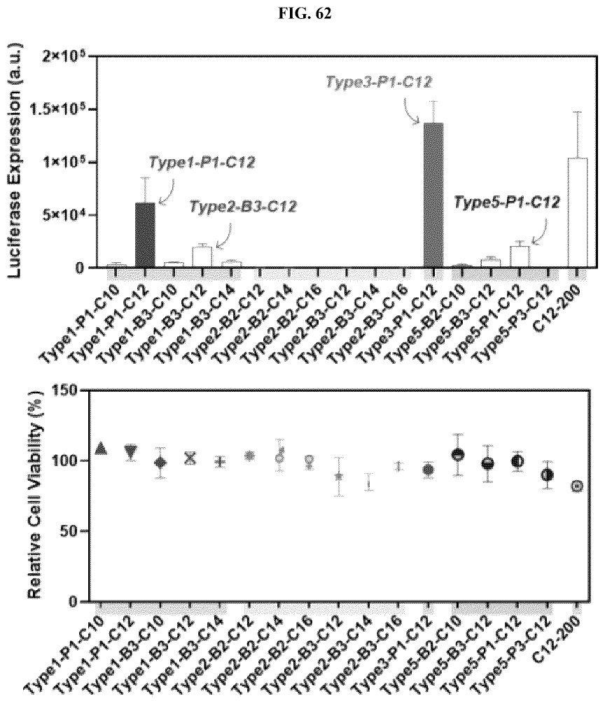

The in vitro experiments were conducted on the BJ cell line, a fibroblast cell type, for the 16 top-performing PIP-BP LNPs selected from the initial screening (FIG. 62). The rationale behind this choice was that most compounds containing hydroxyl groups tend to bind well to liver proteins' active sites, hence the need for the primary screening of these PIP-BP LNPs using a primary cell line of mesenchymal cells. As an overall result, among the different types of PIP-BP ionizable lipids, Type 1 with alkyl chains at the piperazine terminal group designed to disrupt the LNP membrane, and Type 3 with multiple amine groups, were found to be most advantageous for luciferase expression in BJ cells.

Furthermore, this approach provided insights into mRNA transfection based on differences in cell morphology and LNP morphology. For instance, while the polygonal-shaped Hep-G2 cell line exhibited the highest transfection efficiency with ‘Type1-P1-C12 (M.W. 1580.2575 g/mol)’, the spindle-shaped fibroblast BJ cell line showed higher luciferase expression with the relatively larger ‘Type3-P1-C12 (M.W. 2374.5460 g/mol)’. LNPs appear to induce higher interactions with cells having similar shapes.

An important consideration in the manufacturing of these PIP-BP LNPs is the solubility of bisphosphonates in solvents or solutions, as briefly mentioned earlier. Bisphosphonates typically dissolve only in water. Furthermore, when combining bisphosphonates with hydrophobic organic compounds, they exhibit a solubility profile very different from conventional ones. Therefore, the newly synthesized PIP-based BP-linked ionizable lipids did not dissolve in organic solvents but only dissolved in very small quantities in highly polar solvents such as ethanol (FIG. 51). In order to address this solubility issue, a concept based on pH-dependent functional groups was employed. A small amount of diluted 1 N hydrochloric acid (HCl) was added to protonate the hydroxyl group in the bisphosphonate, thereby increasing its solubility in ethanol solution. This led to an average improvement of approximately 20% in encapsulation efficiency, maintaining an overall average encapsulation efficiency of around 60%. Another solution to this solubility issue is to use the newly developed PIP-based BP-linked ionizable lipids in small quantities. Subsequently, a majority of ionizable lipids with relatively smaller molecular sizes, such as ‘C12-200’ ionizable lipid, which shares the chemical structure of piperazine commonly used in research, can be used for more than half of the bone-targeting ionizable lipids to manufacture bone-targeted LNPs. The latter approach can reduce the overall size and/or PDI of PIP-BP LNPs, resulting in stable bone-targeted LNPs (FIGS. 41A-41B). In this study, both methods, using a small amount of 1 N HCl and employing ‘C12-200’ ionizable lipid, were appropriately utilized to manufacture PIP-BP LNPs via lipid formulation.

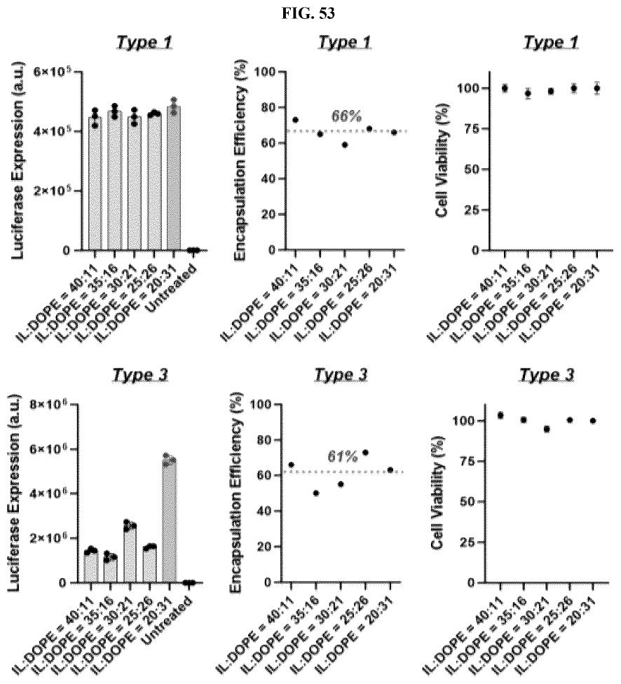

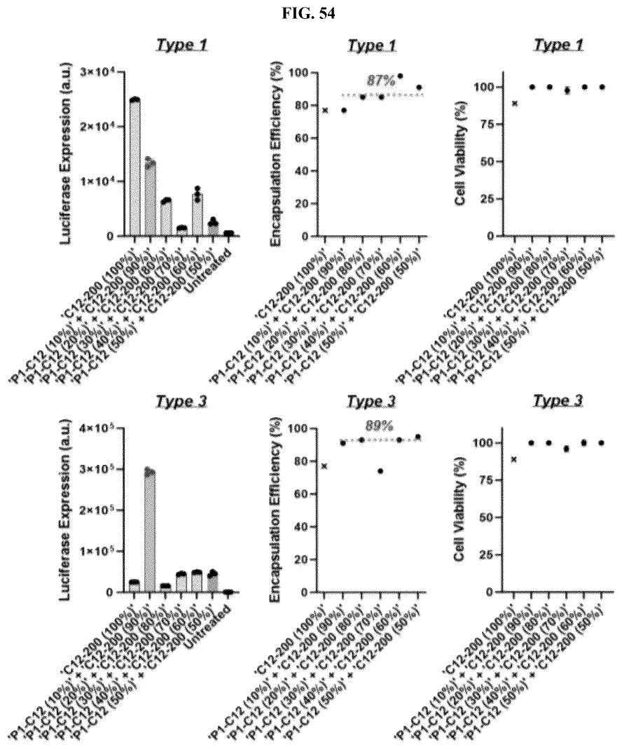

Next, PIP-BP LNPs were formulated using ‘C12-200’ ionizable lipid to reduce their size and improve their stability. The encapsulation efficiency of PIP-BP LNPs composed solely of PIP-based BP-linked ionizable lipids was approximately 60%, with some PDI deviation observed (FIG. 53). In contrast, when ‘C12-200’ ionizable lipid was used in conjunction with PIP-based BP-linked ionizable lipids, the encapsulation efficiency exceeded 85%, with lower PDI deviation (FIG. 54). This indicates that in BP-LNPs composed solely of PIP-based BP-linked ionizable lipids, the high steric tension among the lipids led to decreased stability of the LNPs, resulting in reduced mRNA encapsulation.

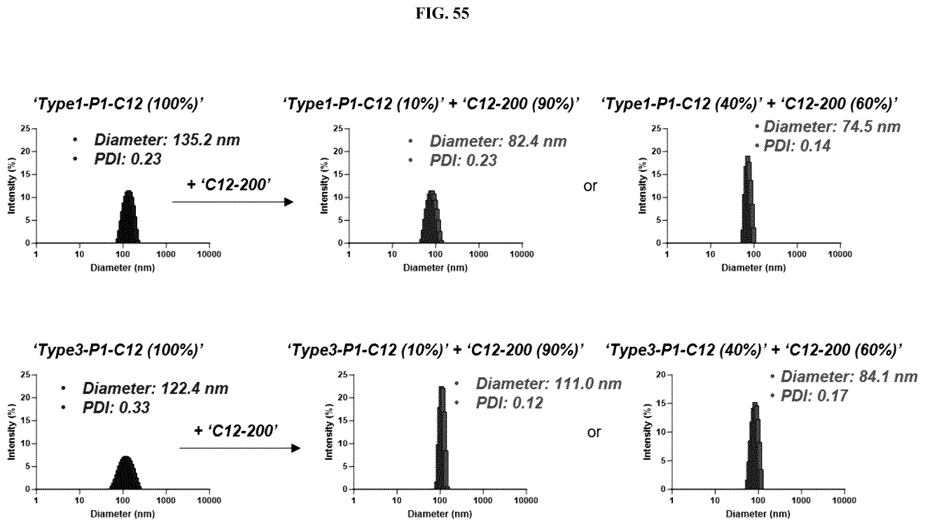

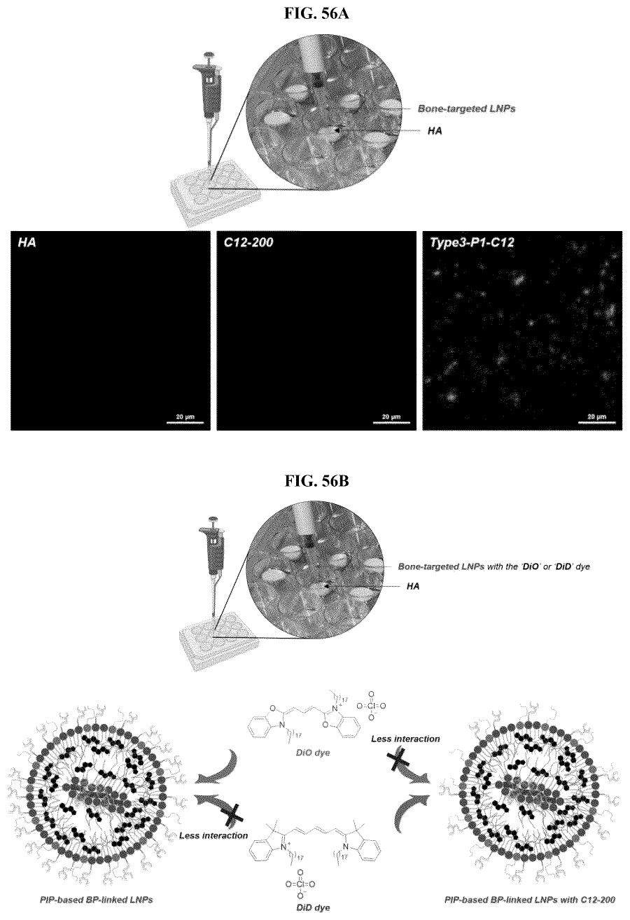

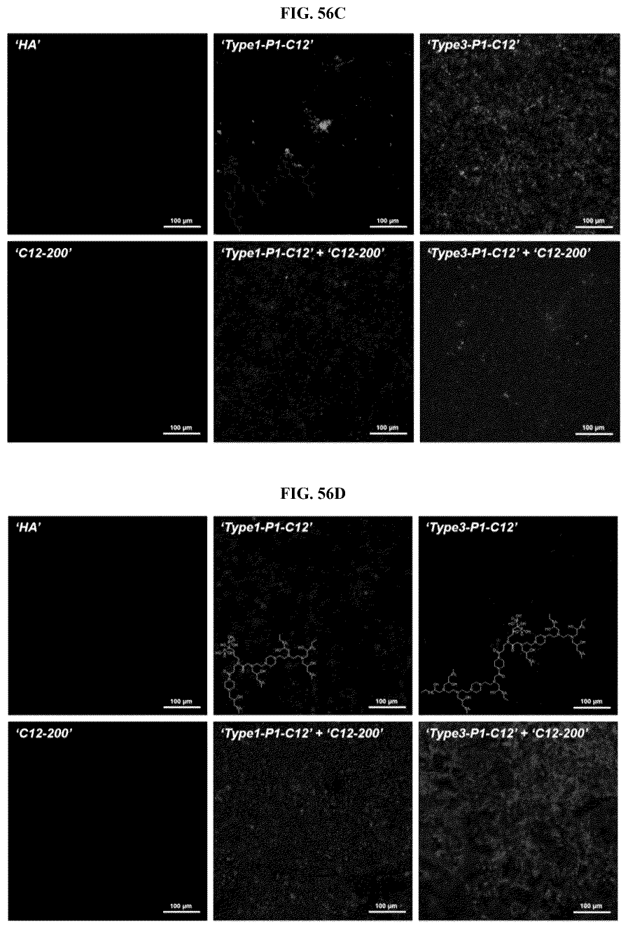

It is intriguing to note that even in vitro experiments showed improved luciferase expression results with fewer PIP-BP lipids used in LNP formulation, indicating the influence of steric tension among lipids on LNP stability (FIGS. 41A-41B). Additionally, when the relatively larger molecule ‘Type3-P1-C12’ was used in lower quantities, mRNA encapsulation decreased (FIGS. 41A-41B). This is because the ionizable lipid forms relatively more well-packed LNPs through low steric interactions with surrounding lipids. This influence of lipids' packing on the overall structure of LNPs was also evident from the hydrodynamic size distribution of these PIP-BP LNPs. The hydrodynamic size of PIP-BP LNPs mixed with ‘C12-200’ ionizable lipid, such as ‘Type1-P1-C12’ or ‘Type3-P1-C12’, became smaller or exhibited a more uniform polydisperse index distribution, indicating an understanding of the impact of steric interactions among lipids on LNPs morphology (FIG. 55). This concept, correlated with simulations in FIGS. 40A-40D and experiments in FIGS. 56A-56D, explains why piperazine-based bisphosphonate-linked ionizable lipids mixed with ‘C12-200’ ionizable lipids were adopted for these in vivo experiments.

### Example 10: Binding Study of PIP-Based BP-Linked LNPs to HA

As evident from FIGS. 40A-40D, Alendronate™ maintains a rigid structure on the surface of PIP-BP LNPs through piperazine. Next, the goal was to experimentally demonstrate the interaction between LNPs and HA visually through confocal microscopy and AFM to confirm whether bone-targeted LNPs effectively bind to the components of bone materials, such as HA (FIGS. 42A-42B, FIGS. 43A-43C, and FIGS. 56A-56D).

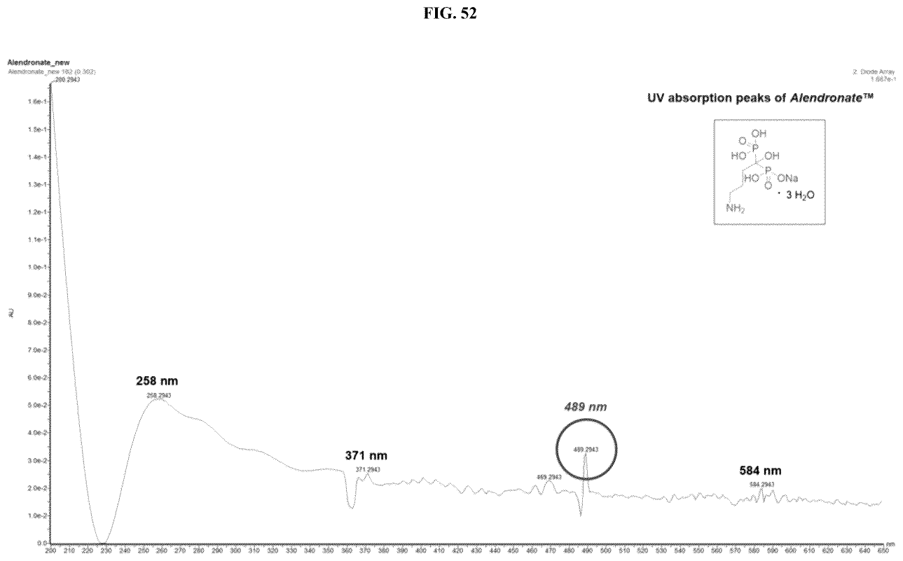

For this experimental validation, commercially available HA discs were utilized. Prior to use, the HA surfaces were activated by washing them with deionized water and sonication at least three times. Subsequently, the binding study between PIP-BP LNPs and HA was conducted. Afterward, the HA surfaces were coated with either negative control group LNPs based on ‘C12-200’ ionizable lipid or PIP-BP LNPs based on ‘Type3-P1-C12’ ionizable lipid. After 24 hours, the surfaces were thoroughly rinsed with deionized water approximately five times to remove any unbound LNPs. The HA surfaces were then imaged using a laser-scanning confocal microscope. The bisphosphonate, Alendronate™, used in the bone-targeted LNPs exhibited absorbance at 488 nm in the photodiode array (PDA) HPLC detector, as shown in FIG. 52. Similarly, absorption occurred at 488 nm in the laser scanning confocal microscope images (FIG. 56A). In contrast, the HA surfaces coated with LNPs based on ‘C12-200’ ionizable lipid, the negative control group, showed no absorbance after thorough washing with deionized water.

As depicted in FIG. 42A, to further elucidate this phenomenon, the surface of the HA disc was analyzed using AFM. The HA surface coated with LNPs based on ‘C12-200’ ionizable lipid exhibited a surface similar to that of the HA surface without any LNP deposition. However, the HA surface coated with PIP—BP LNPs, ‘Type3-P1-C12’ ionizable lipid, exhibited a different morphology. This difference was also evident in the roughness values. The roughness values for the HA surface without any PIP-BP LNP deposition and the HA surface coated with LNPs based on ‘C12-200’ ionizable lipid were 2.33 nm and 2.60 nm, respectively. In contrast, the HA surface coated with ‘Type3-P1-C12’ ionizable lipid exhibited a roughness value of 0.90 nm, indicating that the HA disc surface with rough features was enveloped by PIP-BP LNPs.

In order to investigate whether PIP-BP LNPs properly adsorb onto the HA surface, additional experiments were conducted. Firstly, to maintain consistent molar ratios among the LNP formulations, after manufacturing the LNPs, fluorescent dyes such as DiO or DiD were mixed to allow the dyes to bind to the LNPs through hydrophobic interactions. Therefore, DiO or DiD mixed with LNPs of five types (‘Type1-P1-C12’, ‘Type3-P1-C12’, ‘Type1-P1-C12’+‘C12-200’, ‘Type3-P1-C12’+‘C12-200’, and ‘C12-200’ ionizable lipid-based LNPs) were coated onto the HA surface. After 24 hours, similar to the previous experiment, the HA surface was thoroughly rinsed approximately five times with deionized water. Subsequently, the surface of HA was analyzed using laser scanning confocal microscopy. The results of this experiment also showed consistent findings as mentioned earlier (FIGS. 56A-56D). In other words, LNPs with PIP-BP ionizable lipids adsorbed onto the HA surface, whereas conventional LNPs based on ‘C12-200’ ionizable lipid struggled to adsorb onto the HA surface. Furthermore, some intriguing findings were discovered. As mentioned in FIGS. 41A-41B, ‘PIP-based BP-linked LNPs' with a loose structure exhibited better binding with the relatively small hindrance factor DiO dye. Conversely, ‘PIP-based BP-linked LNPs with C12-200’, which had a relatively compact structure, showed better binding with the larger-sized and hook-shaped DiD dye. These experimental results further indirectly indicated that ‘PIP-based BP-linked LNPs with C12-200’ tended to have a more compact LNP morphology.

Next, it was investigated whether PIP-BP LNPs bind to HA under in vitro experimental conditions (FIG. 42B). For this purpose, six types of PIP-BP LNPs were used, including five PIP-BP LNPs that showed top performance in the BJ cell line and one negative control group based on ‘C12-200’ ionizable lipid-based LNPs. Initially, chambers containing HA discs and 12-well plates with BJ cells were incubated in a CO₂ incubator for approximately 24 hours. Subsequently, the six types of PIP-BP LNPs were added to the chambers and left in the CO₂ incubator for another 24 hours. After 24 hours, the chambers containing the HA discs were removed, and the luciferase expression of the bone-targeted LNPs within the BJ cells attached to the bottom of the 12-well plates was measured using a microplate reader. Most of the ‘C12-200’ ionizable lipid-based LNPs penetrated the HA disc holes and membranes, resulting in cellular uptake by the attached BJ cells on the bottom and exhibiting strong luciferase expression. However, PIP-BP LNPs treated in wells containing HA discs resulted in low luciferase expression. Reduced luminescence indicated the PIP-BP LNPs remained bound to HA and the fraction of freely soluble PIP-BP LNPs was significantly compared to control ‘C12-200’ LNPs. Therefore, the binding of PIP-BP LNPs to HA controls the release and delivery of mRNA to cells. This result also suggests that control of local delivery may prolong transfection in vivo and prevent rapid clearance of LNPs. As mentioned in FIGS. 37A-37F, PIP-BP LNPs of different types have different shapes, so the degree of adhesion to the HA surface varies. However, all PIP-BP LNPs used in the experiment interacted strongly with the HA surface.

Furthermore, as previously mentioned, compared to ‘C12-200’ ionizable lipid-based LNPs, all PIP-BP LNPs showed high cell viability, indicating no cell toxicity issues. Through experiments conducted in cells, it was confirmed that low toxicity and highly efficient PIP-BP LNPs have been developed.

### Example 11: PIP-BP LNP mediated in vivo mRNA delivery to bone

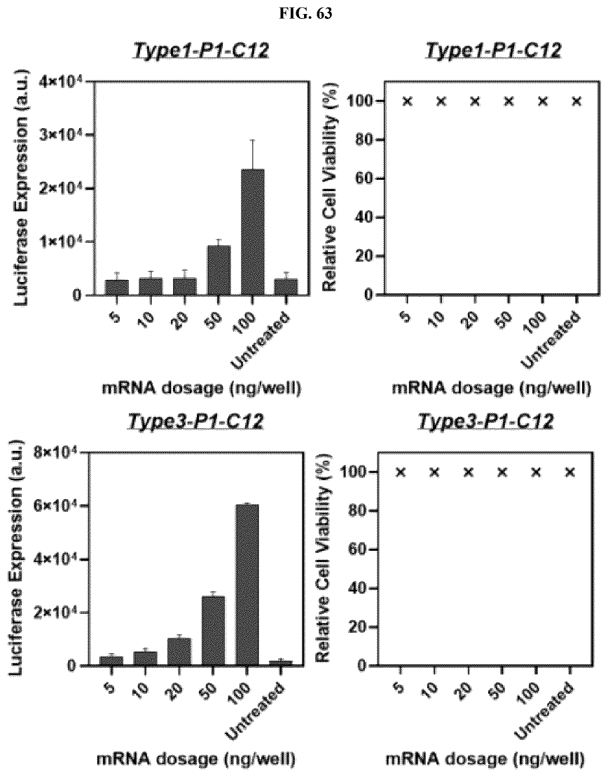

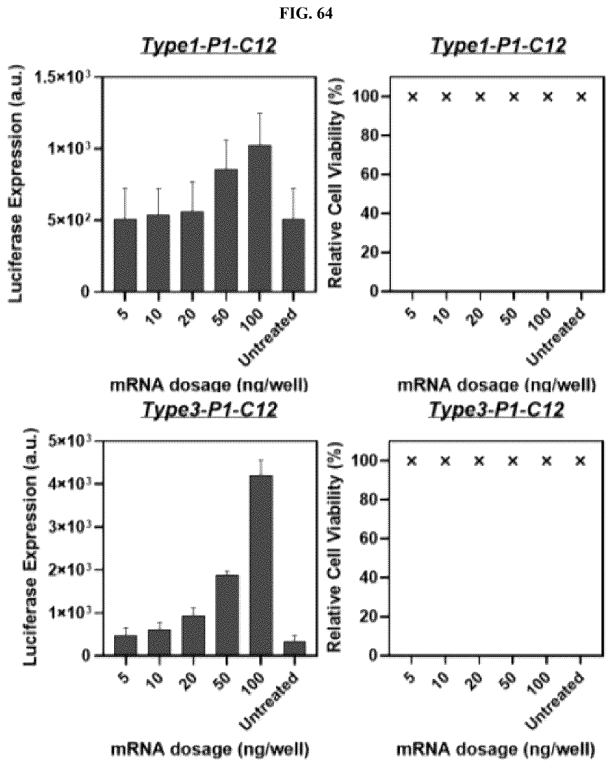

After demonstrating the in vitro efficacy of PIP-BP LNPs for bone targeting, it was further evaluated whether PIP-BP LNPs enhance mRNA delivery in vivo. Therefore, in vivo experiments were conducted using the ‘Type1-P1-C12’ and ‘Type3-P1-C12’ bone-targeting ionizable lipids, which showed top performance in the Hep-G2 or BJ cell lines. Prior to performing in vivo experiments, cell toxicity analysis and dose-dependent transfection studies were conducted for these two different PIP-BP ionizable lipids in vitro to investigate whether these two lipids could effectively deliver mRNA in in vivo environments. The results of these experiments, as shown in FIGS. 63-64, revealed no significant cell toxicity, and successful cell transfection was achieved even with increased mRNA dosage for the PIP-BP LNPs.

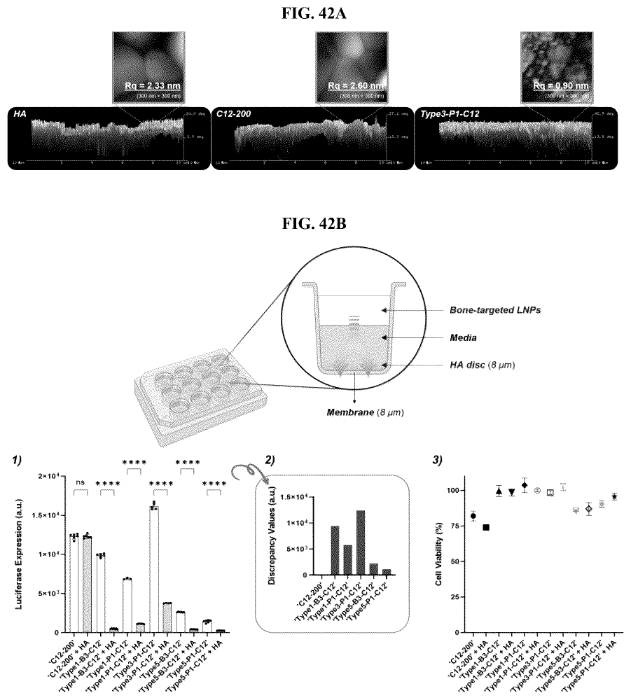

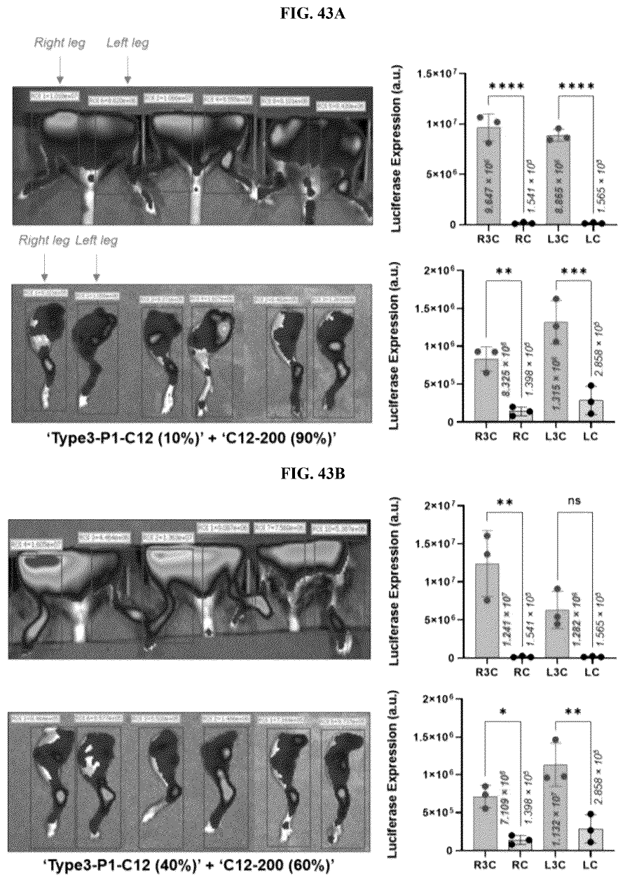

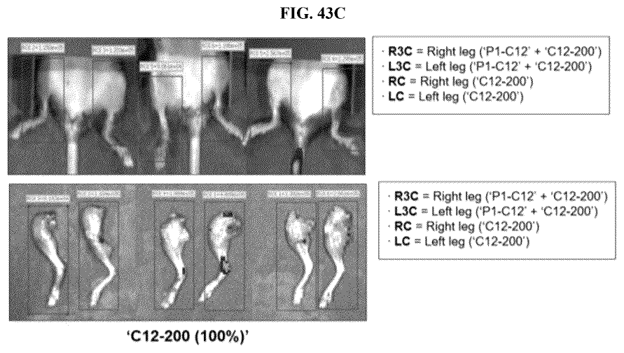

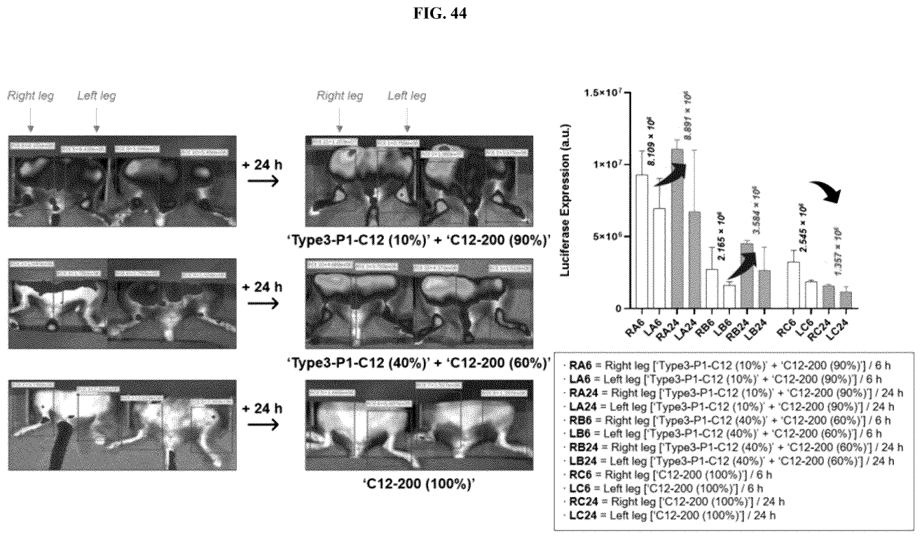

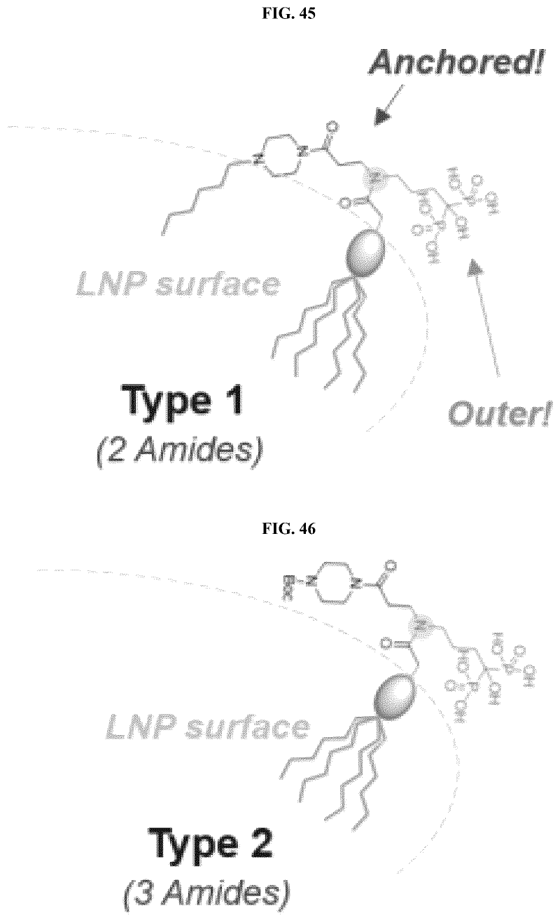

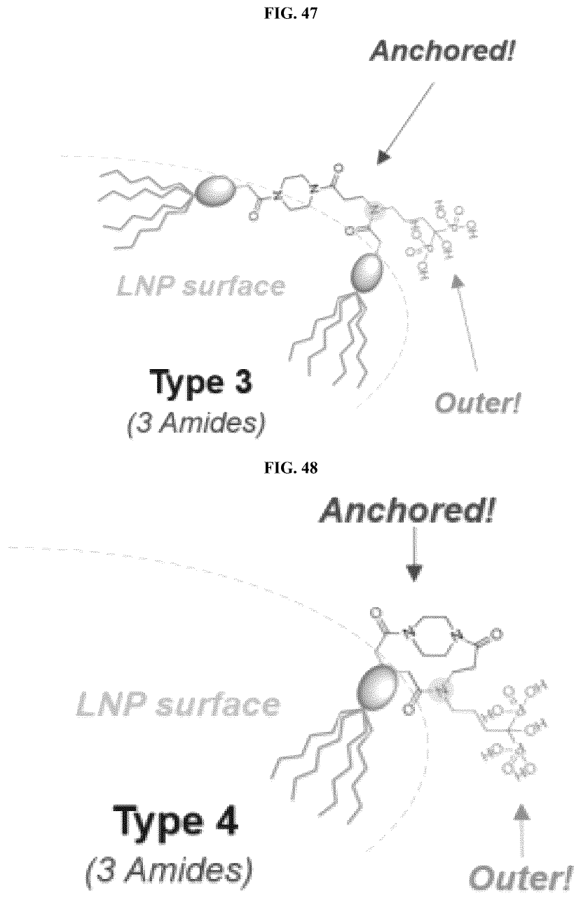

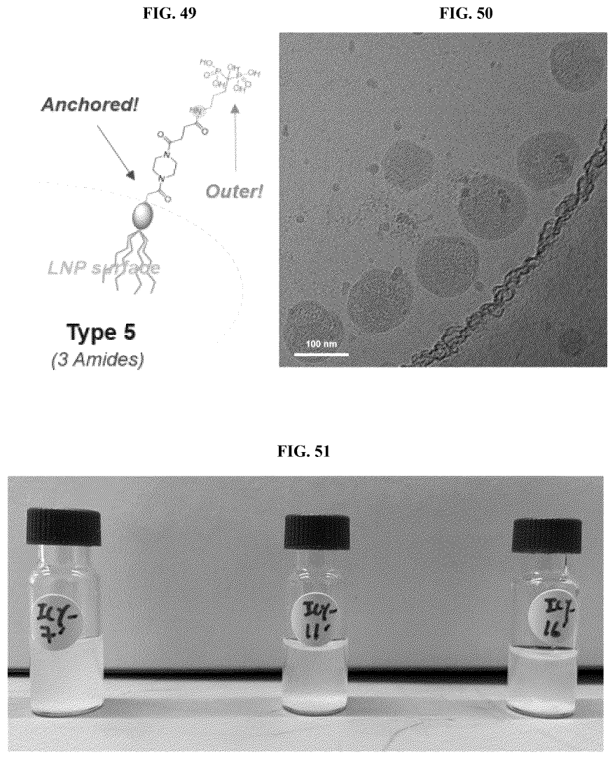

Based on this series of research findings, in vivo experiments were conducted. Each C57BJ/6J mouse was intravenously injected with an mRNA dosage of 0.5 mg/kg. It was necessary to enhance the encapsulation efficiency and stability of PIP-BP LNPs to effectively deliver mRNA to bone cells. Therefore, as mentioned earlier, the goal was to mix ‘C12-200’ ionizable lipid to reduce the size and achieve uniformity in LNPs. In order to employ this approach, preliminary research was required to determine the optimal ratio of ‘C12-200’ ionizable lipid to one of the PIP-based BP-linked LNPs ionizable lipids. However, rather than increasing the proportion of relatively large PIP-based BP-linked LNPs ionizable lipids compared to ‘C12-200’ ionizable lipid, the usage of a PIP-based BP-linked LNPs ionizable lipid was limited to a maximum of 50% relative to ‘C12-200’ ionizable lipid to create relatively stable bone-targeted LNPs. It was observed that bone-targeted LNPs generated by a harmonized ratio of 10% ‘Type3-P1-C12’ PIP-BP lipid and 90% ‘C12-200’ ionizable lipid strongly bound to the surrounding cells near the bone. These in vivo experimental results (FIGS. 43A-43B) closely resembled the findings of the in vitro experiments (FIG. 37D and FIG. 62). LNPs based on ‘Type3-P1-C12’ mixed with ‘C12-200’ ionizable lipid exhibited high luciferase expression in vivo. This could be attributed to the chemical structure of ‘Type 3′, which facilitates easy binding with mRNA due to multiple amine cores and tails, as well as advantages in endocytosis based on electrostatic interactions. Additionally, it appeared to easily disrupt the LNP surface during the lysosomal-endosomal escaping stage.

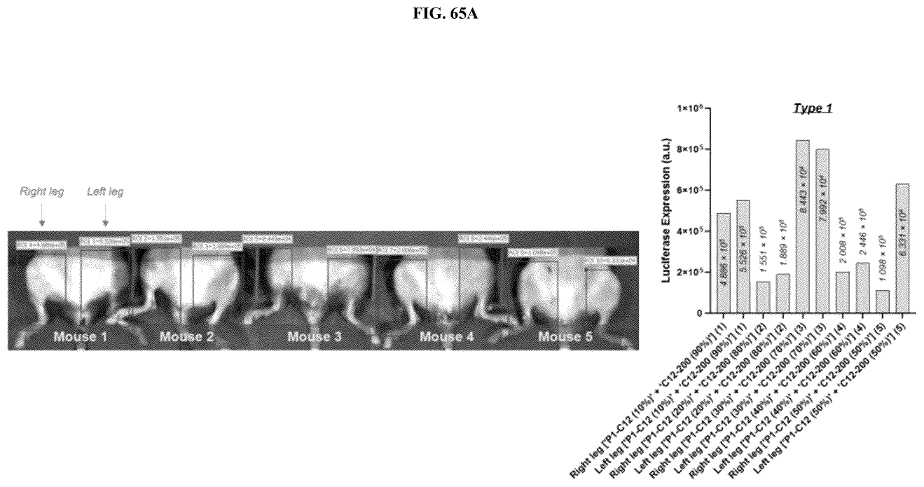

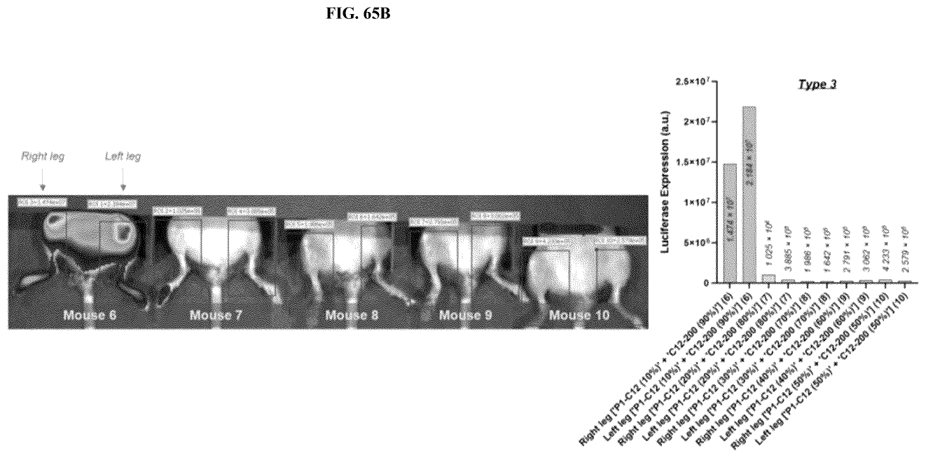

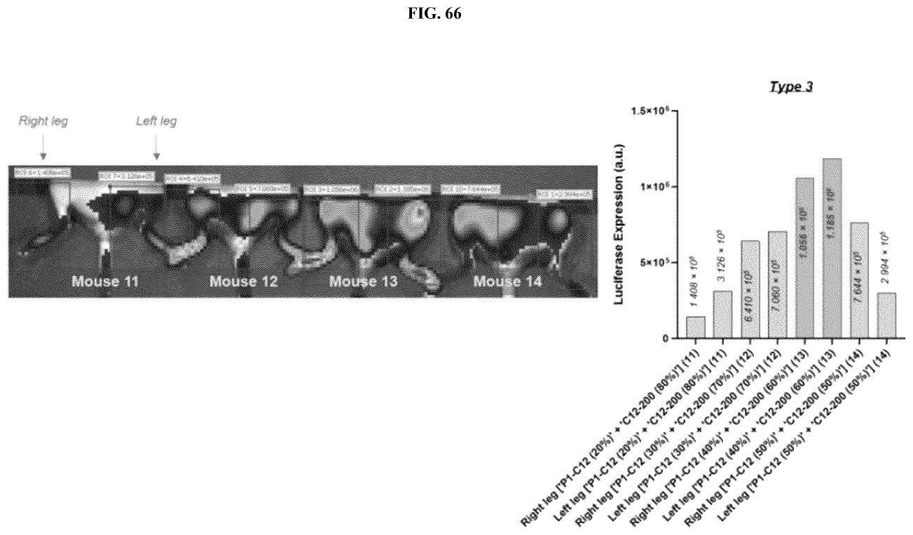

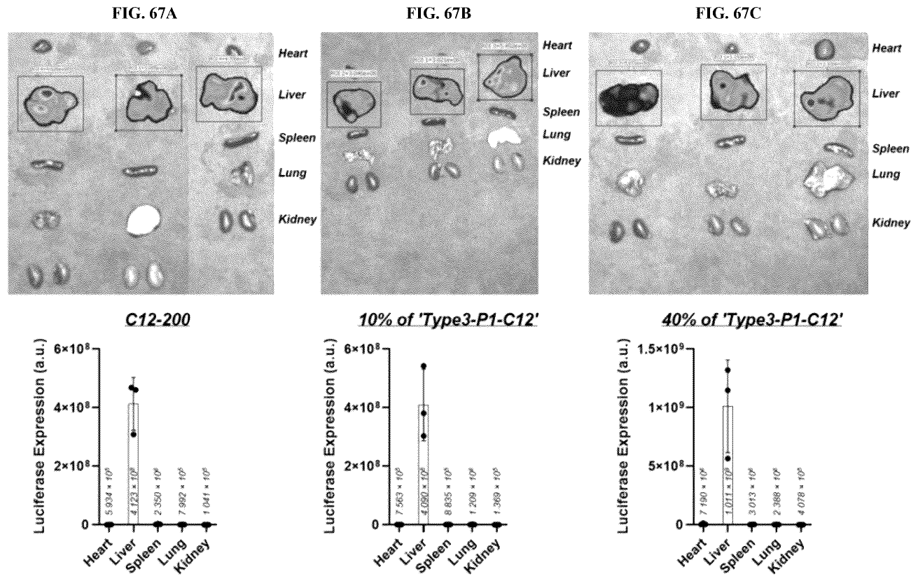

It was found that bone-targeted LNPs based on ‘Type3-P1-C12’ PIP-BP lipid exhibited higher transfection compared to LNPs based on ‘Type1-P1-C12’ PIP-BP lipid (FIGS. 65A-65B). Furthermore, it was observed that bone-targeted LNPs generated by a harmonized ratio of 10% ‘Type3-P1-C12’ PIP-BP lipid and 90% ‘C12-200’ ionizable lipid demonstrated the most efficient transfection. In order to examine protein expression in cells when excess PIP-BP ionizable lipid was used, the proportion of ‘Type3-P1-C12’ PIP-BP lipid was increased relative to ‘C12-200’ ionizable lipid up to 50% and observed its effect on transfection. The results showed that PIP-BP LNPs generated by a harmonized ratio of 40% ‘Type3-P1-C12’ PIP-BP lipid and 60% ‘C12-200’ ionizable lipid exhibited the strongest luciferase expression (FIG. 66). Without wishing to be bound by any theory, this phenomenon suggests that although it may take some time for PIP-BP LNPs to enter the cells around the bone, they gradually accumulate in the adjacent bone cells. In FIGS. 37A-37F, FIGS. 65A-65B, and FIG. 66, it was confirmed that PIP-BP LNPs bind to the bones of mice. Subsequently, in vivo imaging system (IVIS) was conducted for mice whole-body imaging and imaging of the leg portions after sacrificing the mice, as shown in FIGS. 43A-43C. The PIP-BP LNPs generated by the ratio of 10% ‘Type3-P1-C12’ PIP-BP lipid and 90% ‘C12-200’ ionizable lipid, as well as the ratio of 40% ‘Type3-P1-C12’ PIP-BP lipid and 60% ‘C12-200’ ionizable lipid, which exhibited the highest luciferase expression, were subjected to imaging. Through anatomical dissection of the leg portions of the mice, particularly in the femur, patella, and medial fabella, strong luciferase expression was observed. Thus, it was evident from the in vivo experiments that the newly developed PIP-BP LNPs strongly bind to bones.

Previous in vitro results suggest the PIP-BP LNPs control the delivery of mRNA by retention to HA surfaces. To investigate the accumulation of PIP-BP LNPs in bones over time, the PIP-BP LNPs generated by the ratio of 10% ‘Type3-P1-C12’ PIP-BP lipid and 90% ‘C12-200’ ionizable lipid, as well as the ratio of 40% ‘Type3-P1-C12’ PIP-BP lipid and 60% ‘C12-200’ ionizable lipid, which exhibited the top performance, were injected into mice and observed changes in luciferase expression (FIG. 44). Initially, although weak luminescence intensity was observed, over time, strong luciferase expression was evident in the major bone regions, indicating the accumulation of bone-targeted LNPs in these areas. In contrast, ‘C12-200’ ionizable lipid-based LNPs showed a decrease in luminescence intensity over time or little change. Together these results suggest PIP-BP LNPs control targeting to bone microenvironment as well as retention and transfection of cells in vivo over time.

### Bisphosphonate Lipid Nanoparticle (BP-LNP)-Adsorbed Mineralized Tissue Compositions and Methods of Use Thereof

### Materials and Methods

LNP formulation

An ethanol phase comprising all lipid constituents and an aqueous phase containing citrate were combined by vigorous pipette mixing to fabricate LNPs. The ethanol phase consisted of either bisphosphonate ionizable lipid or C12-200 ionizable lipid (Avanti), along with 18:1 (A9-Cis) PE (DOPE) (Avanti), cholesterol (Sigma Aldrich), and 14:0 PEG2000 PE (PEG lipid) (Avanti) in fixed molar ratios of 35% (2.20×e-07 mol), 16% (1.01×e-07 mol), 46.5% (2.92×e-07 mol), and 2.5% (1.57×e-08 mol), respectively. LNPs underwent dialysis in 1×PBS using a microdialysis cassette (Thermo Scientific) for 2 hours, followed by filtration through a 0.22 μm filter.

LNP characterization

mRNA and siRNA concentration in LNPs for in vitro use were quantified using a NanoDrop One Microvolume UV-Vis Spectrophotometer (Thermo Fisher Scientific) and a Qubit HS RNA assay (Invitrogen). Encapsulation efficiency of LNP was evaluated by measuring RNA concentrations in LNPs in 1% in TE buffer and 1% 1× Triton X buffer using a modified Quant-iT R^{1b}oGreen RNA assay (Thermo Fisher Scientific) or a Qubit HS RNA assay (Invitrogen). LNP hydrodynamic diameter, polydispersity (PDI) and zeta potential were measured by Dynamic Light Scattering (DLS) using a DynaPro™ Plate Reader III or Malvern Zetasizer Nano ZS.

Cell culture

BJ cells were cultured in low-glucose Dulbecco's Modified Eagle Medium (DMEM) (Gibco) supplemented with 10% FBS and 0.1% bFGF. tdROSA bone marrow mesenchymal stem cells (tdROSA mMSCs) were isolated from tdROSA mice's long bones. tdROSA mMSCs were cultured in Minimum Essential Medium a (a MEM) (Gibco) supplemented with 10% FBS and 0.1% bFGF. All cell lines were grown at 37° C. under a 5% CO2 humidified atmosphere until confluence.

In vitro transfection screening and cell viability assays

In a white-wall transparent bottom 96-well plate, BJ cells were seeded at a density of 5,000 cells per well in 100 μL growth medium (low-glucose DMEM, 10% FBS), and were incubated at 37° C. in 5% CO2 overnight. The medium was exchanged for fresh growth medium, and then LNPs were treated at a dose of 20 ng firefly luciferase mRNA per well. Luciferase expression was measured 24 h after LNP transfection using a Luciferase Assay System (Promega). The luminescent signal was normalized to growth medium-treated cells. Cell viability was measured using a CellTiter-Glo Luminescent Cell Viability Assay (Promega).

HA surface binding

HA disc (10 mm Diameter×2 mm Thick) was purchased from Clarkson Chromatography Products. 150 μL BP-LNP and control LNP (dissolved in a mixture of 1×PBS and low glucose DMEM) containing 2000 ng mRNA were incubated on HA discs at 37° C. and shaken for 24 h. Empty HA discs and HA discs loaded with a mixture of 1×PBS and DMEM were used as blank control. At the end of incubation time, non-binding LNPs were thoroughly washed off by DI water. Chemical bonding on HA disc surfaces was visualized by FT-IR. FT-IR spectra were recorded with a Thermo Scientific Nicolet™ iS™ 5 FT-IR spectrometer, equipped with an iD7 ATR diamond.

MiTEX binding kinetics on HA

A molar ratio of 0.3% Fluor 594-PE (Avanti) was mixed in lipid formulation to label LNPs. The fluorescent intensities of 150 μL fluorescent labeled MiTEX and control (dissolved in a mixture of 1×PBS and low glucose DMEM) containing 2000 ng mRNA were measured at 635 nm excitation wavelength using a Qubit 4 fluorometer (Thermo Fisher Scientific). Then, these LNPs were incubated on HA discs at 37° C. and shaken for varying lengths of time (10, 30, 60, 120, 240, 480, 720 and 1440 min). At the end of incubation time, non-binding LNPs were thoroughly washed off with 150 μL DI water. The wash-off liquid was collected to measure the fluorescent intensity. The binding kinetics of MiTEX and control were determined by measuring the decrease in the relative concentration (correlating to fluorescence intensity).

In vitro mRNA transfection on HA substrate via MiTEX adsorption

75 μL MiTEX or control (dissolved in a mixture of 1×PBS and low glucose DMEM) containing 2000 ng mRNA that formed semi-spheres on HA disc were incubated at 37° C. and shaken for 24 h. At the end of incubation time, non-binding LNPs were thoroughly washed off with 400 μL DI water. Remaining mRNA on HA disc was measured by Qubit after treating the HA disc with 100 μL 1× Triton X buffer. Non-adsorbed mRNA in wash-off liquid was also mixed with 1× Triton X buffer and measured by Qubit to assess the total mRNA loss after loading. HA discs were preconditioned with low glucose DMEM and 0.2% type I collagen overnight. Then, 150 μL MiTEX or control (dissolved in a mixture of 1×PBS and low glucose DMEM) containing 2000 ng firefly luciferase or Cre-recombinase mRNA were incubated on HA discs at 37° C. and shaken for 24 h. After a thorough wash with DI water, 50,000 BJ cells or 100,000 tdROSA mMSCs were seeded onto HA disc and incubated for 48 h. At the end of incubation time, BJ cells transfected with firefly luciferase mRNA were lysed for performing luciferase expression assay. tdROSA mMSCs were fixed by 4% PFA and stained for cell nuclei (DAPI, blue) and F-actin (phalloidin, green). Cells were imaged by a confocal fluorescent microscope and analyzed by FIJI.

In vitro MiTEX-bone graft transfection

BJ cells were seeded in 96-well plate (20,000 cells per well) and incubated overnight prior to treatment. Allogenic bone graft was mixed with 10 μL MiTEX or control (dissolved in a mixture of 1×PBS and low glucose DMEM) encapsulating firefly luciferase mRNA ranging from 5 to 100 ng per bone graft cluster and transferred to each well. After 24 h co-incubation, BJ cells were lysed, and luciferase expression assay was performed.

STAT3 knockdown

Gingival fibroblasts were seeded in 24-well plate (90,000 cells per well) and incubated overnight before treatment with STAT3 siRNA-LNP. A serial dilution of each siRNA control LNP formulation in low glucose DMEM+10% FBS was prepared at concentrations of 1 to 100 nM siRNA. 100 nM siRNA encapsulated by lipofectamine was treated as a positive control. No LNP-treated group was blank control. After 24 h treatment, condition media was removed. Cells were washed with DPBS and lysed by Beta-mercaptoethanol. Lysis buffer was collected for RNA extraction using RNeasy Mini Kit (Qiagen) and RT-qPCR following the manufacturer's protocol. MiTEX and control encapsulating 50 nM siRNA were treated to gingival fibroblasts and performed gene expression experiments following the same method. Relative cell viability after 24 h siRNA-LNP treatment was measured using a CellTiter-Glo Luminescent Cell Viability Assay (Promega).

LPS stimulation and downstream cytokine modulation

Gingival fibroblasts were seeded in 96-well plate (20,000 cells per well) and in 24-well plate in defined media (low glucose DMEM, 10 μL/mL Penicillin-Streptomycin, 10 μL/mL SITE Liquid Media Supplement, 5 μL/mL Antibiotic-Antimycotic Solution, 5 μL/mL Hydrocortisone, 5 μL/mL FBS, and 0.2 μL/mL bFGF) overnight. Cells were treated with STAT3 siMiTEX or control (50 nM siRNA per well) for 24 h. Then, conditioned media were removed, and cells were exposed to LPS (100 g/mL) for 24 h. For STAT3 gene level measurement, after 24 h treatment of LPS, cells seeded in 24-well plate were washed with DPBS and lysed by Beta-mercaptoethanol. Lysis buffer was collected for RNA extraction using RNeasy Mini Kit (Qiagen) and RT-qPCR following the manufacturer's protocol. For downstream cytokine level measurement, after 24 h treatment of LPS, supernatant was collected, and fresh media were added for another 24 h incubation. Conditioned media were collected 24 h and 48 h post-LPS treatment for ELISA. IL-6 (Biolegend) and IL-8 (Biolegend) levels secreted by gingival fibroblasts were measured by ELISA assessments following the manufacturer's protocol.

### Statistics

All analyses were performed using GraphPad Prism 10 (La Jolla, CA) software; more specifically, statistical analysis was carried out with unpaired two-tailed t-tests or one- or two-way ANOVAs where appropriate. Data were plotted as mean±SEM unless otherwise stated.

### Example 12: Formulation, physicochemical characterization, and in vitro transfection of BP-LNPs

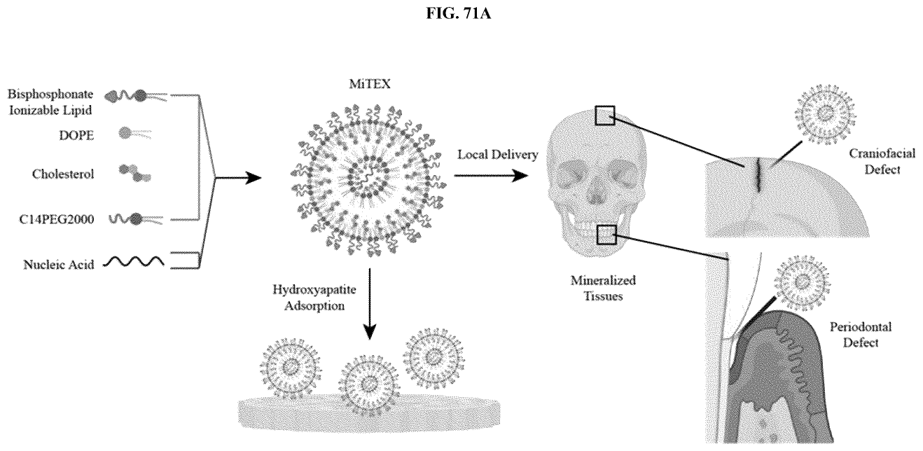

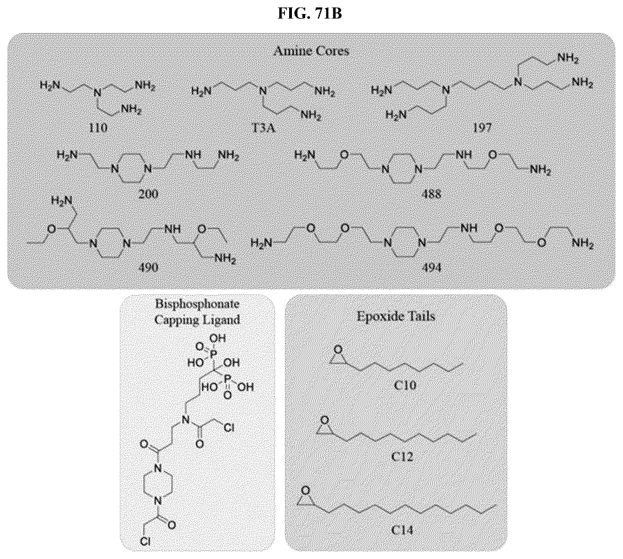

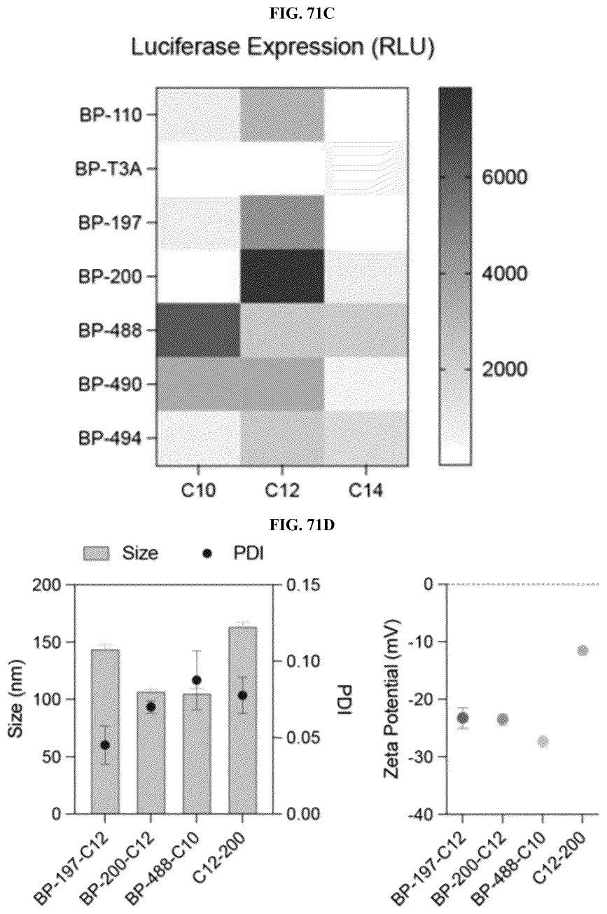

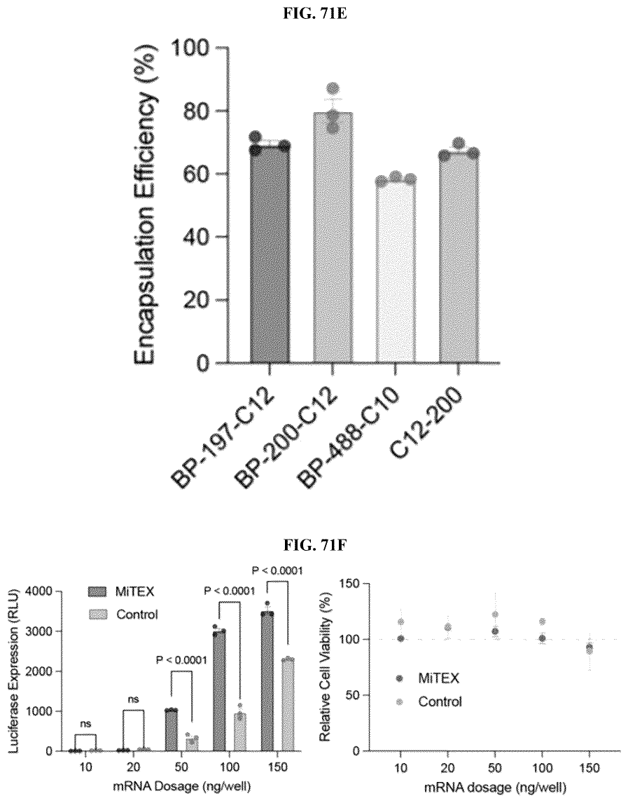

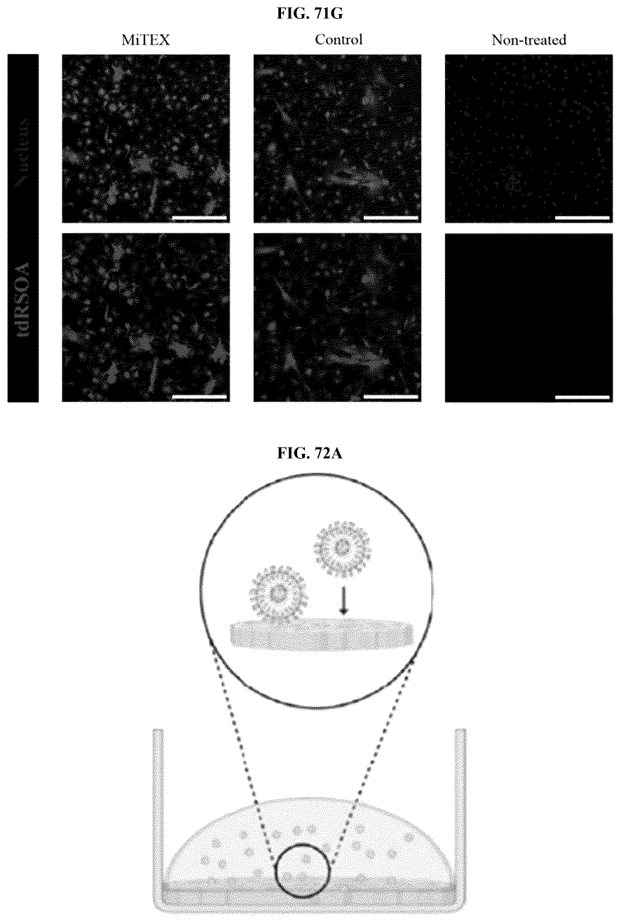

MiTEX and control were initially formulated into LNPs using the commonly utilized molar ratios in lipid formulation, consisting of 35% ionizable lipid, 16% DOPE phospholipid, 46.5% cholesterol, and 2.5% C14-PEG2000 (FIG. 71A). Here, ionizable lipid C12-200 was introduced as a non-targeting LNP control (referred to as ‘control’ in the following paragraphs). Four lipid components were dissolved in an ethanolic phase and then pipette-mixed with an aqueous citrate buffer containing the nucleic acid to formulate LNPs (FIG. 71A). To achieve LNP targeting and accumulation in mineralized tissues, a series of piperazine-linked bisphosphonate-based ionizable lipids consisting of alendronate, a type of bisphosphonate prevalently used for treating osteoporosis, were used in this study. The ionizable lipids consisted of three main parts, a bisphosphonate capping ligand, amine cores, and epoxide chains with varying tail lengths, which were linked together through chemical bonding and coupling reactions(FIG. 71B). The naming of the LNP formulations reflects both the specific bisphosphonate-based amine core component (referred to as BP-110, BP-197, BP-T3A, BP-200, BP-488, BP-490, and BP-494) and the length of the epoxide tail (C10, C12, and C14). Piperazine ring with a chair-form configuration was introduced in these mineralized tissue-targeting ionizable lipids to immobilize the movement of bisphosphonate and further anchor the chemical skeleton on the surface of LNPs. A library of 28 bisphosphonate-based LNPs was constructed, and the naming of the LNP formulations reflects both the specific bisphosphonate-based amine core component (referred to as BP-110, BP-197, BP-T3A, BP-200, BP-488, BP-490, and BP-494) and the length of the epoxide tail (C10, C12, and C14) (FIG. 71B).

To investigate the structure-activity relationships focusing on the chemical structures of amine cores and the alkyl tails, as well as evaluate MiTEXs for their RNA delivery and expression capacity within a mineralized tissue microenvironment, in vitro transfection screening was performed on BP-LNPs that were formulated with firefly luciferase mRNA via vigorous pipette mixing was conducted on BJ cells, a human fibroblast cell type. After treatment with BP-LNPs for 24 h at a concentration of 20 ng/5000 cells, luciferase expression was evaluated via luminescence measurements, which were normalized to an untreated control group. BP-200-C12, BP-197-C12, and BP-488-C10 were the top-performing ionizable lipids with the highest transfection efficiency in BJ cells (FIG. 71C). The heatmap of luciferase expression indicated that amine cores consisting of piperazine structure (200, 488, 490, 494) had higher potency of transfection, which was supported by the wide use of piperazine as a biologically active compound. Comparing amine cores with piperazine structures, the simpler structure of amine cores exhibited a more favorable transfection. Moreover, based on the relationship between tail length and transfection efficiency, it can be inferred that shorter tails (C10 and C12) were more favorable for transfection, compared to longer tails (C14).

Three top-performing MiTEXs were characterized to evaluate particle size, polydispersity index, zeta potential and mRNA encapsulation efficiency. The hydrodynamic diameter of MiTEXs and control ranged from 105.95 to 164.2 nm by intensity measurements using dynamic light scattering (DLS) (FIG. 71D). All the MiTEX and control formulations were highly monodisperse, with a PDI value less or around 0.1(FIG. 71D). Due to the inclusion of negatively charged alendronate molecules within the ionizable lipids, the MiTEXs exhibited a negative zeta potential. ranging from −27.33 to -23.13 mV, while the mean zeta potential of the control was -11.53 mV (FIG. 71D). The mRNA concentration and encapsulation efficiency of MiTEX and control were assessed by a fluorescent R^{1b}oGreen assay, where all efficiencies ranged from 58.33 to 80.03% (FIG. 71E). Based on the in vitro transfection and physicochemical screening, BP-200-C12 LNP presented the best performance and therefore was used to represent BP-LNPs for the following studies.

Further, to investigate the gene delivery efficiency within a gene-edit murine mineralized tissue environment, dose-dependent transfection of MiTEX encapsulating firefly luciferase mRNA in tdROSA mouse bone marrow mesenchymal stem cells (mMSCs) was assessed. MiTEX exhibited a dose-dependent RNA expression in tdROSA mMSC, while the relative cell viability was above 90% in all dose conditions (FIG. 71F). To visualize the transfection and genetic-labeling ability of MiTEX, LNPs encapsulating Cre-recombinase mRNA (200 ng/20,000 cells) were transfected into tdROSA mMSCs. After 24 h of treatment, tdROSA mMSCs were genetically labeled and expressed tdTomato successfully (FIG. 71G). Together, these results laid the foundation for studying the local delivery of Cre recombinase mRNA-LNP to the mineralized tissue interfaces in vivo.

### Example 13: MiTEX in vitro binding with hydroxyapatite substrate

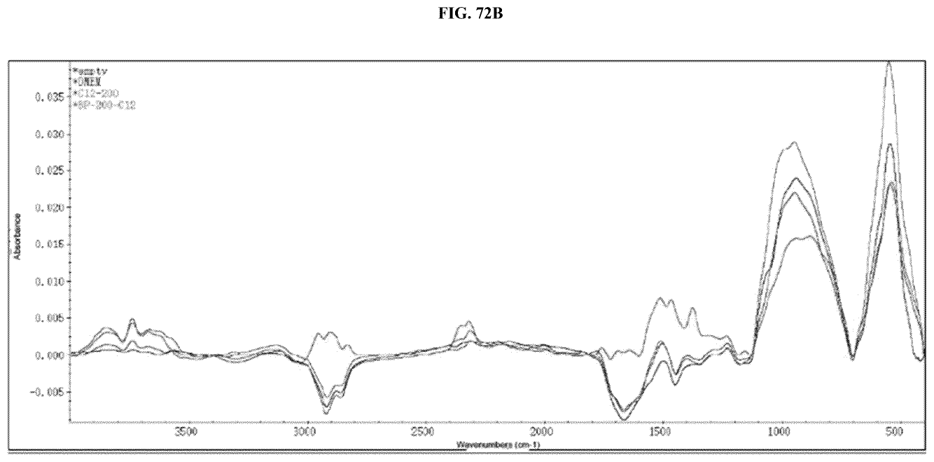

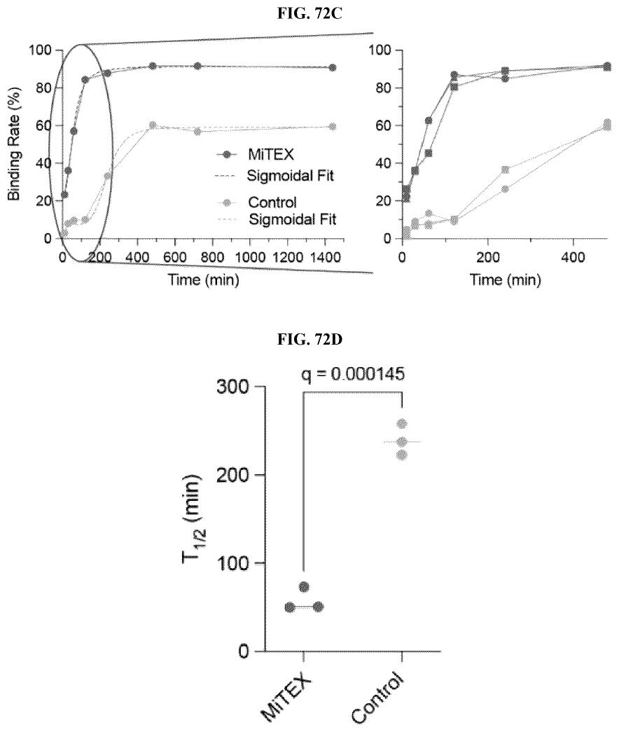

The affinity and interaction between BP-LNPs and hydroxyapatite, one of the mean mineral components of bone materials, were experimentally investigated. MiTEX and control LNP mixed with DMEM were loaded onto a hydroxyapatite disc (diameter: 10 mm) and incubated for 24 h at 37° C. before experimental assessments (FIG. 72A). After incubation, the HA discs were rinsed with DI water thoroughly to remove unadsorbed LNPs on the HA surface. Fourier-transform infrared spectroscopy (FT-IR) was performed on the LNP-adsorbed HA disc surface to record characteristic structures of LNPs on the HA surface. Peaks representing signature groups of BP-200-C12 ionizable lipid, including 2954, 2915, and 2848 [v(—CH₂— and —CH₃)], 1464 [v(—CH₂— and CH₃)], could be identified on the HA disc surface loaded with BP-200-C12 LNPs (FIG. 72B). No distinct phosphate peaks attributable to the bisphosphonate ionizable lipid were observed, likely due to the high phosphate content of the hydroxyapatite substrate, which masked the bisphosphonate signals on the surface.

HA binding kinetics were performed on HA discs with MiTEX compared to control LNPs. Lipid components were mixed with 0.3% Fluor 594-PE, a fluorescent lipid for quantifiable assessment. After loading and incubation on HA discs, MiTEX exhibited fast and efficient binding to HA, driven by the strong chelation between bisphosphonate conjugation outwarding LNPs and HA surface (FIGS. 72C-72D). After 120 min of incubation, MiTEX achieved an HA binding efficiency of 80%, significantly higher than the 10% observed for control. After this time point, the binding rate reached 90% and plateaued with increased incubation time, while control plateaued at a binding efficiency of 60% after 480 min incubation (FIG. 72C). These results demonstrate the ability to locally target mineralized tissues by the rapid and efficient adsorption of MiTEX on HA substrate.

### Example 14: In vitro adsorption of MiTEX on hydroxyapatite substrate provides a local reservoir for RNA loading and delivery

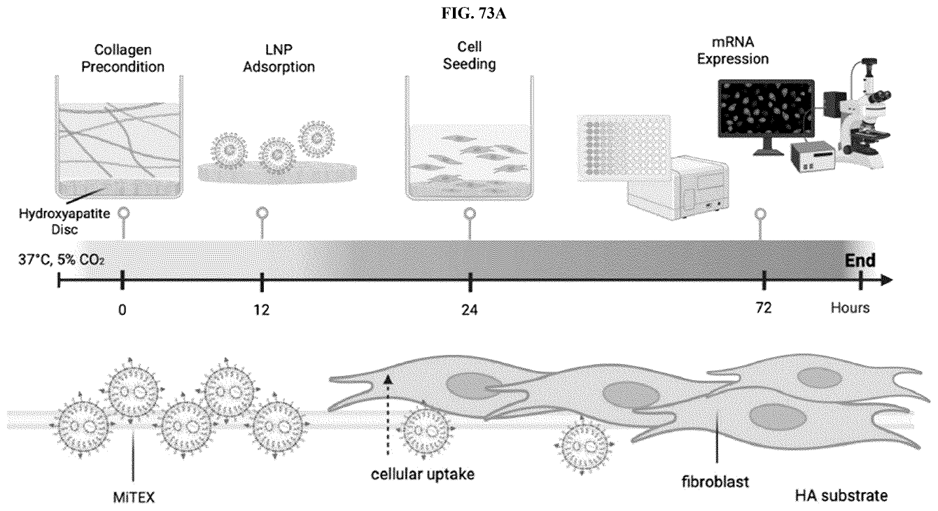

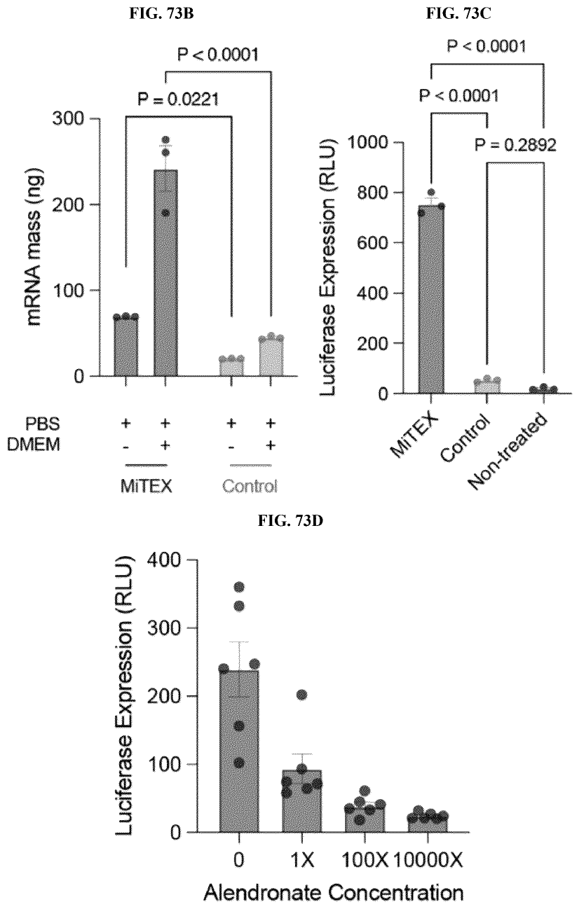

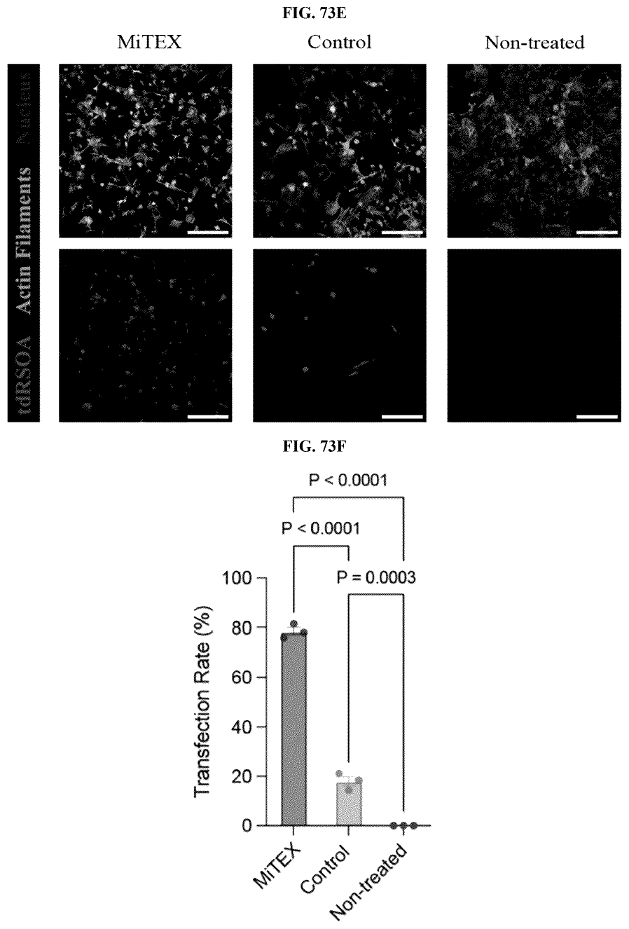

Here, the targeted delivery of mRNA into fibroblasts and mesenchymal stem cells was controlled by leveraging the affinity and adsorption of MiTEX to HA (FIG. 73A). To investigate the effective biological components adsorbed on HA substrate, mRNA accumulation mass after loading 2000 ng mRNA encapsulated in BP-200-C12 LNPs and control LNPs was quantified. Remaining mRNA on HA disc 24 h after loading and wash-off was measured by Qubit after treating the HA disc with 100 μL Triton X-100 detergent. MiTEX showed significantly higher accumulation of mRNA onto HA surfaces compared to control (FIG. 73B). More than 200 ng mRNA was retained on HA surface in MiTEX group, whereas in the control group, less than 50 ng mRNA was detected. The impact of solvent in LNP adsorption was also discussed in this study. BP-LNPs dissolved in 1×PBS showed a lower amount of remaining mRNA compared to 1×PBS and DMEM mixing solvent, suggesting that the phosphate salts in PBS buffer could negatively interfere with the binding interaction between BP-LNPs and HA (FIG. 73B). Therefore, to achieve the highest mRNA accumulation rate on HA substrate, 1×PBS and DMEM mixing solvent were used for the following studies.

Next, BJ cells and tdROSA mMSCs were subsequently cultured on collagen-treated HA substrates after loading of MiTEX or control to investigate the transfection capacity of LNPs after adsorption. No free mRNA was detected in conditioned media, suggesting the transfection was dependent on the surface uptake of adsorbed lipids/mRNA. LNPs encapsulating firefly luciferase mRNA and Cre-recombinase mRNA were loaded separately for quantification and observation, respectively. A significant luciferase expression on the BP-LNPs loaded HA substrate was detected in BJ cells after 48 h of culturing on the collagen-treated LNP-loaded HA disc (FIG. 73C). Conversely, the HA surface was blocked with alendronate sodium, the main targeting moiety of bisphosphonate ionizable lipid, prior to MiTEX adsorption to further demonstrate that bisphosphonate is the key binding element contributing to HA surface uptake and adsorption-mediated RNA delivery. HA substrate was pretreated with alendronate sodium at 1 to 10000 times the concentration of the alendronate in HA-adsorbed BP-LNPs used in previous studies. An alendronate concentration-dependent luciferase expression was observed (FIG. 73D), indicating that the pretreated alendronate has blocked the binding sites on HA substrate and hindered the following MiTEX adsorption and gene expression. tdROSA mMSCs were seeded on Cre-recombinase mRNA-LNP loaded HA substrate for 48 h and imaged by confocal fluorescence microscope, and tdTomato-expressing cells were counted. MiTEX exhibited around 80% genetic labeling of cells, significantly higher than control (17.92%) or blank controls (0%) (FIGS. 73E-73F). Both transfection studies on HA substrate revealed that the adsorbed MiTEXs retained the ability to deliver mRNA and transfect adjacent cells, which indicated that with the adsorption of BP-LNPs, mineral substrate can turn into a local RNA loading and delivery platform. Together, these new data suggest a new mechanism of RNA delivery via surface adsorption and uptake of MiTEX on HA/mineralized substrates.

### Example 15: Bone graft adsorption of MiTEX mediates local gene expression in vivo

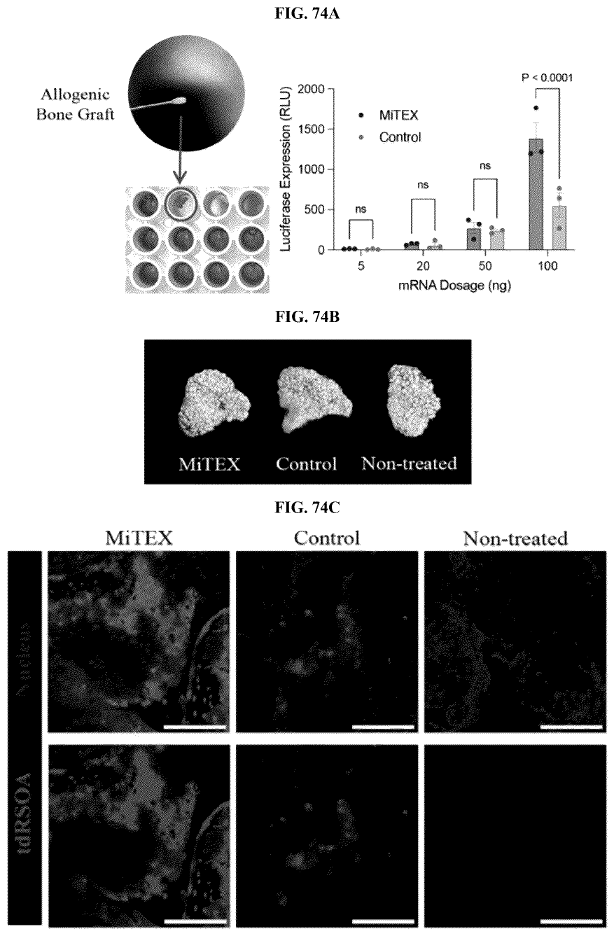

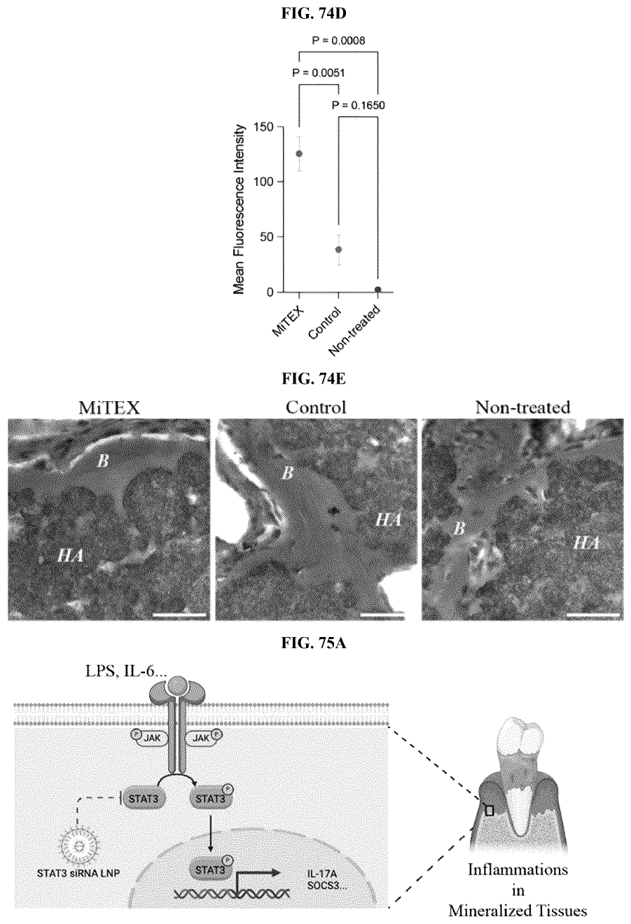

The in vitro findings suggest that MiTEX can be adsorbed to and be retained on a broad range of mineralized tissues and materials, including bone, teeth, and bone graft materials. Here, bone graft materials, a mineral scaffold that could support bone regeneration, were chosen as an MiTEX adsorbing substrate to mediate mRNA delivery for in vitro and in vivo studies. Allogenic bone graft material was rehydrated with firefly luciferase mRNA-LNPs and transferred to transfect BJ cells. By the adsorption of LNPs in dry bone grafts, BJ cells exhibited a dose-dependent luciferase expression in both BP-LNP and control LNP loading bone grafts (FIG. 74A), which again suggested the new RNA delivery and gene expression approach by the adsorption of MiTEX in mineral substrates and proved the translational potential of applying therapeutic MiTEX in current clinically used materials or biomaterials.

Next, to investigate genetic labeling of bone formation in vivo, Cre mRNA-LNPs loaded within bone graft scaffolds, hydroxyapatite tricalcium phosphate, were implanted subcutaneously into immunocompromised mice. 4,000,000 mouse bone marrow MSCs were co-delivered within the bone graft scaffold to provide sufficient cell sources for de novo bone formation. After 60 days of implantation, mice were sacrificed, and newly formed bone tissues were evaluated using micro-computed tomography (yCT) scans and histology (FIG. 74B). Cre-recombinase labeling in newly formed bone tissues was imaged by fluorescent microscope. In vivo tdTomato expression from tdROSA mMSCs demonstrated that the local mRNA delivery approach via adsorbing MiTEXs within bone graft materials was feasible (FIG. 74C). MiTEX-treated bone tissues exhibited a significantly higher mean fluorescent intensity than control-treated bone tissues (FIG. 74D), suggesting that the affinity of MiTEX in mineral substrate led to better RNA adsorption and higher dosage retention in bone grafts. To further validate the bone formation observed in the pCT analysis, H&E staining and alkaline phosphatase immunohistochemical staining were performed. H&E staining results suggest cortical bone-like tissues were formed surrounding bone graft scaffolds in all groups (FIG. 74E), which was also supported by obvious osteoblast activity from ALP immunohistochemical staining, suggesting MiTEX shows no impairment to bone activity. These results confirmed the hypothesis that utilizing mineral substrate as a reservoir to locally deliver RNAs into mineralized tissues in vivo, and also provided a new platform for co-implantation of mineral scaffolds with therapeutic MiTEX to boost bone formation or regeneration.

### Example 16: STAT3 siMiTEX modulates the STAT3 pathway in vitro

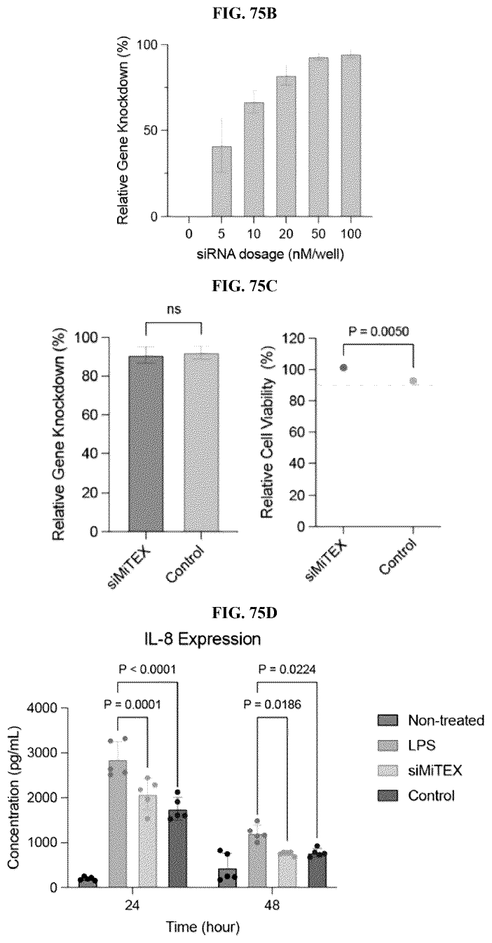

STAT3 is critically involved in the regulation of bone and tooth development. Upon stimulation, STAT3 undergoes phosphorylation and is subsequently translocated into the nucleus, initiating downstream proinflammatory cytokine expression (FIG. 75A). The delivery potential of BP-LNPs encapsulating STAT3 siRNA to modulate STAT3 gene expression and subsequently regulate proinflammatory cytokine release presents a promising strategy for treating bone and dental diseases (FIG. 75A). Therefore, it was investigated whether knockdown STAT3 using MiTEX for siRNA delivery could reduce STAT3 phosphorylation and consequently decrease downstream cytokines release.

Gingival fibroblasts were treated with control STAT3 siMiTEX at concentrations ranging from 1 to 100 nM of STAT3 siRNA. After 24 h treatment, STAT3 expression was evaluated using RT-qPCR, which exhibited a dose-dependent decrease in STAT3 expression (FIG. 75B). siRNA dosage with 50 nM and 100 nM showed above 90% knockdown of STAT3 expression (FIG. 75B). Similarly, siMiTEX and control with a concentration of 50 nM siRNA were separately treated to gingival fibroblasts for 24 h and quantified STAT3 expression and relative cell viability. Both siMiTEX and control exhibited above 90% decreased STAT3 expression without induction of gingival fibroblast cell death (FIG. 75C). Further, whether reducing STAT3 expression using STAT3 siMiTEX affects the release of proinflammatory cytokines was examined in stimulated fibroblasts. Gingival fibroblasts were first treated with STAT3 siMiTEX or control at 50 nM/well. After 24 h, STAT3 siRNA was removed, and cells were exposed to 10 μg/mL lipopolysaccharide (LPS) for 24 h. Conditioned media were collected at 24 h after LPS treatment, and cells were changed to fresh media for another 24 h to collect. LPS significantly elevated the levels of proinflammatory cytokines IL-6 and IL-8 as expected (FIG. 75D). Subsequently, STAT3 STAT3 siMiTEX and control notably reduced IL-6 and IL-8 release levels by suppressing LPS-induced STAT3 gene expression and phosphorylation. These results demonstrated the therapeutic potential of MiTEX for local treatments within mineralized tissues.

### Enumerated Embodiments

The following exemplary embodiments are provided, the numbering of which is not to be construed as designating levels of importance:

Embodiment 1 provides a lipid nanoparticle (LNP). In certain embodiments, the LNP comprises (a) at least one compound having the structure of Formula (I), or a racemate, enantiomer, diastereomer, pharmaceutically acceptable salt, solvate, or a derivative thereof:

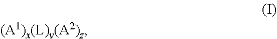

wherein:
each occurrence of A¹ is independently

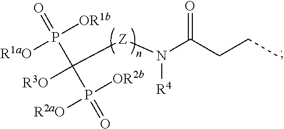

each occurrence of A² is independently

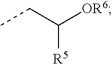

each occurrence of L is an amine linker independently selected from the group consisting of aminoalkyl linker, substituted aminoalkyl linker, diaminoalkyl linker, substituted diaminoalkyl linker, triaminoalkyl linker, substituted triaminoalkyl linker, tetraaminoalkyl linker, substituted tetraaminoalkyl linker, pentaaminoalkyl linker, substituted pentaaminoalkyl linker, polyaminoalkyl linker, substituted polyaminoalkyl linker, aminocycloalkyl linker, substituted aminocycloalkyl linker, diaminocycloalkyl linker, substituted diaminocycloalkyl linker, triaminocycloalkyl linker, substituted triaminocycloalkyl linker, tetraaminocycloalkyl linker, substituted tetraaminocycloalkyl linker, pentaaminocycloalkyl linker, substituted pentaaminocycloalkyl linker, polyaminocycloalkyl linker, substituted polyaminocycloalkyl linker, and any combination thereof;
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof,
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si;
each occurrence of z′ and z″ is independently an integer represented by 0, 1, or 2;
wherein x, y, and z are independently an integer from 0 to 20;
each occurrence of n is independently an integer from 0 to 10; and
the compound is present in a concentration range of about 1 mol % to about 99 mol %.
   In certain embodiments, the LNP comprises (b) at least one neutral phospholipid, wherein the neutral phospholipid is present in a concentration range of about 5 mol % to about 45 mol %. In certain embodiments, the LNP comprises (c) at least one cholesterol lipid, wherein the total cholesterol lipid is in a concentration range of about 5 mol % to about 55 mol %. In certain embodiments, the LNP comprises (d) at least one polymer conjugated lipid (e.g., polyethylene glycol (PEG)-conjugated lipid), wherein the total polymer conjugated lipid is present in a concentration range of about 0.5 mol % to about 12.5 mol %.

Embodiment 2 provides the LNP of Embodiment 1, wherein L is selected from the group consisting of

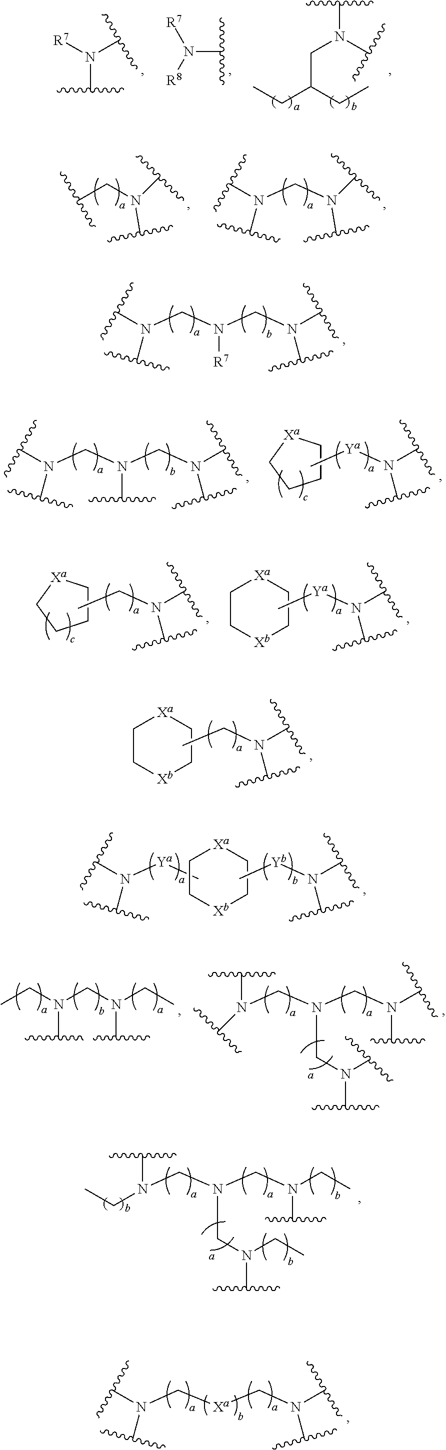

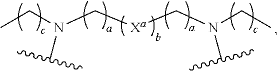

and any combination thereof, wherein:
each occurrence of R⁷ and R⁸ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z′}‘—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof,
each occurrence of X^{a} and X^{b} is independently selected from the group consisting of O, S, N(R⁹)_{z′}, P(R⁹)_{z′}, and any combination thereof,
each occurrence of Y^{a} and Y^{b} is independently selected from the group consisting of C1-C₁₂ alkylenyl, substituted C₁-C₁₂ alkylenyl, C₃-C₈ cycloalkylenyl, substituted C₃-C₈ cycloalkylenyl, —Y′(R⁹)_{z′}(R¹⁰)_{z″}—(C₃-C₈ cycloalkylenyl), substituted —Y′(R⁹)_{z′}(R¹⁰)_{z″}—C₃-C₈ cycloalkylenyl, C₂-C₈ heterocycloalkylenyl, substituted C₂-C₈ heterocycloalkylenyl, —Y′(R⁹)_{z′}(R¹⁰)_{z″}—C₂-C₈ heterocycloalkylenyl, substituted-Y′(R⁹)_{z′}(R¹⁰)_{z″}—C₂-C₈ heterocycloalkylenyl, C₂-C₈ alkenylenyl, substituted C₂-C₈ alkenylenyl, C₅-C₁₀ cycloalkenylenyl, substituted C₅-C₁₀ cycloalkenylenyl, —Y′(R⁹)_{z′}(R¹⁰)_{z″}—C₅-C₁₀ cycloalkenylenyl, substituted —Y′(R⁹)_{z′}(R¹⁰)_{z″}—C₅-C₁₀ cycloalkenylenyl, C₂-C₈ alkynylenyl, substituted C₂-C₈ alkynylenyl, C₈-C₁₂ cycloalkynylenyl, substituted C₈-C₁₂ cycloalkynylenyl, —Y′(R⁹)_{z′}(R¹⁰)_{z″}—C₅-C₁₂ cycloalkynylenyl, substituted —Y′(R⁹)_{z′}(R¹⁰)_{z″}-C5-C12 cycloalkynylenyl, C₆-C₁₀ arylenyl, substituted C₆-C₁₀ arylenyl, —Y′(R⁹)_{z′}(R¹⁰)_{z″}—C₆-C₁₀ arylenyl, substituted —Y′(R⁹)_{z′}(R¹⁰)_{z″}—C₆-C₁₀ arylenyl, C₂-C₁₀ heteroarylenyl, substituted C₂-C₁₀ heteroarylenyl, —Y′(R⁹)_{z′}(R¹⁰)_{z″}—C₂-C₁₀ heteroarylenyl, and substituted —Y′(R⁹)_{z′}(R¹⁰)_{z″}—C₂-C₁₀ heteroarylenyl;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2;
wherein each occurrence of a, b, and c is independently an integer from 0 to 10;
each occurrence of

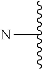 [SMILES: *N]

indicates a bond between a N atom of L and A¹ or A².

Embodiment 3 provides the LNP of Embodiment 1 or 2, wherein L is selected from the group consisting of

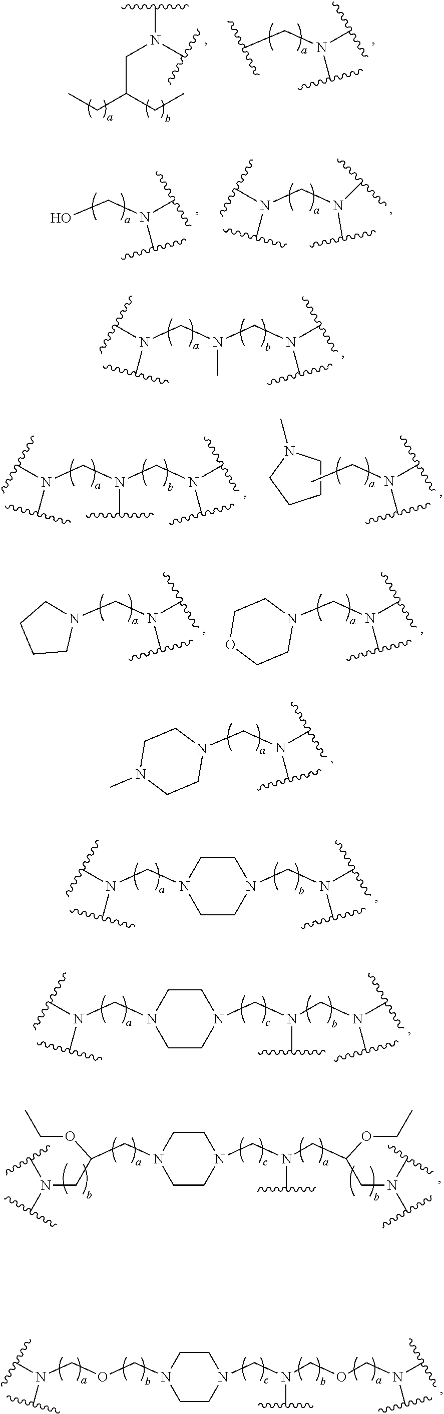

any combination thereof, wherein:
each occurrence of a, b, and c is independently an integer from 0 to 10; and

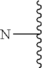 [SMILES: *N]

each occurrence of N indicates a bond between a N atom of L and A¹ or A².

Embodiment 4 provides the LNP of any one of Embodiments 1-3, wherein the compound having the structure of Formula (I) is a compound having the structure selected from the group consisting of:

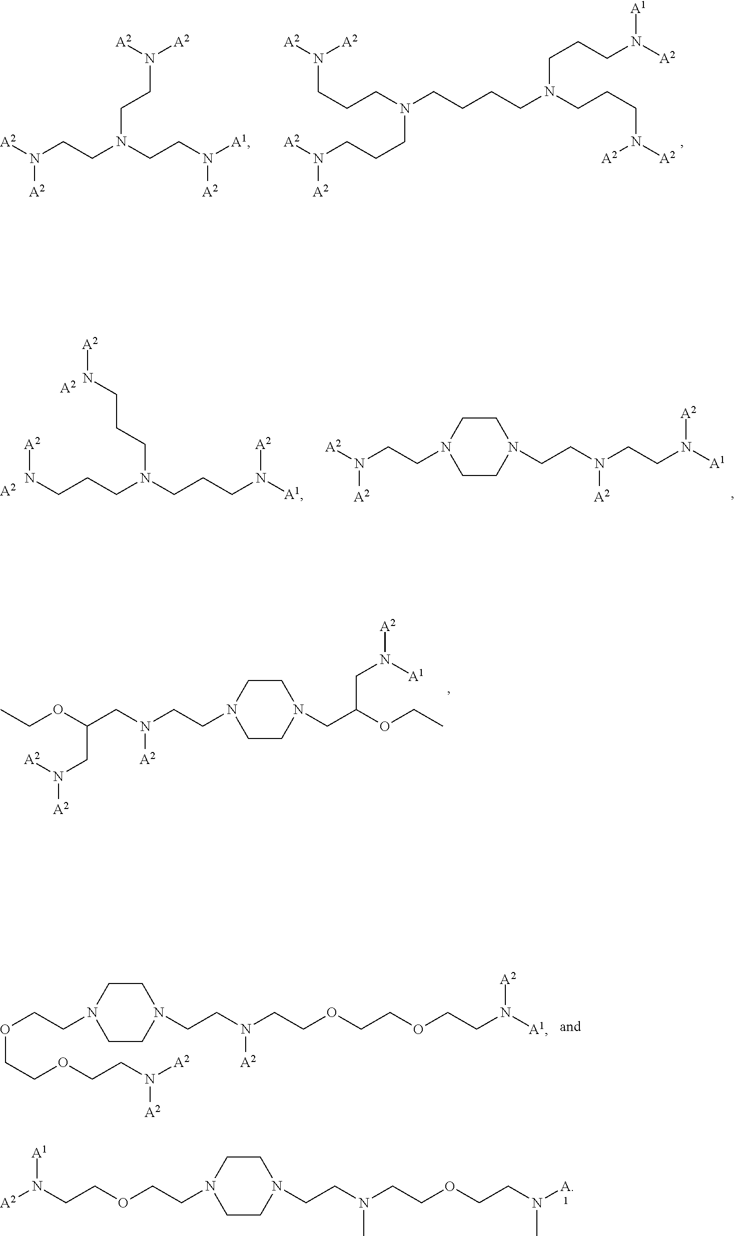

Embodiment 5 provides the LNP of Embodiment 4, wherein A¹ is

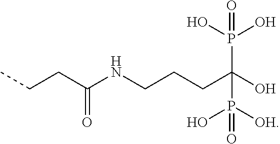

Embodiment 6 provides the LNP of Embodiment 4 or 5, wherein each occurrence of A² is independently selected from the group consisting of

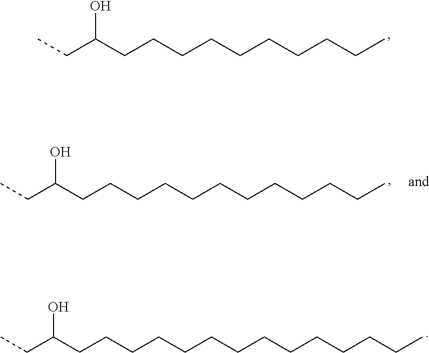

Embodiment 7 provides the LNP of any one of Embodiments 1-7, wherein the compound having the structure of Formula (I) is a compound having the structure selected from the group consisting of:

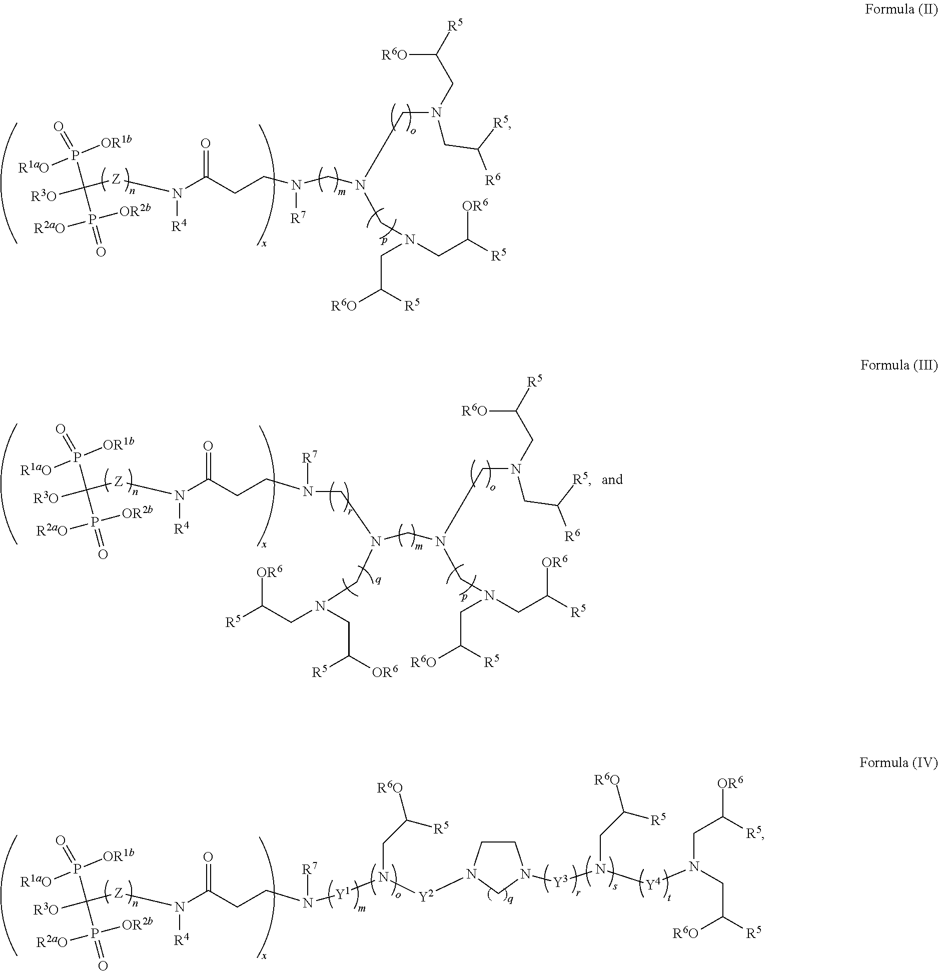

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)>>—(C₃-C₁₂ cycloalkyl), optionally substituted C₂-C₁₂ heterocycloalkyl, optionally substituted-(R⁹)z, (R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)z,(R¹⁰)_{z″}‘—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰_{z″},-—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof,
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof,
each occurrence of Y¹, Y², Y³, and Y⁴ is independently selected from the group consisting of O, S, C(R⁹)_{z′}(R¹⁰)_{z″},N(R⁹)_{z′}, P(R⁹)_{z′}, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

Embodiment 8 provides the LNP of any one of Embodiments 1-7, wherein the compound having the structure of Formula (I) is a compound having the structure selected from the group consisting of:

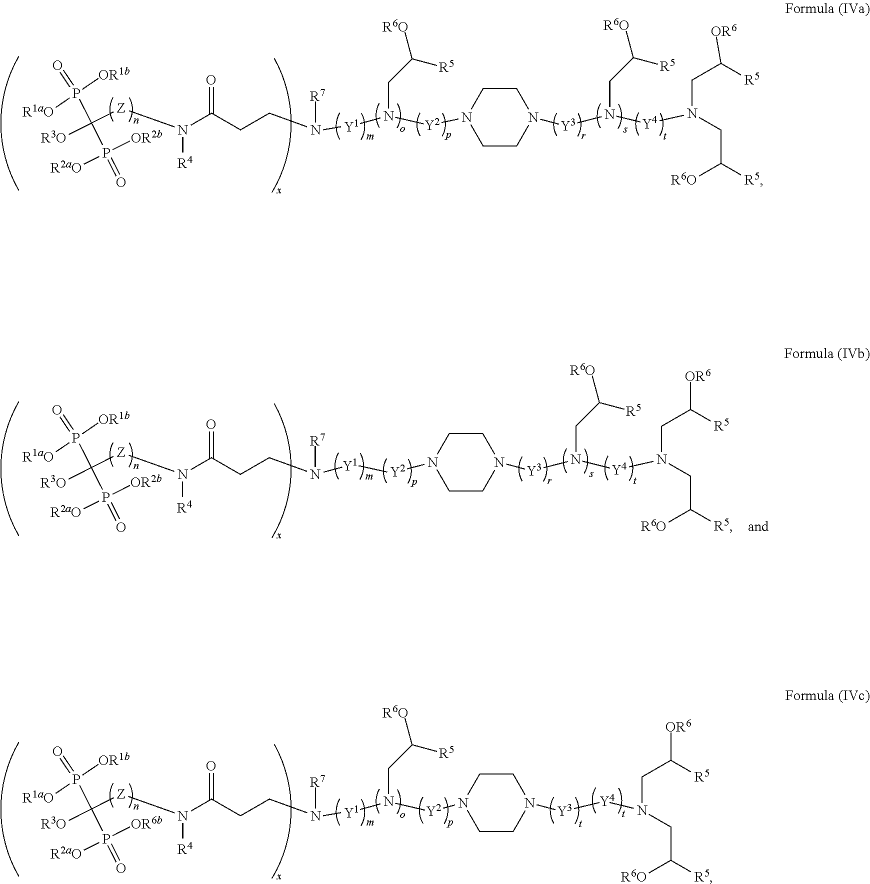

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)z,, —(C₃-C₁₂ cycloalkyl), optionally substituted C₂-C₁₂ heterocycloalkyl, optionally substituted-(R⁹)_{z′}(R¹⁰_{z″}, —(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰_{z″},-ester, —Y(R⁹)_{z′}(R¹⁰)_{z″}, —NO₂, —CN, ═O, ═S, sulfoxy, sulfonate, sulfate, sulfite, and sulfide, and any combination thereof,
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰_{z″}, —(C₃-C₁₂ cycloalkyl), optionally substituted C₂-C₁₂ heterocycloalkyl, optionally substituted-(R⁹)_{z′}(R¹⁰_{z″}, —(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰_{z″},-—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof,
each occurrence of Y¹, Y², Y³, and Y⁴ is independently selected from the group consisting of O, S, C(R⁹)_{z′}(R¹⁰)_{z″},N(R⁹)_{z′}, P(R⁹)_{z′}, and any combination thereof; each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

Embodiment 9 provides the LNP of any one of Embodiments, wherein the compound having the structure of Formula (I) is a compound having the structure selected from the group consisting of:

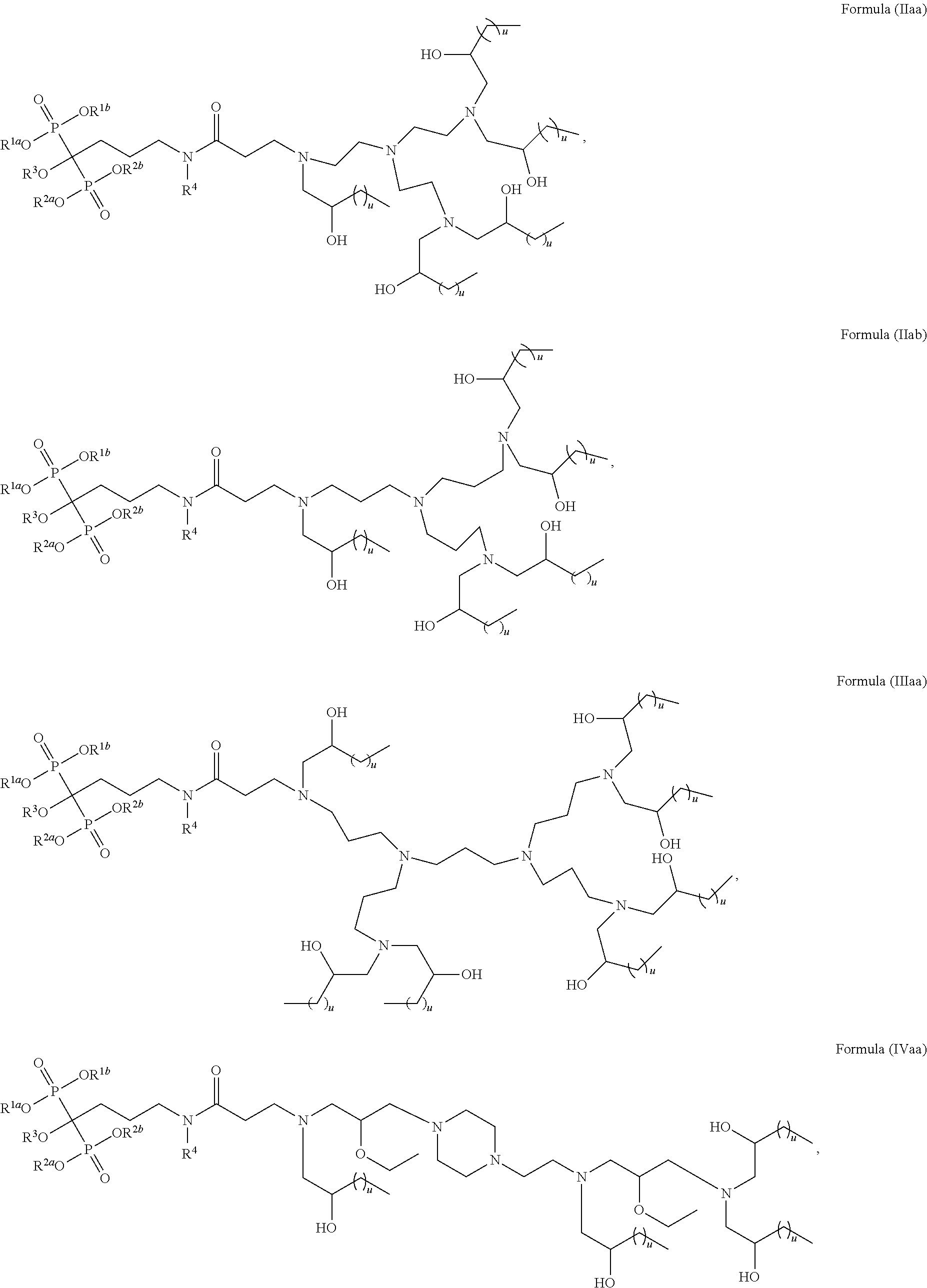

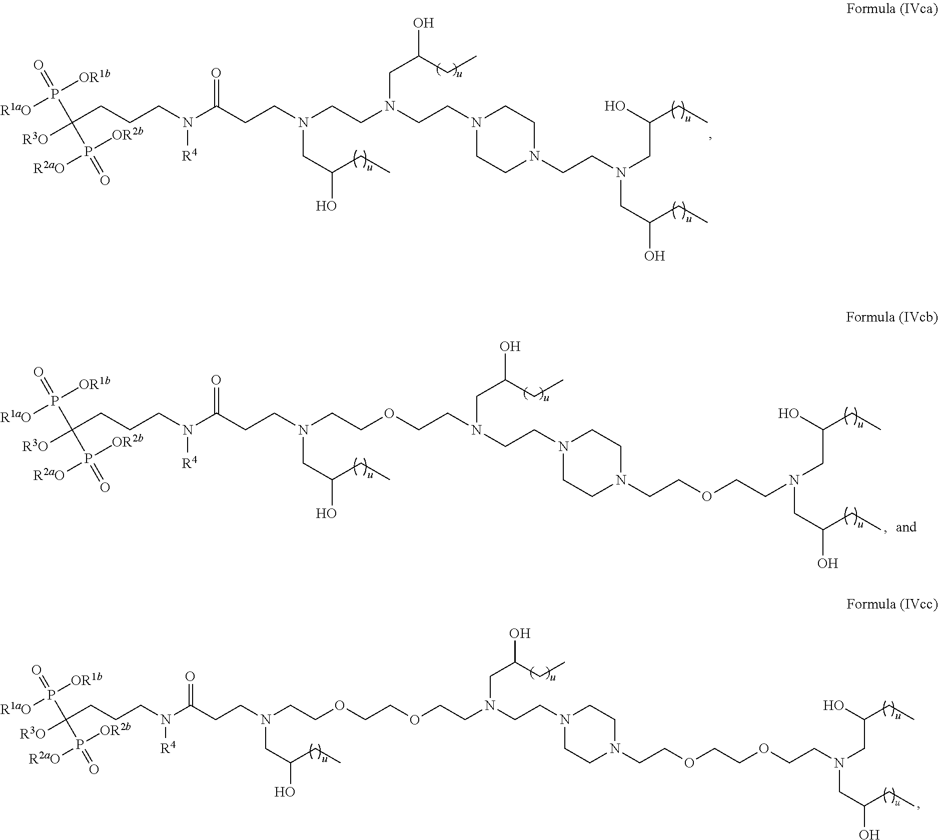

wherein:
each occurrence of R^{1a}, R^{1b}, R^{2a}, R^{2b}, R³, and R⁴ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z′}‘—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z′}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(Cs—C₁₂ cycloalkynyl), optionally substituted C₆-C₁₀ aryl, optionally substituted —Y(R⁹)_{z′}(R¹⁰_{z″}, —(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of R⁹ and R¹⁰ is independently selected from the group consisting of halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein u is an integer from 0 to 20.

Embodiment 10 provides the LNP of any one of Embodiments 1-9, wherein the compound having the structure of Formula (I) is selected from the group consisting of

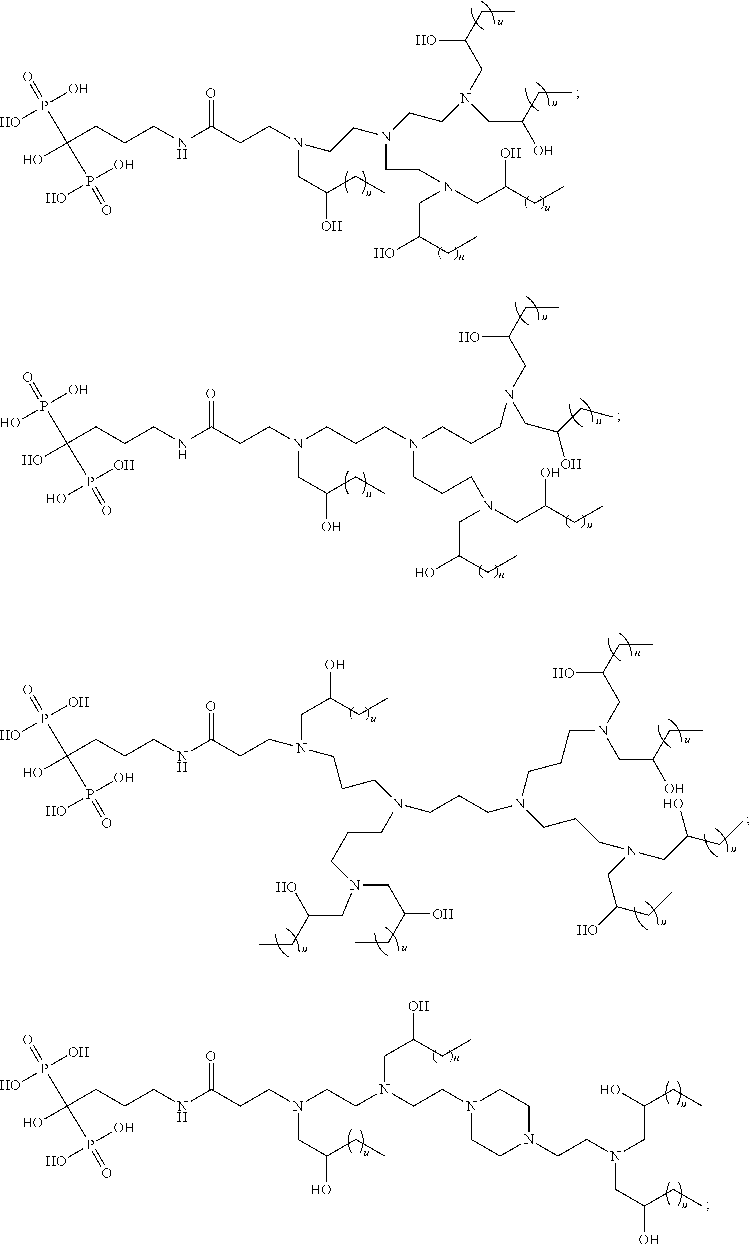

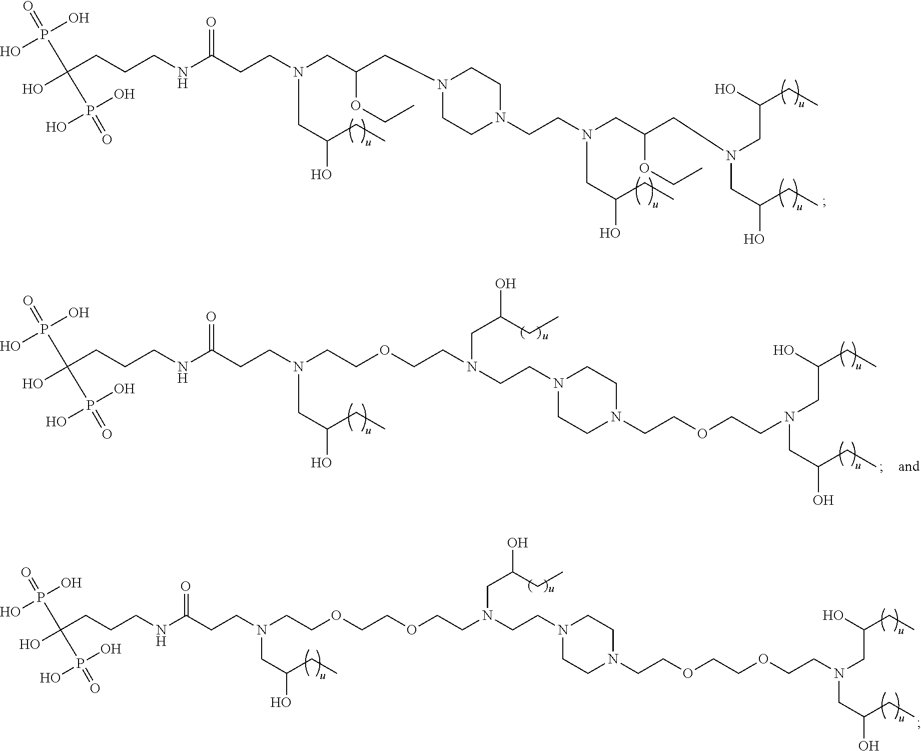

wherein u is an integer from 5 to 15.

Embodiment 11 provides the LNP of any one of Embodiments 1-10, wherein the compound having the structure of Formula (I) is:

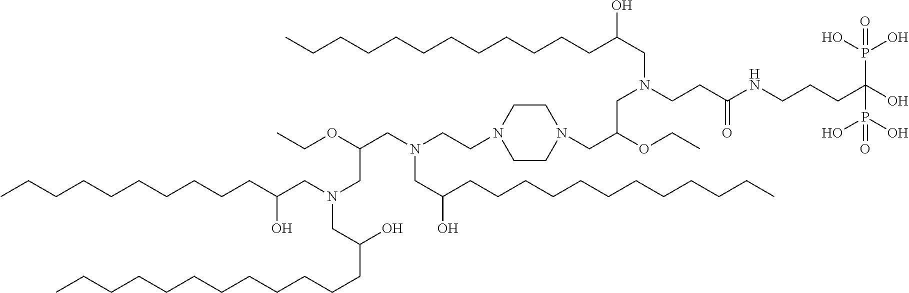 [SMILES: CCCCCCCCCCCCC(O)CN(CCC(=O)NCCCC(O)(P(=O)(O)O)P(=O)(O)O)CC(CN1CCN(CCN(CC(O)CCCCCCCCCCCC)CC(CN(CC(O)CCCCCCCCCC)CC(O)CCCCCCCCCCCC)OCC)CC1)OCC]

(4-(3-((3-(4-(2-((3-(bis(2-hydroxytetradecyl)amino)-2-ethoxypropyl)(2-hydroxytetradecyl)amino)ethyl)piperazin-1-yl)-2-ethoxypropyl)(2-hydroxytetradecyl)amino)propanamido)-1-hydroxybutane-1,1-diyl)bis(phosphonic acid).

Embodiment 12 provides the LNP of any one of Embodiments 1-11, wherein the at least one neutral phospholipid comprises at least one selected from the group consisting of dioleoyl-phosphatidylethanolamine (DOPE), dioleoylphosphatidylcholine (DOPC), distearoylphosphatidylcholine (DSPC), distearoyl-phosphatidylethanolamine (DSPE), 16-O-dimethyl PE, 18-1-trans PE, 1-stearioyl-2-oleoyl-phosphatidyethanol amine (SOPE), stearoyloleoylphosphatidylcholine (SOPC), 2-dioleoyl-3-trimethylammonium-propane (DOTAP), and any combination thereof.

Embodiment 13 provides the LNP of any one of Embodiments 1-12, wherein the at least one cholesterol lipid comprises at least one selected from the group consisting of a cholesterol, cholesteryl derivate, and any combination thereof.

Embodiment 14 provides the LNP of any one of Embodiments 1-13, wherein the at least one polymer conjugated lipid is a PEG conjugated lipid.

Embodiment 15 provides the LNP of Embodiment 14, wherein the PEG-conjugated lipid comprises at least one selected from the group consisting of 1,2-dimyristoyl-sn-glycero-3-phosphoethanolamine-N-[methoxy(polyethylene glycol)-2000](C14-PEG2000), C12-PEG2000, C12-PEG490, 1,2-dimyristoyl-rac-glycero-3-methoxypolyethylene glycol-2000 (DMG-PEG2000), 1,2-distearoyl-sn-glycero-3-phosphoethanolamine-N-[amino(polyethylene glycol)-2000](DSPE-PEG 2000 amine), and any combination thereof.

Embodiment 16 provides the LNP of any one of Embodiments 1-15, wherein the molar ratio of (a): (b): (c): (d) is about 1-80%:5-45%:5-55%:0.5-12.5%.

Embodiment 17 provides the LNP of any one of Embodiments 1-16, wherein the molar ratio of (a): (b): (c): (d) is about 35-45%:5-20%:40-55%:1-2.5%.

Embodiment 18 provides the LNP of any one of Embodiments 1-17, wherein the molar ratio of (a): (b): (c): (d) is about 30-35%:16%:46.5%:2.5%.

Embodiment 19 provides the LNP of any one of Embodiments 1-18, wherein the LNP has a diameter of between about 10 nm to about 1000 nm.

Embodiment 20 provides the LNP of any one of Embodiments 1-19, wherein the LNP has a diameter of between about 50 nm to about 500 nm.

Embodiment 21 provides the LNP of any one of Embodiments 1-20, wherein the LNP selectively binds to at least one target cell of interest.

Embodiment 22 provides the LNP of Embodiment 21, wherein the at least one target cell of interest is selected from the group consisting of a stem cell, bone cell, bone marrow cell, and any combination thereof.

Embodiment 23 provides the LNP of Embodiment 21 or 22, wherein the at least one target cell of interest is selected from the group consisting of a stem cell, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, and any combination thereof.

Embodiment 24 provides the LNP of any one of Embodiments 1-23, wherein the LNP further comprises or encapsulates at least one agent.

Embodiment 25 provides the LNP of Embodiment 24, wherein the weight ratio of (a) the at least one agent is between about 1:1 to about 10:1.

Embodiment 26 provides the LNP of Embodiment 24, wherein the agent is selected from the group consisting of a nucleic acid molecule, a small molecule, a protein, an antibody, and any combination thereof.

Embodiment 27 provides the LNP of Embodiment 26, wherein the nucleic acid molecule is a DNA molecule or an RNA molecule.

Embodiment 28 provides the LNP of Embodiment 26, wherein the nucleic acid molecule is selected from the group consisting of cDNA, mRNA, miRNA, siRNA, sgRNA, modified RNA, CRISPR-Cas9, antagomir, antisense molecule, targeted nucleic acid, and any combination thereof.

Embodiment 29 provides the LNP of Embodiment 24, wherein the LNP has a diameter of between about 10 nm to about 1000 nm.

Embodiment 30 provides the LNP of Embodiment 29, wherein the LNP has a diameter of between about 50 nm to about 500 nm.

Embodiment 31 provides the LNP of any one of Embodiments 26-30, wherein the LNP is suitable for delivering a nucleic acid to a bone cell, bone marrow cell, or a combination thereof.

Embodiment 32 provides a composition comprising at least one LNP of any one of Embodiments 1-31, optionally further comprising at least one pharmaceutically acceptable excipient.

Embodiment 33 provides the composition of Embodiment 32, wherein the composition is suitable for delivering a nucleic acid to a bone cell, bone marrow cell, or a combination thereof.

Embodiment 34 provides a method of delivering an agent to a subject in need thereof, the method comprising administering a therapeutically effectively amount of at least one LNP of any one of Embodiments 1-31 and/or the composition of Embodiment 32 or 33 the same to the subject.

Embodiment 35 provides the method of Embodiment 34, wherein the agent is selected from the group consisting of a nucleic acid molecule, a small molecule, a protein, an antibody, a therapeutic agent, and any combination thereof.

Embodiment 36 provides the method of Embodiment 35, wherein the nucleic acid molecule is a DNA molecule or an RNA molecule.

Embodiment 37 provides the method of Embodiment 35, wherein the nucleic acid molecule is selected from the group consisting of cDNA, mRNA, miRNA, siRNA, sgRNA, modified RNA, CRISPR-Cas9, antagomir, antisense molecule, targeted nucleic acid, and any combination thereof.

Embodiment 38 provides the method of Embodiment 35, wherein the LNP selectively binds to at least one target cell of interest.

Embodiment 39 provides the method of Embodiment 38, wherein the at least one target cell of interest is selected from the group consisting of a stem cell, bone cell, bone marrow cell, and any combination thereof.

Embodiment 40 provides the method of Embodiment 38 or 39, wherein the at least one target cell of interest is selected from the group consisting of a stem cell, stroma cell, osteoblast, osteocyte, osteoclast, bone lining cell, local mesenchymal cell, progenitor cell, mononuclear blood-borne precursor cell, B cell, endothelial cell, granulocytes, T cell, monocytic lineage, B cell lineage, monocytes, cancer cell, tumor cell, tumor cell that metastasize to bone, blood cancer cell, multiple myeloma cell, and any combination thereof.

Embodiment 41 provides the method of Embodiment 34, wherein the agent is encapsulated within the LNP.

Embodiment 42 provides the method of Embodiment 34, wherein the LNP or the composition comprising the same is administered intravenously, intramuscularly, intradermally, subcutaneously, intranasally, by inhalation, or any combination thereof.

Embodiment 43 provides a method of delivering an agent to a bone in a subject, bone marrow, or a combination thereof, the method comprising administering a therapeutically effectively amount of at least one LNP of any one of Embodiments 1-31 and/or the composition of Embodiment 32 or 33 to the subject.

Embodiment 44 provides the method of Embodiment 43, wherein the agent is selected from the group consisting of a nucleic acid molecule, a small molecule, a protein, an antibody, a therapeutic agent, and any combination thereof.

Embodiment 45 provides the method of Embodiment 44, wherein the nucleic acid molecule is a DNA molecule or an RNA molecule.

Embodiment 46 provides the method of Embodiment 44, wherein the nucleic acid molecule is selected from the group consisting of cDNA, mRNA, miRNA, siRNA, sgRNA, modified RNA, CRISPR-Cas9, antagomir, antisense molecule, targeted nucleic acid, and any combination thereof.

Embodiment 47 provides the method of Embodiment 43, wherein the LNP selectively binds to at least one bone cell, bone marrow cell, or a combination thereof.

Embodiment 48 provides the method of Embodiment 43, wherein the agent is encapsulated within the LNP.

Embodiment 49 provides the method of Embodiment 43, wherein the LNP or the composition comprising the same is administered intravenously, intramuscularly, intradermally, subcutaneously, intranasally, by inhalation, or any combination thereof.

Embodiment 50 provides a method of treating, ameliorating, and/or preventing at least one disease, disorder, or condition in a subject in need thereof, the method comprising administering a therapeutically effectively amount of at least one LNP of any one of Embodiments 1-31 or the composition of Embodiment 32 or 33 the same to the subject.

Embodiment 51 provides the method of Embodiment 50, wherein the disease, disorder, or condition is selected from the group consisting of bone disease or disorder, bone marrow disease or disorder, condition associated with bone, condition associated with bone marrow, bone fracture, bone defect, cancer, leukemia, lymphoma, myeloma, other blood cancers, myeloma, sickle cell disease, inflammatory disease or disorder, and any combination thereof.

Embodiment 52 provides a method of inducing a bone regeneration in a subject in need thereof, the method comprising administering a therapeutically effectively amount of at least one LNP of any one of Embodiments 1-31 and/or the composition of Embodiment 32 or 33 the same to the subject.

Embodiment 53 provides the method of Embodiment 52, wherein the subject has at least one selected from the group consisting of bone fracture, bone defect, bone cavity, root canal, and nonunion fracture.

Embodiment 54 provides a method of replacing at least one protein in a subject in need thereof, the method comprising administering a therapeutically effectively amount of at least one LNP of any one of Embodiments 1-31 and/or the composition of Embodiment 32 or 33 the same to the subject.

Embodiment 55 provides a method of gene editing in a subject in need thereof, the method comprising administering a therapeutically effectively amount of at least one LNP of any one of Embodiments 1-31 and/or the composition of Embodiment 32 or 33 the same to the subject.

Embodiment 56 provides a method of inducing an immune response in a subject in need thereof, the method comprising administering a therapeutically effectively amount of at least one LNP any one of Embodiments 1-31 and/or the composition of Embodiment 32 or 33 the same to the subject.

Embodiment 57 provides a compound having the structure of Formula (I), or a racemate, enantiomer, diastereomer, pharmaceutically acceptable salt, solvate, or derivative thereof. (A¹)ₓ(L)_{y}(A²)_{z} (I), wherein:
each occurrence of A¹ is independently

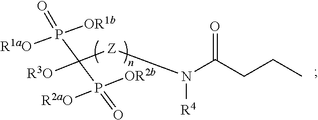

each occurrence of A² is independently

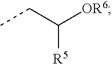

each occurrence of L is an amine linker independently selected from the group consisting of aminoalkyl linker, substituted aminoalkyl linker, diaminoalkyl linker, substituted diaminoalkyl linker, triaminoalkyl linker, substituted triaminoalkyl linker, tetraaminoalkyl linker, substituted tetraaminoalkyl linker, pentaaminoalkyl linker, substituted pentaaminoalkyl linker, polyaminoalkyl linker, substituted polyaminoalkyl linker, aminocycloalkyl linker, substituted aminocycloalkyl linker, diaminocycloalkyl linker, substituted diaminocycloalkyl linker, triaminocycloalkyl linker, substituted triaminocycloalkyl linker, tetraaminocycloalkyl linker, substituted tetraaminocycloalkyl linker, pentaaminocycloalkyl linker, substituted pentaaminocycloalkyl linker, polyaminocycloalkyl linker, substituted polyaminocycloalkyl linker, and any combination thereof;
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(Cs—C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof,
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form=O or ═S;
each occurrence of Y′ is independently selected from the group consisting of C, O, N, S, P, and Si;
each occurrence of z′ and z″ is independently an integer represented by 0, 1, or 2;
wherein x, y, and z are independently an integer from 0 to 20;
each occurrence of n is independently an integer from 0 to 10.

Embodiment 58 provides the compound of Embodiment 57, wherein L is selected from the group consisting of

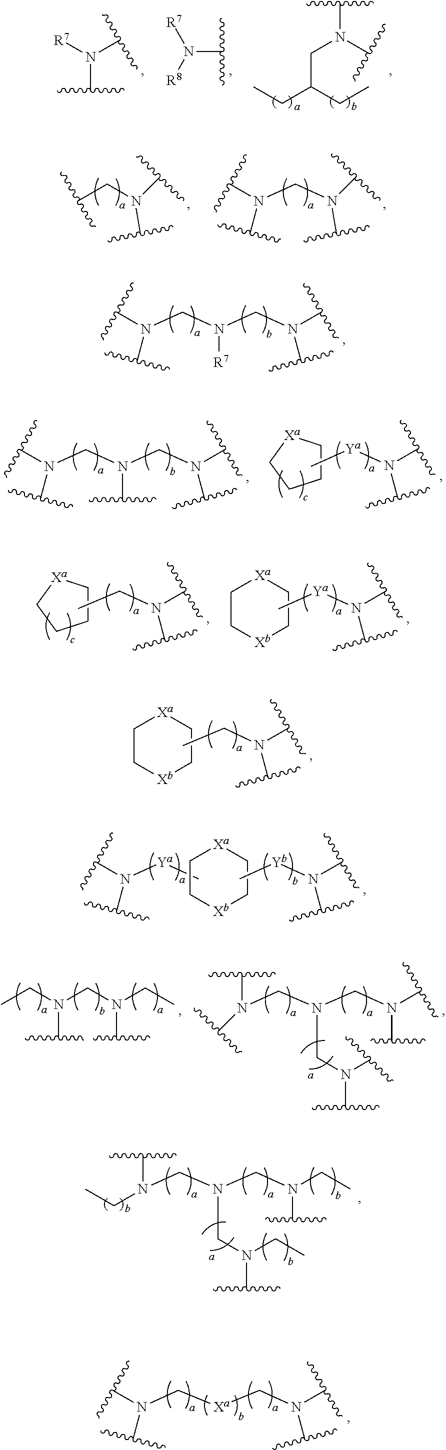

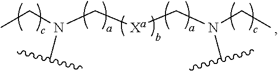

and any combination thereof,
wherein:
each occurrence of R⁷ and R⁸ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z′}—(C₃-C₁₂ cycloalkyl), optionally substituted C₂-C₁₂ heterocycloalkyl, optionally substituted-(R⁹)_{z′}(R¹⁰)_{z′}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z′}‘—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof,
each occurrence of X^{a} and X^{b} is independently selected from the group consisting of O, S, N(R⁹)_{z′}, P(R⁹)_{z′}, and any combination thereof,
each occurrence of Y^{a} and Y^{b} is independently selected from the group consisting of C1-C₁₂ alkylenyl, substituted C₁-C₁₂ alkylenyl, C₃-C₈ cycloalkylenyl, substituted C₃-C₈ cycloalkylenyl, —Y′(R⁹)_{z′}(R¹⁰)_{z″}—(C₃-C₈ cycloalkylenyl), substituted —Y′(R⁹)_{z′}(R¹⁰)_{z″}—C₃-C₈ cycloalkylenyl, C₂-C₈ heterocycloalkylenyl, substituted C₂-C₈ heterocycloalkylenyl, —Y′(R⁹)_{z′}(R¹⁰)_{z″}—C₂-C₈ heterocycloalkylenyl, substituted-Y′(R⁹)_{z′}(R¹⁰)_{z″}—C₂-C₈ heterocycloalkylenyl, C₂-C₈ alkenylenyl, substituted C₂-C₈ alkenylenyl, C₅-C₁₀ cycloalkenylenyl, substituted C₅-C₁₀ cycloalkenylenyl, —Y′(R⁹)_{z′}(R¹⁰)_{z″}—C₅-C₁₀ cycloalkenylenyl, substituted —Y′(R⁹)_{z′}(R¹⁰)_{z″}—C₅-C₁₀ cycloalkenylenyl, C₂-C₈ alkynylenyl, substituted C₂-C₈ alkynylenyl, C₈-C₁₂ cycloalkynylenyl, substituted C₈-C₁₂ cycloalkynylenyl, —Y′(R⁹)_{z′}(R¹⁰)_{z″}—C₈-C₁₂ cycloalkynylenyl, substituted —Y′(R⁹)_{z′}(R¹⁰)_{z″}—C₈-C₁₂ cycloalkynylenyl, C₆-C₁₀ arylenyl, substituted C₆-C₁₀ arylenyl, —Y′(R⁹)_{z′}(R¹⁰)_{z″}—C₆-C₁₀ arylenyl, substituted —Y′(R⁹)_{z′}(R¹⁰)_{z″}—C₆-C₁₀ arylenyl, C₂-C₁₀ heteroarylenyl, substituted C₂-C₁₀ heteroarylenyl, —Y′(R⁹)_{z′}(R¹⁰)_{z″}—C₂-C₁₀ heteroarylenyl, and substituted —Y′(R⁹)_{z′}(R¹⁰)_{z″}—C₂-C₁₀ heteroarylenyl;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2;
wherein each occurrence of a, b, and c is independently an integer from 0 to 10;
each occurrence of

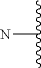 [SMILES: *N]

indicates a bond between a N atom of L and A¹ or A².

Embodiment 59 provides the compound of Embodiment 57 or 58, wherein L is selected from the group consisting of

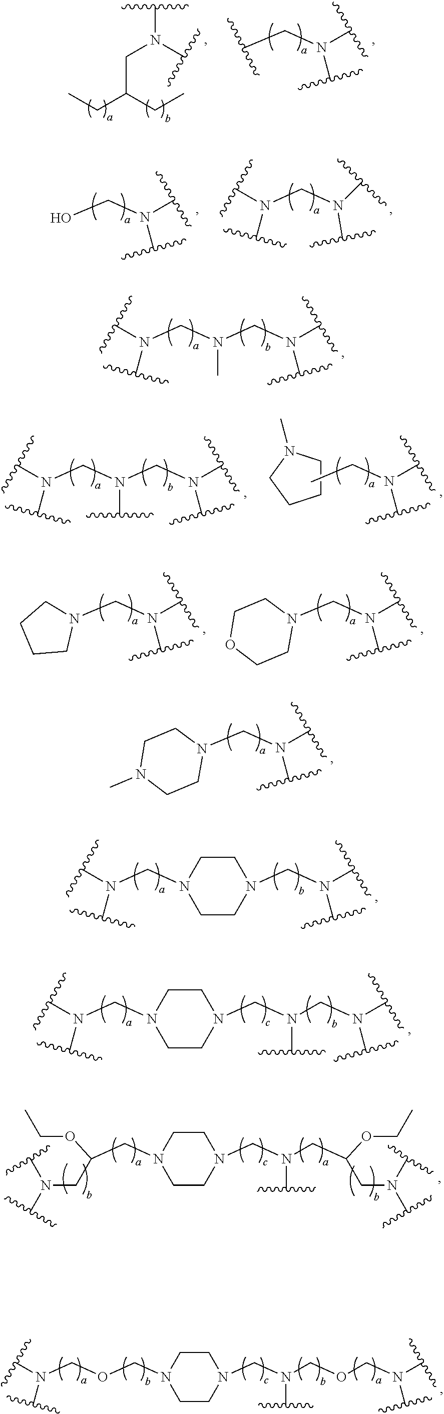

any combination thereof,
wherein:
each occurrence of a, b, and c is independently an integer from 0 to 10; and
each occurrence of

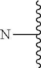 [SMILES: *N]

indicates a bond between a N atom of L and A¹ or A².

Embodiment 60 provides the compound of any one of Embodiments 57-59, wherein the compound having the structure of Formula (I) is a compound having the structure selected from the group consisting of:

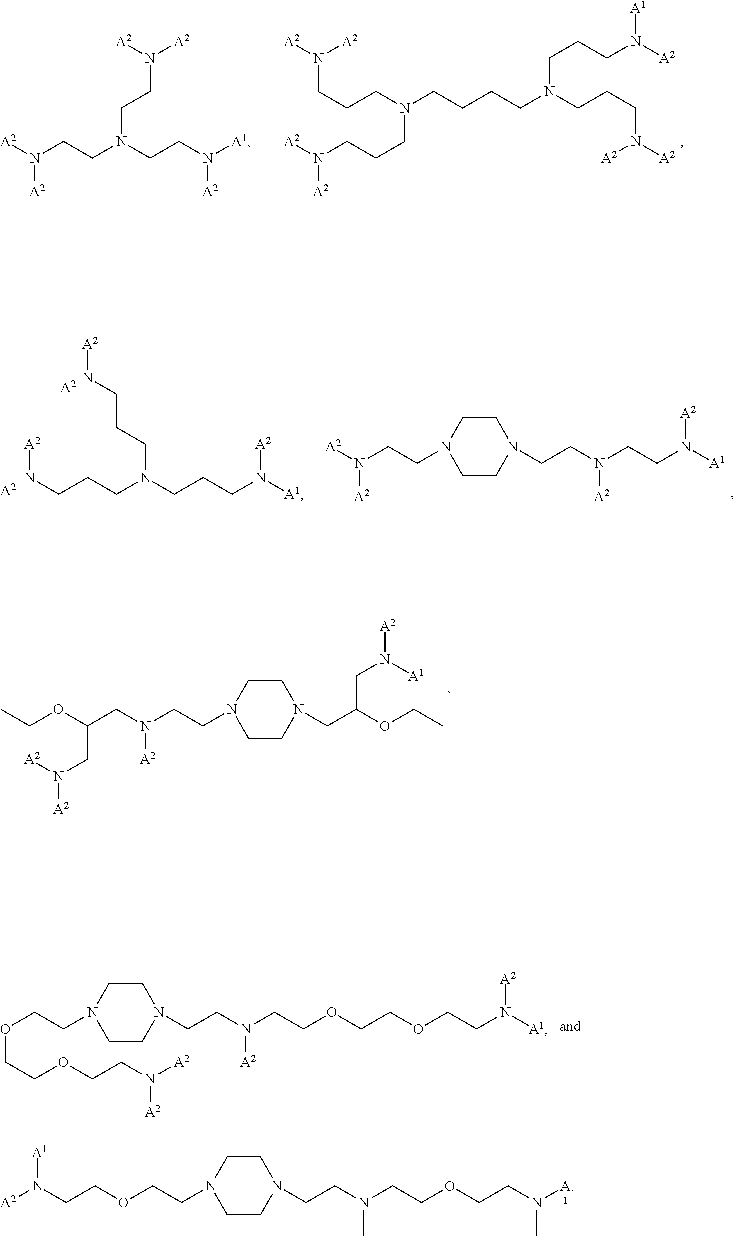

Embodiment 61 provides the compound of Embodiment 60, wherein A¹ is

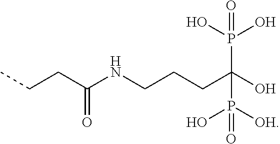

Embodiment 62 provides the compound of Embodiment 60 or 61, wherein each occurrence of A² is independently selected from the group consisting of

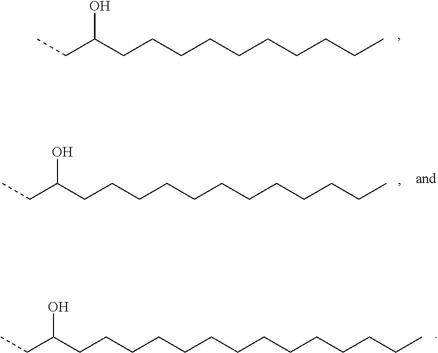

Embodiment 63 provides the compound of any one of Embodiments 57-62, wherein the compound having the structure of Formula (I) is a compound having the structure selected from the group consisting of:

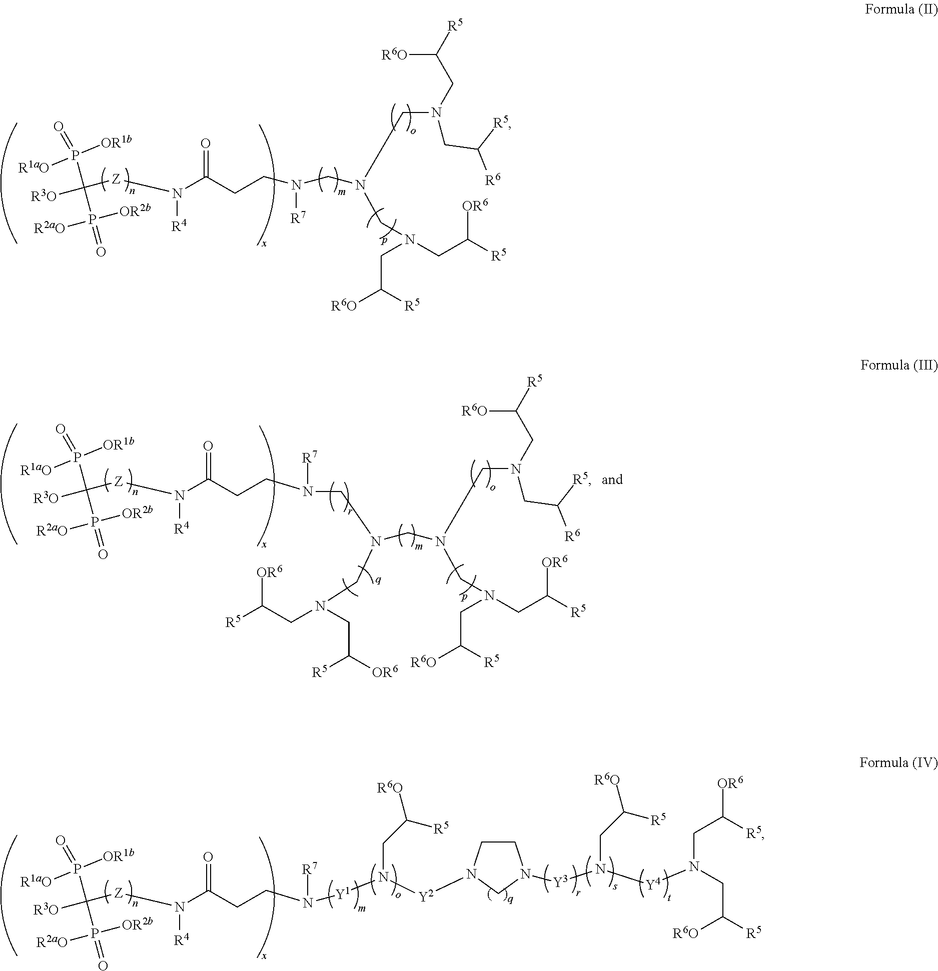

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R¹, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰_{z″},-—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof,
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof,
each occurrence of Y¹, Y², Y³, and Y⁴ is independently selected from the group consisting of O, S, C(R⁹)_{z′}(R¹⁰)_{z″},N(R⁹)_{z′}, P(R⁹)_{z′}, and any combination thereof;
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

Embodiment 64 provides the compound of any one of Embodiments 57-63, wherein the compound having the structure of Formula (I) is a compound having the structure selected from the group consisting of:

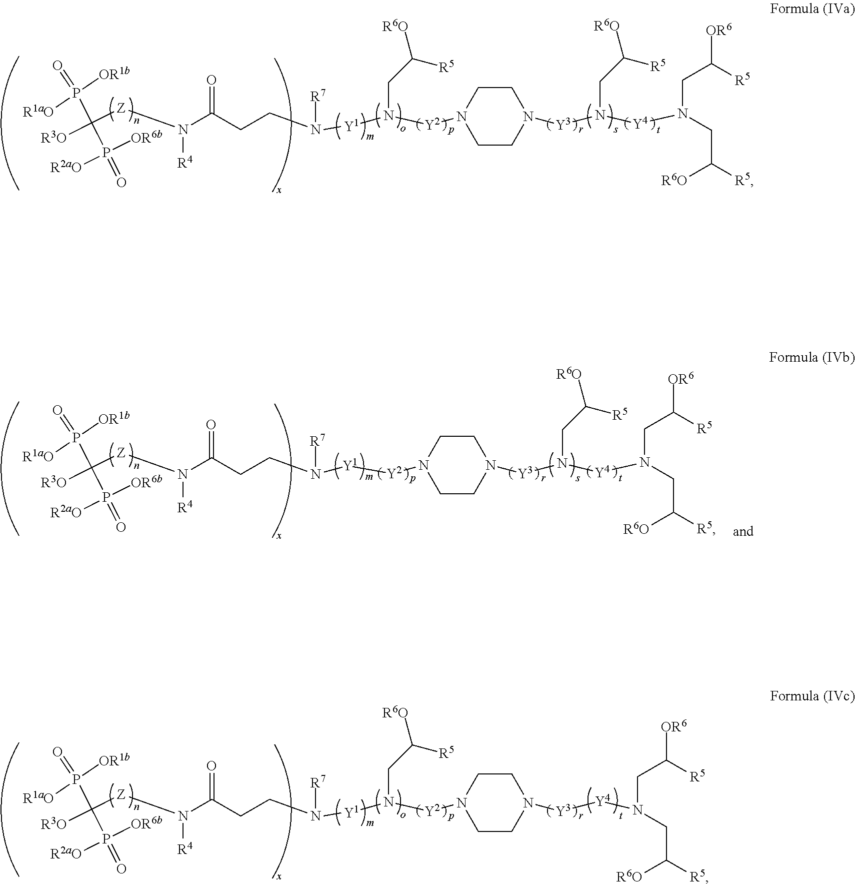

wherein:
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰_{z″}, —(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰_{z″},-ester, —Y(R⁹)_{z′}(R¹⁰)_{z″}, —NO₂, —CN, ═O, ═S, sulfoxy, sulfonate, sulfate, sulfite, and sulfide, and any combination thereof,
R⁷ is selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰_{z″}, —(C₃-C₁₂ cycloalkyl), optionally substituted C₂-C₁₂ heterocycloalkyl, optionally substituted-(R⁹)_{z′}(R¹⁰_{z″}, —(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₈-C₁₂ cycloalkynyl, optionally substituted —Y(R⁹)_{z′}(R¹⁰_{z″},-—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, sulfoxy, sulfonate, sulfate, sulfite, and sulfide, and any combination thereof,
each occurrence of Y¹, Y², Y³, and Y⁴ is independently selected from the group consisting of O, S, C(R⁹)_{z′}(R¹⁰)_{z″},N(R⁹)_{z′}, P(R⁹)_{z′}, and any combination thereof; each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein x is an integer from 0 to 20;
wherein m, o, p, q, r, s, and t are independently an integer from 0 to 10; and
each occurrence of n is independently an integer from 0 to 5.

Embodiment 65 provides the compound of any one of Embodiments 57-64, wherein the compound having the structure of Formula (I) is a compound having the structure selected from the group consisting of:

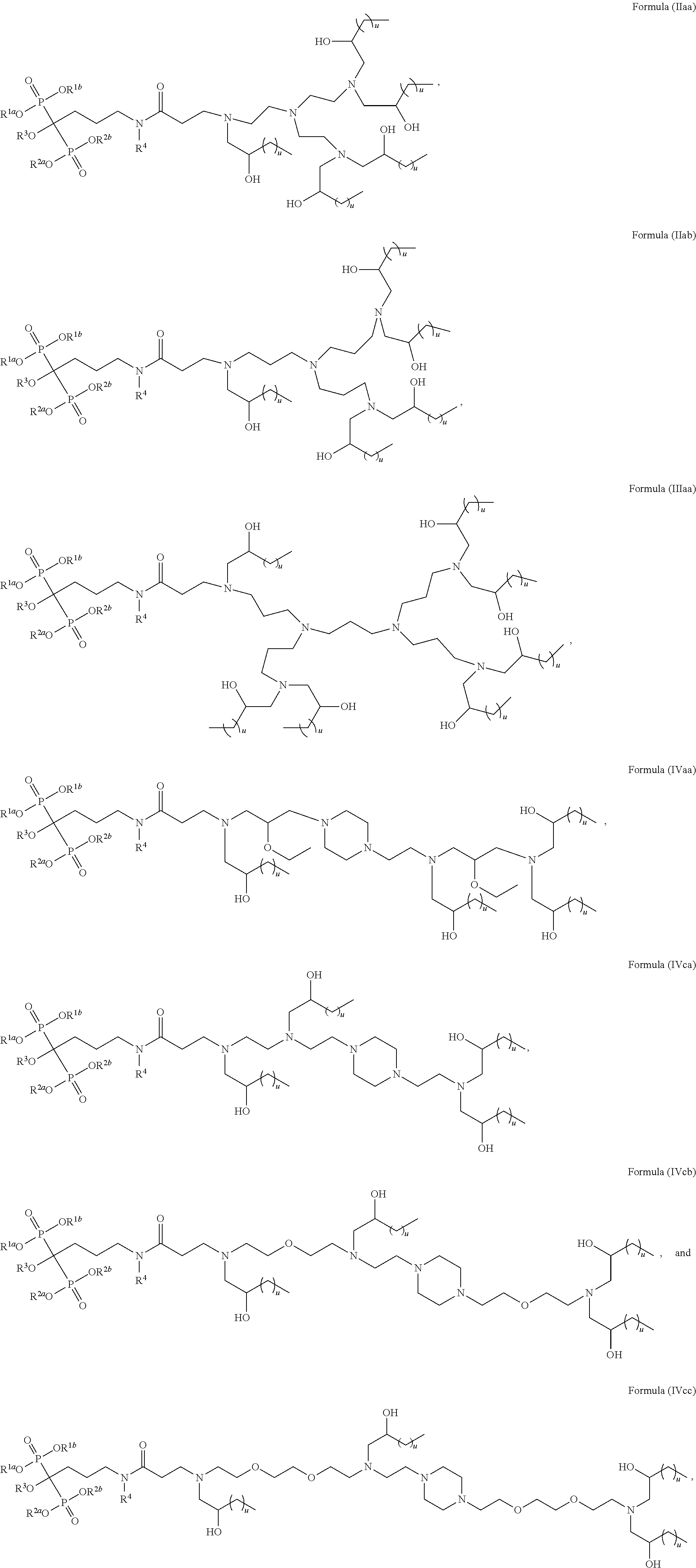

wherein:
each occurrence of R^{1a}, R^{1b}, R^{2a}, R^{2b}, R³, and R⁴ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}—(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof;
each occurrence of R⁹ and R¹⁰ is independently selected from the group consisting of halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si; and
each occurrence of z′ and z″ is independently an integer represented by 0, 1, or 2; and
wherein u is an integer from 0 to 20.

Embodiment 66 provides the compound of any one of Embodiments 57-64, wherein the compound having the structure of Formula (I) is selected from the group consisting of

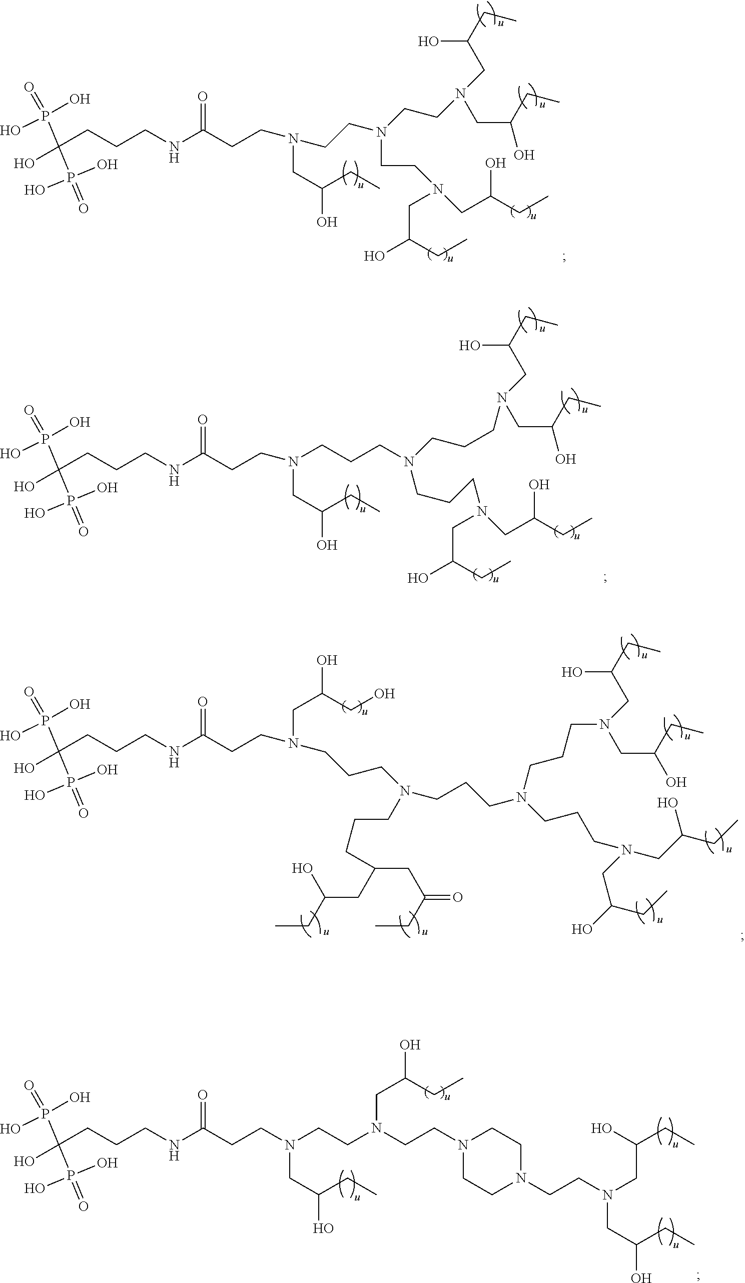

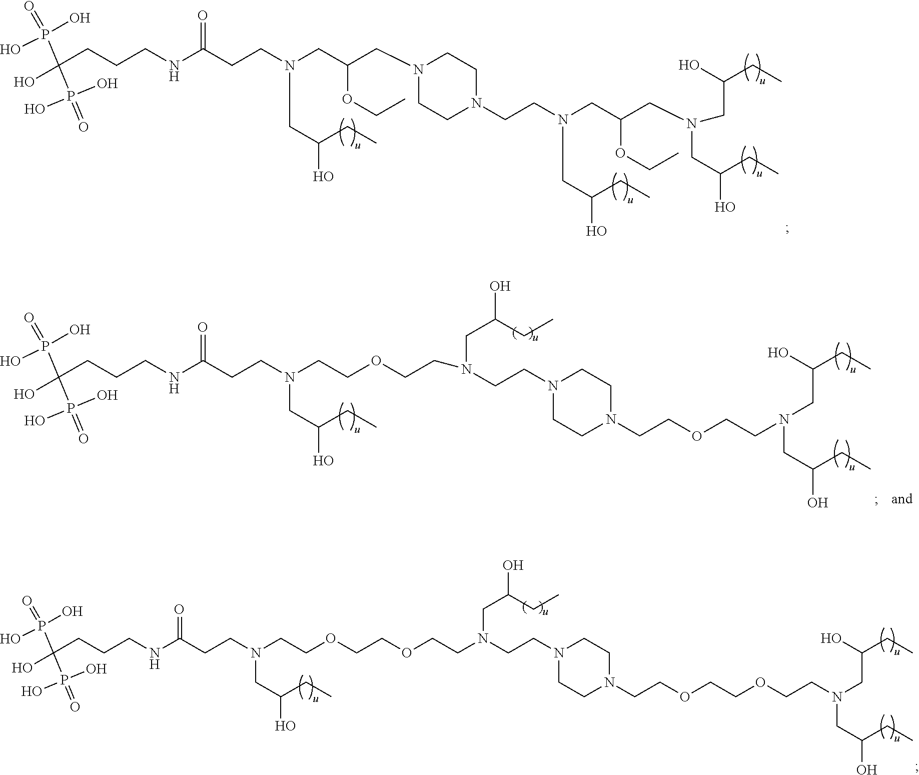

wherein u is an integer from 5 to 15.

Embodiment 67 provides the compound of any one of Embodiments 57-66, wherein the compound having the structure of Formula (I) is:

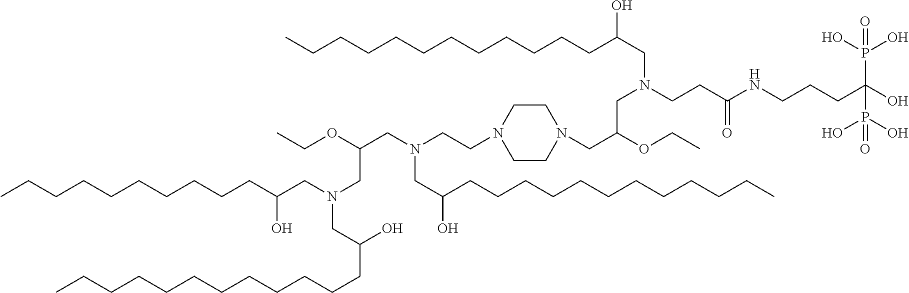 [SMILES: CCCCCCCCCCCCC(O)CN(CCC(=O)NCCCC(O)(P(=O)(O)O)P(=O)(O)O)CC(CN1CCN(CCN(CC(O)CCCCCCCCCCCC)CC(CN(CC(O)CCCCCCCCCC)CC(O)CCCCCCCCCCCC)OCC)CC1)OCC]

(4-(3-((3-(4-(2-((3-(bis(2-hydroxytetradecyl)amino)-2-ethoxypropyl)(2-hydroxytetradecyl)amino)ethyl)piperazin-1-yl)-2-ethoxypropyl)(2-hydroxytetradecyl)amino)propanamido)-1-hydroxybutane-1,1-diyl)bis(phosphonic acid).

Embodiment 68 provides a compound of Formula (V), or a salt, stereoisomer, or isotopologue thereof:

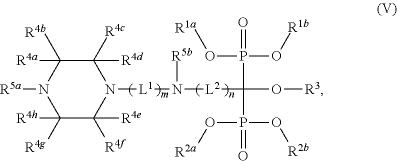

wherein:
R^{1a}, R^{1b}, R^{2a}, R^{2b}, and R³ are each independently selected from the group consisting of H, C(═O)R^{A}, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
R^{4a}, R^{4b}, R^{4c}, R^{4d}, R^{4e}, R^{4f}, R^{4g}, and R^{4h} are each independently selected from the group consisting of H, halogen, and optionally substituted C₁-C₆ alkyl;
R^{5a} and R^{5b} are each independently selected from the group consisting of H, optionally substituted C₁-C₂₄ alkyl, —C(═O)(optionally substituted C₁-C₂₄ alkyl), —C(═O)O(optionally substituted C₁-C₂₄ alkyl), and R⁶,
   wherein at least one of R^{5a} and R^{5b} is R⁶;
each occurrence of R⁶ is independently

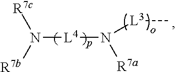

or two occurrences of R⁶ can combine with the atoms to which they are bound to form

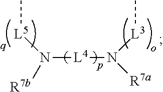

each occurrence of L¹, L², L³, L⁵, and L⁶, if present, is independently selected from the group consisting of -(optionally substituted C₁-C₃ alkylenyl)-, —C(═O)—, —O—, and —N(R^{A})—;
each occurrence of L⁴ is independently selected from the group consisting of —X—, -(optionally substituted C₁-C₁₂ alkylenyl)-, -(optionally substituted C₂-C₁₂ alkenylenyl)-, -(optionally substituted C₁-C₁₂ alkynylenyl)-, -(optionally substituted C₁-C₁₂ heteroalkylenyl)-, -(optionally substituted C₃-C₈ cycloalkylenyl)-, -(optionally substituted C₂-C₈ heterocyloalkylenyl)-, -(optionally substituted C₆-C₁₀ arylenyl)-, and -(optionally substituted C₂-C₈ heteroarylenyl)-;
each occurrence of X, if present, is independently selected from the group consisting of —N(R^{7d})—, —N(R⁸)—, —C(═O)—, and —O—;
each occurrence of R^{7a}, R^{7b}, R^{7c}, R^{7d}, R^{7e}, and R^{7f}, if present, are each independently selected from the group consisting of H, optionally substituted C₁-C₂₄ alkyl and optionally substituted C₁-C₂₄ heteroalkyl;
each occurrence of R⁸ is independently

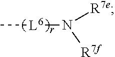

each occurrence of m and n, o, p, q, and r, if present, are each independently an integer selected from the group consisting of 1, 2, 3, 4, 5, 6, 7, 8, 9, and 10; and
each occurrence of R^{A} is independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

Embodiment 69 provides the compound of Embodiment 68, wherein:
(a) at least one of R^{1a}, R^{1b}, R^{2a}, R^{2b}, and R³ is H;
(b) at least two of R^{1a}, R^{1b}, R^{2a}, R^{2b}, and R³ are H;
(c) at least three of R^{1a}, R^{1b}, R^{2a}, R^{2b}, and R³ are H;
(d) at least four of R^{1a}, R^{1b}, R^{2a}, R^{2b}, and R³ are H; and
(e) each of R^{1a}, R^{1b}, R^{2a}, R^{2b}, and R³ are H.

Embodiment 70 provides the compound of Embodiment 68 or 69, wherein each occurrence of Li is independently selected from the group consisting of —C(═O)— and —(CH₂)—.

Embodiment 71 provides the compound of any one of Embodiments 68-70, wherein -(L¹)ₘ- is selected from the group consisting of —C(═O)(CH₂)₂— and —C(═O)(CH₂)₂C(=O)—.

Embodiment 72 provides the compound of any one of Embodiments 68-71, wherein each occurrence of L² is independently —(CH₂)—.

Embodiment 73 provides the compound of any one of Embodiments 68-72, wherein -(L²)n- is —(CH₂)₃—.

Embodiment 74 provides the compound of any one of Embodiments 68-73, wherein at least one of the following applies:
(a) at least one of R^{4a}, R^{4b}, R⁴c, R^{4d}, R⁴e, R^{4f}, R⁴⁹, and R⁴h is H;
(b) at least two of R^{4a}, R^{4b}, R⁴c, R^{4d}, R⁴e, R^{4f}, R⁴⁹, and R⁴h are H;
(c) at least three of R^{4a}, R^{4b}, R⁴c, R^{4d}, R⁴e, R^{4f}, R⁴⁸, and R⁴h are H;
(d) at least four of R^{4a}, R^{4b}, R⁴c, R^{4d}, R⁴e, R^{4f}, R^{4g}, and R⁴h are H;
(e) at least five of R^{4a}, R^{4b}, R⁴c, R^{4d}, R⁴e, R^{4f}, R^{4g}, and R⁴h are H;
(f) at least six of R^{4a}, R^{4b}, R⁴c, R^{4d}, R⁴e, R^{4f}, R^{4g}, and R⁴h are H;
(g) at least seven of R^{4a}, R^{4b}, R⁴c, R^{4d}, R⁴e, R^{4f}, R⁴⁸, and R⁴h are H; and
(h) each of R^{4a}, R^{4b}, R⁴c, R^{4d}, R^{4e}, R^{4f}, R^{4g},and R⁴h is H.

Embodiment 75 provides the compound of any one of Embodiments 68-74, wherein R^{5a} and R^{5b} are each independently selected from the group consisting of H, C(═O)O(optionally substituted C₁-C₁₂ alkyl), optionally substituted C₁-C₁₂ alkyl, and R⁶.

Embodiment 76 provides the compound of any one of Embodiments 68-75, wherein each occurrence of L³ and L⁵ is independently selected from the group consisting of —C(═O)— and —(CH₂)—.

Embodiment 77 provides the compound of any one of Embodiments 68-76, wherein each occurrence of -(L³)-₀ and -(L⁵)q- is —C(═O)(CH₂)—.

Embodiment 78 provides the compound of any one of Embodiments 68-77, wherein each occurrence of L⁴ is independently selected from the group consisting of —(CH₂)₁₋₃—, —O—, —N(R^{7d})—, —N(R⁸)—, and

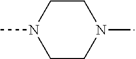

Embodiment 79 provides the compound of any one of Embodiments 68-78, wherein each occurrence of L⁶ is independently —(CH₂)₁₋₃—.

Embodiment 80 provides the compound of any one of Embodiments 68-79, wherein each occurrence of R⁶ is independently selected from the group consisting of

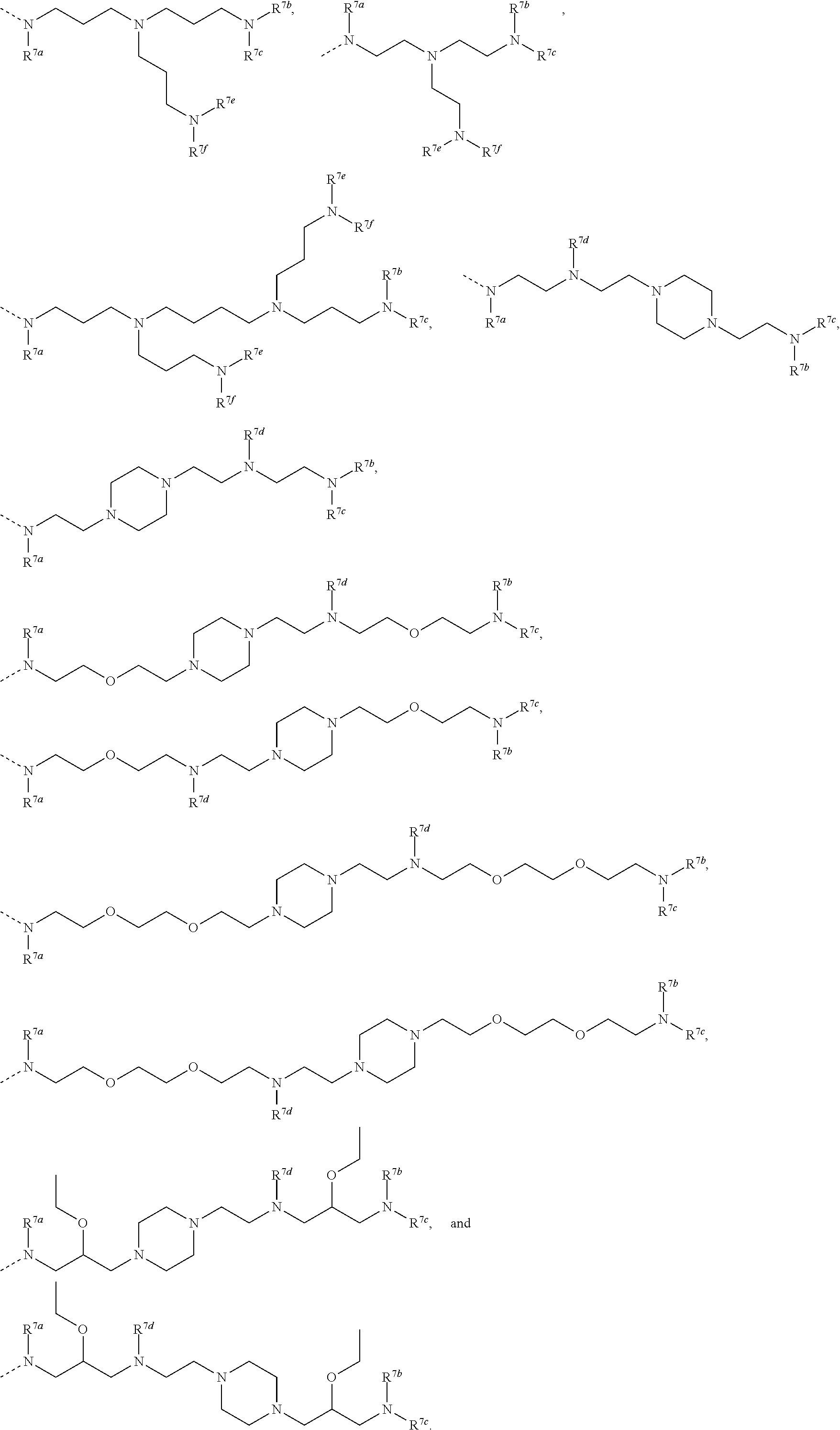

Embodiment 81 provides the compound of any one of Embodiments 68-79, wherein two occurrences of R⁶ combine with the atoms to which they are bound to form a moiety selected from the group consisting of

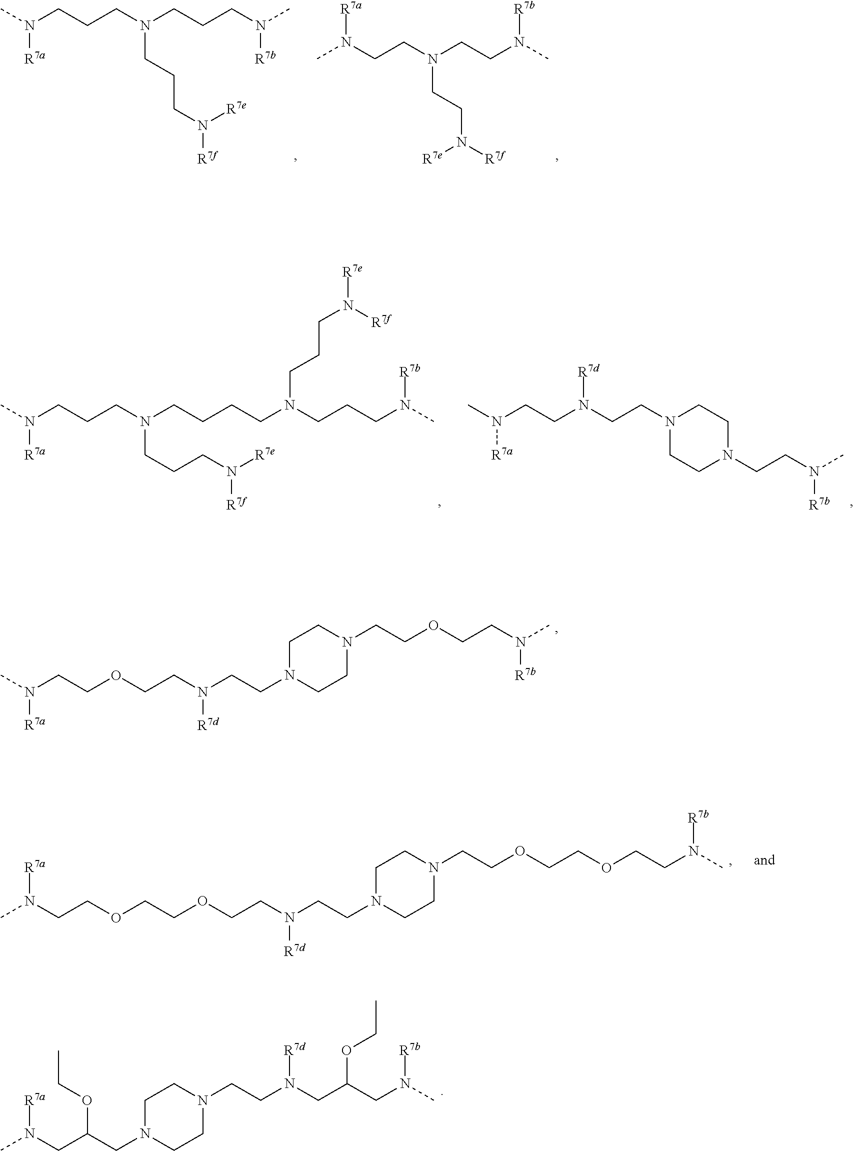

Embodiment 82 provides the compound of any one of Embodiments 68-81, wherein each occurrence of R^{7a}, R^{7b}, R^{7c}, R^{7d}, R^{7c}, and R^{7f}, if present, is independently selected from the group consisting of —(CH₂)CH(OH)(optionally substituted C₁-C₂₂ alkyl).

Embodiment 83 provides the compound of any one of Embodiments 68-82, wherein each occurrence of R^{7a}, R^{7b}, R^{7c}, R^{7d}, R^{7c}, and R^{7f}, if present, is independently selected from the group consisting of

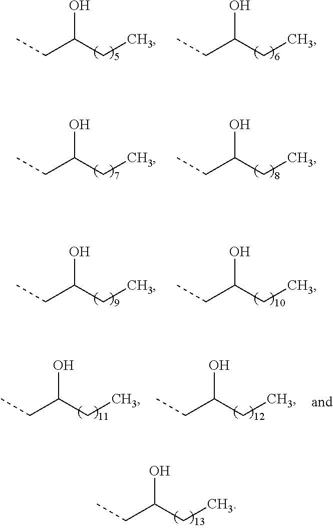

CH₃ CH₃ CH₃ CH₃ CH₃

Embodiment 84 provides the compound of any one of Embodiments 68-83, wherein the compound is selected from the group consisting of Type1-B1-C10, Type1-B1-C12, Type1-B1-C14, Type1-B1-C16, Type1-B2-C10, Type1-B2-C12, Type1-B2-C14, Type1-B2-C16, Type1-B3-C10, Type1-B3-C12, Type1-B3-C14, Type1-B3-C16, Type1-P1-C10, Type1-P1-C12, Type1-P1-C14, Type1-P1-C16, Type1-P2-C10, Type1-P2-C12, Type1-P2-C14, Type1-P2-C16, Type1-P3-C10, Type1-P3-C12, Type1-P3-C14, Type1-P3-C16, Type1-P4-C10, Type1-P4-C12, Type1-P4-C14, Type1-P4-C16, Type2-B1-C10, Type2-B1-C12, Type2-B1-C14, Type2-B1-C16, Type2-B2-C10, Type2-B2-C12, Type2-B2-C14, Type2-B2-C16, Type2-B3-C10, Type2-B3-C12, Type2-B3-C14, Type2-B3-C16, Type2-P1-C10, Type2-P1-C12, Type2-P1-C14, Type2-P1-C16, Type2-P2-C10, Type2-P2-C12, Type2-P2-C14, Type2-P2-C16, Type2-P3-C10, Type2-P3-C12, Type2-P3-C14, Type2-P3-C16, Type2-P4-C10, Type2-P4-C12, Type2-P4-C14, Type2-P4-C16, Type3-B1-C10, Type3-B1-C12, Type3-B1-C14, Type3-B1-C16, Type3-B2-C10, Type3-B2-C12, Type3-B2-C14, Type3-B2-C16, Type3-B3-C10, Type3-B3-C12, Type3-B3-C14, Type3-B3-C16, Type3-P1-C10, Type3-P1-C12, Type3-P1-C14, Type3-P1-C16, Type3-P2-C10, Type3-P2-C12, Type3-P2-C14, Type3-P2-C16, Type3-P3-C10, Type3-P3-C12, Type3-P3-C14, Type3-P3-C16, Type3-P4-C10, Type3-P4-C12, Type3-P4-C14, Type3-P4-C16, Type4-B1-C10, Type4-B1-C12, Type4-B1-C14, Type4-B1-C16, Type4-B2-C10, Type4-B2-C12, Type4-B2-C14, Type4-B2-C16, Type4-B3-C10, Type4-B3-C12, Type4-B3-C14, Type4-B3-C16, Type4-P1-C10, Type4-P1-C12, Type4-P1-C14, Type4-P1-C16, Type4-P2-C10, Type4-P2-C12, Type4-P2-C14, Type4-P2-C16, Type4-P3-C10, Type4-P3-C12, Type4-P3-C14, Type4-P3-C16, Type4-P4-C10, Type4-P4-C12, Type4-P4-C14, Type4-P4-C16, Type5-B1-C10, Type5-B1-C12, Type5-B1-C14, Type5-B1-C16, Type5-B2-C10, Type5-B2-C12, Type5-B2-C14, Type5-B2-C16, Type5-B3-C10, Type5-B3-C12, Type5-B3-C14, Type5-B3-C16, Type5-P1-C10, Type5-P1-C12, Type5-P1-C14, Type5-P1-C16, Type5-P2-C10, Type5-P2-C12, Type5-P2-C14, Type5-P2-C16, Type5-P3-C10, Type5-P3-C12, Type5-P3-C14, Type5-P3-C16, Type5-P4-C10, Type5-P4-C12, Type5-P4-C14, and Type5-P4-C16.

Embodiment 85 provides a lipid nanoparticle (LNP) composition comprising:
(a) at least one ionizable lipid, wherein the at least one ionizable lipid comprises at least one compound of formula (V) of any one of Embodiments 68-84;
(b) at least one neutral lipid;
(c) at least one cholesterol lipid and/or a modified derivative thereof; and
(d) at least one polymer-conjugated lipid and/or a modified derivative thereof.

Embodiment 86 provides the LNP of Embodiment 85, wherein the at least one ionizable lipid compound comprises about 10 mol % to about 90 mol % of the LNP, optionally wherein the at least one ionizable lipid compound comprises about 35 mol % of the LNP.

Embodiment 87 provides the LNP of any one of Embodiment 85 or 86, wherein the ionizable lipid further comprises C12-200.

Embodiment 88 provides the LNP of Embodiment 87, wherein the at least one ionizable lipid compound of formula (V) and the C12-200 have a ratio ranging from about 10:1 to about 1:10 (formula (V):C12:200), optionally wherein the at least one ionizable lipid compound of formula (V) and the C12-200 have a ratio of about 2:3 (40% formula (V)).

Embodiment 89 provides the LNP of any one of Embodiments 85-88, wherein the at least one neutral lipid comprises about 1 mol % to about 40 mol % of the LNP, optionally wherein the at least one neutral lipid comprises about 16 mol % of the LNP.

Embodiment 90 provides the LNP of any one of Embodiments 85-89, wherein the neutral lipid comprises or consists essentially of at least one neutral lipid selected from the group consisting of dioleoylphosphatidylethanolamine (DOPE), distearoylphosphatidylcholine (DSPC), and dioleoylphosphatidylcholine (DOPC), optionally wherein the neutral lipid comprises or consists essentially of dioleoylphosphatidylethanolamine (DOPE).

Embodiment 91 provides the LNP of any one of Embodiments 85-90, wherein the at least one cholesterol lipid and/or modified derivative thereof comprises about 20 mol % to about 75 mol % of the LNP, optionally wherein the at least one cholesterol lipid and/or modified derivative thereof comprises about 46.5 mol % of the LNP.

Embodiment 92 provides the LNP of any one of Embodiments 85-91, wherein the at least one cholesterol lipid and/or modified derivative thereof comprises or consists essentially of cholesterol.

Embodiment 93 provides the LNP of any one of Embodiments 85-92, wherein the at least one polymer-conjugated lipid comprises about 0.1 mol % to about 15 mol % of the LNP, optionally wherein the at least one polymer-conjugated lipid comprises about 2.5 mol %.

Embodiment 94 provides the LNP of any one of Embodiments 85-93, wherein the at least one polymer-conjugated lipid comprises or consists essentially of C14-PEG2000.

Embodiment 95 provides the LNP of any one of Embodiments 85-94, wherein the LNP has a molar ratio of (a): (b): (c): (d) of about 35:16:46.5:2.5.

Embodiment 96 provides the LNP of any one of Embodiments 85-95, wherein the LNP further comprises at least one cargo selected from the group consisting of a nucleic acid molecule and a therapeutic agent.

Embodiment 97 provides the LNP of Embodiment 96, wherein the therapeutic agent is at least one selected from the group consisting of a small molecule, a protein, and an antibody.

Embodiment 98 provides the LNP of Embodiment 97, wherein the LNP comprises a nucleic acid molecule.

Embodiment 99 provides the LNP of Embodiment 98, wherein the nucleic acid molecule is a DNA molecule or an RNA molecule.

Embodiment 100 provides the LNP of Embodiment 98 or 99, wherein the nucleic acid molecule is selected from the group consisting of cDNA, mRNA, miRNA, siRNA, modified RNA, antagomir, antisense molecule, and a targeted nucleic acid, or any combination thereof.

Embodiment 101 provides the LNP of any one of Embodiments 98-100, wherein the nucleic acid molecule encodes a chimeric antigen receptor (CAR).

Embodiment 102 provides the LNP of Embodiment 101, wherein the CAR is specific for binding to a surface antigen of a pathogenic cell.

Embodiment 103 provides the LNP of any one of Embodiments 98-102, wherein the nucleic acid molecule encodes at least one selected from the group consisting of mRNA and sgRNA, optionally wherein the ionizable lipid and mRNA have a weight ratio of about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1.

Embodiment 104 provides the LNP of Embodiment 103, wherein the mRNA encodes a therapeutic protein, optionally wherein the therapeutic protein is a CRISPR-associated protein, and optionally wherein the CRISPR-associated protein is CRISPR-associated protein 9 (Cas9).

Embodiment 105 provides the LNP of Embodiment 96, wherein the therapeutic agent is a CRISPR-associated protein, optionally wherein the CRISPR-associated protein is CRISPR-associated protein 9 (Cas9).

Embodiment 106 provides a pharmaceutical composition comprising the lipid nanoparticle (LNP) of any one of Embodiments 85-105 and at least one pharmaceutically acceptable carrier.

Embodiment 107 provides a method of delivering an agent to a bone of a subject, the method comprising administering at least one LNP of any one of Embodiments 85-105 or the pharmaceutical composition of Embodiment 106 to the subject.

Embodiment 108 provides the method of Embodiment 107, wherein the therapeutic cargo is delivering to a bone cell, bone tissue, or bone marrow of the subject.

Embodiment 109 provides the method of Embodiment 107 or 108, wherein the agent is selected from the group consisting of a nucleic acid molecule, a small molecule, a protein, an antibody, a therapeutic agent, and any combination thereof.

Embodiment 110 provides a method of treating, ameliorating, and/or preventing at least one disease, disorder, or condition in a subject in need thereof, the method comprising administering a therapeutically effectively amount of at least one LNP of any one of Embodiments 85-105 and/or the pharmaceutical composition of Embodiment 106 to the subject.

Embodiment 111 provides the method of Embodiment 110, wherein the disease, disorder, or condition is selected from the group consisting of bone disease or disorder, bone marrow disease or disorder, condition associated with bone, condition associated with bone marrow, bone fracture, bone defect, cancer, leukemia, lymphoma, myeloma, other blood cancers, myeloma, sickle cell disease, inflammatory disease or disorder, and any combination thereof.

Embodiment 112 provides a method of inducing a bone regeneration in a subject in need thereof, the method comprising administering a therapeutically effectively amount of at least one LNP of any one of Embodiments 85-105 and/or the composition of Embodiment 106 to the subject.

Embodiment 113 provides a composition comprising a mineralized tissue and the lipid nanoparticle (LNP) of any one of Embodiments 1-31 and 85-105, wherein the LNP is adsorbed to a surface of the mineralized tissue.

Embodiment 114 provides the composition of Embodiment 113, wherein the mineralized tissue comprises hydroxyapatite.

Embodiment 115 provides the composition of Embodiment 113 or 114, wherein the mineralized tissue is selected from the group consisting of bone, dentin, enamel, cementum, and a calcified cartilage.

Embodiment 116 provides the composition of any one of Embodiments 113-115, wherein the LNP further comprises at least one cargo selected from the group consisting of a nucleic acid molecule and a therapeutic agent.

Embodiment 117 provides the composition of Embodiment 116, wherein the nucleic acid molecule is selected from the group consisting of cDNA, mRNA, miRNA, siRNA, modified RNA, antagomir, antisense molecule, and a targeted nucleic acid, or any combination thereof.

Embodiment 118 provides a method for treating, preventing, and/or ameliorating an orthopedic or dental disease in a subject in need thereof, the method comprising contacting a mineralized tissue of the subject with the composition of any one of Embodiments 113-117 under conditions effective to graft the mineralized tissue of the composition to the mineralized tissue of the subject.

Embodiment 119 provides a method for delivering a nucleic acid molecule and/or therapeutic agent to a mineralized tissue of a subject in need thereof, the method comprising contacting the mineralized tissue of the subject with the composition of any one of Embodiments 113-117 under conditions effective to graft the mineralized tissue of the composition to the mineralized tissue of the subject.

Embodiment 120 provides a method for repairing, restoring, or reducing degradation of a mineralized tissue in a subject in need thereof, the method comprising contacting the mineralized tissue of the subject with the composition of any one of Embodiments 113-117 under conditions effective to graft the mineralized tissue of the composition to the mineralized tissue of the subject.

Embodiment 121 provides the method of any one of Embodiments 118-120, wherein the mineralized tissue comprises hydroxyapatite.

Embodiment 122 provides the method of any one of Embodiments 118-121, wherein the mineralized tissue is selected from the group consisting of bone, dentin, enamel, cementum, and a calcified cartilage.

Embodiment 123 provides the method of any one of Embodiments 118-122, wherein the mineralized tissue surface is an allograft, autograft, xenograft, or synthetic graft.

Embodiment 124 provides the method of any one of Embodiments 118-123, wherein the composition enhances osteointegration, bone deposition, remineralization, or cellular uptake of the nucleic acid molecule and/or therapeutic agent.

Embodiment 125 provides the method of any one of Embodiments 118 and 121-123, wherein the orthopedic or dental disease is selected from the group consisting of a bone injury (e.g., bone fracture, stress fracture, non-union or bone defect, or compression injury), bone degeneration (e.g., osteoporosis, osteopenia, osteomalacia, or avascular necrosis), inflammation (e.g., osteoarthritis, rheumatoid arthritis, synovitis, periostitis, tendinitis, or bursitis), tooth or periodontal tissue damage (e.g., caries, enamel demineralization, dentin hypersensitivity, enamel erosion, or cracked tooth), periodontal and gingival disease (e.g., periodontitis, gingivitis, peri-implant mucositis, or peri-implantitis), inflammation (e.g., pulpitis, apical periodontitis, periapical abscesses, osteomyelitis, or inflammatory resorption).

Embodiment 126 provides the method of any one of Embodiments 118-125, wherein the subject is a mammal.

Embodiment 127 provides the method of Embodiment 126, wherein the mammal is a human.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed is:

1. A lipid nanoparticle (LNP) comprising:
(a) at least one compound having the structure of Formula (I), or a racemate, enantiomer, diastereomer, pharmaceutically acceptable salt, solvate, or a derivative thereof: 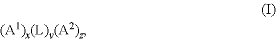
wherein:
each occurrence of A¹ is independently 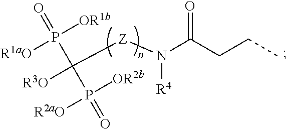
each occurrence of A² is independently 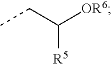
each occurrence of L is an amine linker independently selected from the group consisting of aminoalkyl linker, substituted aminoalkyl linker, diaminoalkyl linker, substituted diaminoalkyl linker, triaminoalkyl linker, substituted triaminoalkyl linker, tetraaminoalkyl linker, substituted tetraaminoalkyl linker, pentaaminoalkyl linker, substituted pentaaminoalkyl linker, polyaminoalkyl linker, substituted polyaminoalkyl linker, aminocycloalkyl linker, substituted aminocycloalkyl linker, diaminocycloalkyl linker, substituted diaminocycloalkyl linker, triaminocycloalkyl linker, substituted triaminocycloalkyl linker, tetraaminocycloalkyl linker, substituted tetraaminocycloalkyl linker, pentaaminocycloalkyl linker, substituted pentaaminocycloalkyl linker, polyaminocycloalkyl linker, substituted polyaminocycloalkyl linker, and any combination thereof;
each occurrence of Z is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, optionally substituted C₂-C₈ heterocyloalkylenyl, and optionally substituted phenyl;
each occurrence of R^{1a}, R^{1b}, R^{2a}, R^{2b}, R³, R⁴, R⁵, and R⁶ is independently selected from the group consisting of hydrogen, halogen, optionally substituted C₁-C₁₂ alkyl, optionally substituted C₃-C₁₂ cycloalkyl, optionally substituted —Y(R⁹)_{z′}(R¹⁰)_{z″}—(C₃-C₁₂ cycloalkyl), optionally substituted C₂-C₁₂ heterocycloalkyl, optionally substituted-(R⁹)_{z′}(R¹⁰)_{z″}—(C₂-C₁₂ heterocycloalkyl), optionally substituted C₂-C₁₂ alkenyl, optionally substituted C₅-C₁₂ cycloalkenyl, optionally substituted —Y(R⁹)_{z′}(R¹⁰)_{z″}—(C₅-C₁₂ cycloalkenyl), optionally substituted C₂-C₁₂ alkynyl, optionally substituted C₅-C₁₂ cycloalkynyl, optionally substituted —Y(R⁹)_{z′}(R¹⁰)_{z″}—(C₈-C₁₂ cycloalkynyl), optionally substituted C₆-C₁₀ aryl, optionally substituted —Y(R⁹)_{z′}(R¹⁰)_{z″}—(C₆-C₁₀ aryl), optionally substituted C₂-C₁₂ heteroaryl, optionally substituted —Y(R⁹)_{z′}(R¹⁰)_{z″}-(C₂-C₁₂ heteroaryl), alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —Y(R⁹)_{z′}(R¹⁰)_{z″}-ester, —Y(R⁹)_{z′}(R¹⁰)_{z″}, —NO₂, —CN, ═O, ═S, sulfoxy, sulfonate, sulfate, sulfite, and sulfide, and any combination thereof,
each occurrence of R⁹ and R¹⁰ is independently selected from the group consisting of hydrogen, halogen, optionally substituted C₁-C₁₂ alkyl, optionally substituted C₃-C₁₂ cycloalkyl, optionally substituted C₂-C₁₂ heterocycloalkyl, optionally substituted C₂-C₁₂ alkenyl, optionally substituted C₅-C₁₂ cycloalkenyl, optionally substituted C₂-C₁₂ alkynyl, optionally substituted C₂-C₁₂ cycloalkynyl, optionally substituted C₆-C₁₀ aryl, optionally substituted C₂-C₁₂ heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, —NO₂, —CN, sulfoxy, sulfonate, sulfate, sulfite, and sulfide, and any combination thereof, or two geminal R⁹ and R¹⁰ groups can combine to form ═O or ═S;
each occurrence of Y′ is independently selected from the group consisting of C, O, N, S, P, and Si;
each occurrence of z′ and z″ is independently an integer represented by 0, 1, or 2;
wherein x, y, and z are independently an integer from 0 to 20;
each occurrence of n is independently an integer from 0 to 10; and
the compound is present in a concentration range of about 1 mol % to about 99 mol %;
(b) at least one neutral phospholipid, wherein the neutral phospholipid is present in a concentration range of about 5 mol % to about 45 mol %;
(c) at least one cholesterol lipid, wherein the total cholesterol lipid is in a concentration range of about 5 mol % to about 55 mol %; and
(d) at least one polymer conjugated lipid (e.g., polyethylene glycol (PEG)-conjugated lipid), wherein the total polymer conjugated lipid is present in a concentration range of about 0.5 mol % to about 12.5 mol %.

2. A composition comprising at least one LNP of claim 1, optionally further comprising at least one pharmaceutically acceptable excipient.

3. A method of delivering an agent to a subject in need thereof, the method comprising administering a therapeutically effectively amount of at least one LNP of claim 1, or a pharmaceutical composition thereof, to the subject.

4. A method of delivering an agent to a bone in a subject, bone marrow, or a combination thereof, the method comprising administering a therapeutically effectively amount of at least one LNP of claim 1, or a pharmaceutical composition thereof, to the subject.

5. A method of treating, ameliorating, or preventing at least one disease, disorder, or condition in a subject in need thereof, the method comprising administering a therapeutically effectively amount of at least one LNP of claim 1, or a pharmaceutical composition thereof, to the subject

6. A method of inducing a bone regeneration in a subject in need thereof, the method comprising administering a therapeutically effectively amount of at least one LNP of claim 1, or a pharmaceutical composition thereof, to the subject.

7. A method of replacing at least one protein in a subject in need thereof, the method comprising administering a therapeutically effectively amount of at least one LNP of claim 1, or a pharmaceutical composition thereof, to the subject.

8. A method of gene editing in a subject in need thereof, the method comprising administering a therapeutically effectively amount of at least one LNP of claim 1, or a pharmaceutical composition thereof, to the subject.

9. A method of inducing an immune response in a subject in need thereof, the method comprising administering a therapeutically effectively amount of at least one LNP of claim 1, or a pharmaceutical composition thereof, to the subject.

10. A compound of Formula (V), or a salt, stereoisomer, or isotopologue thereof: 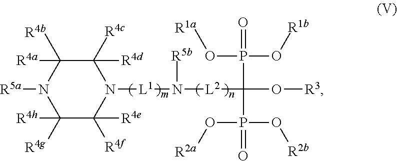
wherein:
R^{1a}, R^{1b}, R^{2a}, R^{2b}, and R³ are each independently selected from the group consisting of H, C(═O)R^{A}, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
R^{4a}, R^{4b}, R^{4c}, R^{4d}, R^{4e}, R^{4f}, R^{4g}, and R^{4h} are each independently selected from the group consisting of H, halogen, and optionally substituted C₁-C₆ alkyl;
R^{5a} and R^{5b} are each independently selected from the group consisting of H, optionally substituted C₁-C₂₄ alkyl, —C(═O)(optionally substituted C₁-C₂₄ alkyl), —C(═O)O(optionally substituted C₁-C₂₄ alkyl), and R⁶,
wherein at least one of R^{5a} and R^{5b} is R⁶; 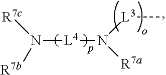
each occurrence of R⁶ is independently
two occurrences of R⁶ can combine with the atoms to which they are
bound to form 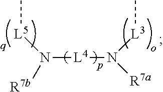
each occurrence of L¹, L², L³, L⁵, and L⁶, if present, is independently selected from the group consisting of -(optionally substituted C₁-C₃ alkylenyl)-, —C(═O)—, —O—, and —N(R^{A})—;
each occurrence of L⁴ is independently selected from the group consisting of —X—, -(optionally substituted C₁-C₁₂ alkylenyl)-, -(optionally substituted C₂-C₁₂ alkenylenyl)-, -(optionally substituted C₁-C₁₂ alkynylenyl)-, -(optionally substituted C₁-C₁₂ heteroalkylenyl)-, -(optionally substituted C₃-C₈ cycloalkylenyl)-, -(optionally substituted C₂-C₈ heterocyloalkylenyl)-, -(optionally substituted C₆-C₁₀ arylenyl)-, and -(optionally substituted C₂-C₈ heteroarylenyl)-;
each occurrence of X, if present, is independently selected from the group consisting of —N(R^{7d})—, —N(R⁸)—, —C(═O)—, and —O—;
each occurrence of R^{7a}, R^{7b}, R^{7c}, R^{7d}, R^{7c}, and R^{7f}, if present, are each independently selected from the group consisting of H, optionally substituted C₁-C₂₄ alkyl and optionally substituted C₁-C₂₄ heteroalkyl;
each occurrence of R⁸ is independently 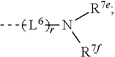
each occurrence of m and n, o, p, q, and r, if present, are each independently an integer selected from the group consisting of 1, 2, 3, 4, 5, 6, 7, 8, 9, and 10; and
each occurrence of R^{A} is independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

11. A lipid nanoparticle (LNP) composition comprising:
(a) at least one ionizable lipid comprising at least one compound of Formula (V), or a salt, stereoisomer, or isotopologue thereof: 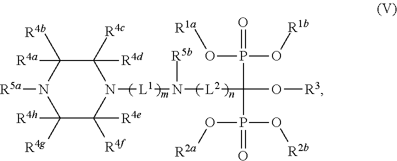
wherein:
R^{1a}, R^{1l}, R^{2a}, R^{2b}, and R³ are each independently selected from the group consisting of H, C(═O)R^{A}, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
R^{4a}, R^{4b}, R^{4c}, R^{4d}, R^{4e}, R^{4f}, R^{4g}, and R^{4h} are each independently selected from the group consisting of H, halogen, and optionally substituted C₁-C₆ alkyl;
R^{5a} and R^{5b} are each independently selected from the group consisting of H, optionally substituted C₁-C₂₄ alkyl, —C(═O)(optionally substituted C₁-C₂₄ alkyl), —C(═O)O(optionally substituted C₁-C₂₄ alkyl), and R⁶,
wherein at least one of R^{5a} and R^{5b} is R⁶;
each occurrence of R⁶ is independently 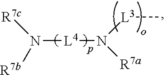
or
two occurrences of R⁶ can combine with the atoms to which they are bound to form 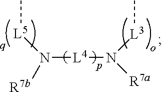
each occurrence of L¹, L², L³, L⁵, and L⁶, if present, is independently selected from the group consisting of -(optionally substituted C₁-C₃ alkylenyl)-, —C(═O)—, —O—, and —N(R^{A})—;
each occurrence of L⁴ is independently selected from the group consisting of —X—, -(optionally substituted C₁-C₁₂ alkylenyl)-, -(optionally substituted C₂-C₁₂ alkenylenyl)-, -(optionally substituted C₁-C₁₂ alkynylenyl)-, -(optionally substituted C₁-C₁₂ heteroalkylenyl)-, -(optionally substituted C₃-C₈ cycloalkylenyl)-, -(optionally substituted C₂-C₈ heterocyloalkylenyl)-, -(optionally substituted C₆-C₁₀ arylenyl)-, and -(optionally substituted C₂-C₈ heteroarylenyl)-;
each occurrence of X, if present, is independently selected from the group consisting of —N(R^{7d})—, —N(R⁸)—, —C(═O)—, and —O—;
each occurrence of R^{7a}, R^{7b}, R^{7c}, R^{7d}, R^{7c}, and R^{7f}, if present, are each independently selected from the group consisting of H, optionally substituted C₁-C₂₄ alkyl and optionally substituted C₁-C₂₄ heteroalkyl;
each occurrence of R⁸ is independently 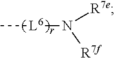
each occurrence of m and n, o, p, q, and r, if present, are each independently an integer selected from the group consisting of 1, 2, 3, 4, 5, 6, 7, 8, 9, and 10; and
each occurrence of R^{A} is independently selected from the group consisting of H, optionally substituted C₁-C₆ alkyl, optionally substituted C₁-C₆ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
(b) at least one neutral lipid;
(c) at least one cholesterol lipid and/or a modified derivative thereof, and
(d) at least one polymer-conjugated lipid and/or a modified derivative thereof.

12. A pharmaceutical composition comprising the lipid nanoparticle (LNP) of claim 11 and at least one pharmaceutically acceptable carrier.

13. A method of delivering an agent to a bone of a subject, the method comprising administering at least one LNP of claim 11, or a pharmaceutical composition thereof, to the subject.

14. A method of treating, ameliorating, or preventing at least one disease, disorder, or condition in a subject in need thereof, the method comprising administering a therapeutically effectively amount of at least one LNP of claim 11, or a pharmaceutical composition thereof, to the subject.

15. A method of inducing bone regeneration in a subject in need thereof, the method comprising administering a therapeutically effectively amount of at least one LNP of claim 11, or a pharmaceutical composition thereof, to the subject.

16. A composition comprising a mineralized tissue and the lipid nanoparticle (LNP) of claim 11, wherein the LNP is adsorbed to a surface of the mineralized tissue.

17. A method for treating, preventing, or ameliorating an orthopedic or dental disease in a subject in need thereof, the method comprising contacting a mineralized tissue of the subject with the composition of claim 16 under conditions effective to graft the mineralized tissue of the composition to the mineralized tissue of the subject.

18. A method for delivering a nucleic acid molecule or therapeutic agent to a mineralized tissue of a subject in need thereof, the method comprising contacting the mineralized tissue of the subject with the composition of claim 16 under conditions effective to graft the mineralized tissue of the composition to the mineralized tissue of the subject.

19. A method for repairing, restoring, or reducing degradation of a mineralized tissue in a subject in need thereof, the method comprising contacting the mineralized tissue of the subject with the composition of claim 16 under conditions effective to graft the mineralized tissue of the composition to the mineralized tissue of the subject.
